(19) United States
(12) Patent Application Publication
(10) Pub. No.: US 2013/0260436 A1
(43) Pub. Date: Oct. 3, 2013

(54) Methods of Identifying and Characterizing Natural Product Gene Clusters

(71) Applicants: Davis Francis Ackerley, Wellington (NZ); Jeremy Owen, Wellington (NZ)

(72) Inventors: Davis Francis Ackerley, Wellington (NZ); Jeremy Owen, Wellington (NZ)

(73) Assignee: Victoria LInk Limited, Wellington (NZ)

(21) Appl. No.: 13/822,590

(22) PCT Filed: Sep. 14, 2011
(86) PCT No.: PCT/NZ11/00189

§ 371 Date: Jun. 11, 2013

(30) Foreign Application Priority Data
Sep. 14, 2010 (NZ) 587972

(51) Int. Cl. C12N 9/10

(52) U.S. Cl. CPC C12N 9/104
USPC 435/193; 506/11

(57) ABSTRACT

The invention relates to methods and compositions for identifying a candidate nucleic acid (CNA) comprising a polynucleotide sequence encoding at least a part of a natural product gene cluster (NPGC), a secondary metabolite biosynthesis cluster (SMBC), a non ribosomal peptide (NRP), a polyketide (PK) biosynthesis cluster, a protein involved in NRP and/or PK biosynthesis, a protein involved in other secondary metabolite biosynthesis, and/or a phosphopantetheinyl transferase (PPTase), by expressing the candidate nucleic acid (CNA) to form at least one PPTase, incubating the PPTase with a non ribosomal peptide synthetase (NRPS), and detecting activation of the NRPS, wherein activation indicates that said CNA comprises a polynucleotide sequence encoding at least one of the above.

### FIELD OF THE INVENTION

This invention relates to methods for identifying and characterizing natural product gene clusters, including secondary metabolite biosynthesis clusters related to fatty acid (FA), polyketide (PK) and non-ribosomal peptide (NRP) biosynthesis, as well as discovery and characterisation of chemicals that modulate activity of a PPTase.

### BACKGROUND

Non ribosomal peptide synthetases (NRPS) are enzymes found in many bacteria and fungi that catalyse the production of biologically active small peptides from amino acid precursors without the need for a nucleic acid template (Finking and Marahiel, 2004; Challis and Naismith, 2004; Marahiel and Essen, 2009). In non ribosomal peptide (NRP) synthesis, the NRPS proteins themselves form the template that directs the number, order and identity of amino acid substrates found ill the peptide product (Marahiel et al. 1997). NRPS proteins consist of a series of discrete modules, each responsible for the recognition, activation and incorporation of a single amino acid residue (Stachelhaus and Marahiel, 1995). The number of modules in a NRPS system generally corresponds to the number of amino acids in the molecule produced. The order of these modules in the genome of the producer organism therefore generally reflects the order of amino acid residues in the NRP product. This phenomenon is known as the co-linearity rule (Marahiel et al. 1997; Stachelhaus and Marahiel, 1995). Modules within bacterial NRPS biosynthetic systems are usually spread out over a number of interacting proteins; each protein typically containing 1-5 modules, although up to 18 modules have been known to comprise a single NRPS protein in eukaryotes (Vizcaíno et al. 2005). NRPS enzymes are particularly interesting from a biotechnology perspective due to the immense diversity in structure and function of the molecules they synthesise. Unlike ribosomal peptide synthesis, non-ribosomal peptide synthesis can utilise over 400 molecules as substrates (Caboche et al., 2007) to produce biologically active linear, cyclic and branched cyclic molecules (Marahiel and Essen, 2009; Meier and Burkart, 2009; Koglin and Walsh, 2009; and Caboche et al., 2009). NRPS derived natural products include molecules with antibiotic, cytostatic, bio-surfactant and immunosuppressive activity.

There is therefore significant research interest in identifying new non-ribosomal peptides (and the polynucleotide sequences that encode the non-ribosomal peptide synthetases that produce non ribosomal peptides) which may be useful new antibiotics, cytostatics, bio-surfactants and immunosuppressants, at least.

Certain previous methods that have been used to identify novel NPGCs have relied on identification or amplification of conserved motifs in DNA sequence (Ginolhac et al. 2004). Certain other methods have employed phage screening techniques to identify NPGCs (Yin et al. 2007; Sunbul et al. 2009). However, such phage panning methods can be quite time consuming and prone to false positives. For example, known phage panning methods are only able to identify small gene fragments, requiring painstaking primer-walking or other such methods to identify and characterize any putatively identified NPGCs.

Additionally, phage screening methods have the disadvantage of only being able to identify a partial fraction of the NPGCs in a sample (Yin et al. 2007). This is shown in the published controls of where the authors, using phage panning methods, were able to demonstrate the ability to pick up only 18% and 44% of NRPS and PKS genes known to be present in two genome sequenced species of bacteria (Yin et al. 2007).

Additionally, certain previous methods used to identify new non-ribosomal peptides (and the gene clusters that encode them) have been limited to entirely in vitro screening, where such in vitro methods may not be readily adaptable for high throughput screens. Consequently, there is a need for alternative, non-sequence based, high throughput methods of identifying natural product gene clusters, particularly secondary metabolite biosynthesis clusters encoding polyketides (PK) or non-ribosomal peptides (NRP).

It should be noted that NRPS genes are frequently found in association with polyketide synthetase (PKS) genes, and that mixed NRP/PK products are common. NRPS and PKS enzymes share a common requirement for activation from an inactive apo to an active holo form by attachment of a 4′-phosphopantetheine (4′-PP) cofactor. This cofactor attachment is catalyzed by a cognate 4′-phosphopantetheinyl transferase (PPTase) enzyme. Genes encoding PPTase and NRPS and/or PKS and/or other biosynthetic enzymes are frequently linked in arrangements known as natural product gene clusters (NPGCs). Thus, a high throughput screening assay capable of recovering PPTase genes from DNA samples would, by association, also enrich for NPGCs.

It should also be noted that PPTase enzymes represent an attractive target for the development of new antibiotics due to their integral role in both primary and secondary metabolism. In primary bacterial metabolism PPTases are essential for viability as they activate acyl carrier proteins (ACPs) involved in fatty acid biosynthesis. In secondary metabolism they serve to activate the carrier protein domains (CP, or CP-domains, also referred to as T-domains) of NRPS and PKS enzymes, which are often implicated in the synthesis of virulence factors in bacteria and fungi. Humans have a single PPTase that serves to activate ACPs for fatty acid synthesis. Traditionally PPTase activity has been measured in vitro using a high performance liquid chromatography (HPLC) based assay. In assays of this sort purified CP is incubated with a target PPTase, coenzyme A (CoA) and Mg²⁺. Following incubation, apo and holo CP are separated by reverse phase HPLC and the relative abundance of each measured. While this assay allows accurate determination of CP modification rates by a PPTase, it is technically challenging to run, time-consuming and not at all amenable to high throughput screening (HTS). The above-mentioned disadvantages of conventional assays have limited the use of phosphopantetheinylation for inhibitor screening. In particular, these disadvantages have significantly limited the potential for high-throughput screening.

More recently, fluorescence resonance energy transfer (FRET) (Foley et al, 2009, FEBS J. 276:7134-7145; Yasgar et al, 2010, Mol. Biosyst. 6:365-375) and fluorescence polarization (Duckworth et al, 2010, Anal. Biochem. 403:13-19) assays that utilize fluorophore labelled CoA conjugates have been developed. These assays allow PPTase activity to be monitored spectrophotometrically and are amenable to high-throughput screening applications; for example, the FRET-based technique has been used to screen the LOPAC¹²⁸⁰ compound library for inhibitors of B. subtilis Sfp (Yasgar et al, 2010, Mol. Biosyst. 6:365-375). However, these approaches are also technically challenging and do not enable measurement of PPTase kinetic parameters with natural substrates or evaluation of the relative activity of different PPTase/carrier protein combinations.

PPTases have also been applied to site specific labelling of proteins with fluorescently labelled CoA conjugates. By using two different PPTases, for example AcpS and Sfp, which differ in their specificity for protein fusion tags (either carrier proteins or short peptides), it is possible to carry out orthogonal labelling of two alternatively tagged proteins expressed on the surface of a cell or present in solution (Zou and Yin, 2009, J. Am. Chem. Soc. 131:7548-7549). This technology could be expanded to orthogonal labelling of more than two proteins if appropriate PPTase/fusion tag combinations were available. Accordingly there is a need for a method for rapidly determining the rate of modification of a variety of carrier protein substrates by a PPTase that is applicable to the discovery of new PPTase/protein fusion tags for orthogonal labelling of proteins.

It is an object of the present invention to at least provide an improved method that can be used for high throughput screening and identification of at least a part of a: natural product gene cluster (NPGC) and/or a secondary metabolite biosynthesis cluster (SMBC) encoding at least one polyketide synthetase (PKS) or at least one non-ribosomal peptide synthetase (NRPS), and/or that encode at least one protein involved in PK and/or NRP synthesis, and/or that mitigate or eliminate the disadvantages associated with conventional methods of using PPTases as a screening tool and/or for a method for investigating phosphopantetheinylation and/or determining PPTase activity which is simple, fast, high-throughput and cost-efficient and/or that will at least provide the public with a useful choice.

### SUMMARY OF THE INVENTION

In one aspect the invention provides a method of identifying a candidate nucleic acid (CNA) comprising one or more of the polynucleotide sequences selected from the group consisting of
a) at least a part of a natural product gene cluster (NPGC),
b) at least a part of a secondary metabolite biosynthesis cluster (SMBC)
c) at least a part of a non ribosomal peptide (NRP), and/or polyketide (PK) biosynthesis cluster,
d) a polynucleotide sequence encoding at least one protein involved in NRP and/or PK biosynthesis,
e) a polynucleotide sequence encoding at least one protein involved in other secondary metabolite biosynthesis, and
f) a polynucleotide sequence encoding at least one phosphopantetheinyl transferase (PPTase), the method comprising,
   expressing said candidate nucleic acid (CNA) or polynucleotide sequence to form at least one PPTase,
   incubating said at least one PPTase with a non ribosomal peptide synthetase (NRPS), and detecting activation of said NRPS,
   wherein said activation indicates that said CNA comprises at least one of a)-f).

Preferably the CNA comprises a PPTase and at least one of a) to e). Preferably detecting activation of said NRPS is determining the presence or absence of a reporter product formed due to the activation of said NRPS. Preferably determining the presence or absence of a reporter product comprises detection of the reporter product, more preferably direct detection.

In one embodiment, the method comprises the additional step of further characterizing the CNA to identify at least one of a)-f). In a preferred embodiment the method identifies a PPTase and at least one of a) to e). Preferably the further characterizing comprises determining at least part of the nucleotide sequence of the CNA to identify at least one of a)-f). Preferably the further characterizing also includes bioinformatics analysis of the nucleotide sequence to identify at least one of a)-f).

In various embodiments of the method, the NRPS is selected from the group consisting of an endogenous NRPS, an exogenous NRPS, a naturally occurring NRPS, a non-naturally occurring NRPS, a modified NRPS (mNRPS) or a combination thereof. Preferably, the NRPS is an endogenous or exogenous, naturally-occurring NRPS. Alternatively the NRPS is a chemically evolved mNRPS.

Preferably, expressing is in vitro. Preferably expressing is in vivo. Preferably expressing is in an isolated host cell Preferably said isolated host cell is a prokaryotic cell or a eukaryotic cell. Preferably said eukaryotic host cell is a fungal cell. Preferably said prokaryotic host cell is a bacterial cell. Preferably said bacterial cell is a Gram negative bacterial cell. Preferably said bacterial cell is an E. coli cell. Preferably said E. coli cell contains a deleted or otherwise inactive form of the native PPTase entD gene to enhance assay sensitivity. Preferably said reporter product formed due to the activation of said NRPS is a pigment or dye. Preferably said NRPS is BpsA. Preferably said dye is indigoidine. Preferably said CNAs are nucleic acid constructs or clones present in a DNA library. Preferably said DNA library is a genomic DNA library or a cDNA library. Preferably said DNA library is an environmental DNA (eDNA) library.

In one embodiment, a method of the invention comprises the additional step of characterizing at least one secondary metabolite produced due to the expression of said CNA. Preferably characterizing is by nuclear mass resonance (NMR), thin-layer chromatography (TLC) or chromatographic analysis. Preferably characterizing is by nuclear mass resonance (NMR). Preferably characterizing is by thin-layer chromatography (TLC). Preferably chromatographic analysis comprises analysis by mass spectrometry (MS). Preferably analysis by MS is by gas chromatography-MS (GC-MS). Preferably said secondary metabolite is a non ribosomal peptide, polyketide, or a mixed molecule containing both non ribosomal peptide and polyketide elements.

Preferably characterizing comprises determining the molecular weight, or at least part of the chemical structure of said secondary metabolite. Preferably determining the molecular weight or least part of the chemical structure comprises comparing said secondary metabolite to a library of known chemical structures of secondary metabolites.

In one embodiment of the method, a NRPS used in a method according to the invention is encoded by at least one of:
a. a polynucleotide encoding a BpsA synthetase,
b. a polynucleotide comprising a nucleotide sequence having at least 70% sequence identity with SEQ ID NO: 1,
c. a polynucleotide comprising SEQ ID NO: 1,
d. a polynucleotide consisting of a nucleotide sequence having at least 70% sequence identity with SEQ ID NO: 1,
e. a polynucleotide consisting of SEQ ID NO: 1,
f. a polynucleotide of any one of a-e above comprising a T-domain comprising at least 70% sequence identity with SEQ ID NO: 20
g. a polynucleotide of any one of i-v above comprising a T-domain consisting of at least 70% sequence identity with SEQ ID NO: 20

In one embodiment, the NRPS used in a method of the invention is a modified NRPS (mNRP) as described herein.

In one aspect the invention provides a method of identifying a candidate nucleic acid (CNA) comprising a polynucleotide sequence encoding a functional PPTase, the method comprising, expressing said candidate nucleic acid (CNA) or polynucleotide sequence to form a PPTase, incubating said PPTase with a NRPS, and detecting activation of said NRPS, wherein said activation indicates that said CNA comprises a polynucleotide sequence encoding a functional PPTase.

In one embodiment, a method of the invention is performed using a NRPS according to the invention to identify a CNA comprising a polynucleotide sequence that encodes a broad spectrum unidentified PPTase. Preferably the method comprises using a range of NRP synthetases. Preferably the NRP synthetases are mNRP synthetases as described herein.

In one aspect the invention provides a modified NRPS (mNRPS) that is encoded by:
a. a polynucleotide sequence encoding a modified BpsA synthetase,
b. a polynucleotide sequence variant of SEQ ID NO: 1 wherein said variant comprises at least 70% nucleotide sequence identity with SEQ ID NO: 1, or
c. a polynucleotide sequence encoding a modified NRPS comprising a modified T-domain, wherein said T-domain is selected from the group consisting of
   (1) a heterologous T-domain,
   (2) a homologous T-domain,
   (3) an exogenous T-domain,
   (4) an endogenous T-domain,
   (5) a T-domain encoded by a nucleotide sequence comprising at least 70% sequence identity with the T-domain of any one of SEQ ID NO: 1, 2, 4, 6, 8, 10, 12, 14, 16 and 18, and
   (6) a T-domain encoded by a nucleotide sequence of the T-domain of any one of the nucleotide sequences selected from the group consisting of SEQ ID NO: 1, 2, 4, 6, 8, 10, 12, 14, 16, and 18.
      Preferably the mNRPS is encoded by (a.). Preferably the mNRPS is encoded by (b.). Preferably the mNRPS is encoded by (c.).

In one embodiment the invention provides a modified NRPS (mNRPS) having:
i) an amino acid sequence encoding a modified BpsA synthetase,
ii) an amino acid sequence variant of SEQ ID NO: 3 wherein said variant comprises at least 70% amino acid sequence identity with SEQ ID NO: 3, or
iii) an amino acid sequence encoding a modified NRPS comprising a modified T-domain, wherein said T-domain is selected from the group consisting of:
   (1) a heterologous T-domain,
   (2) a homologous T-domain,
   (3) an exogenous T-domain,
   (4) an endogenous T-domain,
   (5) a T-domain encoded by an amino acid sequence comprising at least 70% sequence identity with the T-domain of any one of SEQ ID NO: 3, 5, 7, 9, 11, 13, 15, 17 and 19, and
   (6) a T-domain encoded by a nucleotide sequence of the T-domain of any one of the nucleotide sequences selected from the group consisting of SEQ ID NO: 3, 5, 7, 9, 11, 13, 15, 17 and 19.
      Preferably the mNRPS is encoded by i. Preferably the mNRPS is encoded by ii.
      Preferably the mNRPS is encoded by iii.

In one embodiment, the invention relates to a polynucleotide sequence that encodes a mNRPS according to the invention. Preferably the mNRPS is a modified BpsA synthetase. Preferably the polynucleotide comprises at least 70% nucleotide sequence identity to SEQ ID NO: 1, but is not SEQ ID NO: 1. Preferably the polynucleotide consists of a nucleotide sequence having at least 70% nucleotide sequence identity to SEQ ID NO: 1, but is not SEQ ID NO: 1. Preferably a polynucleotide comprises a nucleotide sequence selected from the group consisting of SEQ ID NO: 2, 4, 6, 8, 10, 14, 16, and 18. Preferably the polynucleotide consists of a nucleotide sequence selected from the group consisting of SEQ ID NO: 2, 4, 6, 8, 10, 14, 16 and 18. Preferably the polynucleotide comprises a T-domain comprising at least 70% sequence identity with SEQ ID NO: 20. Preferably the polynucleotide comprises a T-domain consisting of at least 70% sequence identity with SEQ ID NO: 20.

Preferably said polynucleotide sequence is comprised in an expression cassette. Preferably said expression cassette is part of an expression construct. Preferably said expression construct is comprised in a vector. Preferably the vector is selected from the group consisting of phage, phagemids, P1 artificial chromosomes (PAC), plasmids, cosmids, fosmids, yeast artificial chromosomes (YAC) and bacterial artificial chromosomes (BAC). Preferably the vector is a plasmid. Preferably the plasmid is at least one of the plasmids listed in Table 4.2. Preferably the plasmid is pET28(a) or a variant or derivative thereof. In one embodiment the invention provides a mNRPS comprising a polypeptide sequence selected from the group consisting of a polypeptide sequence comprising at least 70% identity to SEQ ID NO: 3, 5, 7, 9, 11, 13, 15, 17, or 19, and a polypeptide sequence comprising SEQ ID NO: 3, 5, 7, 9, 11, 13, 15, 17 or 19. Preferably the mNRPS consists of a polypeptide sequence selected from the group consisting of a polypeptide sequence comprising at least 70% identity to SEQ ID NO: 3, 5, 7, 9, 11, 13, 15, 17 or 19, and a polypeptide sequence of SEQ ID NO: 3, 5, 7, 9, 11, 13, 15, 17 or 19.

In another aspect, the invention provides a method of making a mNRPS of the invention comprising
a. modifying the nucleotide sequence encoding the T-domain of a polynucleotide encoding an NRP synthetase to make a modified polynucleotide, and
b. expressing said modified polynucleotide under suitable conditions to form a mNRPS.
   Preferably the method of making a mNRPS includes the additional step of
c. isolating and purifying said mNRPS.
   Preferably the method of making a mNRPS includes the additional steps of
d. incubating said mNRPS with a PPTase, and
e. detecting the activation of said mNRPS by the PPTase,
wherein activation of said mNRPS confirms that said mNRPS is a modified NRPS that is activated by the PPTase.
   Preferably the method of making a mNRPS includes the additional steps of
f. characterizing the catalytic activity and specificity of a given PPTase for 4′-PP attachment to said mNRPS, and
g. comparing said catalytic activity and specificity to the catalytic activity and specificity of the same PPTase for 4′-PP attachment to another NRPS,
   thereby identifying that the PPTase has different catalytic activity and/or specificity for the mNRPS.

Preferably the given PPTase is a known PPTase. Preferably the another NRPS is a wild type NRPS. Alternatively, the another NRPS is a mNRPS according to the invention or made according to a method of the invention.

In another aspect, the invention provides a method of characterizing the ability of a PPTase or suspected PPTase, to activate an NRPS, the method comprising
a. incubating said PPTase or suspected PPTase with an NRPS, and
b. detecting the activation of said NRPS,
   thereby characterizing the PPTase or suspected PPTase as capable of activating said NRPS.

Preferably detecting activation of said NRPS is determining the presence or absence of a reporter product formed due to the activation of said NRPS. Preferably characterizing is characterizing the binding activity and/or specificity of the PPTase or suspected PPTase for the NRPS. Preferably said NRPS is selected from the group consisting of an endogenous NRPS, an exogenous NRPS, a naturally occurring NRPS, a non-naturally occurring NRPS, a modified NRPS (mNRP) as described herein or a combination thereof. Preferably, the NRPS is an endogenous or exogenous, naturally-occurring NRPS. Alternatively the NRPS is a chemically evolved mNRPS. Preferably the PPTase is a known PPTase.

Preferably in an embodiment of the method that uses a mNRPS, the method includes an additional step of
c. comparing the binding activity and specificity of the PPTase or suspected PPTase for the mNRPS with the binding activity and specificity of a corresponding wild type NRPS.
   Preferably characterizing activity and binding specificity comprises kinetic characterization. In another aspect, the invention provides a method of making a modified PPTase, the method comprising
a. expressing a modified PPTase from a polynucleotide sequence to form an expressed PPTase,
b. incubating the expressed PPTase with an NRPS, and
c. detecting the activation of the NRPS,
   wherein activation of the NRPS confirms that said expressed PPTase is a functional modified PPTase.

Preferably the polynucleotide sequence encoding the modified PPTase has been modified by error-prone PCR, targeted mutagenesis, or DNA shuffling. Preferably detecting activation of the NRPS is determining the presence or absence of a reporter product formed due to the activation of the NRPS. Preferably said NRPS is selected from the group consisting of an endogenous NRPS, an exogenous NRPS, a naturally occurring NRPS, a non-naturally occurring NRPS, a modified NRPS (mNRP) as described herein or a combination thereof. Preferably, the NRPS is an endogenous or exogenous, naturally-occurring NRPS. Alternatively the NRPS is a chemically evolved mNRPS. Preferably the method includes a further step of characterizing the modified PPTase. Preferably characterizing is characterizing the binding activity and/or specificity of the PPTase or suspected PPTase for the NRPS or for members of the group of NRPSs as described above. Preferably the modified PPTase has modified PPTase activity or specificity for the NRPS.

In another aspect of the present invention, provided is an assay platform wherein a pigment synthesising enzyme acts as a reporter for PPTase activity in vivo.

In another aspect of the present invention, provided is an assay platform wherein a pigment synthesising enzyme acts as a reporter for PPTase activity in vitro.

In another aspect of the present invention there is provided a method of characterising the rate of reaction of PPTases, the method comprising the steps of:
combining a pigment producing enzyme with a PPTase and a substrate and co-factors required for both phosphopantetheinylation and pigment production,
using a measurement tool to measure the pigment level, and
computing the rate of pigment produced over a time period,
wherein the change in rate of pigment produced is proportional to the rate of reaction of the PPTase to be characterized.

In another aspect of the present invention, there is provided a method of detecting a modifier, the method comprising the steps of
combining a pigment producing enzyme with a PPTase and a substrate and co-factors required for both phosphopantetheinylation and pigment production in the presence of a chemical compound to be characterized
using a measurement tool to measure the pigment produced,
computing the rate of pigment produced over a time period,
wherein if the rate of the reaction for the PPTase slows in the presence of the chemical, it is an inhibitor, or if the rate of reaction increases, the chemical is an accelerator.

In another aspect of the invention there is provided a method for determining the rate of modification of any carrier protein or peptide substrate by any PPTase, the method comprising the steps of:
combining a PPTase, a pigment producing enzyme and the necessary substrates for phosphopantetheinylation in the presence of a variety of known concentrations of a carrier protein or peptide that acts as a competitor for one or more of the phosphopantetheinylation substrates which is in limited supply,
incubating the resulting reaction,
adding the necessary substrates for the pigment production reaction,
   using a measurement tool to measure the pigment level, and
   computing the rate of pigment produced over a time period,
wherein the rate of pigment production is indicative of the amount of pigment producing enzyme converted from apo to holo form and allows determination of the relative rate of carrier protein or peptide modification by the PPTase.

Preferably the pigment producing enzyme is an NRPS or a PKS enzyme. Preferably the pigment producing enzyme is BpsA. Preferably the PPTase may be any PPTase. Preferably the PPTase may be capable of recognising and activating the T-domain of BpsA (or other NRPS/PKS enzyme).

Alternatively, the PPTase may be modified, by swapping T-domains and evolving the resulting mBpsA that allows it to be converted into a substrate for any PPTase.

In one embodiment, the PPTases may be one of Sfp of B. subtilis subsp. spizizenii ATCC6633, PcpS of P. aeruginosa PAO1 and the putative PPTase PP1183 of P. putida KT2440.

Preferably the substrate and cofactors may include CoA, Mg²⁺ and L-glutamine and Adenosine-5′-triphosphate (ATP). Preferably the measurement tool may be anything capable of characterising the rate of change of a pigment or a change in fluorescence.

Preferably said pigment is indigoidine. Preferably the measurement tool may be a microplate reader. In a preferred embodiment, the method described may be used to test for chemical inhibitors. Preferably the inhibitors may be any inhibitor of the reaction between BpsA and a PPTase described herein. Preferably, the assay is suitable for both in vivo, ex vivo and in vitro screening. Preferably the assay is used for in vitro screening.

In another aspect of the present invention, there is provided a method to evaluate PPTase activity by monitoring acceleration of the rate of indigoidine synthesis. Preferably, the acceleration of the rate of indigoidine synthesis may be used as a measure of the rate of 4′-PP attachment to apo-BpsA. Preferably, the methods provided may allow for rapid and reproducible assessment of PPTase kinetic parameters and substrate specificity. Preferably assessment of a chemical inhibitor may be performed using a PPTase capable of recognition and activation of the native PCP-domain of BpsA. Preferably this can be rapidly assessed in vivo by simple co-transformation of the PPTase in question with a BpsA in E. coli. Preferably this can be rapidly assessed in vitro by co-incubation of the PPTase in question as purified protein with purified BpsA protein in the presence of exogenously added substrates. Running the assay with BpsA which has been pre-activated by a PPTase in the absence of inhibitor provides a means of counter-screening a potential chemical inhibitor to ensure that its primary target is the PPTase and not BpsA.

### BRIEF DESCRIPTION OF THE DRAWINGS

The invention will now be described with reference to the figures in the accompanying drawings.

FIG. 1—BpsA.

A—BpsA is an NRPS enzyme with unusual domain organisation. It contains a single A, T and TE-domain with an Ox-domain integrated into the A-domain.

B—BpsA synthesises the blue pigment indigoidine from two molecules of L-Gln. The function of each of the domains is shown. It is unknown if the oxidation reaction occurs before or after cyclization. The mechanism of dimerisation is also unknown. Note the essential role of the 4′PP group attached to the T-domain (indicated with a grey arrow). This group is attached to BpsA by the action of a cognate PPTase enzyme.

FIG. 2—Activation of BpsA Expressed in E. coli by Three Different PPTase Enzymes

(A) No exogenous PPTase expressed. (B) Co-expression of BpsA and PP1183. (C) Co-expression of BpsA and PcpS. (D) Co-expression of BpsA and Sfp.

Single colonies of E. coli co-expressing BpsA and an activating PPTase were inoculated onto pigment production agar plate using a sterile pipette tip and incubated for 16 hours at 37° C. Protein expression was then induced by IPTG addition as described in Section 4.9.2 and the plate incubated for a further 24 h at room temperature (18-25° C.).

FIG. 3—SDS Page Analysis of Purified BpsA and PPTases Purified, Buffer Exchanged Enzymes are Shown.

(1) BpsA. (2) NEB broad range MW marker (New England Biolabs, Ipswich, Mass.) (3) PcpS. (4) PP1183. (5) Sfp.

FIG. 4—Preliminary Analysis of Indigoidine Formation Catalysed by BpsA

Reactions were set up in a 96wp as described in Section 4.11.2 and initiated by addition of purified BpsA. Product concentration was then monitored by measuring A590 every 10 s in a microplate reader. The final concentration of L-Gln present in each reaction is indicated, the concentration of all other reactants was constant between reactions. Notice that absorbance decreases after a peak value is reached, and that this decrease is proportional to the previous rate of increase. A similar pattern was seen when using increasing concentrations of ATP at a set concentration of L-Gln.

FIG. 5—The Effect of pH on Indigoidine Stability and BpsA Reaction Rate

(A) Diluted sterile supernatant from an indigoidine producing culture was incubated for 20 min with 50 mM sodium phosphate buffer at each of the indicated pH values in triplicate. Loss of colouration was then assessed by measuring A590. Error bars are presented as +/− one standard deviation. (B) Indigoidine reactions differing only in the pH of the buffer used are shown. Deviation from pH 7.8 by as little as 0.2 units was found to noticeably impair function.

FIG. 6—Kinetic Analysis of BpsA

(A) Relative activity of BpsA over pH range of 7.3-9.0 (Tris-Cl). Six replicates were analysed for each pH value, error bars are presented as +/− one standard error. (B) BpsA reaction velocity as a function of L-gln concentration showing good fit with Michaelis Menten equation (R²=0.98). Six replicates were analysed for each L-Gln concentration, error bars are presented as +/− one standard error. (C) Experimental confirmation of linear relationship (R²=0.99) between concentration of active BpsA and reaction rate at constant substrate concentration. Three replicates were analysed for each BpsA concentration, error bars are presented as +/− one standard error.

FIG. 7—Measurement of the Maximal Velocity of the PPTase-Catalysed Activation of BpsA

(A) Abs 590 nm of reactions containing inactive apo-BpsA and an activating PPTase is read every 10 s. (B) The gradient between every 2-8 points of the curve is measured as an estimate of instantaneous velocity; this is proportional to the amount of BpsA that has been activated by the PPTase. (C) Instantaneous velocity vs. time is graphed, and the steepest portion of the linear section of curve determined, the gradient of this is taken as a measure of the maximum rate of BpsA activation by the PPTase. Rates of BpsA modification are determined for a range of substrate (CoA) concentrations; these are used to derive kinetic parameters for the activating PPTase. Data analysis of this sort can be automated easily using the slope function of Microsoft Excel.

FIG. 8—Derivation of PPTase Kinetic Parameters Using BpsA as a Reporter

Demonstration of how velocity values for PPTases can be derived by measuring acceleration of BpsA reaction. (A) Selection of Raw data (A590 values) for indigoidine production as BpsA is progressively converted from apo to holo form by PcpS in the presence of 3.1 (⋄) 1.56 (▪) 0.78 (♦) and 0.39 (Δ) μM CoA, remaining concentrations are omitted for clarity. (B) Velocity vs. time values derived for the same four data sets, PPTase velocity values were derived from the linear portion of each curve (i.e. BpsA acceleration). (C) Michaelis-Menten plot of all PPTase velocity values derived from this experiment (0.078-50 μM CoA), enabling derivation of PcpS kinetic parameters (Data presented is for a single replicate at twelve CoA concentrations, 6-9 such replicates were analysed to derive the final data presented in Table 1.2).

FIG. 9—Kinetic Data for PcpS, Sfp and PP1183 Derived Using BpsA Coupled Assay

Pooled data from 6-9 replicates at twelve substrate concentrations is shown for PcpS (◯) Sfp (Δ) and PP1183 (). Curves indicate fit to Michaelis Menten equation and were generated using Graphpad Prism®. Error bars are presented as +/− one standard error.

FIG. 10—Carrier Protein Competition Assay

Representative data for a selection of carrier protein competition assays is shown illustrating the use of the assay to determine the affinity of a single PPTase for different carrier protein substrates (A) or different PPTases for the same carrier protein substrate (B). Pooled data for 3-6 replicates at each substrate concentration is shown. Error bars are presented as +/− one standard error. Four parameter dose response curves were generated using Graphpad Prism®

(A) IC₅₀ curve for Sfp competition assay with mPCP (solid) and bPCP (dashed).

(B) IC₅₀ curve for mPCP inhibition of Sfp (solid) and PcpS (dashed).

FIG. 11—Inhibition of PPTases and BpsA by 6-NOBP

Data is from three replicates at each concentration of 6-NOBP (n=36), CoA concentration was fixed at 2.5 μM for data presented. Error bars are presented as +/− one standard error. Inset: Structure of 6-NOBP.

FIG. 12—A Representative Plate from Screening of the Lopac¹²⁸⁰ Chemical Library to Identify Novel Inhibitors of the P. aeruginosa PPTase PcpS.

1.8 μL of each compound from a Lopac¹²⁸⁰ library plate was added to 30 μL of 10% DMSO in MQ using a CyBio CyBi-well, giving a final compound concentration of approximately 18 μM. Next 50 μl, of a master mix containing 5 μM Co-enzyme A, 1.66 μM BpsA, 20 mM MgCl₂, 5 mM ATP, 8 mM L-Gln, 100 mM Tris-HCl pH 7.8 and MQ was added. To initiate the reaction 20 μL of PcpS at a final reaction concentration of 0.18 μM and MQ was added rapidly, using an automatic dispensing Pipette. The plate was then shaken for 10 seconds at 1000 rpm to mix the compounds. The plate was then read 25 times to measure the Absorbance at 590 nm (with 20 second intervals between each read) in an EnSpire plate reader at 25° C. Each plate had 80 compounds added to rows 2-11 leaving the first and last column empty. Negative and positive controls were established in wells in these columns, and used to monitor the reaction. The negative controls had 20 μL of MQ added instead of PcpS. The positive controls had 1.8 μL of DMSO added instead of a compound. Both control reactions were run in triplicate. The reaction traces corresponding to the wells containing 6-NOBP, an unidentified strong PcpS inhibitor, and the three negative control reactions are indicated.

FIG. 13—Inhibition of the Pseudomonas aeruginosa PPTase PcpS by the Compound Bay11-7085.

A. IC₅₀ Curve for Bay11-7085 Mediated Inhibition of PcpS.

The candidate PcpS inhibitor Bay11-7085 was identified in a screen similar to that depicted in FIG. 12. A master mix containing: 5 μM CoA, 5 mM ATP, 20 mM MgCl₂, 8 mM L-Gln, 100 mM Tris-Cl pH 7.8, 1.66 μM BpsA was used to establish a reaction in 24 wells in a 96 well plate. Bay 11-7085 was then serially diluted from 20 μM to 0.625 μM across the wells. The reaction was initiated by the addition of PcpS to a final concentration of 0.18 μM and indigoidine production was then monitored at 590 nm. Each concentration was tested in triplicate and the average of the three wells was taken to calculate the maximum velocity. The maximum velocity in each well is expressed as a percentage of the maximum velocity in the wells where no Bay11-7085 was present.

B. Qualitative Assessment of Bay11-7085 Mediated Inhibition of PcpS Relative to 6-NOBP.

Duplicate wells with different inhibitors added. The No PcpS wells have ddH₂O added instead of PcpS. The BpsA only wells have DMSO added instead of an inhibitor. The 6-NOBP wells have had 6-NOBP added at a final concentration of 20 μM. The Bay 11-7085 wells have had Bay 11-7085 added to a final concentration of 20 μM.

FIG. 14—Screening eDNA Libraries using BpsA as a Reporter.

(1) Environmental DNA libraries are transformed into cells harboring a bpsA reporter gene. (2) The cells are then plated on selective pigment production medium and colonies in which the eDNA fragment taken up encodes a PPTase are identified by blue colouration. The blue colouration arises due to activation of the BpsA reporter by an eDNA encoded PPTase (as depicted in the inset box).

FIG. 15—Identification of PPTase Encoding Nucleic Acids from eDNA Libraries by Screening with BpsA

E. coli colonies harboring a bpsA reporter which have been transformed with eDNA encoding a PPTase enzyme are readily discernable by virtue of dark blue colouration on pigment production agar. One such colony, indicated by an arrow, can be seen in the photo above. Subsequent isolation and sequencing of the eDNA fragment captured in this colony revealed the presence of a previously uncharacterized PPTase gene.

FIG. 16—Genomic DNA Fragments Isolated from Screening of eDNA Library 1 Using Wild Type bpsA as a Reporter Gene

Arrangement and identity of partial and complete genes found in the three unique eDNA fragments isolated (fragment numbering as indicated to the left of each insert diagram). Refer to Table 2.1 for gene annotations. The shading of the genes identified in fragment 1 indicates that they were likely recovered from a secondary metabolite cluster, whereas the lack of shading for fragments 2 and 3 indicates that there is no strong evidence these were derived from a secondary metabolite cluster.

FIG. 17—Schematic Overview of Directed Evolution Procedure

(1) T-domain is amplified by error prone PCR and amplicon is ligated into a plasmid borne copy of the bpsA gene (pBPSA3). (2) The resulting library of T-domain variants is transformed into cells harbouring an activating PPTase. (3) Transformed cells are plated on pigment development agar and improved clones are recovered. (4) Improved clones are subjected to a second, quantitative screen for activity in a 96 wp format

FIG. 18—Pigmentation of Single Colonies of E. coli as a Result of Expression of BpsA and an Activating PPTase

(A) Close up of single colonies expressing BpsA and PcpS on a pigment production agar plate eight hours after induction of protein expression by IPTG addition.

(B) Colonies of the same strain of E. coli containing empty plasmids in place of the BpsA and PcpS expression constructs following the same treatment. Darker grey colonies reflect increased blue pigmentation due to indigoidine production.

FIG. 19—Second Tier Screening Results from First Round Evolution of slPvdD1

In vivo activity of improved clones was determined by measuring A590 of supernatant for quadruplicate cultures of each clone grown in a 96 well plates (wp) as described in Section 4.9.3. The white bar is unimproved slPvdD1 included as a negative control. The black bar is WT BpsA included as a positive control. Clones for which sequence was determined are indicated by striped bars. Clones for which sequence was not determined are indicated by grey bars. Error bars are presented as +/− one standard deviation for quadruplicate cultures. Absolute (not adjusted against a media only control) supernatant A590 values are shown. The arrow indicates clone 3kF0, the T-domain of which was used as template for the second round evolution. In this and all subsequent graphs, the dashed horizontal line indicates the pigmentation level of the negative control, against which other conditions were compared.

FIG. 20—Second Tier Screening Results for Second Round Evolution of slPvdD1

In vivo activity of improved clones was determined by measuring A590 of supernatant for quadruplicate cultures of each clone grown in a 96 wp as described in Section 4.9.3. The white bar (Neg) is 3KF0, the best improved clone form the first round of evolution, included as a negative control. The black bar (Pos) is WT BpsA included as a positive control. Clones for which sequence was determined are indicated by striped bars. Putative false positives, for which sequence was not determined, are indicated by grey bars. Error bars are presented as +/− one standard deviation for quadruplicate cultures. Absolute (not adjusted against a media only control) supernatant A590 values are shown.

FIG. 21—Second Tier Screening Results from First Round Evolution of slEntF

In vivo activity of improved clones was determined by measuring A590 of supernatant for quadruplicate cultures of each clone grown in a 96 wp as described in Section 4.9.3. The white bar is unimproved slPvdD1 included as a negative control. The black bar is WT BpsA included as a positive control. Clones for which sequence was determined are indicated by striped bars. Clones for which sequence was not determined are indicated by grey bars. Error bars are presented as +/− one standard deviation for quadruplicate cultures. Absolute (not adjusted against a media only control) supernatant A590 values are shown.

FIG. 22—Genomic DNA Fragments Isolated from Screening of eDNA Library 1 Using Wild Type bpsA, PR2H6, or ENTFR1 as a Reporter Gene

Arrangement and identity of partial and complete genes found in the seven unique eDNA fragments isolated from eDNA library 1 (fragment numbering as indicated to the left of each insert diagram). Refer to Table 3.2 for gene annotation. The shading of the genes identified in fragments 1 and 7 indicates that there is strong evidence they were recovered from a secondary metabolite cluster, whereas the lack of shading for fragments 2-6 indicates that there is no clear evidence these were derived from a secondary metabolite cluster.

FIG. 23—Genomic DNA Fragments Isolated from Screening of eDNA Library 2 Using Wild Type bpsA, PR2H6, or ENTFR1 as a Reporter Gene

Arrangement and identity of partial and complete genes found in the 14 unique eDNA fragments isolated from eDNA library 2 (fragment numbering as indicated to the left of each insert diagram). Refer to Table 3.4 for gene annotation. The light shading of the genes identified in fragments 1, 3, 4, 6 and 7 indicates that there is strong evidence they were recovered from a secondary metabolite cluster, whereas the lack of shading for fragments 2, 5, 8, 9, 10, 11, 12 and 13 indicates that there is no clear evidence these were derived from a secondary metabolite cluster. The dark shading for the open reading frame on fragment 14 indicates that this gene likely encodes a member of an entirely new family of PPTase.

FIG. 24—Design and Construction of pBPSA3.

Amplification of the regions up and downstream of the bpsA T-domain using primers A+B and C+D respectively allowed generation of the plasmid pBPSA3, a construct in which the bpsA gene lacked its native T-domain and contained an NsiI and a SpeI site into which foreign domains could be introduced. Primers E and F for amplification of substitute T-domains (PvdD T-domain in this example) had 5′ sequence elements added that would replace the missing BpsA sequence indicated by the light and dark grey lines. Ligation of T-domains into BpsA resulted in an upstream NsiI-PstI hybrid site and a downstream XbaI-SpeI hybrid site, indicated by vertical black bars. Introduction of these sites did not alter the amino acid sequence encoded at these points. The result of this is a seamless transition between BpsA sequence (light grey) and T-domain sequence (dark grey).

FIG. 25—Wild Type BpsA Polynucleotide

SEQ ID NO: 1: bpsA gene from S. lavendulae ATCC11924. Bold underline sequence indicates the T-domain.

FIG. 26—slBpsA Polynucleotide

SEQ ID NO: 2: bpsA gene sequence (SEQ ID NO: 1) with silent mutations due to restriction site introduction. Underline sequences differ from wild type due to introduction of silent restriction sites. Upstream (ATGCAG)=Nsi1−Pst1 hybrid site, downstream (TCTAGT)=Xba1−Spe1 hybrid site. These sites flank the T-domain. Sequence between the sites can be substituted for foreign sequence. The region indicated in bold underline is substituted for foreign sequence in mBpsAs. The sequence between the silent restriction sites and the substituted sequence is added to PCR primers used to amplify foreign sequence. Amount of foreign sequence can be increased as far as restriction sites or decreased as far as desired by altering primers used for amplification of foreign sequence.

FIG. 27—BpsA Polypeptide

SEQ ID NO: 3: Amino acid translation of SEQ ID NO: 1 and 2. Bold underline sequence is the T-domain of BpsA. This region is replaced by foreign T-domain sequence in mBpsAs

FIG. 28—s1PvdD Polynucleotide

SEQ ID NO: 4: bpsA gene in which T-domain sequence has been replaced with sequence from the first module of the P. aeruginosa PAO1 gene pvdD. Underline sequence indicates hybrid restriction sites that result from introduction of foreign sequence. Bold underline sequence is foreign T-domain sequence.

FIG. 29—s1PvdD polypeptide

SEQ ID NO: 5: Amino acid translation of SEQ ID NO: 4. Bold underline sequence is foreign T-domain sequence that replaces the native T-domain of BpsA.

FIG. 30—s1PvdD2 polynucleotide

SEQ ID NO: 6: bpsA gene in which T-domain sequence has been replaced with sequence from the second module of the P. aeruginosa PAO1 gene pvdD. Underline sequence indicates hybrid restriction sites that result from introduction of foreign sequence. Bold underline sequence is foreign T-domain sequence.

FIG. 31—s1PvdD2 Polypeptide

SEQ ID NO: 7: Amino acid translation of SEQ ID NO: 6. Bold underline sequence is foreign T-domain sequence that replaces the native T-domain of BpsA.

FIG. 32—s1Pst Polynucleotide

SEQ ID NO: 8: bpsA gene in which T-domain sequence has been replaced with sequence from the first module of the P. syringae 1448a gene pspph1926. Underlined sequence indicates hybrid restriction sites that result from introduction of foreign sequence. Bold underline sequence is foreign T-domain sequence.

FIG. 33—s1Pst Polypeptide

SEQ ID NO: 9: Amino acid translation of SEQ ID NO: 8. Bold underline sequence is foreign T-domain sequence that replaces the native T-domain of BpsA.

FIG. 34—s1EntF Polynucleotide

SEQ ID NO: 10: bpsA gene in which T-domain sequence has been replaced with sequence from the E. coli W3110 gene entF. Underlined sequence indicates hybrid restriction sites that result from introduction of foreign sequence. Bold underline sequence is foreign T-domain sequence.

FIG. 35—s1EntF Polypeptide

SEQ ID NO: 11: Amino acid translation of SEQ ID NO: 10. Bold underline sequence is foreign T-domain sequence that replaces the native T-domain of BpsA.

FIG. 36—s1DhbF Polynucleotide

SEQ ID NO: 12: bpsA gene in which T-domain sequence has been replaced with sequence from the first module of the B. subtilis (subs. Sp. subtilis) gene dhbF. Underlined sequence indicates hybrid restriction sites that result from introduction of foreign sequence. Bold underline sequence is foreign T-domain sequence.

FIG. 37—s1DhbF Polypeptide

SEQ ID NO: 13: Amino acid translation of SEQ ID NO: 12. Bold underline sequence is foreign T-domain sequence that replaces the native T-domain of BpsA.

FIG. 38—5k5 Polynucleotide

SEQ ID NO: 14: This variant was derived from SEQ ID NO: 10 by one round of random mutagenesis and selection (directed evolution). This is the clone with the highest pigment synthesis capacity in vivo. Underlined sequence indicates hybrid restriction sites that result from introduction of foreign sequence. Bold underline sequence is foreign T-domain sequence.

FIG. 39—5k5 Polypeptide

SEQ ID NO: 15: Amino acid translation of SEQ ID NO: 14. Bold underline sequence is foreign T-domain sequence that replaces the native T-domain of BpsA.

FIG. 40—Pr2H6 Polynucleotide

SEQ ID NO: 16: This variant was derived from SEQ ID NO: 4 by one round of random mutagenesis and selection (directed evolution). This is the clone with the highest pigment synthesis capacity in vivo. Underlined sequence indicates hybrid restriction sites that result from introduction of foreign sequence. Bold+underlined sequence is foreign T-domain sequence.

FIG. 41—Pr2H6 Polypeptide

SEQ ID NO: 17: Amino acid translation of SEQ ID NO: 16. Bold underline sequence is foreign T-domain sequence that replaces the native T-domain of BpsA

FIG. 42—oBpsA Polynucleotide

SEQ ID NO: 18: A mutant bpsA gene having reduced activity (79% reduction) as compared to SEQ ID NO: 1.

FIG. 43—oBpsA Polypeptide

SEQ ID NO: 19: Amino acid translation of SEQ ID NO: 18.

FIG. 44—BpsA T-Domain Polynucleotide

SEQ ID NO: 20 is the nucleotide sequence of the T-domain from the bpsA gene (SEQ ID NO: 1).

FIG. 45—BpsA T-Domain Polypeptide

SEQ ID NO: 21: Amino acid translation of SEQ ID NO: 20.

### DETAILED DESCRIPTION OF THE INVENTION

### Definitions

The term “comprising” as used in this specification and claims means “consisting at least in part of”; that is to say when interpreting statements in this specification and claims which include “comprising”, the features prefaced by this term in each statement all need to be present but other features can also be present. Related terms such as “comprise” and “comprised” are to be interpreted in similar manner.

The term “candidate nucleic acid” (CNA) as used herein refers to an unidentified polynucleotide sequence that has been cloned into an appropriate vector. An appropriate vector can be any vector that will function within the methods of the invention. For example, a plasmid, cosmid, fosmid, or bacterial artificial chromosome (BAC) but not limited thereto. For example, a CNA is an unidentified polynucleotide that is to be screened according to a method of the invention. The unidentified polynucleotide sequence may be present in a DNA library, for example a genomic DNA library or a cDNA library, but not limited thereto. In one embodiment, a CNA is an unidentified polynucleotide present in a genomic DNA library prepared from DNA that has been isolated from an environmental sample.

An “environmental DNA library” as used herein refers to a library of polynucleotide sequences that has been prepared from DNA that has been isolated from an organism or multiple organisms in an environmental sample. In one embodiment, the environmental DNA library is prepared from DNA isolated from soil, air or water, but not limited thereto. Preferably the environmental DNA library is prepared from soil or seawater.

In one embodiment, a CNA is an unidentified polynucleotide present in a cDNA library prepared from RNA that has been isolated from a prokaryotic or eukaryotic organism. Preferably the cDNA library is prepared from RNA isolated from a eukaryotic organism, preferably a fungus, a plant or an animal, but not limited thereto.

The term “candidate nucleic acid expression construct” as used herein (CNAec) refers to a CNA that has been cloned into a vector that that is capable of expressing a “coding region” or “open reading frame” (ORF) that may be present in the CNA.

The term “candidate nucleic acid expression product” as used herein (CNAep) refers to a polypeptide that has been expressed from a CNAec as described herein.

The term “genetic construct” refers to a polynucleotide molecule, usually double-stranded DNA, which may have cloned or inserted into it another polynucleotide molecule. For example, a genetic construct may have an unidentified polynucleotide insert that is prepared from an environmental sample or as a cDNA, but not limited thereto. A genetic construct may contain the necessary elements that permit transcription of a cloned or inserted polynucleotide molecule, and, optionally, for translating the transcript into a polypeptide. The insert polynucleotide molecule may be derived from the host cell, or may be derived from a different cell or organism and/or may be a recombinant polynucleotide. Once inside the host cell the genetic construct may become integrated in the host chromosomal DNA. The genetic construct may be linked to a vector.

The term “polynucleotide(s),” as used herein, means a single or double-stranded deoxyribonucleotide or ribonucleotide polymer of any length, and include as non-limiting examples, coding and non-coding sequences of a gene, sense and antisense sequences, exons, introns, genomic DNA, cDNA, pre-mRNA, in RNA, rRNA, siRNA, miRNA, tRNA, ribozymes, recombinant polynucleotides, isolated and purified naturally occurring DNA or RNA sequences, synthetic RNA and DNA sequences, nucleic acid probes, primers, fragments, genetic constructs, vectors and modified polynucleotides. Reference to nucleic acids, nucleic acid molecules, nucleotide sequences and polynucleotide sequences is to be similarly understood.

The term “gene” as used herein refers to gene the biologic unit of heredity, self-reproducing and located at a definite position (locus) on a particular chromosome. In one embodiment the particular chromosome is a bacterial chromosome. The term bacterial chromosome is used interchangeably herein with the term bacterial genome.

The term “gene cluster” as used herein refers to a group of genes located closely together on the same chromosome whose products play a coordinated role in a specific aspect of cellular primary or secondary metabolism.

The term “primary metabolism” refers to metabolic processes which are essential for the viability of the organism. The absence of a primary metabolic process or pathway results in death of the organism. Primary metabolic processes/pathways include but are not limited to: Synthesis of essential biological macromolecules including nucleic acids (DNA/RNA), proteins and lipids, energy metabolism (both anabolic and catabolic) and cell division.

The term “secondary metabolite” as used herein refers to compounds that are not involved in primary metabolism, and therefore differ from the more prevalent macromolecules such as proteins and nucleic acids that make up the basic machinery of life. Many thousands of secondary metabolites have been described from various eukaryotic and prokaryotic organisms (Donadio et al. 2007). Frequently, secondary metabolites are species or strain specific, or are specific within a taxonomically-related group of organisms (Donadio et al. 2007). Consequently, secondary metabolites are known to adopt very wide array of chemical structures (chemical diversity) (Donadio et al. 2007; Walsh, 2007).

Many secondary metabolites find important biotechnological applications in biomedical and drug discovery research, and in the agricultural, aquaculture and chemical industries (Walsh, 2007).

Accordingly, a “secondary metabolite biosynthesis cluster” (SMBC) as used herein refers to a cluster of biosynthetic genes (alternatively termed a biosynthetic gene cluster), that comprises polynucleotide sequences encoding the functions required for synthesis and activity of a secondary metabolite.

The term “meta secondary metabolite biosynthesis cluster” (mSMBC) refers to a secondary metabolite biosynthesis cluster that comprises polynucleotide sequences that encode for more than one secondary metabolite.

The term “natural products gene cluster” (NPGC) as used herein refers to a SMBC or an mSMBC comprising a polynucleotide sequence (or polynucleotide sequences) encoding the production of a particular natural product or family of natural products.

The term “non-ribosomal peptide biosynthesis cluster” (NRPBC) as used herein refers to a secondary metabolite biosynthesis cluster that comprises polynucleotide sequences that encode for at least one secondary metabolite that is a non ribosomal peptide.

The term “polyketide biosynthesis cluster” (PKB) as used herein refers to a secondary metabolite biosynthesis cluster that comprises polynucleotide sequences that encode for at least one secondary metabolite that is a polyketide.

As used herein, the term “DNA library” takes the common meaning as known and used in the art.

The term “PPTase” is used herein as an abbreviation for a phosphopantetheinyl transferase. A PPTase catalyzes the attachment of a 4′-phosphopantetheine (4′-PP) cofactor to a non-ribosomal peptide synthetase, or a polyketide synthetase, or a fatty acid synthase.

The term “PPTase” also encompasses any protein, peptide and/or polypeptide that can catalyze the attachment of a 4′-phosphopantetheine (4′-PP) cofactor to a non-ribosomal peptide synthetase, or a polyketide synthetase, or a fatty acid synthase.

An NRPS, PKS or FAS is considered activated for the purposes of the invention, when it has had a 4′-phosphopantetheine (4′-PP) cofactor attached by “a PPTase”. Activation of an NRPS, PKS or FAS means the same thing.

As used herein, “a functional PPTase” and “a functional modified PPTase” are a PPTase that can activate an NRPS, PKS, or FAS namely by catalyzing the attachment of a 4′-phosphopantetheine (4′-PP) cofactor as above.

The term “T-domain” refers to the NRPS, PKS, or FAS domain that is the site of attachment of the 4′-PP cofactor, as above. The term T-domain is used interchangeably with peptidyl carrier protein domain (PCP-domain), carrier protein domain (CP domain) or carrier protein (CP).

The term “expressing” refers to the expression of a nucleic acid transcript from a nucleic acid template and is used herein as commonly used in the art.

The term “incubating” refers to the placing together of elements so they may interact and is used herein as commonly used in the art.

The term “NRPS” as used herein refers to a non ribosomal peptide synthetase that can be activated by a PPTase as described herein.

The term “a range of NRP synthetases” refers to a group or series of NRP synthetases, wherein each NRPS within the range, differs from each other NRPS within the range. For example, a range of NRP synthetases may be a series of modified NRP synthetases as described herein, but not limited thereto.

Activation refers to any enzymatic modification or action that causes the substrate of a given enzyme to adopt a functional conformation or perform a functional role that the substrate was not capable of performing before being activated. For example, a NRPS as described herein may be considered a substrate that is activated by a PPTase.

The term “reporter product” as used herein refers to a detectable product formed due to the activity of an activated NRPS.

The term “modified NRPS (mNRP)” means a NRPS that is not a naturally occurring variant of a wild type NRPS (wtNRP synthetase).

The term “further characterizing the CNA” refers to methods of analyzing and profiling polynucleotides including, but not limited to, determination of nucleotide sequence, sequence based motifs, structural motifs and other bioinformatics based analyses.

The term “bioinformatics analysis” as used herein refers to the analysis of a nucleic acid or amino acid sequence using any method, tool or protocol to obtain information about that nucleic or amino acid and takes its standard meaning as known and used in the art.

The term “endogenous” as used herein refers to a constituent of a cell, tissue or organism that originates or is produced naturally within that cell, tissue or organism. An “endogenous” constituent may be any constituent including but not limited to a polynucleotide, a polypeptide including a non-ribosomal polypeptide, a fatty acid or a polyketide, but not limited thereto.

The term “exogenous” as used herein refers to any constituent of a cell, tissue or organism that does not originate or is not produced naturally within that cell, tissue or organism. An exogenous constituent may be, for example, a polynucleotide sequence that has been introduced into a cell, tissue or organism, or a polypeptide expressed in that cell, tissue or organism from that polynucleotide sequence.

“Naturally occurring” as used herein with reference to a polynucleotide sequence according to the invention refers to a primary polynucleotide sequence that is found in nature. A synthetic polynucleotide sequence that is identical to a wild polynucleotide sequence is, for the purposes of this disclosure, considered a naturally occurring sequence. What is important for a naturally occurring polynucleotide sequence is that the actual sequence of nucleotide bases that comprise the polynucleotide is found or known from nature. For example, a wild type polynucleotide sequence is a naturally occurring polynucleotide sequence, but not limited thereto. A naturally occurring polynucleotide sequence also refers to variant polynucleotide sequences as found in nature that differ from wild type. For example allelic variants and naturally occurring recombinant polynucleotide sequences due to hybridization or horizontal gene transfer, but not limited thereto.

“Non-naturally occurring” as used herein with reference to a polynucleotide sequence according to the invention refers to a polynucleotide sequence that is not found in nature. Examples of non-naturally occurring polynucleotide sequences include artificially produced mutant and variant polynucleotide sequences, made for example by point mutation, insertion, or deletion, but not limited thereto. Non-naturally occurring polynucleotide sequences also include chemically evolved sequences. What is important for a non-naturally occurring polynucleotide sequence according to the invention is that the actual sequence of nucleotide bases that comprise the polynucleotide is not found or known from nature.

The term, “wild type” when used herein with reference to a polynucleotide refers to a naturally occurring; non-mutant form of a polynucleotide. A mutant polynucleotide means a polynucleotide that has sustained a mutation as known in the art, such as point mutation, insertion, deletion, substitution, amplification or translocation, but not limited thereto.

The term, “wild type” when used herein with reference to a polypeptide refers to a naturally occurring, non-mutant form of a polypeptide. A wild type polypeptide is a polypeptide that is capable of being expressed from a wild type polynucleotide. In one embodiment, a wild type polypeptide is a wild type non-ribosomal peptide that is expressed from a wild type polynucleotide.

The terms “chemically evolved” and “chemical evolution” refer to the artificial manipulation and selection of any particular polynucleotide or polypeptide sequence to generate modified polynucleotide or polypeptide sequences and take their standard meaning as known and used in the art

The term “vector” as used herein refers to a polynucleotide molecule, usually double stranded DNA, which is used to replicate or express a genetic construct. The vector may be used to transport a genetic construct into a given host cell.

The terms, “DNA library”, “genomic DNA library”, “cDNA library” and “environmental DNA library” as used herein refer to such libraries that comprise a plurality of genetic constructs wherein each genetic construct comprises an insert polynucleotide sequence. Each of these terms takes their common meaning as known and used in the art.

The term “non-ribosomal peptide” refers to biologically active small peptides or molecules derived from biologically active small peptides, that are synthesized by non-ribosomal peptide synthetases (NRPS) or polyketide synthetases (PKS) from amino acid precursors wherein the non-ribosomal peptide itself is not directly encoded by a polynucleotide template. A “non-ribosomal peptide” is also a polypeptide as described herein.

The term non-ribosomal peptide synthetase (NRPS) refers to a biosynthetic enzyme that catalyzes the addition of a constituent to a non ribosomal peptide, for example an amino acid constituent.

The term “modifying the nucleotide sequence encoding the T-domain” refers to any modification of the primary nucleotide sequence that affects the functionality of the T-domain of a NRP, where functionality refers to the specificity or activity of said T-domain, but is not limited thereto.

The term “characterizing the binding activity and specificity” of an NRP or a mNRP product “for a given PPTase” refers to methods of analyzing and profiling polypeptide binding to and specificity for a ligand. In one embodiment, characterization is determination enzyme kinetics as described herein and as known and used in the art, but not limited thereto.

The terms a “corresponding wild type NRP” and “corresponding wild type NRPS” are used herein to refer to the wild type sequence of a mNRPs or mNRPS before it has been modified as described herein.

The term a “a broad spectrum PPTase” refers to a PPTase that has been identified according to a method of the invention that is capable of activating at least two, preferably at least three, preferably at least four, preferably at least 5 or more different NRP substrates. In one embodiment, the broad spectrum PPTase is a PPTase that has been identified according to a method of the invention from a CNA, but not limited thereto.

The term “modified PPTase activity or specificity” refers to the modification of the activity or specificity of a PPTase as defined herein, for a particular NRPS, where activity refers to the extent of activation as described herein and specificity refers to the binding specificity of the PPTase for a given ligand, which may be a NRPS, but not limited thereto.

The term “IC₅₀” refers to a 50% inhibitory concentration for a compound, specifically the concentration of a compound that is required to bring about a 50% decrease in the rate of an enzyme-catalyzed reaction compared to an otherwise identical reaction where the test compound is not added. An IC₅₀ assay is an assay performed to establish the IC₅₀ value for a test compound.

The term “expression cassette” refers to a genetic construct that includes the necessary elements that permit the transcription of a polynucleotide molecule that has been cloned or inserted into the genetic construct. Optionally the expression cassette may comprise some or all of the necessary elements for translating the transcript produced from the expression cassette into a polypeptide. An expression cassette typically comprises in a 5′ to 3′ direction:
a) a functional promoter to allow expression of an insert polynucleotide,
b) the insert polynucleotide to be expressed, and
c) a functional terminator.

In one embodiment, an expression cassette forms part of a CNAep, wherein the insert polynucleotide is a CNA according to the invention.

The term “coding region” or “open reading frame” (ORF) refers to the sense strand of a genomic DNA sequence or a cDNA sequence that is capable of producing a transcription product and/or a polypeptide under the control of appropriate regulatory sequences. The coding sequence is identified by the presence of a 5′ translation start codon and a 3′ translation stop codon. When inserted into a genetic construct or an expression cassette, a “coding sequence” is capable of being expressed when it is operably linked to promoter and terminator sequences and/or other regulatory elements.

“Operably-linked” means that the sequence to be expressed is placed under the control of regulatory elements.

“Regulatory elements” as used herein refers to any nucleic acid sequence element that controls or influences the expression of a polynucleotide insert from a vector, genetic construct or expression cassette and includes promoters, transcription control sequences, translation control sequences, origins of replication, tissue-specific regulatory elements, temporal regulatory elements, enhancers, polyadenylation signals, repressors and terminators. Regulatory elements can be “homologous” or “heterologous” to the polynucleotide insert to be expressed from a genetic construct, expression cassette or vector as described herein. When a genetic construct, expression cassette or vector as described herein is present in a cell, a regulatory element can be “endogenous”, “exogenous”, “naturally occurring” and/or “non-naturally occurring” with respect to cell.

The term “noncoding region” refers to untranslated sequences that are upstream of the translational start site and downstream of the translational stop site. These sequences are also referred to respectively as the 5′ UTR and the 3′ UTR. These regions include elements required for transcription initiation and termination and for regulation of translation efficiency.

Terminators are sequences, which terminate transcription, and are found in the 3′ untranslated ends of genes downstream of the translated sequence. Terminators are important determinants of mRNA stability and in some cases have been found to have spatial regulatory functions.

The term “promoter” refers to nontranscribed cis-regulatory elements upstream of the coding region that regulate the transcription of a polynucleotide sequence. Promoters comprise cis-initiator elements which specify the transcription initiation site and conserved boxes. In one non-limiting example, bacterial promoters may comprise a “Pribnow box” (also known as the −10 region), and other motifs that are bound by transcription factors and promote transcription. Promoters can be homologous or heterologous with respect to polynucleotide sequence to be expressed. When the polynucleotide sequence is to be expressed in a cell, a promoter may be an endogenous or exogenous promoter. Promoters can be constitutive promoters, inducible promoters or regulatable promoters as known in the art.

“Homologous” as used herein with reference to polynucleotide regulatory elements, means a polynucleotide regulatory element that is a native and naturally-occurring polynucleotide regulatory element. A homologous polynucleotide regulatory element may be operably linked to a polynucleotide of interest such that the polynucleotide of interest can be expressed from a, vector, genetic construct or expression cassette according to the invention.

“Heterologous” as used herein with reference to polynucleotide regulatory elements, means a polynucleotide regulatory element that is not a native and naturally-occurring polynucleotide regulatory element. A heterologous polynucleotide regulatory element is not normally associated with the coding sequence to which it is operably linked. A heterologous regulatory element may be operably linked to a polynucleotide of interest such that the polynucleotide of interest can be expressed from a, vector, genetic construct or expression cassette according to the invention. Such promoters may include promoters normally associated with other genes, ORFs or coding regions, and/or promoters isolated from any other bacterial, viral, eukaryotic, or mammalian cell.

The term “polypeptide”, as used herein, encompasses amino acid chains of any length, but preferably at least 2 amino acid residues, preferably at least 3, 4, 5 6, 7, 8, 9, 10, 11, 12, 13, 14, 15, 16, 17, 18, 19, 20, 30, 40, 50, 100, 200, 500, 1000, 2000, 3000, 4000, 5000, 10,000 or greater amino acid residues of a specified polypeptide sequence or any combination thereof, wherein the amino acid residues are linked by covalent peptide bonds.

The term “polypeptide” may refer to a polypeptide that is a purified natural product, or that has been produced partially or wholly using recombinant or synthetic techniques. The term may refer to an aggregate of a polypeptide such as a dimer or other multimer, a fusion polypeptide, a polypeptide fragment, a polypeptide variant or functional derivative thereof.

The term “polypeptide” is used interchangeably herein with the terms “peptide” and “protein”.

The term, a “polypeptide” may also refer to a “non-ribosomal peptide”.

A “fragment” of a polypeptide is a subsequence of the polypeptide that performs a function that is required for the biological activity or binding and/or provides three dimensional structure of the polypeptide. The term may refer to a polypeptide, an aggregate of a polypeptide such as a dimer or other multimer, a fusion polypeptide, a polypeptide fragment, a polypeptide variant, or functional polypeptide derivative thereof that is capable of performing the polypeptide activity. The term “full length” as used herein with reference to a wild type polypeptide sequence means a polypeptide that comprises a contiguous sequence of amino acid residues where each amino acid residue has been expressed from each of its corresponding codons in the polynucleotide over the entire length of the coding region and resulting in a fully functional polypeptide, peptide or protein. As will be appreciated by a person of ordinary skill in the art, a “full length” polypeptide contains the amino acid sequence that corresponds to and has been expressed from each and every codon encoded by the polynucleotide comprising the entire coding region of the polypeptide, wherein each of said codons is located between the start codon and the termination codon normally associated with that coding region.

As used herein, “BpsA protein”, “BpsA peptide” and “BpsA polypeptide” mean the same thing and are used interchangeably. Also, according to the inventor's work disclosed herein, and without being bound by theory, due to the unique configuration of BpsA, a BpsA peptide is also an NRPS as used herein, that may be activated by a PPTase.

The terms BpsA protein, BpsA peptide and BpsA polypeptide as used herein also refer to

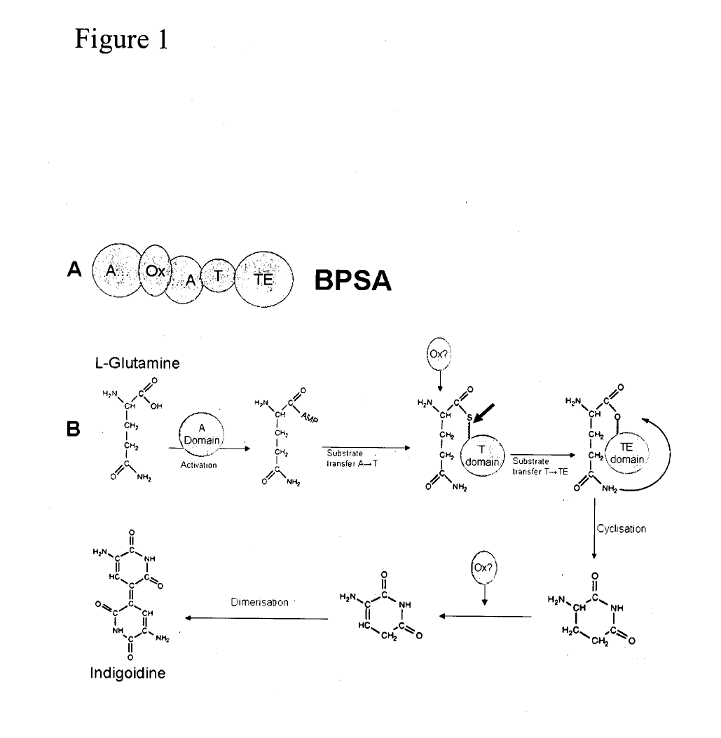

(i) An NRPS that is capable of synthesising indigoidine from two molecules of L-glutamine, for example, as shown in FIG. 1B,
(ii) An enzyme capable of synthesising indigoidine that shares at least 70% sequence identity with BpsA from Streptomyces lavendulae, preferably at least 75%, more preferably at least 80%, preferably at least 85%, preferably at least 90%, preferably at least 95%, preferably at least 96%, preferably at least 97%, preferably at least 98%, preferably at least 99%, or preferably at least 100%, and
(iii) An NRPS that contains at least one A-domain, one oxidation domain, one T-domain (also known as a PCP-domain), and one TE-domain, and that is capable of synthesising indigoidine from two molecules of L-glutamine. The oxidation domain may be located entirely within an A-domain of the NRPS enzyme, as for BpsA from S. lavendulae, for example as shown in FIG. 1.

The terms “A-domain”; “Ox domain”, “oxidation domain”, “T-domain”, “PCP-domain” and “TE-domain” as used herein refer to peptide domains that can be defined as regions of amino acid sequence within NRPS or PKS enzymes that contain a majority of the motif sequences for each domain type as defined by Marahiel et al. 1997 (Chemical Reviews, 1997, 97:2651-2673).

As used herein, a “T-domain” refers to all possible T-domains from both NRPS and PKS enzymes including peptidyl, aryl and acyl carrier proteins, but not limited thereto.

As used herein “Oxidation domain” refers to a peptide domain within an NRPS module that binds one or more flavin mononucleotide or flavin adenine dinucleotide cofactors, or that contains either or both of the Ox-1 and Ox-2 oxidation motifs as defined by Du et al. (2000). “Complementary”, as used herein describes a first nucleotide sequence in relation to a second nucleotide sequence and refers to the ability of a polynucleotide comprising the first nucleotide sequence to hybridize and form a duplex structure under certain conditions with a polynucleotide comprising the second nucleotide sequence. Such conditions can be, for example, moderately stringent conditions, stringent conditions or highly stringent conditions, as will be understood by a skilled person. Other conditions include, but are not limited to, any physiologically relevant conditions encountered inside an organism. The skilled person will be able to determine the specific appropriate conditions for testing the complementarity of two polynucleotide sequences as required for any particular hybridization application. Complementary sequences, as used herein, may also include, or be formed entirely from, non-Watson-Crick base pairs and/or base pairs formed from non-natural and modified nucleotides, as long as the hybridization requirements outlined above are met.

“Fully complementary” as used herein, refers to base-pairing of a first nucleotide sequence to a second nucleotide sequence over the entire length of the first and second nucleotide sequences.

Where a first polynucleotide is referred to as “substantially complementary” with respect to a second polynucleotide sequence, the two sequences retain the ability to hybridize under certain conditions and can be fully complementary, or may form one or more mismatched base pairs upon hybridization, but generally not more than 4, 3 or 2 mismatched base pairs upon hybridization.

The terms “complementary”, “fully complementary” and “substantially complementary” as used herein may refer, but are not limited to, base pairing between sense and antisense strands of a dsRNA, between the antisense strand of a dsRNA and a target sequence or between an antisense compound and a target sequence.

“Target sequence” as used herein refers to a contiguous portion of the nucleotide sequence of an mRNA molecule formed during the transcription of a gene or fragment or variant thereof, including mRNA that is a product of RNA processing of a primary transcript.

The term “primer” refers to a short polynucleotide, usually having a free 3′OH group that is hybridized to a template and used for priming polymerization of a polynucleotide complementary to the target.

“Probe” as used herein refers to a short polynucleotide that is used to detect a polynucleotide sequence that is complementary to the probe, in a hybridization-based assay. The probe may consist of a “fragment” of a polynucleotide as defined herein.

A “fragment” of a polynucleotide sequence provided herein includes a subsequence of contiguous nucleotides that is capable of specific hybridization to a target of interest, e.g., a sequence that is at least 8 nucleotides in length. The fragments of the invention comprise or consist of 8, preferably 10, preferably 12, preferably 15, preferably 16, preferably 17, preferably 18, preferably 19, preferably 21, preferably 22, preferably 23, preferably 24, preferably 25, preferably 26, preferably 27, preferably 28, preferably 29, preferably 30, preferably 31, preferably 32, preferably 33, preferably 34, preferably 35, preferably 36, preferably 37, preferably 38, preferably 39, preferably 40, preferably 41, preferably 42, preferably 43, preferably 44, preferably 45, preferably 46, preferably 47, preferably 48, preferably 49, preferably 50, preferably 51, preferably 52, preferably 53, preferably 54, preferably 55, preferably 56, preferably 57, preferably 58, preferably 59, preferably 60, preferably 61, preferably 62, preferably 63, preferably 64, preferably 65, preferably 66, preferably 67, preferably 68, preferably 69, preferably 70, preferably 71, preferably 72, preferably 73, preferably 74, preferably 75, preferably 76, preferably 77, preferably 78, preferably 79, preferably 80, contiguous nucleotides of a specified polynucleotide sequence, but not limited thereto. A fragment of a polynucleotide sequence can be used as a primer, a probe, included in a microarray, or used in polynucleotide-based selection or chemical evolution methods as described herein, but not limited thereto.

“Isolated” as used herein with reference to polynucleotide or polypeptide sequences describes a sequence that has been removed from its natural cellular environment. An isolated molecule may be obtained by any method or combination of methods as known and used in the art, including biochemical, recombinant, and synthetic techniques. The polynucleotide or polypeptide sequences may be prepared by at least one purification step.

“Isolated” when used herein in reference to a cell or host cell describes to a cell or host cell that has been obtained or removed from an organism or from its natural environment and is subsequently maintained in a laboratory environment as known in the art. The term encompasses single cells, per se, as well as cells or host cells comprised in a cell culture and can include a single cell or single host cell.

The term “recombinant” refers to a polynucleotide sequence that is removed from sequences that surround it in its natural context and/or is recombined with sequences that are not present in its natural context. A “recombinant” polypeptide sequence is produced by translation from a “recombinant” polynucleotide sequence.

As used herein, the term “variant” refers to polynucleotide or polypeptide sequences different from the specifically identified sequences, wherein one or more nucleotides or amino acid residues is deleted, substituted, or added. Variants may be naturally occurring allelic variants, or non-naturally occurring variants. Variants may be from the same or from other species and may encompass homologues, paralogues and orthologues. In certain embodiments, variants of the polypeptides useful in the invention have biological activities that are the same or similar to those of a corresponding wild type molecule; i.e., the parent polypeptides or polynucleotides. In certain embodiments, variants of the polypeptides of the invention have biological activities that differ from their corresponding wild type molecules. In certain embodiments the differences are altered activity and/or binding specificity.

The term “variant” with reference to polynucleotides and polypeptides encompasses all forms of polynucleotides and polypeptides as defined herein.

Variant polynucleotide sequences preferably exhibit at least 50%, at least 60%, preferably at least 70%, preferably at least 71%, preferably at least 72%, preferably at least 73%, preferably at least 74%, preferably at least 75%, preferably at least 76%, preferably at least 77%, preferably at least 78%, preferably at least 79%, preferably at least 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, and preferably at least 99% identity to a sequence of the present invention. Identity is found over a comparison window of at least 8 nucleotide positions, preferably at least 10 nucleotide positions, preferably at least 15 nucleotide positions, preferably at least 20 nucleotide positions, preferably at least 27 nucleotide positions, preferably at least 40 nucleotide positions, preferably at least 50 nucleotide positions, preferably at least 60 nucleotide positions, preferably at least 70 nucleotide positions, preferably at least 80 nucleotide positions and most preferably over the entire length of a polynucleotide used in or identified according to a method of the invention.

Polynucleotide sequence identity may be calculated over the entire length of the overlap between a candidate and subject polynucleotide sequences using global sequence alignment programs (e.g. Needleman, S. B. and Wunsch, C. D. (1970) J. Mol. Biol. 48, 443-453). A full implementation of the Needleman-Wunsch global alignment algorithm is found in the needle program in the EMBOSS package (Rice, P. Longden, I. and Bleasby, A. EMBOSS: The European Molecular Biology Open Software Suite, Trends in Genetics June 2000, vol 16, No 6. pp. 276-277) which can be obtained from http://www.hgmp.mrc.ac.uk/Software/EMBOSS/. The European Bioinformatics Institute server also provides the facility to perform EMBOSS-needle global alignments between two sequences on line at http:/www.ebi.ac.uk/emboss/align/.

Alternatively the GAP program may be used which computes an optimal global alignment of two sequences without penalizing terminal gaps. GAP is described in the following paper: Huang, X. (1994) On Global Sequence Alignment. Computer Applications in the Biosciences 10, 227-235.

Polynucleotide variants also encompass those which exhibit a similarity to one or more of the specifically identified sequences that is likely to preserve the functional equivalence of those sequences and which could not reasonably be expected to have occurred by random chance. This program finds regions of similarity between the sequences and for each such region reports an “E value” which is the expected number of times one could expect to see such a match by chance in a database of a fixed reference size containing random sequences. The size of this database is set by default in the b12seq program. For small E values, much less than one, the E value is approximately the probability of such a random match.

Variant polynucleotide sequences preferably exhibit an E value of less than 1×10⁻⁵, more preferably less than 1×10⁻⁶, more preferably less than 1×10⁻⁹, more preferably less than 1×10⁻¹², more preferably less than 1×10⁻¹⁵, more preferably less than 1×10⁻¹⁸ and most preferably less than 1×10⁻²¹ when compared with any one of the specifically identified sequences.

Use of BLASTN is preferred for use in the determination of sequence identity for polynucleotide variants according to the present invention.

The identity of polynucleotide sequences may be examined using the following UNIX command line parameters:

b12seq -i nucleotideseq1 -j nucleotideseq2 -F F -p blastn

The parameter -F F turns off filtering of low complexity sections. The parameter -p selects the appropriate algorithm for the pair of sequences. The b12seq program reports sequence identity as both the number and percentage of identical nucleotides in a line “Identities=”.

Polynucleotide sequence identity and similarity can also be determined in the following manner. The subject polynucleotide sequence is compared to a candidate polynucleotide sequence using sequence alignment algorithms and sequence similarity search tools such as in Genbank, EMBL, Swiss-PROT and other databases. Nucleic Acids Res 29:1-10 and 11-16, 2001 provides examples of online resources. BLASTN (from the BLAST suite of programs, version 2.2.13 Mar. 2007 in b12seq (Tatiana et al, 1999; Altschul et al, 1997), which is publicly available from NCBI (ftp://ftp.ncbi.nih.gov/blast/) or from NCB1 at Bethesda, Md., USA. The default parameters of b12seq are utilized except that filtering of low complexity parts should be turned off.

Variant polynucleotides also encompasses polynucleotides that differ from the sequences of the invention but that, as a consequence of the degeneracy of the genetic code, encode a polypeptide having similar activity to a polypeptide encoded by a polynucleotide of the present invention. A sequence alteration that does not change the amino acid sequence of the polypeptide is a “silent variation”. Except for ATG (methionine) and TGG (tryptophan), other codons for the same amino acid may be changed by art recognized techniques, e.g., to optimize codon expression in a particular host organism.

Polynucleotide sequence alterations resulting in conservative substitutions of one or several amino acids in the encoded polypeptide sequence without significantly altering its biological activity are also included in the invention. A skilled artisan will be aware of methods for making phenotypically silent amino acid substitutions (see, e.g., Bowie et al., 1990). Variant polynucleotides due to silent variations and conservative substitutions in the encoded polypeptide sequence may be determined using the b12seq program via the tblastx algorithm as described above.

The term “variant” with reference to polypeptides also encompasses naturally occurring, recombinantly and synthetically produced polypeptides. Variant polypeptide sequences preferably exhibit at least 50%, preferably at least 60%, preferably at least 70%, preferably at least 71%, preferably at least 72%, preferably at least 73%, preferably at least 74%, preferably at least 75%, preferably at least 76%, preferably at least 77%, preferably at least 78%, preferably at least 79%, preferably at least 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, and preferably at least 99% identity to a sequence of the present invention. Identity is found over a comparison window of at least 2 amino acid positions, preferably at least 3 amino acid positions, preferably at least 4 amino acid positions, preferably at least 5 amino acid positions, preferably at least 7 amino acid positions, preferably at least 10 amino acid positions, preferably at least 15 amino acid positions, preferably at least 20 amino acid positions and most preferably over the entire length of a polypeptide used in or identified according to a method of the invention. Polypeptide variants also encompass those which exhibit a similarity to one or more of the specifically identified sequences that is likely to preserve the functional equivalence of those sequences and which could not reasonably be expected to have occurred by random chance.

Polypeptide sequence identity and similarity can be determined in the following manner. The subject polypeptide sequence is compared to a candidate polypeptide sequence using BLASTP (from the BLAST suite of programs, version 2.2.14 [May 2006]) in b12seq, which is publicly available from NCBI (ftp://ftp.ncbi.nih.gov/blast/). The default parameters of b12seq are utilized except that filtering of low complexity regions should be turned off.

The similarity of polypeptide sequences may be examined using the following UNIX command line parameters:

b12seq -i peptideseq1 -j peptideseq2 -F F -p blastp

Variant polypeptide sequences preferably exhibit an E value of less than 1×10⁻⁵, more preferably less than 1×10⁻⁶, more preferably less than 1×10⁻⁹, more preferably less than 1×10⁻¹², more preferably less than 1×10⁻¹⁵, more preferably less than 1×10⁻⁵ and most preferably less than 1×10²¹ when compared with any one of the specifically identified sequences.

The parameter -F F turns off filtering of low complexity sections. The parameter -p selects the appropriate algorithm for the pair of sequences. This program finds regions of similarity between the sequences and for each such region reports an “E value” which is the expected number of times one could expect to see such a match by chance in a database of a fixed reference size containing random sequences. For small E values, much less than one, this is approximately the probability of such a random match.

Polypeptide sequence identity may also be calculated over the entire length of the overlap between a candidate and subject polypeptide sequences using global sequence alignment programs. EMBOSS-needle (available at http:/www.ebi.ac.uk/emboss/align/) and GAP (Huang, X. (1994) On Global Sequence Alignment. Computer Applications in the Biosciences 10, 227-235.) as discussed above are also suitable global sequence alignment programs for calculating polypeptide sequence identity.

Use of BLASTP as described above is preferred for use in the determination of polypeptide variants according to the present invention. A variant polypeptide includes a polypeptide wherein the amino acid sequence differs from a polypeptide herein by one or more conservative amino acid substitutions, deletions, additions or insertions which do not affect the biological activity of the peptide. Conservative substitutions typically include the substitution of one amino acid for another with similar characteristics, e.g., substitutions within the following groups: valine, glycine; glycine, alanine; valine, isoleucine, leucine; aspartic acid, glutamic acid; asparagines, glutamine; serine, threonine; lysine, arginine; and phenylalanine, tyrosine. Non-conservative substitutions will entail exchanging a member of one of these classes for a member of another class.

Analysis of evolved biological sequences has shown that not all sequence changes are equally likely, reflecting at least in part the differences in conservative versus non-conservative substitutions at a biological level. For example, certain amino acid substitutions may occur frequently, whereas others are very rare. Evolutionary changes or substitutions in amino acid residues can be modelled by a scoring matrix also referred to as a substitution matrix. Such matrices are used in bioinformatics analysis to identify relationships between sequences, one example being the BLOSUM62 matrix shown below (Table 1).

Table 1: The BLOSUM62 matrix containing all possible substitution scores [Henikoff and Henikoff, 1992].

TABLE 1

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | −1 | −2 | −2 | 0 | −1 | −1 | 0 | −2 | −1 | −1 | −1 | −1 | −2 | −1 | 1 | 0 | −3 | −2 | 0 |
| R | −1 | 5 | 0 | −2 | −3 | 1 | 0 | −2 | 0 | −3 | −2 | 2 | −1 | −3 | −2 | −1 | −1 | −3 | −2 | −3 |
| N | −2 | 0 | 6 | 1 | −3 | 0 | 0 | 0 | 1 | −3 | −3 | 0 | −2 | −3 | −2 | 1 | 0 | −4 | −2 | −3 |
| D | −2 | −2 | 1 | 6 | −3 | 0 | 2 | −1 | −1 | −3 | −4 | −1 | −3 | −3 | −1 | 0 | −1 | −4 | −3 | −3 |
| C | 0 | −3 | −3 | −3 | 9 | −3 | −4 | −3 | −3 | −1 | −1 | −3 | −1 | −2 | −3 | −1 | −1 | −2 | −2 | −1 |
| Q | −1 | 1 | 0 | 0 | −3 | 5 | 2 | −2 | 0 | −3 | −2 | 1 | 0 | −3 | −1 | 0 | −1 | −2 | −1 | −2 |
| E | −1 | 0 | 0 | 2 | −4 | 2 | 5 | −2 | 0 | −3 | −3 | 1 | −2 | −3 | −1 | 0 | −1 | −3 | −2 | −2 |
| G | 0 | −2 | 0 | −1 | −3 | −2 | −2 | 6 | −2 | −4 | −4 | −2 | −3 | −3 | −2 | 0 | −2 | −2 | −3 | −3 |
| H | −2 | 0 | 1 | −1 | −3 | 0 | 0 | −2 | 8 | −3 | −3 | −1 | −2 | −1 | −2 | −1 | −2 | −2 | 2 | −3 |
| I | −1 | −3 | −3 | −3 | −1 | −3 | −3 | −4 | −3 | 4 | 2 | −3 | 1 | 0 | −3 | −2 | −1 | −3 | −1 | 3 |
| L | −1 | −2 | −3 | −4 | −1 | −2 | −3 | −4 | −3 | 2 | 4 | −2 | 2 | 0 | −3 | −2 | −1 | −2 | −1 | 1 |
| K | −1 | 2 | 0 | −1 | −3 | 1 | 1 | −2 | −1 | −3 | −2 | 5 | −1 | −3 | −1 | 0 | −1 | −3 | −2 | −2 |
| M | −1 | −1 | −2 | −3 | −1 | 0 | −2 | −3 | −2 | 1 | 2 | −1 | 5 | 0 | −2 | −1 | −1 | −1 | −1 | 1 |
| F | −2 | −3 | −3 | −3 | −2 | −3 | −3 | −3 | −1 | 0 | 0 | −3 | 0 | 6 | −4 | −2 | −2 | 1 | 3 | −1 |
| P | −1 | −2 | −2 | −1 | −3 | −1 | −1 | −2 | −2 | −3 | −3 | −1 | −2 | −4 | 7 | −1 | −1 | −4 | −3 | −2 |
| S | 1 | −1 | 1 | 0 | −1 | 0 | 0 | 0 | −1 | −2 | −2 | 0 | −1 | −2 | −1 | 4 | 1 | −3 | −2 | −2 |
| T | 0 | −1 | 0 | −1 | −1 | −1 | −1 | −2 | −2 | −1 | −1 | −1 | −1 | −2 | −1 | 1 | 5 | −2 | −2 | 0 |
| W | −3 | −3 | −4 | −4 | −2 | −2 | −3 | −2 | −2 | −3 | −2 | −3 | −1 | 1 | −4 | −3 | −2 | 11 | 2 | −3 |
| Y | −2 | −2 | −2 | −3 | −2 | −1 | −2 | −3 | 2 | −1 | −1 | −2 | −1 | 3 | −3 | −2 | −2 | 2 | 7 | −1 |
| V | 0 | −3 | −3 | −3 | −1 | −2 | −2 | −3 | −3 | 3 | 1 | −2 | 1 | −1 | −2 | −2 | 0 | −3 | −1 | 4 |

The BLOSUM62 matrix shown is used to generate a score for each aligned amino acid pair found at the intersection of the corresponding column and row. For example, the substitution score from a glutamic acid residue (E) to an aspartic acid residue (D) is 2. The diagonal show scores for amino acids which have not changed. Most substitutions changes have a negative score. The matrix contains only whole numbers.

Determination of an appropriate scoring matrix to produce the best alignment for a given set of sequences is believed to be within the skill of in the art. The BLOSUM62 matrix in Table 1 is also used as the default matrix in BLAST searches, although not limited thereto.

Other variants include peptides with modifications which influence peptide stability. Such analogs may contain, for example, one or more non-peptide bonds (which replace the peptide bonds) in the peptide sequence. Also included are analogs that include residues other than naturally occurring L-amino acids, e.g. D-amino acids or non-naturally occurring synthetic amino acids, e.g. beta or gamma amino acids and cyclic analogs.

Substitutions, deletions, additions or insertions may be made by mutagenesis methods known in the art. A skilled worker will be aware of methods for making phenotypically silent amino acid substitutions. See for example Bowie et al (1990).

A polypeptide as used herein can also refer to a polypeptide that has been modified during or after synthesis, for example, by biotinylation, benzylation, glycosylation, phosphorylation, amidation, by derivatization using blocking/protecting groups and the like. Such modifications may increase stability or activity of the polypeptide.

The terms “modulate(s) expression”, “modulated expression” and “modulating expression” of a polynucleotide or polypeptide, are intended to encompass the situation where genomic DNA corresponding to a polynucleotide to be expressed according to the invention is modified thus leading to modulated expression of a polynucleotide or polypeptide of the invention. Modification of the genomic DNA may be through genetic transformation or other methods known in the art for inducing mutations. The “modulated expression” can be related to an increase or decrease in the amount of messenger RNA and/or polypeptide produced and may also result in an increase or decrease in the activity of a polypeptide due to alterations in the sequence of a polynucleotide and polypeptide produced.

The terms “modulate(s) activity”, “modulated activity” and “modulating activity” of a polynucleotide or polypeptide, are intended to encompass the situation where genomic DNA corresponding to a polynucleotide to be expressed according to the invention is modified thus leading to modulated expression of a polynucleotide or modulated expression or activity of polypeptide of the invention. Modification of the genomic DNA may be through genetic transformation or other methods known in the art for inducing mutations. The “modulated activity” can be related to an increase or decrease in the amount of messenger RNA and/or polypeptide produced and may also result in an increase or decrease in the functional activity of a polypeptide due to alterations in the sequence of a polynucleotide and polypeptide produced.

As used herein, “modulates PPTase gene expression or activity” or “PPTase gene expression is modulated” and grammatical equivalents thereof refers to where any of transcription of a PPTase mRNA from a polynucleotide encoding a PPTase amino acid sequence, translation of a PPTase mRNA that encodes a PPTase polypeptide amino acid sequence, functional activity of a PPTase polypeptide in a cell or cell pathway is “modulated” or “altered.”

The term “high throughput screening” as used herein refers to a significant increase in number of results that can be generated by a given method, in comparison to other methods used to generate the same, or same type of results. For example, the methods of the invention disclosed herein provide a high throughput screening method that may be used to screen about 1000 to about 100,000 CNAs per day, preferably at least 5,000 CNAs per day, preferably at least 10,000 CNAs per day, preferably at least 20,000 CNAs per day, preferably at least 50,000 CNAs per day, preferably at least 100,000 CNAs per day, but not limited thereto. In this example, colonies of E. coli may be visualized directly on agar plates and distinguishing by bacterial colony colour; i.e., blue colonies indicate that a functional PPTase has been expressed from a CNA and has activated the NRPS, BpsA, to form the reporter product, indigoidine. Alternatively, indigoidine production can be visualized using a spectrophotometer and 96- or 384-well plates as known in the art.

As used herein, the term high throughput screening also encompasses the automation of a method according to the invention as is known in the art. For example, HAMILTON Life Science Robotics® design automated systems for high throughput transformation of DNA libraries into E. coli, and the detection of blue colonies on agar plates could be conducted by image analysis software in conjunction with colony picking robotics. Such technologies could potentially enable the screening of at least 1,000,000 CNAs per day.

It is intended that reference to a range of numbers disclosed herein (for example 1 to 10) also incorporates reference to all related numbers within that range (for example, 1, 1.1, 2, 3, 3.9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

### DETAILED DESCRIPTION OF THE INVENTION

The inventors have determined that a CNA identified following a method of their invention frequently comprises polynucleotide sequences that are part of a cluster of polynucleotide coding sequences (i.e. ORFs) that encode one or more natural products (i.e., natural product gene clusters), or that are secondary metabolite gene clusters, or that encode the enzymes involved in the biosynthesis of secondary metabolites. This is because the present invention provides a functional screen that identifies a PPTase.

The inventors have shown that the functional PPTases required to catalyze non ribosomal peptide and polyketide synthesis, which are identified according to a method of their invention, are encoded by polynucleotide sequences that are frequently associated with other polynucleotide sequences located within the same region of the genome. Preferably the same region of a genome is comprised within at least 1, at least 2, at least 3, at least 4, at least 5, at least 10 or at least 20 kb of the polynucleotide sequence that encodes the PPTase.

The invention disclosed herein also provides what the inventors believe is the first effective method of high throughput, in vivo screening of genomic libraries for functional PPTase enzymes. Although certain other methods have been used for PPTase gene discovery, to the inventor's knowledge, all have been limited by an ability to test candidate genes on an individual basis only (Finking et al. 2002; Seidle et al. 2006). Prior to the disclosure of the invention herein, it is believed that no technology previously existed that would enable a person of skill in the art to search for and identify natural products gene clusters (NPGCs) (including secondary metabolite biosynthesis clusters and/or non ribosomal peptide or polyketide biosynthesis clusters) by identifying novel PPTases.

The invention disclosed herein also provides a versatile method of high throughput, in vitro screening of chemical libraries for inhibitors of a target PPTase enzyme. PPTase activity has traditionally been measured by unwieldy HPLC assays, sampling multiple time points and using mass spectrometry to establish the relative levels of substrate with and without PPT cofactor attached. Such assays are not amenable to high-throughput screening for inhibitors. More recently, high-throughput fluorescence-based PPTase activity screens have been used to identify novel inhibitors of the prototypical PPTase Sfp from B. subtilis (Foley et al, 2009; Yasgar et al, 2010; Duckworth et al, 2010). However, these screens are technically challenging, expensive, and not broadly applicable to other PPTase targets. In contrast the invention disclosed herein enables targeting of any PPTase that is capable of activating BpsA. The inventors also describe methods for modifying the bpsA gene to enable recognition of the encoded BpsA protein by target PPTases that might not efficiently activate the unmodified BpsA enzyme.

Generally speaking, the present invention is based on the inventor's finding that NRPS T-domains enter into specific interactions with other NRPS domains and that disruption of these interactions may be a cause of inactivity in recombinant NRPS proteins. The inventors have determined that a recently characterized pigment-synthesising NRPS protein, BpsA, may be employed as a reporter gene in domain swapping and directed evolution experiments which were conducted in order to determine key residues involved in T-domain interactions with other domains in BpsA.

The inventors believe that a method of the instant invention is useful for identifying additional NPGCs beyond those identified by certain phage display methods that have been used in the art. Without wishing to be bound by theory, the inventors believe that this ability is due to the functional nature of their method, in which a PPTase that is able to activate a non ribosomal peptide synthetase, particularly BpsA or a functional derivative or functional variant thereof, is identified by the ability of that PPTase to phosphopantetheinylate, and thereby activate BpsA.

Again, without wishing to be bound by theory, the inventors believe that the ability to identify entire NPGCs is limited only by available vector technology. It is to be understood that the choice of vector technology is believed to be within the skill of one in the art. Some natural product gene clusters span over 100 kb of DNA (Schwecke et al, 1995; Miao et al, 2005). By way of non-limiting example, identification of such clusters can be performed using bacterial artificial chromosomes (BAC) or yeast artificial chromosomes (YAC) following a method of the invention. In one example, a vector used in a method of the invention can comprise a CNA insert of 10-300 kb DNA, preferably 20-200 kb, preferably 30-150 kb, preferably 50-100 kb, but not limited thereto. The choice of CNA insert size and an appropriate vector that can comprise and express the inserted CNA is believed to be within the skill in the art (Sambrook et al. Supra).

Again, without wishing to be bound by theory, the inventors believe that recovery of only part of a SMC will enable the remainder of the cluster to be recovered, as it will be understood by a person of ordinary skill in the art that once a key marker sequence has been recovered it is likely that the remaining biosynthetic enzymes can be recovered by association (e.g. by TAR; Kim et al, 2010).

Again, without wishing to be bound by theory, the inventors believe that recovery of an entire SMC will then enable that SMC to be tested for expression of novel secondary metabolite product(s) in multiple different host organisms, as it is recognized by one of ordinary skill in the art that not all SMCs are active in all possible host strains, and that no one host strain will be suitable for expression of all recovered SMCs. Accordingly, the applicants have determined that a method according to their invention provides a means for rapidly recovering nucleic acid sequences encoding at least one protein involved in secondary metabolite biosynthesis (SMB), including meta secondary metabolite biosynthesis (mSMB), or associated with a natural products gene clusters (NPGCs) from an unidentified candidate nucleic acid sequence (CNA). In one embodiment such nucleic acid sequences are recovered from a plurality of CNAs. Preferably the polynucleotides form at least part of a cluster of nucleic acid sequences that encode the enzymes required for biosynthesis of at least part of a secondary metabolite, preferably the entire secondary metabolite.

A method according to the invention allows, for example, the screening of large and small insert candidate nucleic acid libraries, for example BAC-type libraries, and plasmid libraries, but not limited thereto. Consequently, a method of the invention may be used to identify entire NPGCs from a pool of CNAs. CNAs from a large-insert library that are identified according to the invention, (i.e., that definitely contain a functional PPTase as shown by activation of a NRPS, particularly BpsA), can be further screened to identify those clones containing a CNA that encodes an entire NPGC that is, by definition, active in synthesising novel natural products in E. coli. A method according to the invention thereby provides the potential to identify “Jackpot”-type discoveries of novel secondary metabolites that are, by virtue of being expressed in E. coli, able to be produced at commercially relevant scales.

As a result of their work, the inventors have demonstrated that BpsA is a powerful tool for gene discovery. The inventors have also shown that bpsA may be used as a reporter gene for directed evolution experiments by virtue of its ability to synthesise a readily detectable pigment in E. coli. The inventors have also shown that purified apo BpsA protein may be incubated with a target PPTase protein in vitro and used to screen for chemical inhibitors of that PPTase by virtue of the ability of an inhibitor to slow the rate of conversion of BpsA into the activated holo form, hence slowing the rate of pigment synthesis. At the core of the inventor's invention is the determination that synthesis of this pigment is dependent on BpsA being activated by 4′-PP attachment to its T-domain, a reaction which can only be catalysed by PPTase capable of recognising the BpsA T-domain. The dependence of BpsA activity on PPTase activation enables this enzyme to also serve as a reporter for characterisation and discovery of PPTase enzymes.

The experiments described herein have utilized the inventive concepts described above to provide systems and methods for identification of NPGCs, SMBCs and PPTases from CNAs, to characterize known and newly identified PPTases, to modify PPTase specificity and activity, and to discover chemical modifiers of PPTase specificity and activity, based on their recognition of how the unique properties of BpsA may be employed.

Accordingly, in one embodiment, a method according to the invention provides a means for identifying a nucleic acid that encodes a PPTase or a protein with PPTase activity from a CNA, preferably from plurality of CNAs. The recovered nucleic acid sequences may be used to optimize the production and yield of wild type and recombinantly produced secondary metabolites in cells of interest by modifying the specificity or activity or both of an encoded protein having PPTase activity. Identified and modified PPTase proteins may also be used for site specific labelling of proteins, expanding the range of the technology described by Sunbul et al and Zou and Yin (Sunbul et al., 2009; Zou and Yin, 2009) and in combination with existing phage display technologies to recover partial secondary metabolite biosynthesis gene clusters from unidentified nucleic acid sequences, including from a plurality of unidentified nucleic acid sequences (Zhou et al. 2006; Yin et al. 2007; Sunbul et al. 2009).

In one embodiment, the invention relates to a means for identifying at least part of a SMBC. In some embodiments, the identified SMBC may be part of a larger, mSMBC. The term mSMBC is used interchangeably herein with the term natural products gene clusters (NPGC). Accordingly, the methods of the invention provide a means of rapidly recovering nucleic acids encoding SMBCs and/or NPGCs from a CNA sequence, preferably a plurality of CNA sequences.

The metabolite products of SMBCc and mSMBCs are potentially valuable sources of new pharmaceuticals. For example, the currently valuable antibiotics daptomycin, bacitracin, tyrocidine, and vancomycin are NRPS and/or PKS derived secondary metabolites (Walsh et al, 2004). Recovery of SMBCs and NPGCs may also allow a means for more economical synthesis of existing secondary metabolite pharmaceuticals. NRPS and PKS gene contained in a SMBC or NPGC may also be useful for constructing synthetic secondary metabolite biosynthesis clusters useful for the production of designer secondary metabolites (Nguyen et al. 2010; Doekel et al. 2008; Gu et al. 2007; Nguyen et al. 2006; Miao et al. 2006; Gal et al. 2006 and Baltz et al. 2006).

In one embodiment a method of the invention is used to identify a CNA from a plurality of CNAs. Preferably the plurality of CNAs is comprised in a nucleic acid library, for example a genomic DNA or cDNA library, but not limited thereto. In one embodiment the genomic library is an environmental nucleic acid library, preferably an environmental DNA library (eDNA).

According to one embodiment, total DNA is isolated from an environmental sample. The term environment as used herein takes a broad meaning. An environmental sample may therefore comprise DNA extracted from any physical environment, for example, soil, water or air, or from any biological environment, for example, that is part of, or associated with an organism, but not limited thereto. Examples of biological environments include human skin, the rhizosphere of plants, algal blooms and all types of symbiotic associations such as the bovine rumen, termite hindguts and microbial mats, but not limited thereto.

The isolated DNA is then randomly fragmented or digested with restriction enzymes as known in the art or described herein to generate a plurality of unidentified nucleic acid sequence fragments (Sambrook et al. Molecular Cloning: A Laboratory Manual. 1993 Cold Spring Harbor Press). The plurality of fragmented or restricted DNA fragments may then be cloned into an appropriate expression vector to make an expression library to be screened according to the invention. An appropriate expression vector refers to any vector that can be used to generate a library of eDNA expression constructs (the “eDNA library”) comprising a CNA insert of an appropriate size range. By way of non-limiting example, an appropriate vector can comprise a 10-100 kb insert and may be a phage, phagemid, PAC, BAC, YAC, plasmid, cosmid or fosmid, as known in the art. It is believed that choice of an appropriate expression vector suitable for screening a CNA insert of a desired size range is within the skill in the art.

In one embodiment, the vector used to construct the eDNA library has an origin of replication initiation (ORI) that is the same as, or that is compatible with the ORI of a vector used to make a NRPS reporter construct according to the invention. Preferably the NRPS expressed from the reporter construct is BpsA. In one non-limiting embodiment, the ORI is the same as, or is compatible with the ORI on the pCDFduet plasmid (Novagen, Merck Biosciences, Darmstadt, Germany).

In one embodiment, the eDNA library is constructed using the plasmid pRSET as described herein and contains a plurality of eDNA expression constructs, said constructs each comprising a polynucleotide insert that is a small environmental DNA sequence fragment in a size range of from about 1.5-3.0 kb. The eDNA expression constructs present in an eDNA library prepared as above are also referred to herein as candidate nucleic acid (CNA) expression constructs.

A plurality of eDNA constructs present in the above eDNA library may be screened for candidate nucleic acids (CNAs) of interest using an appropriate NRPS. The screening of an eDNA library comprising a plurality of CNAs may be carried out in a high throughput manner according to a method of the invention. The screening of an eDNA library comprising a plurality of CNAs may also be carried out in vitro as described herein.

In one embodiment the NRPS is expressed in a bacterial cell or strain. Such a bacterial cell or strain is termed a bacterial “reporter cell” or “reporter strain” herein. The NRP can be a naturally occurring NRP of the bacterial reporter strain, or the NRP can be an NRP expressed from a candidate nucleic acid expression construct. In one embodiment, the reporter strain comprises an NRP reporter construct that expresses an NRPS. Preferably the expressed NRPS is a BpsA protein or variant thereof. Preferably the reporter strain of this embodiment is a strain of E. coli. In one embodiment, the reporter cell or strain comprises a naturally occurring NRPS that is expressed from the genome of said cell or strain. In an alternative embodiment, the reporter cell or strain comprises a non-naturally occurring NRPS that is expressed from the genome of said strain. Preferably the non-naturally occurring NRPS is BpsA.

### Non Ribosomal Peptide Synthetase (NRPS) Reporter Constructs

An NRPS reporter construct as used herein refers to a nucleic acid expression construct that comprises a polynucleotide sequence that encodes a NRPS operatively linked to a promoter that allows expression of said polynucleotide sequence to form the NRPS. In one embodiment, said expression is in vitro.

In one embodiment, said expression is in vivo in a suitable host cell or strain. According to this embodiment, a suitable host cell or strain may be any suitable prokaryotic or eukaryotic cell in which the NRPS may be expressed wherein the NRPS is not activated in said cell by any endogenous activity of said cell. In a particular embodiment, a suitable host cell or strain may be any suitable prokaryotic or eukaryotic cell in which a BpsA polypeptide may be expressed, wherein said BpsA polypeptide is not activated by any endogenous activity of said cell. In one embodiment, the suitable host cell or strain is a bacterial cell or strain or a fungal cell or strain. Preferably the bacterial cell or strain is a Gram negative bacterial cell or strain. Preferably, the bacterial cell or strain is a cell or strain of E. coli, preferably E. coli BL21(DE3) or a functional variant thereof, but not limited thereto.

A bpsA Reporter Construct

In one embodiment, a NRPS reporter construct is a bpsA reporter construct. The bpsA reporter construct is a nucleic acid expression construct comprising a polynucleotide sequence encoding a BpsA polypeptide operatively linked to a promoter that allows expression of said polynucleotide sequence. Without wishing to be bound by theory, the inventors believe that the unique ability to synthesise a readily detectable pigment in E. coli makes the single-module NRPS BpsA a powerful tool for probing NRPS domain interactions. In order for pigment synthesis to occur, BpsA must first be activated by post-translational attachment of a 4′-PP group to the active site serine of its T-domain. This activation is catalysed by a cognate PPTase enzyme, which is able to recognise specific sequence elements in the T-domain of BpsA. Since the naturally occurring PPTase enzymes of E. coli are unable to fulfil this role effectively, co-expression of an activating PPTase enzyme is necessary for BpsA-mediated pigment synthesis to occur. The dependence on PPTase-mediated activation allows the development of new assays for characterisation of PPTase enzymes, comparing the efficiency of different PPTases both in vivo and in vitro.

In one embodiment, a BpsA reporter construct is used for the discovery of new SMBCs, NPGCs and PPTase enzymes from an environmental DNA library or libraries according to the invention. In one embodiment, the invention relates to methods of using wild type and modified BpsA enzymes to assess PPTase specificity in vivo. This embodiment provides a functional qualitative assay of T-domain activation by a particular PPTase.

In one embodiment, the invention relates to an in vitro assay for accurate quantification of PPTase efficiency in which the rate of pigment synthesis by BpsA is monitored as an indirect measure of T-domain activation by a given PPTase as described herein. This quantitative in vitro assay allows the relative rates of T-domain activation to be assigned for a given PPTase to be examined. Other detailed kinetic analyses may also carried out for particular PPTases.

In one embodiment, the invention relates to a method of high throughput screening of environmental DNA for the presence of novel PPTase enzymes. Using this method, the inventors have identified 21 previously uncharacterized PPTase enzymes capable of activating BpsA from two environmental DNA libraries. Analysis of several of the sequences recovered from these screens, that encoded a previously uncharacterized PPTase, also revealed characteristic features of a secondary metabolite biosynthesis cluster, indicating that a screening method according to the invention can be used to find previously unidentified and novel SMBCs, NPGCs and PPTases from uncultured organisms and environmental nucleic acid libraries, preferably eDNA libraries, but not limited thereto.

The polynucleotide sequence encoding the NRPS may be any suitable NRPS polynucleotide sequence from any organism. Preferably the organism comprises a bacterial or archaeal cell or strain. In one non-limiting embodiment, the polynucleotide sequence encoding the BpsA synthetase may be any naturally occurring bpsA polynucleotide sequence from any bacterial cell or strain. Preferably, the polynucleotide sequence encoding the BpsA synthetase is a naturally occurring (i.e., wild type) or modified S. lavendulae polynucleotide sequence. Alternatively, the polynucleotide sequence encoding the BpsA polypeptide is a wild type or modified Erwinia chrysanthemum polynucleotide sequence.

In one embodiment, a bpsA reporter construct is made by cloning a polynucleotide sequence encoding a wild type or modified BpsA polypeptide as above into an appropriate vector. An appropriate vector is any vector that comprises a promoter operatively linked to the cloned, inserted BpsA polynucleotide sequence that allows inducible expression of the BpsA polypeptide from the vector. Preferably expression is in a suitable host cell or strain. In one embodiment, the host cell or strain may be a cell or strain of E. coli. A skilled worker appreciates that different vectors may be employed in the methods of the invention. In addition methods for constructing vectors, including the choice of an appropriate vector, and the cloning and expression of a polynucleotide sequence inserted into an appropriate vector as described above is believed to be within the capabilities of a person of skill in the art (Sambrook et al., supra).

Alternatively, the expression vector is chosen to allow inducible expression of a NRPS in a non-E. coli host cell or strain. Preferably the NRPS is bpsA. In one embodiment, the bpsA reporter construct is a bpsA reporter plasmid, said plasmid comprising the pCDFduet plasmid (Novagen, Merck Biosciences, Darmstadt, Germany) and a nucleic acid sequence encoding a BpsA protein. In another embodiment the bpsA reporter construct is a bpsA reporter plasmid, said plasmid comprising the pCDFduet plasmid (Novagen, Merck Biosciences, Darmstadt, Germany) and a nucleic acid sequence encoding a modified BpsA protein (mBpsA).

Expression of BpsA or mBpsA from a bpsA reporter plasmid is inducible with IPTG as described herein. The person of skill in the art recognizes that there are also many suitable alternative expression systems available that may be used in the methods of the invention to express a BpsA polypeptide.

In another embodiment, polynucleotide sequences encoding BpsA polypeptides may be integrated into the chromosome of an appropriate host organism as described herein, to produce a reporter strain useful in the invention. In one embodiment, a bpsA reporter construct comprises a nucleotide sequence encoding a BpsA protein and a suitable regulatory promoter that is integrated into the chromosome of E. coli (or other host organism) in an appropriate orientation to allow expression of the BpsA polypeptide in the cell. In one embodiment, a CNA expression construct and a NRPS reporter construct are expressed in vitro.

In another embodiment, a CNA expression construct and a NRPS reporter construct are expressed in vivo. Preferably, a CNA expression construct and a bpsA reporter construct are expressed in vivo. Preferably, said expression is in the same host cell or strain. To achieve this dual expression within the same host cell or strain, the nucleotide sequence encoding the CNA expression product and the NRPS are cloned into suitable, separate expression vectors. Suitable vectors must have the same or compatible ORIs in order to be stably maintained in the same host cell or strain. Preferably the NRPS reporter construct is a bpsA reporter construct. The person of skill in the art understands that other ORI compatible vector combinations may also be used according to the methods of the invention and are contemplated herein.

The term “compatible origins of replication” as used herein refers to origins of replication as known in the art, that function to stably maintain the separate vectors within which they are comprised, in a given host cell or strain. Compatible origins of replication are not required to be identical, although they may be. What is important is that compatible origins of replication function as above, to maintain separate vectors within a given host cell or strain.

In one embodiment, expression of the bpsA expression construct and the CNA expression construct is inducible by the same promoter. The promoter may be any suitable promoter as known and used in the art, that will allow expression of a CNA expression product from a CNA expression construct. The choice of an appropriate promoter for use in a method according to the invention is believed to be within the skill in the art.

In one non-limiting embodiment of the invention, the vector is a plasmid. Preferably the plasmid is the pCDFduet plasmid that is used to make expression constructs for polynucleotide sequences encoding wild type (wt) and recombinant BpsA proteins as described herein. In this embodiment, the plasmid pET28a (+) is used for expression of CNA expression constructs. In this embodiment, expression from the pCDFduet and pET28a (+) vectors is regulated by the T7 promoter which is inducible by IPTG or lactose.

In another non-limiting embodiment, expression of each construct is inducible by a different promoter. Use of different inducible promoters on the bpsA reporter construct and the CNA expression construct allows for independent regulation of expression levels of the product from each. In one non-limiting example according to the invention, the plasmid pCDFduet comprises a T7 promoter operatively linked to a polynucleotide sequence encoding a wild type (wt) or a recombinant BpsA polypeptide as described herein. In this embodiment, the CNA expression construct comprises a CNA in operative linkage with the arabinose inducible pBAD promoter (Guzman et al., 1995). Use of a vector comprising the pBAD inducible promoter allows for independent regulation of expression levels of the BpsA expression product and the CNA expression product.

### Host Strains and Test Strains

Introduction of an NRPS expression construct into an appropriate host cell or strain may be achieved using any of a number of available standard protocols and/or as described herein as known and used in the art (Sambrook et al., supra). Preferably the NRPS construct is a bpsA reporter construct as described herein. Preferably the NRPS construct is inserted into an appropriate host cell or strain that is an appropriate strain of E. coli. Such insertion may be achieved using any of a number of available standard transformation or transduction protocols as known and used in the art (Sambrook et al., supra).

As used herein, a reporter cell or strain (RS) refers to a host cell or strain that that expresses an NRPS that can be activated by a CNA expression product. In one embodiment, the host cell or strain is a fungal or bacterial, preferably bacterial, host cell or strain, but not limited thereto. Preferably the bacterial cell or strain is a Gram negative bacterial cell or strain, preferably E. coli, more preferably E. coli BL21(DE3), but not limited thereto.

In one embodiment, the NRPS is an exogenous NRPS expressed in the reporter cell or strain from an NRP reporter construct according to the invention, but not limited thereto. Alternatively, the NRPS is expressed from the genome of the reporter cell or strain. In this embodiment the NRPS may be endogenous or exogenous, naturally occurring or non-naturally occurring with respect of the reporter cell or strain. By way of non-limiting example, the NRPS may be an exogenous polypeptide expressed from an NRPS expression construct. Preferably the NRPS so expressed is a BpsA polypeptide. In this embodiment, the NRPS is an endogenous NRPS that synthesizes a pigment or dye. Preferably the pigment or dye is indigoidine.

Reporter cells and strains useful in the invention are not limited to strains of E. coli. Numerous alternative host organisms may be used to construct a reporter cell or strain useful in the methods according to the invention, wherein each constructed cell or strain may provide a different or additional benefit or utility. The choice of an appropriate host strain will affect choice of reporter construct used based on the genetic makeup of the host. A suitable reporter cell or strain may be made from any given host cell or strain as described herein, provided that the host cell or strain does not comprise an activity that will activate the NRPS to form a pigment or dye. Preferably the reporter cell or strain does not activate said NRPS because said reporter cell or strain does not comprise an enzyme that will activate the NRPS. Preferably the enzyme is a PPTase. Preferably the NRPS is a wild type BpsA polypeptide that is activated by a PPTase to form a pigment or dye.

In another embodiment, the BpsA polypeptide is modified to make a modified BpsA polypeptide (mBpsA) as described herein, wherein said mBpsA is not activated by an activity in an appropriate reporter cell or strain as described herein. Preferably the activity of the chosen reporter strain that is absent is PPTase activity. In other words, the reporter cell or strain does not comprise an endogenous PPTase that will activate the mBpsA. A key benefit of using an alternative reporter strain is that due to the functional nature of the assays according to the invention, any expressed protein involved in natural products or small metabolite biosynthesis, which is identified due to activation of the reporter construct comprised in the reporter strain, is necessarily active. Not all proteins can be expressed effectively in E. coli due to promoter inactivity, codon bias, protein insolubility or other factors. Therefore, the use of different reporter strains provides alternative hosts suitable for use in subsequent production of any protein identified according to a method of the invention.

In one embodiment, a NRPS reporter construct is introduced into an appropriate host cell or strain by any means as known and used in the art, for example, transformation or transduction, but not limited thereto (Sambrook et al., supra). In one embodiment, transformation is by electroporation. In one embodiment the NRPS is a BpsA polypeptide expressed from a bpsA reporter plasmid as described herein. Preferably the appropriate host cell or strain is E. coli BL21(DE3) or a functional variant thereof, but not limited thereto. In one embodiment, E. coli BL21 (DE3) contains the genetic elements (T7 RNA polymerase) necessary for proper function of a pCDFduet expression vector and other expression vectors under the control of a T7 promoter. In one embodiment, E. coli BL21 (DE3) is optimized for yield and recovery of recombinant proteins including recombinant PPTases identified and/or characterized according to the methods of the invention.

The generation of appropriate alternative reporter strains and cells according to the invention is also contemplated herein. An appropriate alternative reporter cell or strain is an E. coli and non-E. coli host cell or strain, wherein said cell or strain has a different genotype than any of the specific host cells or strains disclosed herein, that expresses a NRPS or peptide that is not activated by an endogenous activity of said host cell or strain. Preferably the endogenous activity that is absent from the alternative host cell or strain is PPTase activity. Preferably the alternative host cell or strain lacks an endogenous polynucleotide sequence that encodes a PPTase or a protein or peptide with PPTase activity. Alternatively the alternative host cell or strain comprises endogenous polynucleotide sequence that encodes a PPTase or a protein or peptide with PPTase activity, but is incapable of expressing said PPTase or a protein or peptide with PPTase activity. In this embodiment, the alternative host cell or strain may be incapable of expressing PPTase or a protein or peptide with PPTase activity for any reason, including but not limited to nucleic acid mutations, deletions, transpositions and/or insertions and expression of an endogenous or exogenous inhibitor of said PPTase or a protein or peptide with PPTase activity. Alternative E. coli and non-E. coli host cells and strains as contemplated herein are useful according to the invention for identifying and assessing the activity of a CNA expression product that will activate a NRPS.

In another embodiment a single host organism could be modified to allow expression of multiple different BpsA polypeptides in the cell, including modified BpsA synthetases, to maximise the ability of the host to recognise PPTase-containing CNA constructs.

In one embodiment a suitable alternative NRPS reporter strain is made by introducing a polynucleotide sequence encoding a wild-type or modified NRPS reporter peptide or protein as described herein into an appropriate host cell or strain. Preferably the NRPS is BpsA. Preferably the activity of a CNA expression product that will activate said NRPS is PPTase activity. Preferably the CNA expression product is a PPTase.

### Screening CNAs

Once an appropriate reporter cell or strain is constructed, expression products may be prepared for use in in vitro and in vivo methods as described herein and as known in the art. In one embodiment, a method of the invention is performed in vivo in cells of an appropriate reporter strain comprising a CNA as described herein. The CNA may be introduced into the cells as known in the art, for example by transformation or transduction using standard electroporation or chemical methods (Sambrook et al., supra). Preferably, CNAs may be transformed into competent cells by electroporation or chemical methods, for example, by transformation or transduction. Cells of a reporter strain comprising CNA expression constructs are then selected for by plating on selective media/pigment development agar as described herein.

After overnight growth, plasmid expression of the reporter product, preferably an NRPS, preferably BpsA is induced with IPTG and the plates further incubated for 12-24 h with regular observation. Single colonies that develop blue pigmentation within this timeframe (hits) are candidates for containing CNA expression construct that comprises a polynucleotide encoding a peptide or protein having PPTase activity. Preferably the protein or peptide is a PPTase enzyme.

Once blue transformants are observed, plasmid DNA may be isolated as known in the art and used for DNA sequencing of the identified insert. Sequenced polynucleotides may be analyzed bioinformatically to confirm the identity of the expressed peptide or protein as either a PPTase or as a peptide or protein having PPTase activity. Polynucleotide sequencing of the entire CNA insert also allows flanking coding nucleic acid sequences to be identified bioinformatically using standard analyses and methodologies as known in the art and as described herein. In one embodiment, blue transformants may be cultured in media that allows expression of CNA expression products. Total natural products and/or secondary metabolites may then be isolated and prepared from the cultured transformants for analysis by nuclear magnetic resonance (NMR), liquid chromatography mass spectrometry (LCMS) or thin-layer chromatography (TLC). The person of skill in the art recognizes that there are many suitable methods available that may be used in the methods of the invention to detect novel CNA expression products. In one embodiment, NMR is used to identify at least part of the chemical structure of a natural product or secondary metabolite produced by the transformant, preferably to identify an entire natural product or secondary metabolite.

The selective media/pigment development agar (also referred to herein as the selection medium) may be any appropriate growth medium supplemented with the appropriate selective agents that allow for selection of only those cells comprising both the reporter construct and the CNA expression construct. Preferably the growth medium is liquid or is solid or semi-solid due to the presence of an appropriate concentration of agar. In one embodiment, the concentration of agar in the selection medium ranges from 0.5 to 1.5%. In one embodiment, a selective agent may be an antibiotic such as ampicillin or rifampicin, but not limited thereto. The selection medium may also be supplemented with a substrate for the NRPS polypeptide. In one embodiment the NRPS is a BpsA polypeptide that is expressed from the reporter construct. In this embodiment, the selection medium is supplemented with L-glutamine (L-Gln). L-Gln is the BpsA substrate that allows formation of the blue pigment indigoidine. The inclusion of L-Gln in the selection medium promotes pigmentation due to BpsA activity, and is advantageous for detection of activation, including partial activation, of this NRPS. Preferably activation of the NRPS is activation of BpsA by a PPTase or peptide or protein having PPTase activity that is a CNA expression product.

In one embodiment, the selection medium is Luria Broth (LB) comprising L-Gln and an appropriate antibiotic. Preferably the antibiotic is Spectinomycin (Spec) or Ampicillin (Amp).

In one embodiment, the LB comprises about 75-125 mM L-Gln, about 40-60 μg/ml Spec. and about 75-125 μg/ml Amp. Preferably the LB comprises about 100 mM L-Gln, about 50 μg/ml Spec., and about 100 μg/ml Amp. The choice of various concentrations of constituents, such as those described above, but not limited thereto, for use in a selection medium according to the invention is believed to be within the skill in the art.

### Induction of BpsA Expression

In one embodiment, activation of the NRPS BpsA then produces the dye, indigoidine. This dye is toxic to E. coli over time. Due to this toxicity, E. coli cells expressing high levels of active BpsA in the presence of excess L-Gln for an extended period of time are not viable. Toxicity may be avoided by plating an E. coli reporter strain comprising a CNA expression construct on a selection medium lacking an inducer of BpsA expression. In one embodiment the inducer is IPTG.

For example, cells of the reporter strain comprising the CNA expression product may be incubated at a permissive temperature under conditions that allow the development of visible colonies. Typically, colonies of sufficient size for screening will develop following an overnight incubation. In one embodiment, incubation is from about 6 to about 48 hours, preferably from about 8 to about 36 hours, preferably from about 12 to about 24 hours. Once visible colonies have developed, the selective media is treated with an inducer of bpsA expression. The bpsA expression inducer is any inducer that will induce the expression of a BpsA protein from a reporter construct according to the invention, due to the nature of the expression used to make the reporter construct. In one embodiment the bpsA inducer is IPTG.

In one embodiment the IPTG is the inducer and is uniformly distributed across the bottom surface of a sterile Petri plate. An agar slab of a selection medium according to the invention, comprising visible colonies as described above is placed into the Petri plate on top of the inducer. This allows the inducer to diffuse into the selection medium over time, slowly inducing the expression of BpsA in the reporter strain, and consequently, the production of indigoidine due to the activation of BpsA in those reporter cells comprising a CNA expression product that is a PPTase. Coloration of colonies develops between 1 and 24 h after induction. Without wishing to be bound by theory, the inventors believe that the timing and extent of the development of coloration is directly correlated to the expression level of a CNA encoded PPTase, and the efficiency with which said PPTase activates BpsA.

In another embodiment, the toxicity of indigoidine to E. coli is avoided by employing auto-induction agar plates, prepared by addition of 1.5% agar to Studier ZYP-5052 auto-induction medium (Studier 2005). Use of auto-induction agar plates allows for cells to induce expression of BpsA only after exhausting the available glucose from their growth medium, by which time colonies have already achieved a visible size. This allows for both rapid growth and efficient induction of pigment production following extended incubation at room temperature. Development of blue pigmentation by colonies of cells of a reporter strain comprising a CNA expression construct indicates that the CNA expression construct comprises a polynucleotide sequence that encodes at least one protein involved in NRP synthesis, PK synthesis or FA synthesis. Preferably the at least one protein is a PPTase. Such cells and colonies are herein referred to as “PPTase positive clones”. In one embodiment, PPTase positive clones are selected by eye for further propagation and maintenance. In a high throughput embodiment according to the invention, selection may be by eye, or may be automated to further increase throughput.

In one non-limiting example, PPTase positive clones are selected using a sterile toothpick and inoculated into maintenance medium (LB supplemented with the appropriate antibiotics and glucose) and grown overnight. In a preferred embodiment, the maintenance medium comprises about 0.5-3.0 ml of LB, about 0.4% glucose, about 50 μg/ml Spec., and about 100 μg/ml Amp. Again, it is to be understood that choice of various concentrations of constituents, such as those described above but not limited thereto, will be suitable for use in a maintenance medium according to the invention. The choice of such constituents and their concentrations is believed to be within the skill in the art. Use of a maintenance medium prevents, or at least minimizes the loss of plasmid encoded sequences due to recombination. In this embodiment, bpsA expression is repressed by glucose (due to regulation from an IPTG-inducible promoter) and the reporter plasmid and CNA expression construct are maintained in the cell due to the presence of the appropriate antibiotics as known in the art and/or as described herein.

In some cases it may be desirable to conduct an additional colony isolation step, for example, to separate single PPTase positive clones for sequencing. In such cases PPTase positive clones may be selected and resuspended in an appropriate amount of GYT medium, preferably about 2004 GYT medium. About 1-10 μl of the resulting suspension is then spread onto an agar plate comprised of selective medium/pigment development agar. This process typically results in resolution of single colonies on the agar plate. Plates are then induced with IPTG as described herein, single pigmented colonies selected and grown in maintenance medium as previously described.

PPTase positive clones inoculated into maintenance medium are grown overnight under suitable conditions, and plasmid DNA isolated and prepared for sequence from the resulting cultures using standard protocols (Sambrook et al., supra). In one embodiment, plasmid DNA for sequencing is isolated from PPTase positive clones that have been grown for 12 to 16 h at 37° C. The isolated plasmid DNA is suitable for sequencing of the identified CNAs to determine the nucleotide sequence and confirm the identity of PPTase identified according to the invention. Sequencing of the entire CNA insert from a PPTase positive clone also allows the identification of polynucleotide sequences that encode additional proteins involved in NRP synthesis, PK synthesis and/or FA synthesis that are comprised in the CNA expression product. In one embodiment, said polynucleotide sequences encode non ribosomal peptide synthetases and/or polyketide synthetases that catalyze the production of a peptide and/or polyketide natural product or peptide and/or polyketide secondary metabolite. Preferably, said polynucleotide sequence encodes at least part of a secondary metabolite biosynthesis cluster (SMBC), at least part of a meta secondary metabolite biosynthesis cluster (mSMBC) and/or at least part of a natural products gene cluster (NPGC). Preferably, said polynucleotide sequence encodes an entire secondary metabolite biosynthesis cluster (SMBC), an entire meta secondary metabolite biosynthesis cluster (mSMBC) and/or an entire natural products gene cluster (NPGC).

In Vitro Determination of PPTase Kinetic Parameters with Respect to CoA or BpsA as a Variable Substrate.

In one aspect of the present invention, L-glutamine, CoA, Mg²⁺ and ATP, apo-BpsA and a buffering agent are combined in solution. Also added to the solution is a PPTase of exogenous origin, the PPTase under investigation. When present to the active holo form, BpsA catalyses the conversion of two molecules of L-glutamine to indigoidine, a pigment that can be readily detected either in vivo or in vitro through its characteristic blue coloration and strong absorbance at 590 nm. It is also possible to use a range of wavelengths close to 590 nM also. Like all NRPS, BpsA activity is dependent on PPTase-mediated activation from an apo to a holo form. As the endogenous PPTases of E. coli are ineffective in this role, recombinant (6His-tagged) BpsA purified from this host is found almost exclusively in the inactive, apo form. The velocity of indigoidine synthesis by BpsA, as assessed by change in absorbance at 590 nM over time (A590/s), is directly proportional to the concentration of holo-BpsA in a reaction. For reactions established using apo-BpsA in the presence of a PPTase, the velocity of indigoidine synthesis (A590/s) at a given time is therefore indicative of the amount apo-BpsA that has been converted to holo-BpsA by the PPTase under investigation, with the change in velocity of pigment synthesis over time, i.e. acceleration, being indicative of the velocity of reaction of the PPTase under investigation (FIG. 7). Multiple reactions can be set up with either BpsA or CoA as a variable component and all other components kept at fixed concentration. In the case where CoA is the variable component, this system enables derivation of kinetic parameters for a PPTase with respect to CoA by continuously measuring the rate of indigoidine synthesis by BpsA as it is progessively converted from apo to holo-form by that PPTase. In the case where BpsA is the variable component, this system enables derivation of kinetic parameters for a PPTase with respect to BpsA by continuously measuring the rate of indigoidine synthesis by BpsA as it is progressively converted from apo to holo-form by that PPTase. In the examples given this method is used to determine kinetic parameters for the PPTases Sfp of B. subtilis subsp. spizizenii ATCC6633, PcpS of P. aeruginosa PAO1 and the putative PPTase PP 1183 of P. putida KT2440 with respect to both CoA and BpsA as a variable substrate. The assays developed are particularly suited to two technologically relevant applications.

### Chemical Screen

The assay outlined for determination of kinetic parameters may be adapted for HTS of chemical libraries for inhibitors of a particular PPTase, by monitoring the rate of colour formation. PPTases are attractive targets for antibiotic development, owing to their central role in both primary and secondary bacterial metabolism (Mootz et al. 2001; Finking et al. 2002). Of particular note, the pathogenic bacteria Mycobacterium tuberculosis and Pseudomonas aeruginosa contain PPTases that play roles in both primary and secondary metabolism (Barekzi et al, 2004; Chalut et al, 2006). Studies aimed at uncovering inhibitors of the B. subtilis PPTase Sfp have already met with some success (Foley et al, 2009; Yasgar et al, 2010; Duckworth et al, 2010). The inventors recognize that the methods they outline require only commonly available laboratory reagents and could provide the basis for a robust and economical screening platform. Furthermore, the ability of the assays to rapidly measure PPTase velocities at a variety of CoA and/or inhibitor concentrations means they may be used to investigate efficacy and mode of inhibition for any lead compounds uncovered. The assays provided could also constitute a useful and rapid secondary screen for inhibitors identified using previously known FRET and fluorescence polarisation HTS techniques, as they do not make use of artificial substrates, and therefore more closely mimic the true physiological conditions under which PPTases act. The fact that the assays provided do not depend on fluorophore-CoA conjugates means they could also be applied to the study of PPTases which do not tolerate such substrates in place of CoA. For HTS applications, reactions of the type already described can be established in a multi well plate, in which each well contains a separate chemical compound to be interrogated for inhibitory activity. Compounds that result in a reduction in the velocity of a PPTase reaction relative to a no compound control are inhibitor candidates, and are subjected to further secondary screening. One such secondary screen, which is described as an example in this application, involves determining PPTase velocity at a variety of inhibitor concentrations and deriving an IC₅₀ value for inhibition from the resulting data. The derived IC₅₀ value can then be converted to a K, value using the Cheng-Prusoff equation, with the PPTase Km value required for this conversion being derived using the assays already described. This assay was used to derive IC₅₀ and K, values for the inhibition of Sfp, PcpS and PP 1183 by the previously identified PPTase inhibitor 6-nitroso-1,2-benzopyrone (6-NOBP; FIG. 11). Another such secondary assay which is also described as an example, involves determining the rate of indigoidine synthesis by holo-BpsA in the presence of a variety of inhibitor concentrations. In assays of this sort conversion apo to holo-BpsA is achieved using a PPTase in the absence of any inhibitory compound, and the resulting holo-BpsA is employed in the assay. Assays of this type enable compounds which are inhibitors of BpsA and compounds which inhibit both BpsA and the PPTase under investigation to be differentiated from compounds which inhibit only the PPTase under investigation. Another such secondary assay might involve conducting kinetic analysis of a PPTase with either BpsA or CoA as a variable substrate, in the presence of various concentrations of an inhibitor. Assays of this sort would enable the mode of inhibitor action (for example competitive, uncompetitive or mixed) to be determined.

### Compatibility

Another aspect of the invention described is a CoA competition assay for PPTase characterization. Assays of this type consist of two phases. In the first phase is the competition phase in which apo-BpsA and a purified carrier protein (CP) to be characterized compete for a limited pool of CoA which is attached to either the CP to be characterized or apo-BpsA by a PPTase. During this first phase, only the substrates necessary for phosphopantetheinylation are present. Following the competition phase, the production phase is initiated. In the production phase, the substrates required for pigment synthesis by holo-BpsA (L-gln and ATP) are added to reaction vessels and the velocity (A590/s) of the resulting reaction measured. As previously outlined, the velocity of this reaction is directly proportional to the concentration of holo-BpsA in the reaction and provides a measure of the amount of holo-BpsA produced during the competition phase of the reaction. Conducting such competition/production assays in the presence of a variety of concentrations of a CP to be characterized allows determination of an IC₅₀ value, indicating the concentration of CP which is required to compete for 50% of the available CoA during the competition phase of the assay. In order for a CP to compete for 50% of the available CoA, its average velocity of modification must be equal to that of apo-BpsA modification during the competition phase of the assay. The IC₅₀ value therefore indicates the concentration of CP required to achieve an average velocity of modification equal to that of apo-BpsA. Since the concentration of BpsA in an assay is known and it is possible to derive kinetic parameters for a PPTase with respect to BpsA as a variable substrate, it is possible to convert IC₅₀ values for a CP with a particular PPTase into estimates of k_{cat}/Kₘ for a particular CP-PPTase combination, as outlined in the methods and examples detailed in this application.

In the example given the CoA competition assay allowed for rapid determination of relative efficiency of modification of different CP substrates by the PPTases examined experimentally. The assay employed assessed the ability of a given CP to compete with BpsA for a limited pool of CoA. Under these conditions, CPs which are modified more quickly by a PPTase would consume more of the limited pool of CoA and as such should yield lower IC₅₀ values. Lower IC₅₀ values are therefore indicative of a higher k_{cat}/Kₘ for a particular CP/PPTase combination. Our results indicate that all three PPTases were capable of using each of the four CPs tested as a substrate, although with widely varying efficiency.

The CoA competition assay outlined is a convenient tool for determination of PPTase/CP compatibility and kinetic parameters. The assay could also be applied to the characterisation of short peptides which are potential PPTase substrates. This tool could be applied to the discovery of new tags and PPTases for site specific labelling of proteins. Existing technology readily allows for orthogonal labelling of two proteins with fluorescent CoA conjugates based on the differing efficiency of Sfp and AcpS catalysed modification of specific peptide or CP fusion tags (Sunbul et al., 2009; Zou and Yin, 2009). The inventors note that their idea provides a basis for rapid determination of relative modification efficiency by a PPTase. This could easily be adapted to provide a basis for uncovering new combinations of PPTase and CP/peptide tag to expand the capacity for orthogonal labelling beyond two separate proteins. The inventors also note that they have proven the ability of different modified BpsA reporters to recover different PPTase clones from the same eDNA library (e.g. Table 3.3). This demonstrates that the different PPTases have different affinities for the T-domains of the different modified BpsA reporters used to recover them, which immediately provides a basis for developing CP/peptide tags that are specific for different PPTases.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents; or such sources of information, in any jurisdiction, are prior art, or form part of the common general knowledge in the art.

The invention will now be illustrated in a non-limiting way by reference to the following examples.

### EXAMPLES

Example 1—in vivo characterisation and in vitro kinetic analysis of phosphopantetheinyl transferases using the single module NRPS protein BpsA . . .
1.1—Overview: . . .
1.2—Qualitative assessment of PPTase activity in vivo by co-expression with a BpsA reporter in E. coli . . .
1.3—Purification of BpsA and PPTases from E. coli . . .
1.4—Preliminary in vitro analysis of BpsA . . .
1.5—Derivation of kinetic parameters for BpsA . . .
1.6—Derivation of kinetic parameters for PPTase enzymes using a BpsA coupled enzyme assay . . .
1.7 Estimation of kinetic parameters for other carrier protein substrates using a BpsA coupled assay . . .
1.8 Evaluation of PPTase inhibition by 6-nitroso-1,2-benzopyrone . . .
1.9 Recovery of 6-NOBP and novel PcpS inhibitors from the LOPAC¹²⁸⁰ compound library . . .
   Example 2—Recovery of PPTase and secondary metabolite genes from a soil derived small insert environmental DNA library using the unmodified bpsA gene as a reporter . . .
2.1—Overview: . . .
2.2—Recovery of PPTase genes and associated SMC markers from a previously constructed soil derived eDNA library . . .
2.3—Sequence analysis and gene identification . . .
   Example 3—Generation of modified BpsA derivatives and their application to screening of environmental DNA libraries . . .
3.1—Overview: . . .
3.2—Generation of modified BpsA derivatives by T-domain substitution . . .
3.2—Development of a high throughput screening process for directed evolution of recombinant bpsA genes . . .
   3.2.1—Development of a first tier agar plate based screening system . . .
   3.2.2—Optimisation of library generation for directed evolution . . .
3.3—Improvement of slPvdD1 function by directed evolution . . .
   3.3.1—Second round evolution of slPvdD1 . . .
3.4—Improvement of slEntF function by directed evolution . . .
3.5—Re-screening of a soil derived eDNA library using an evolved modified BpsA reporter . . .
3.6—Sequence analysis of hits recovered from screening of a soil library using an evolved modified BpsA reporter . . .
3.7—Testing cross-reactivity of different eDNA clones in each reporter strain . . .
3.8—Construction and preliminary screening of a second soil derived eDNA library . . .
   4—Material and methods . . .
4.1—General reagents and materials . . .
4.2—Enzymes . . .
4.3—Bacterial strains and plasmids . . .
   4.3.1—Bacterial strains . . .
   4.3.2—Plasmids . . .
4.4—Oligonucleotide primers . . .
4.5—Media . . .
4.5.1—Media supplements . . .
4.6—Growth and maintenance of bacteria . . .
4.7—Routine molecular biology . . .
   4.7.1—PCR protocols . . .
   4.7.2—Isolation purification and manipulation of DNA . . .
   4.7.3—Preparation and transformation of competent cells . . .
4.8—Protein expression and purification . . .
   4.8.1—Protein expression . . .
   4.8.2—Protein purification by Ni-NTA affinity chromatography . . .
   4.8.3—SDS-polyacrylamide gel electrophoresis . . .
4.9—Directed evolution protocols . . .
   4.9.1—Library generation . . .
   4.9.2—First tier screening . . .
   4.9.3—Second tier screening . . .
4.10—Determination of in vivo pigment synthesis activity of mBpsA derivatives relative to wild type BpsA
4.11—Enzyme kinetics . . .
   4.11.1—Activation of BpsA by 4′-PP attachment . . .
   4.11.2—Determination of kinetic parameters for BpsA . . .
   4.11.3—Determination of kinetic parameters for PPTases . . .
   4.11.4—Carrier protein competition assay . . .
4.12—Generation of a soil derived eDNA library . . .
   4.12.1—Extraction and purification environmental DNA from a soil sample . . .
   4.12.2—Partial DNAase digest size fractionation and end repair of purified eDNA . . .
   4.12.2—Vector preparation . . .
   4.12.3—Library generation . . .
4.13—Screening of eDNA library . . .
4.14—Isolation of plasmid DNA containing eDNA fragments from hits . . .
4.15—Design and construction of modified BpsA derivatives and staging vector for directed evolution . . .
   4.15.1—Design and construction of T-domain swapping vector pBPSA3 . . .
   4.15.2—Delineation of T-domains by structural modelling and sequence alignment . . .
4.16—Detailed explanation of PPTase kinetic calculations . . .
   4.16.1—Unit conversion for PPTase kinetic parameters . . .
   4.16.2—Conversion of IC₅₀ to values to estimates of k_{cat} and Kₘ for Carrier Protein/PPTase combinations . . .
4.17—Discovery and characterization of PPTase inhibitors . . .
   4.17.1—Assessment of PPTase inhibition by 6-NOBP . . .
   4.17.2—Screening of the Lopac¹²⁸⁰ chemical library to identify novel inhibitors of the P. aeruginosa PPTase PcpS . . .
   4.17.3—Assessment of PcpS inhibition by Bay11-7085 . . .

### Example 1

### In Vivo Characterisation and In Vitro Kinetic Analysis of Phosphopantetheinyl Transferases Using the Single Module NRPS Protein BpsA

### 1.1—Overview:

In the following examples, the inventors outline the development of a novel system for characterisation of PPTases that uses the single module NRPS protein BpsA (Takahashi et al, 2007) as a reporter for determination of PPTase activity in vivo and determination of PPTase kinetic parameters in vitro. As outlined in FIG. 1, BpsA catalyses the conversion of L-glutamine into the blue pigment indigoidine. In order to carry out this pigment synthesis reaction, BpsA must first be recognized and activated by a cognate PPTase enzyme. This dependence on PPTase activation allows BpsA to be used as a reporter to monitor PPTase activity. Using BpsA as a reporter, it is possible to qualitatively discern PPTase activity in vivo and accurately derive kinetic parameters in vitro in a 96 well plate (wp) format. Analysis of a previously uncharacterized PPTase from Pseudomonas putida KT2440 is also reported.

1.2—Qualitative Assessment of PPTase Activity In Vivo by Co-Expression with a BpsA Reporter in E. coli

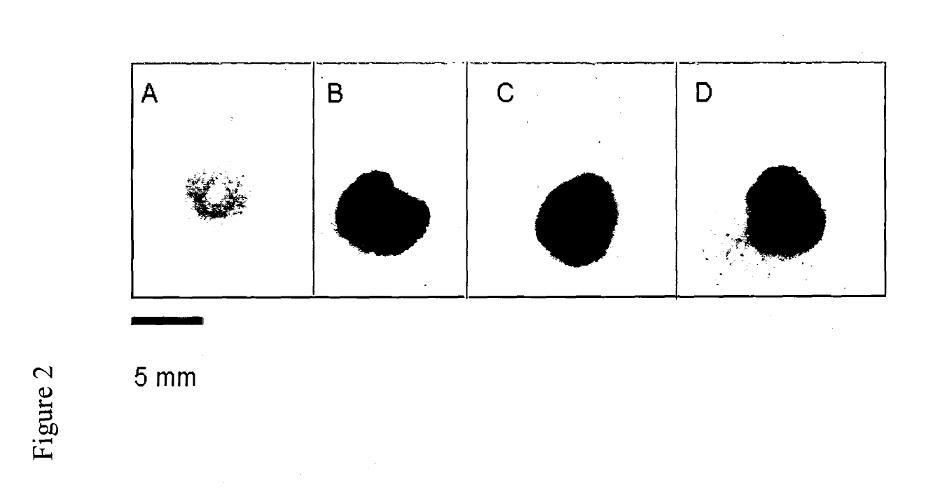

It has previously been demonstrated that the endogenous PPTases of E. coli are not effective in activating the PCP-domain of BpsA and that co-expression of a cognate PPTase is necessary for production of the blue pigment indigoidine (Takahashi et al. 2007). This suggested that we might be able to use recombinant E. coli strains co-expressing BpsA and individual PPTases to quickly ascertain whether candidate PPTases are capable of post-translationally modifying BpsA, prior to more detailed characterisation in vitro. We constructed plasmids that allowed IPTG regulated co-expression of a candidate PPTase and BpsA in E. coli BL21(DE3). To enable IPTG regulated co-expression the BpsA gene of S. lavendulae ATCC 11924 was cloned into pCDFDUET-1 (Novagen) and three candidate PPTases into the (origin of replication compatible) vector pET28a(+). Initial efforts to generate strains co-expressing BpsA and either Sfp, PcpS, or PP1183 indicated that it was not possible to recover cells containing functional copies of both genes on media containing IPTG. In contrast, if IPTG was absent from the medium viable co-transformed cells were recovered with good efficiency (not shown). However, these co-transformed cells exhibited severe growth retardation when sub-cultured on plates containing IPTG. As it had previously been shown that E. coli can indeed be induced to produce indigoidine (Takahashi et al, 2007), our observations suggested that the apparent toxicity of this compound to our cells was a result of longer-term exposure to high levels of indigoidine. In order to circumvent this problem we employed auto-induction agar plates, prepared by addition of 1.5% agar to Studier ZYP-5052 auto-induction medium (Studier 2005). Use of auto-induction agar plates allowed for both rapid growth and efficient induction of pigment production following extended incubation at room temperature. We found that co-expression of BpsA with any of the three PPTases selected (Sfp, PcpS and PP1183) resulted in substantial production of indigoidine under these conditions (FIG. 2).

1.3—Purification of BpsA and PPTases from E. coli

For purification of BpsA, expression was conducted using liquid auto-induction medium at 16° C. The procedures for expression and subsequent purification of BpsA are described in Sections 4.8.1 and 4.8.2 respectively. Owing to the FMN group bound to the Ox domain, BpsA enzyme preparations have a characteristic bright yellow colouration (Takahashi et al. 2007), a feature which assisted the monitoring of enzyme solubility and purification. Following lysis and separation, the soluble fraction was discernibly yellow, indicating good yield of soluble protein. This indication was confirmed by subsequent SDS page analysis which revealed a large band corresponding to BpsA in the soluble fraction and very little BpsA in the insoluble fraction (not shown).

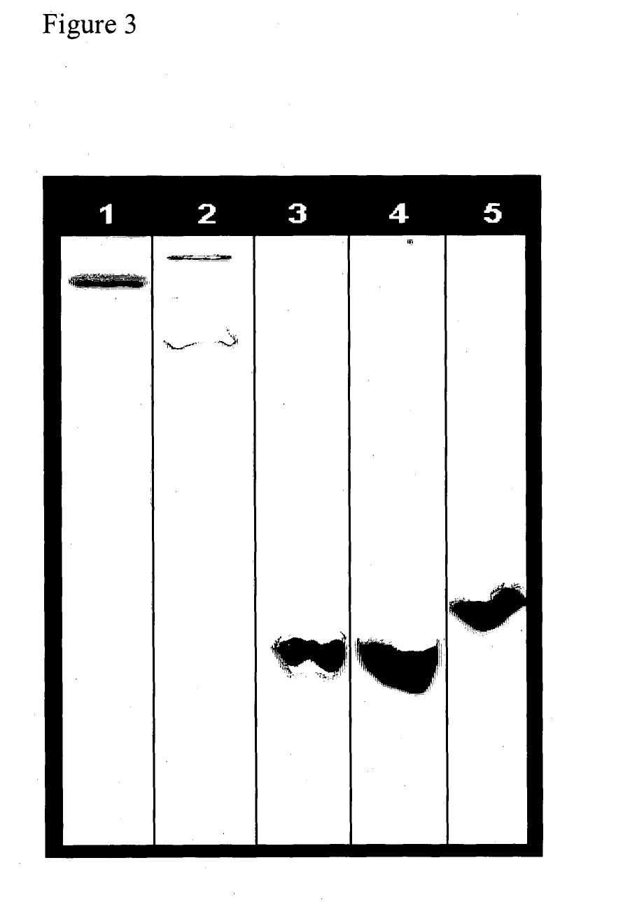

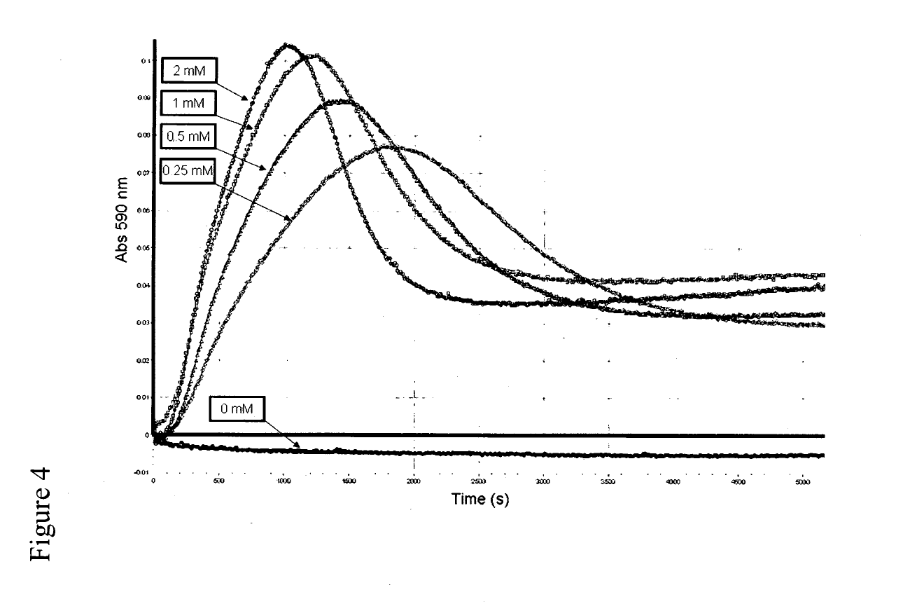

An initial purification was carried out using 200 ml of induced culture. However, difficulty was encountered in separating BpsA from major contaminating species without also eluting column bound BpsA. Subsequent attempts used 2000 ml of a scaled up expression culture. This allowed production of soluble protein in vast excess to the binding capacity of the column used. This excess of target protein resulted in a much lower level of contaminants, with the final protein appearing sufficiently pure for subsequent biochemical analyses (FIG. 3, lane 1). Three broad specificity PPTase enzymes were also purified using Ni-NTA affinity chromatography (FIG. 3, lanes 3-5). These were B. subtilis Sfp, P. aeruginosa PcpS, and P. putida PP1183, as described in Owen et al (2011). Expression of PPTase enzymes was conducted at 16° C. using auto-induction medium as described in Section 4.8.1 with subsequent purification as described in Section 4.8.2.

### 1.4—Preliminary in Vitro Analysis of BpsA

For in vitro analysis of indigoidine synthesis, purified BpsA was first incubated with purified PP1183 and appropriate substrates to bring about 4′-PP attachment as described in Section 4.11.1. Indigoidine synthesis reactions were then established in a 96wp as described in Section 4.11.2 except ATP concentration was kept at 2 mM for all reactions and L-Gln concentration was varied. Reactions were initiated by addition of activated enzyme solution followed continuous measurement of A₅₉₀ in a Versamax™ microplate reader. As shown in FIG. 4, reactions exhibited a decrease in absorbance after the maximum value was reached. The rate of decrease in product concentration after the peak value was proportional to the rate of increase before. The reason for this phenomenon was investigated in more detail.

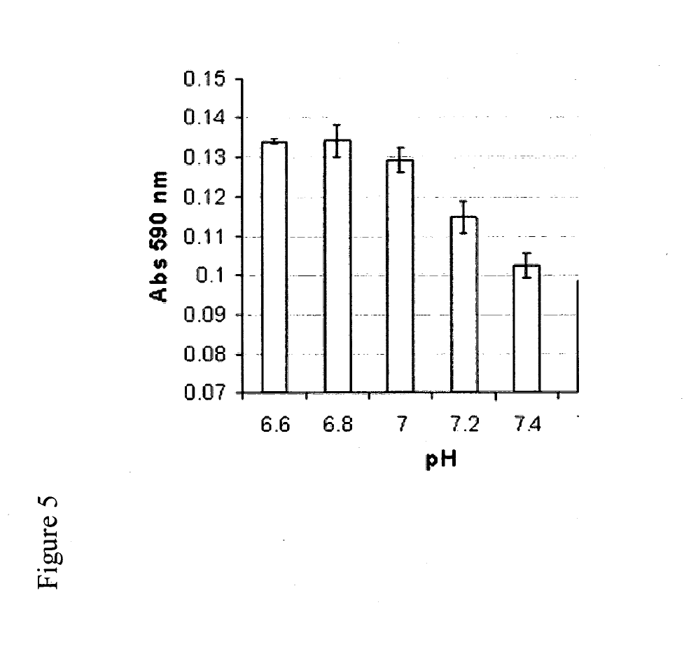

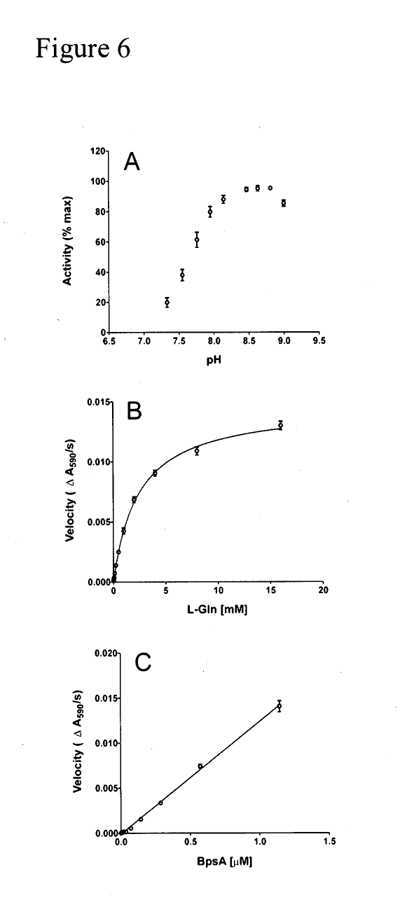

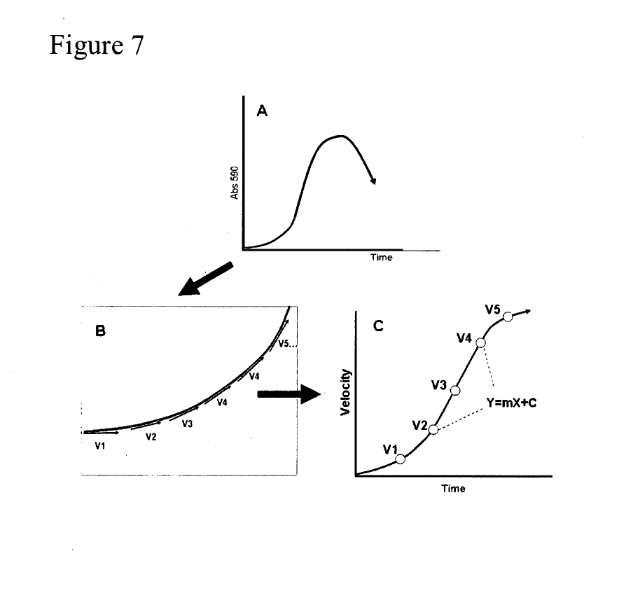

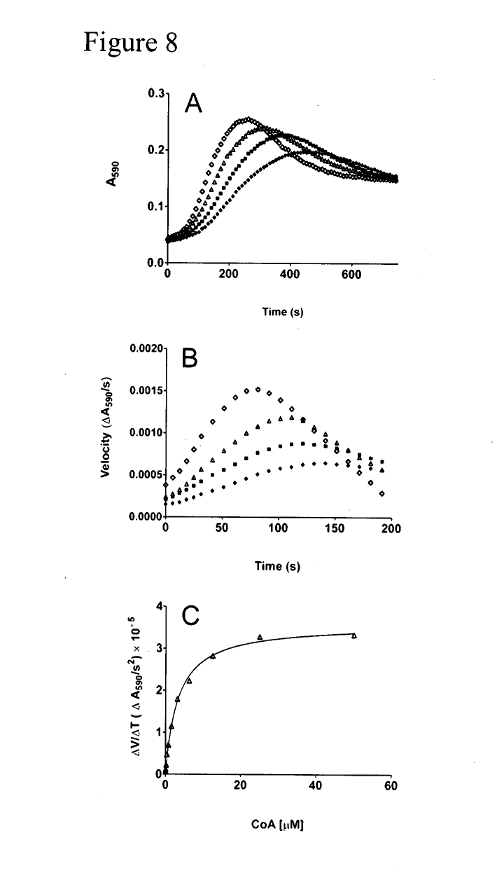

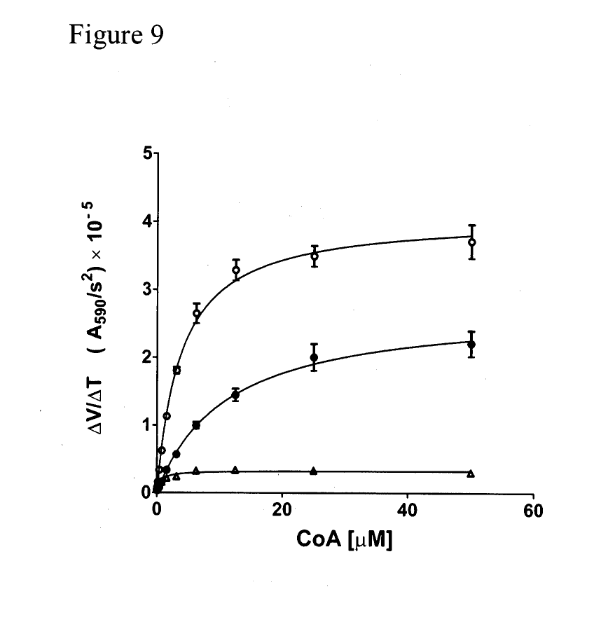

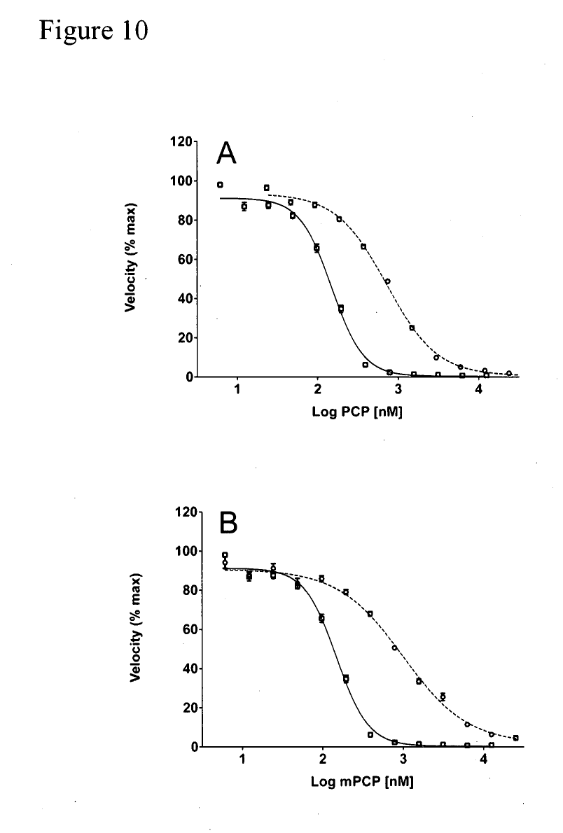

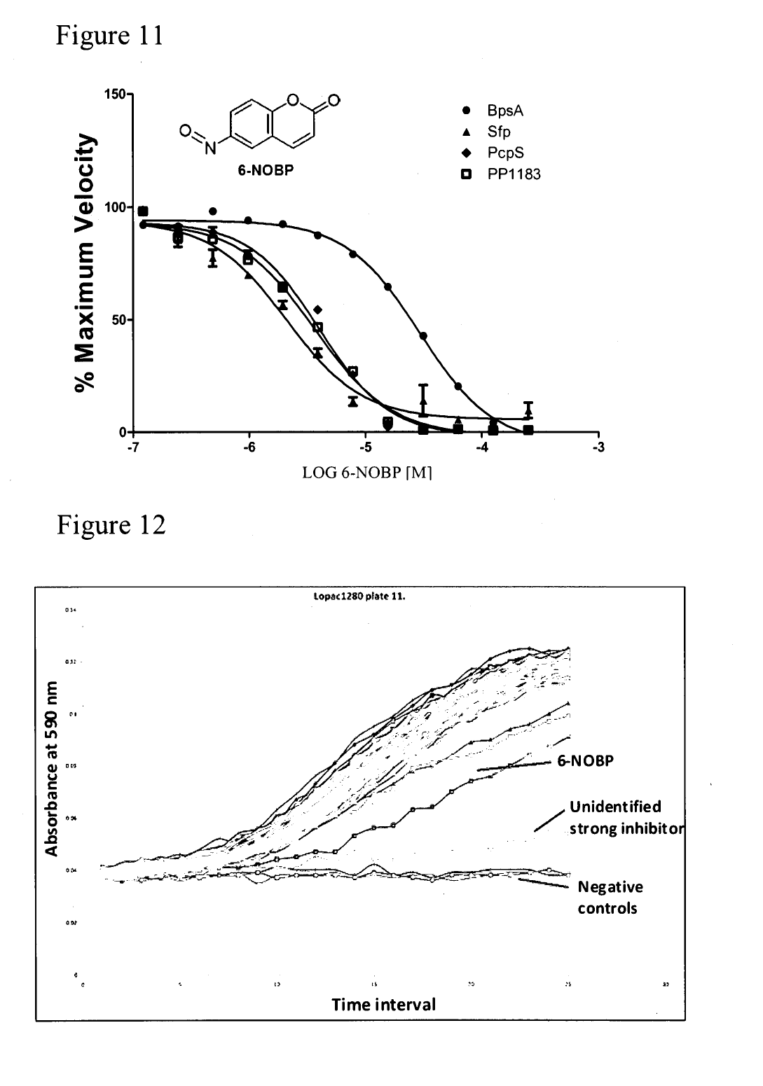

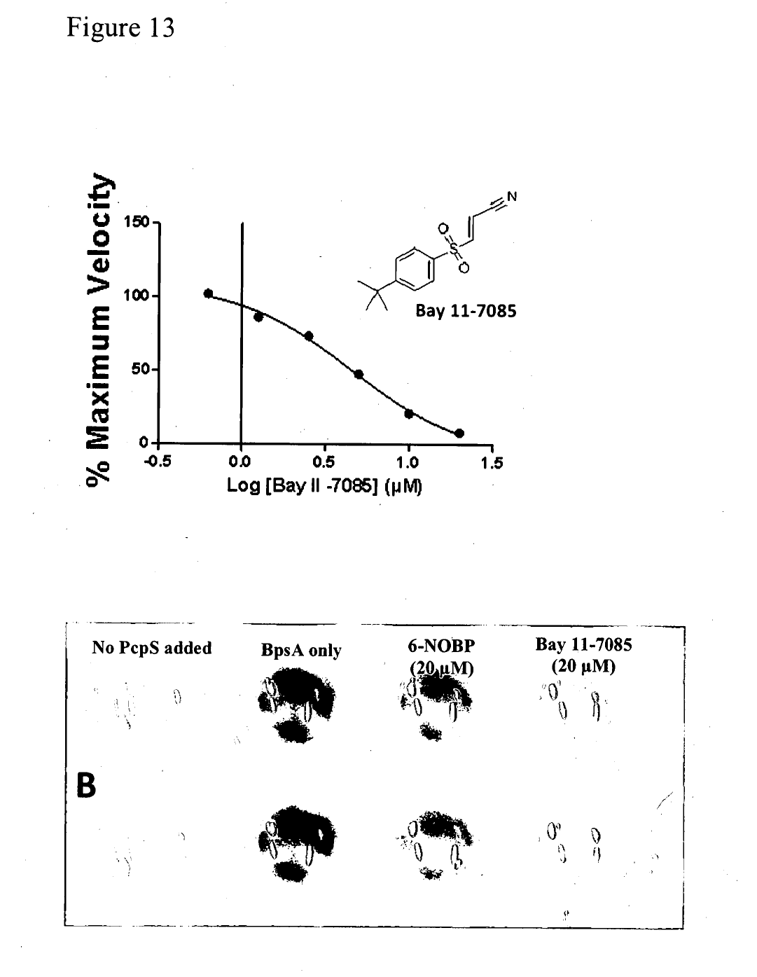

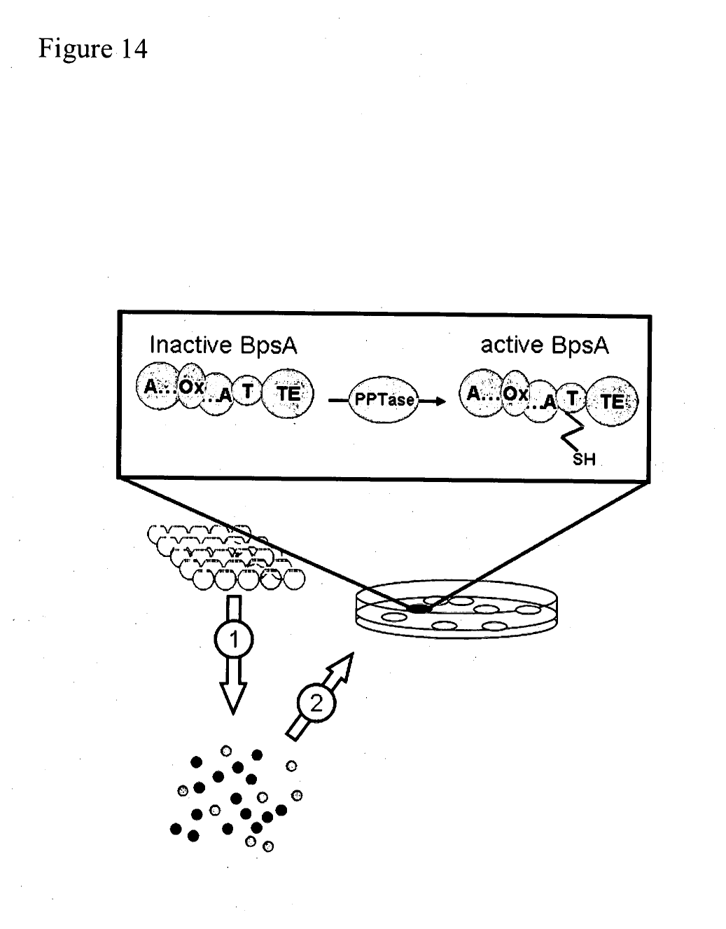

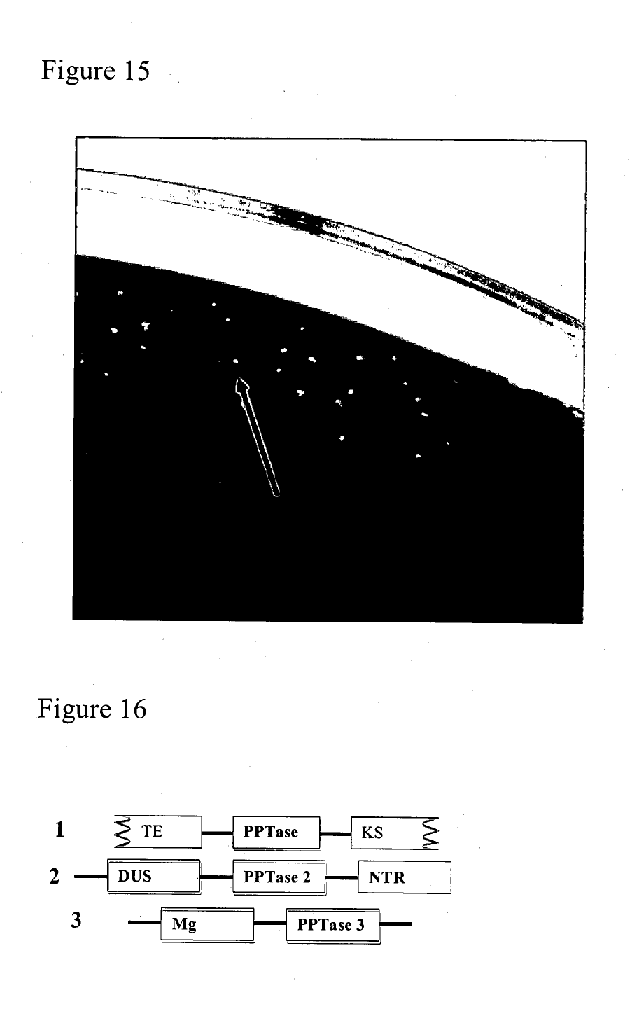

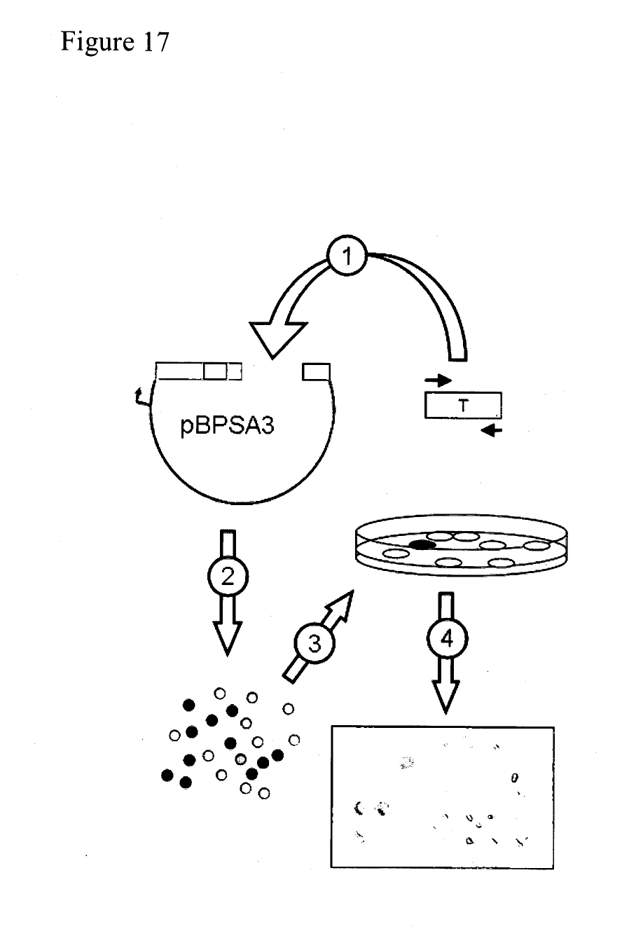

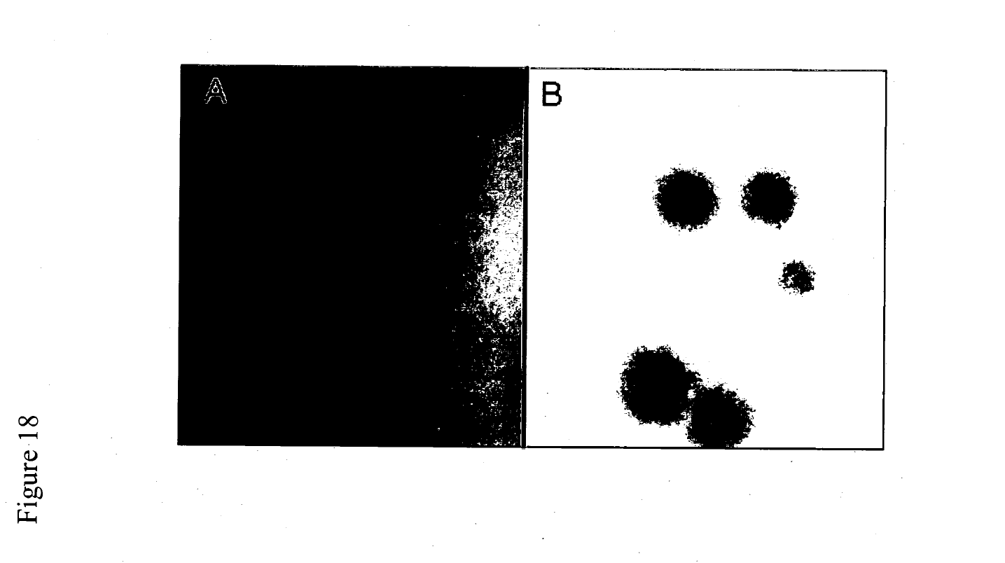

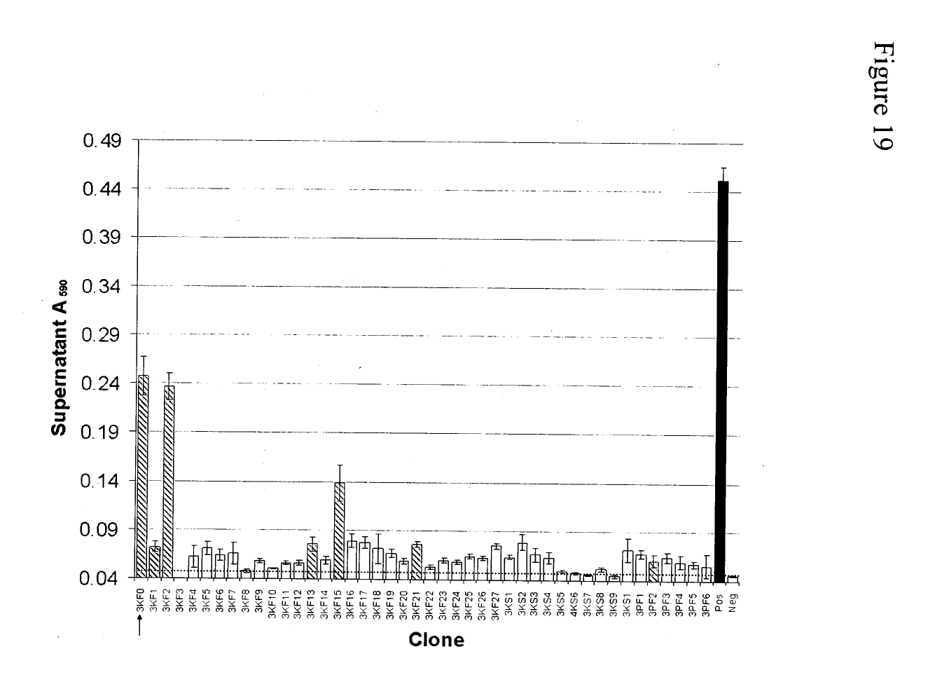

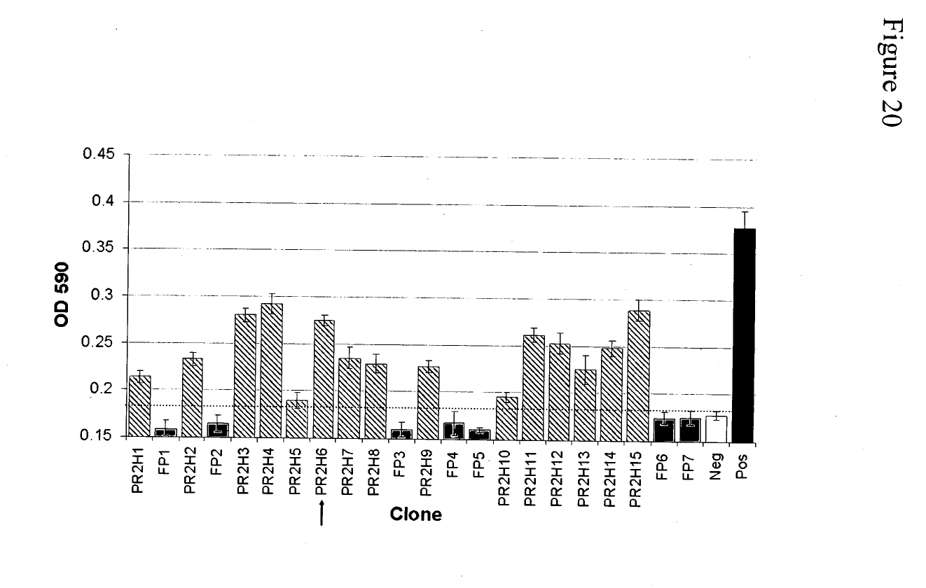

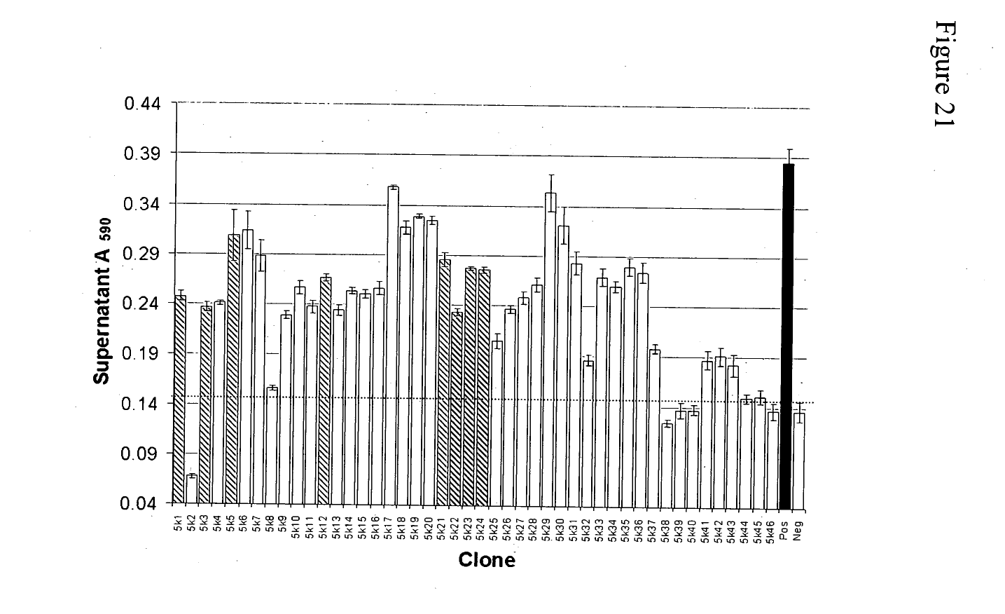

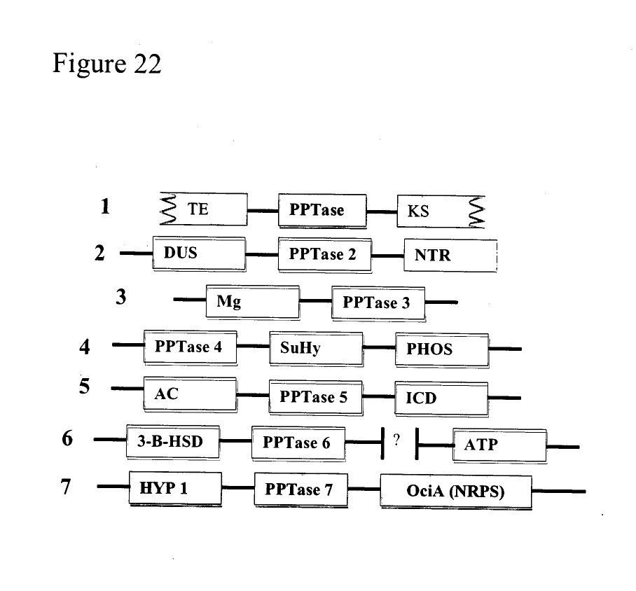

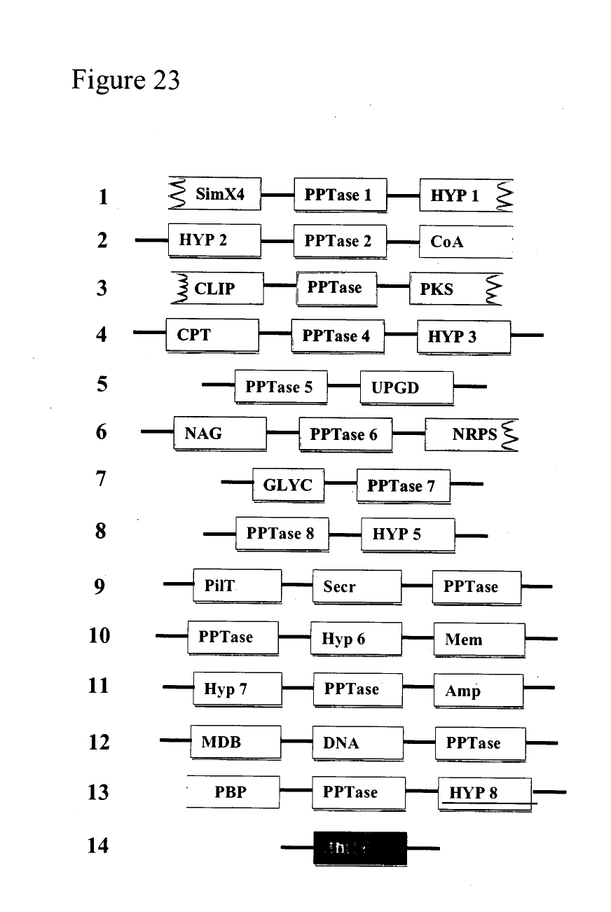

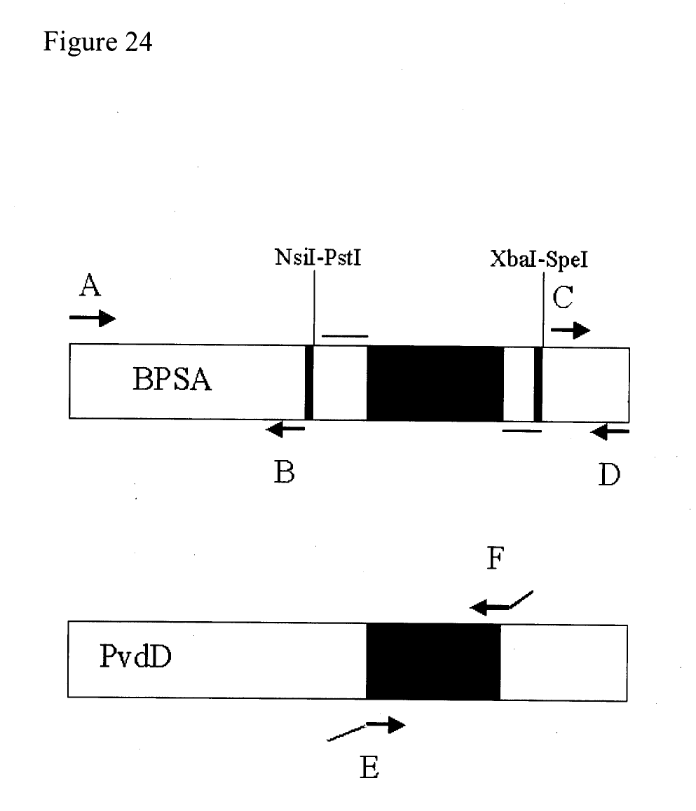

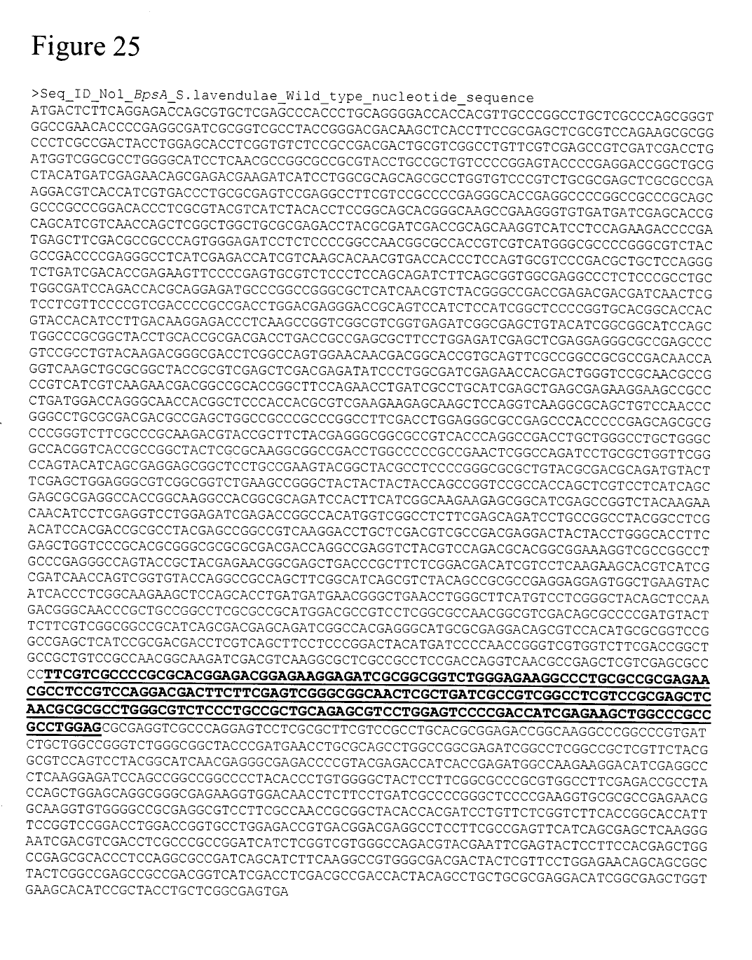

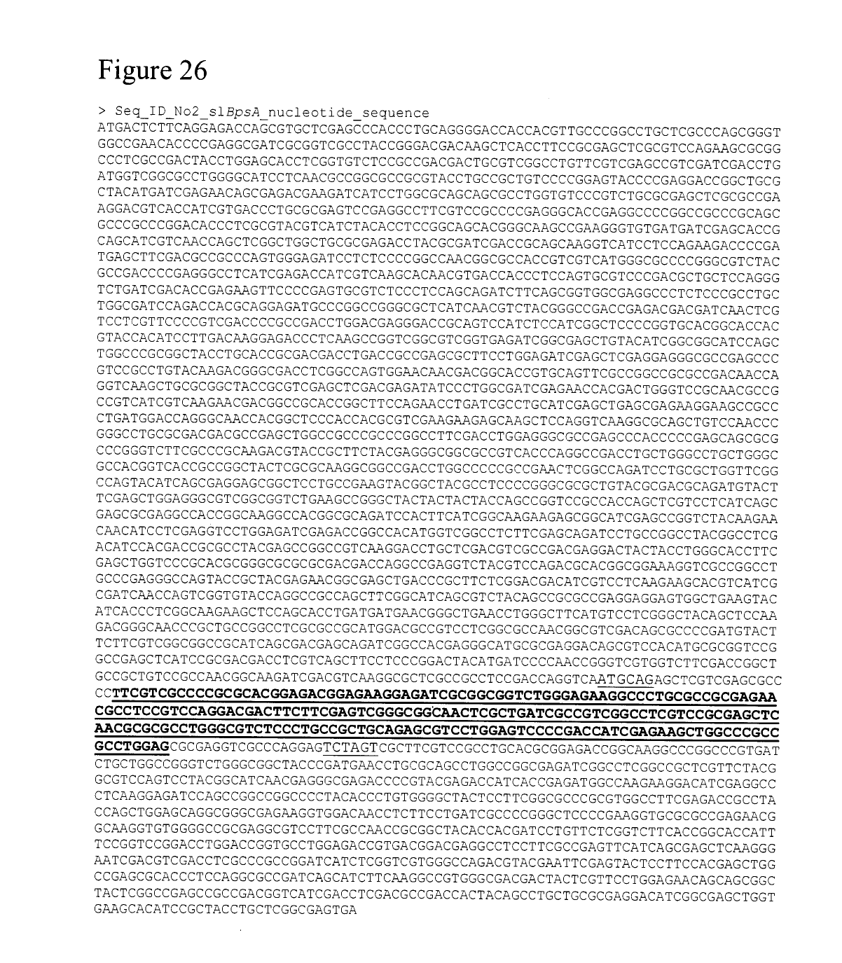

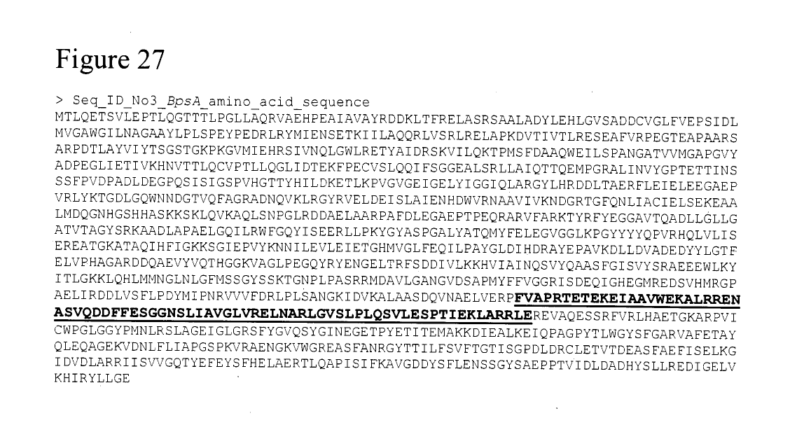

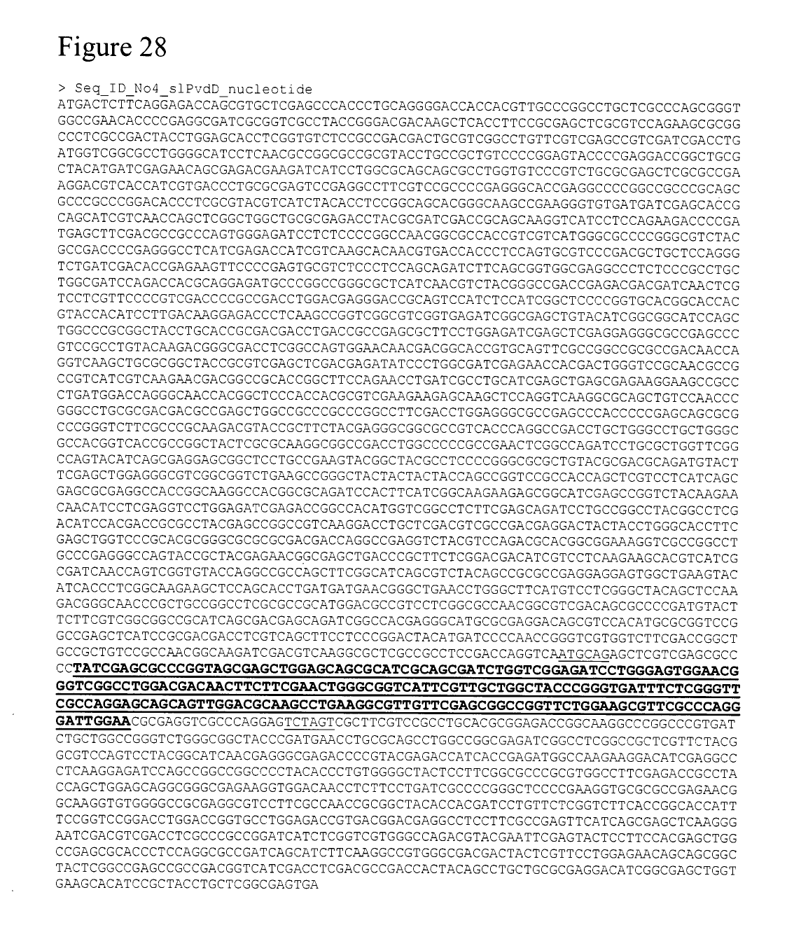

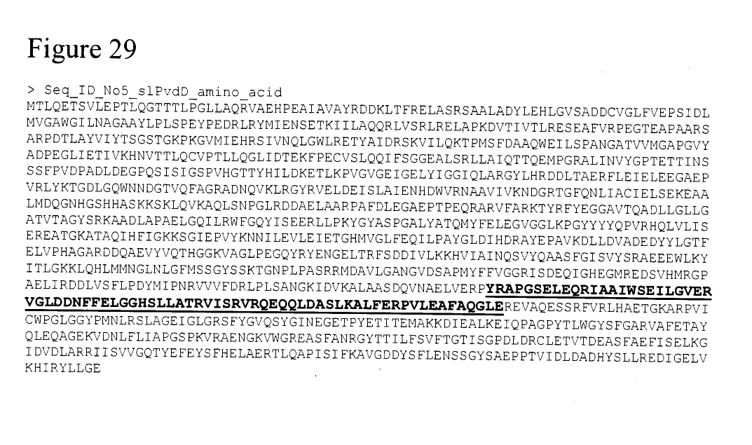

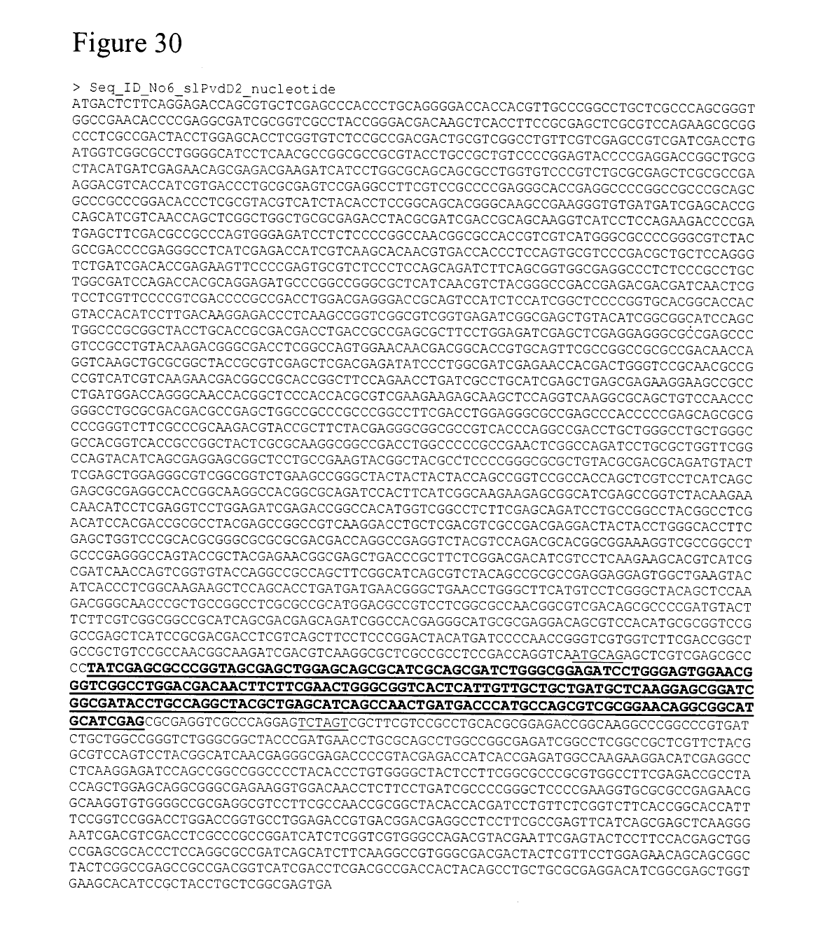

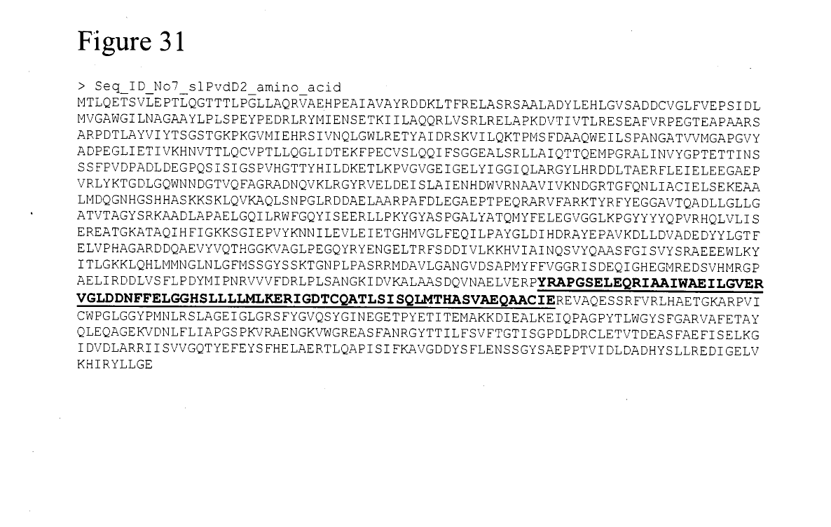

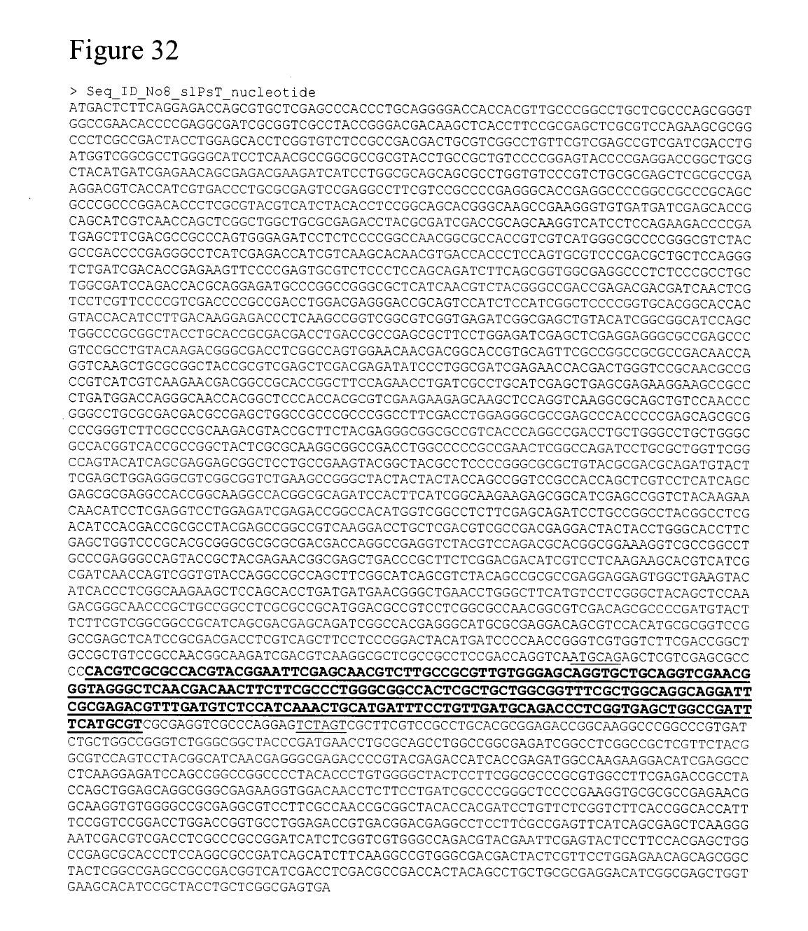

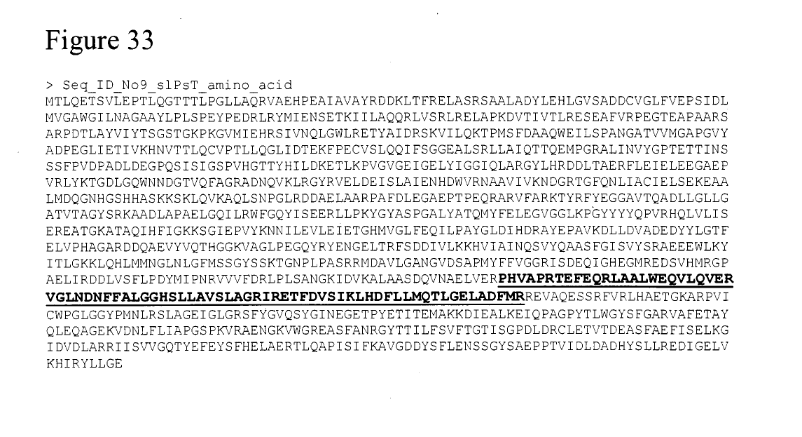

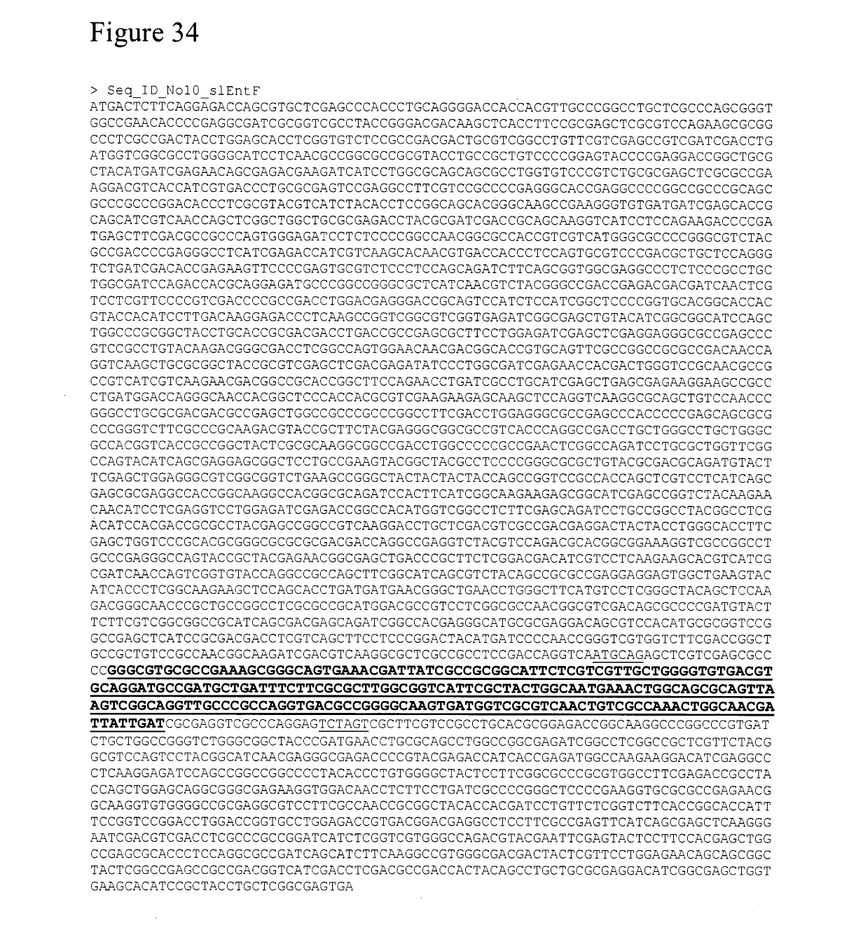

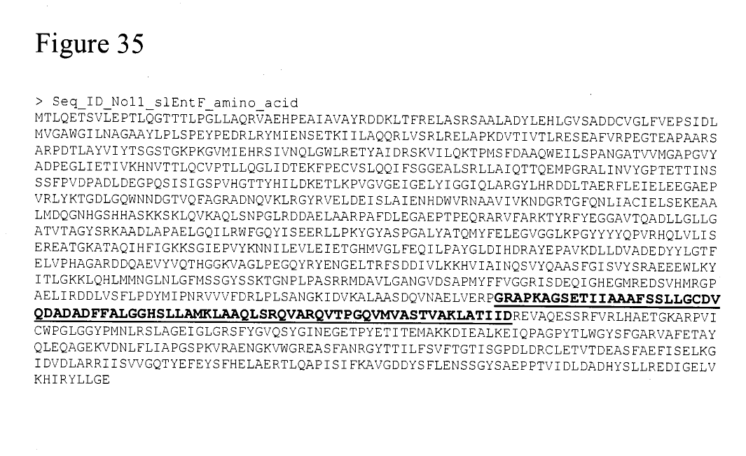

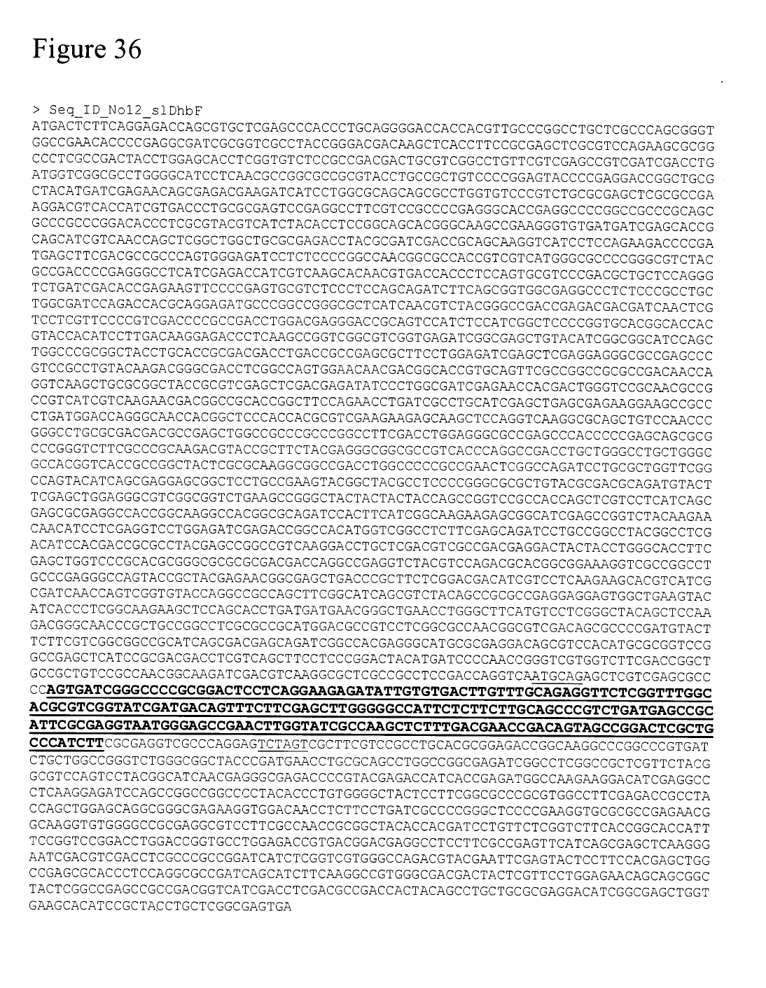

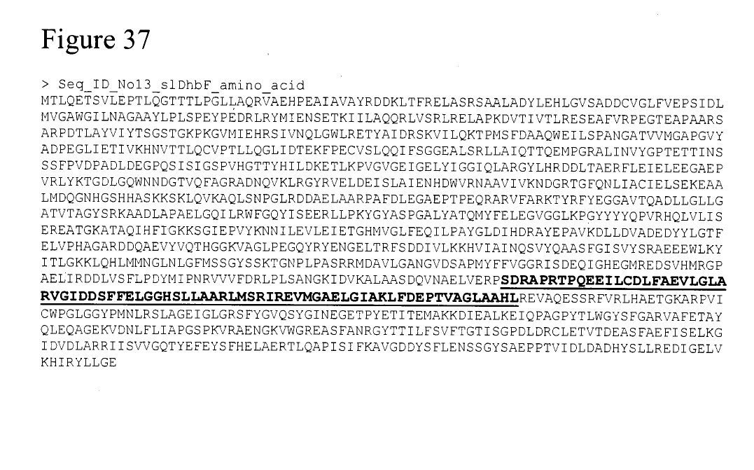

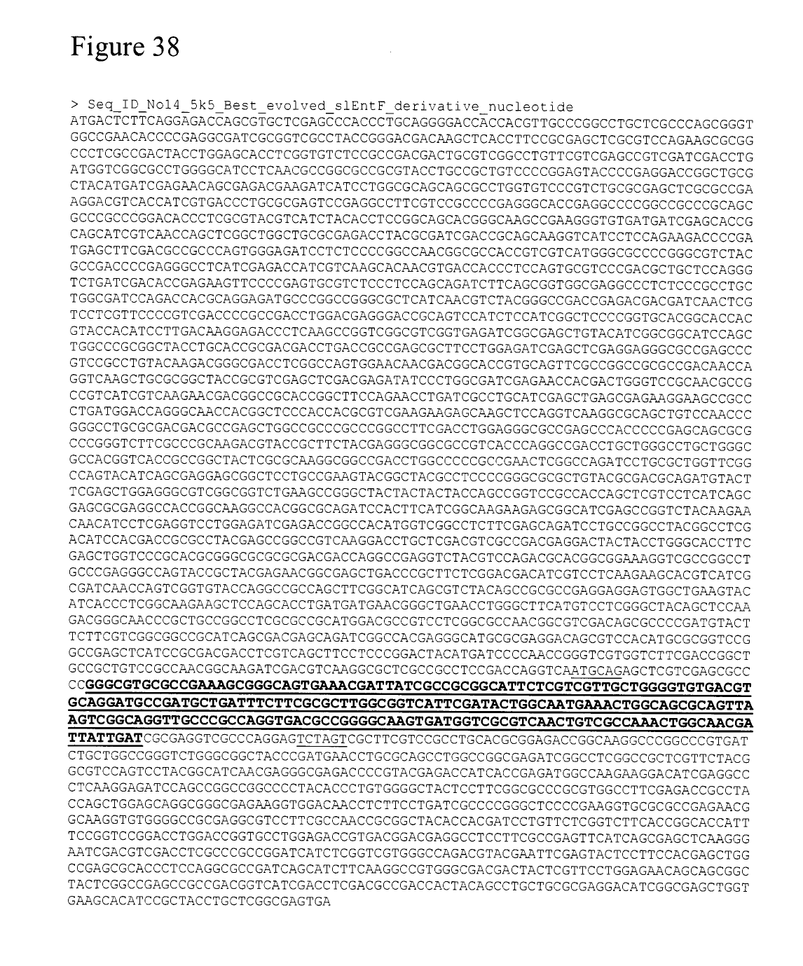

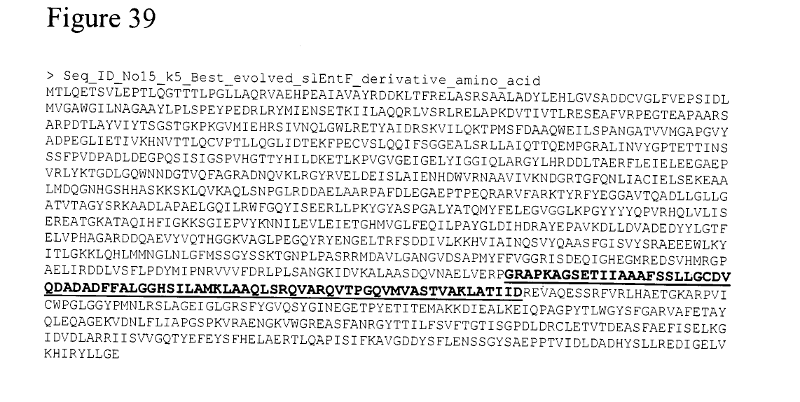

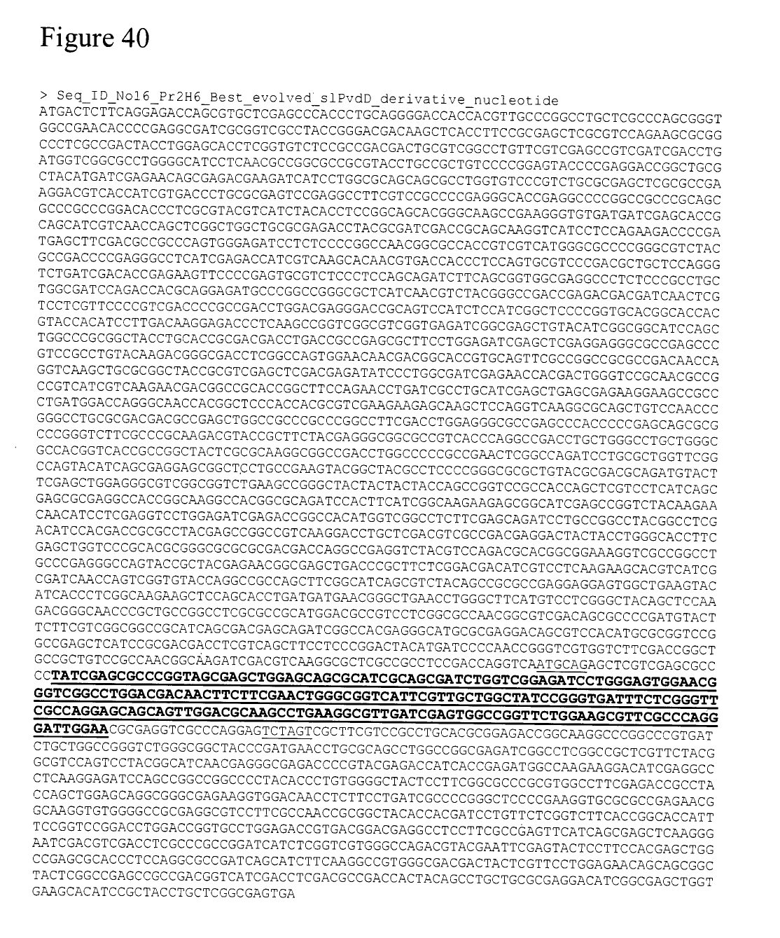

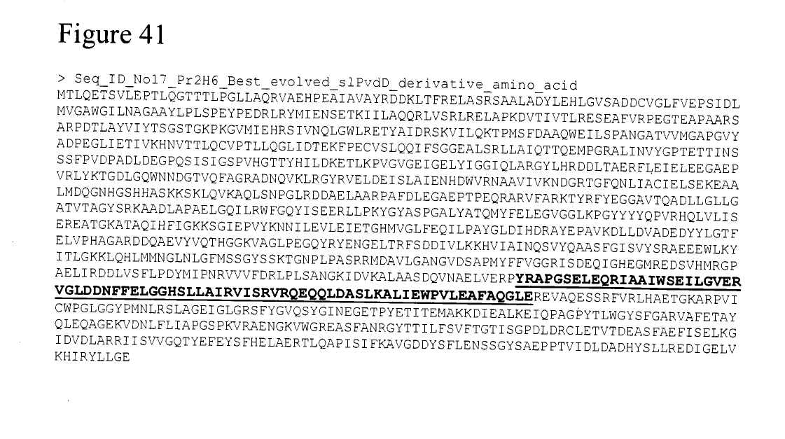

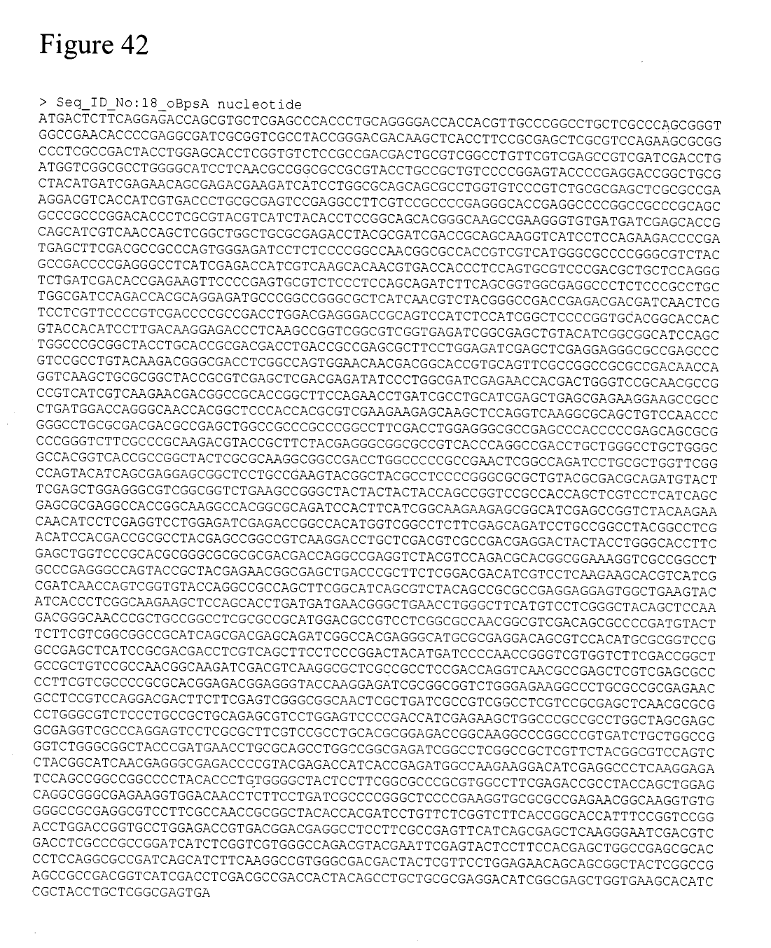

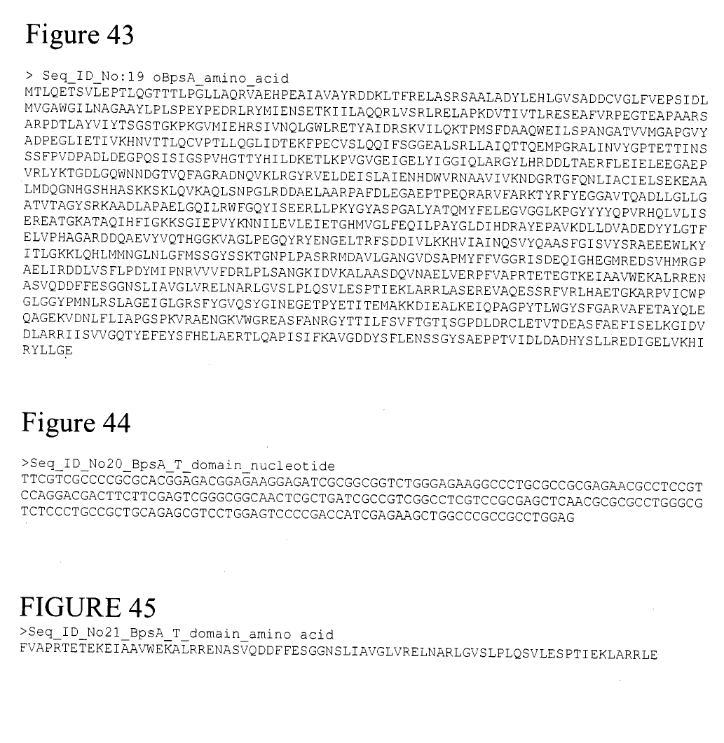

The supernatant from BpsA-expressing cultures reached a much higher A₅₉₀ (˜1.2) than the maximum achieved from any in vitro experiments (˜0.3) (not shown). Without wishing to be bound by theory, the inventors hypothesized that that since indigoidine produced and exported from a cell in vivo was effectively removed from exposure to intracellular enzymes, the loss of in vitro product may have been due to enzymatic degradation in the in vitro assay preparations. This could be due to BpsA itself, the activating PPTase or an undetected contaminating species. To test this possibility, filter sterilized culture supernatant containing indigoidine was incubated with various concentrations of each enzyme preparation used for in vitro assays. No difference in pigment levels resulted from this treatment. It was concluded that the loss of pigmentation seen in vitro was not enzyme catalysed. Each of the other assay reagents was subjected to similar analysis and it was found that only addition of sodium phosphate buffer (pH 7.8) to a final concentration of 50 mM resulted in decolouration of culture supernatant. A more detailed analysis of the relationship between pH and pigment stability was then conducted. The results of this analysis, illustrated in FIG. 5 showed that decolouration occurred to a greater extent at higher pH. This finding was consistent with previous observations that indigoidine is unstable at alkaline pH (Kuhn et al, 1965). Although the optimal pH for BpsA function is reported to be 7.8 (Takahashi et al, 2007), the sensitivity of this enzyme to changes in solution pH was now tested in the hope that it might be possible to use a lower pH buffer. As illustrated in FIG. 6A, BpsA activity was found to be very pH sensitive, with deviations of 0.2 units either side of 7.8 resulting in a severe reduction of activity. Without wishing to be bound by theory, the inventors believe that the loss of pigmentation observed corresponds to the aggregation of indigoidine to form an insoluble precipitate. Such precipitate was consistently observed in plate wells following BpsA assays. Removal of aqueous supernatant followed by resuspension of this precipitate in and equal volume of DMSO was found to yield a deep blue solution, the A₅₉₀ of which was at least two fold higher than the previously observed aqueous peak value. Without wishing to be bound by theory, the inventors believe that some precipitation of indigoidine, leading to decolouration in aqueous solution, is unavoidable at the optimal pH for BpsA function.

### 1.5—Derivation of Kinetic Parameters for BpsA

Analysis of BpsA reaction rate over a replicate two fold serial dilution series (0.78 μM-16 mM) of L-gln was conducted at both pH 7.8 (sodium phosphate) and 8.0 (Tris-Cl). Non-linear regression analysis of the resulting maximum velocity values (ΔA₅₉₀/s) revealed an excellent fit to the Michaelis Menten equation (FIG. 6B). As such the ΔA₅₉₀ was expected to be directly proportional to the concentration of holo-BpsA present in a reaction. This assumption was experimentally verified by monitoring reaction velocity at ten different concentrations of holo-BpsA (360 nM-1.14 μM) at constant L-gln/ATP concentrations; as shown in FIG. 6C the relationship between enzyme concentration and velocity is linear within this range (R²=0.989). Owing to the lack of an extinction co-efficient for indigoidine in water, coupled with formation of insoluble precipitate, we were unable to derive a k_{cat} value for BpsA. We were however able to discern a reproducible maximum velocities which are expressed with the units ΔA₅₉₀/s. Kinetic parameters for BpsA are presented in Table 1.1 below. These parameters were subsequently employed for the derivation of PPTase kinetics as outlined in section 4.11.

TABLE 1.1
Kinetic parameters for BpsA with respect to L-Gln as a variable
substrate at two different buffer compositions
Values were derived from velocity data for twelve different L-gln
concentrations (two fold serial dilution series 16-0.0078 mM). ATP
concentration was fixed at 2.5 mM. For pH 7.8 data six replicates at
each substrate concentration were analysed (n = 72) for pH 8.0 data
three replicates at each substrate concentration were analysed (n = 36)

| | Kₘ | Vₘₐₓ |
|---|---|---|
| Assay conditions | (mM) | (ΔA₅₉₀ · S⁻¹ · μM⁻¹) × 10⁻² |
| NaPh pH 7.8 | 2.43 +/− 0.12 | 1.8 +/− 0.03 |
| Tris-Cl pH 8.0 | 3.33 +/− 0.15 | 6.5 +/− 0.1 |

### 1.6—Derivation of Kinetic Parameters for PPTase Enzymes Using a BpsA Coupled Enzyme Assay

Having established a linear relationship between concentration of holo-BpsA and the apparent rate of indigoidine synthesis in aqueous solution, we next wanted to test whether it was possible to evaluate PPTase activity by using acceleration of the rate of indigoidine synthesis as a measure of the rate of 4′-PP attachment to apo-BpsA. Replicate solutions containing PPTase and all of the substrates necessary for both phosphopantetheinylation and indigoidine synthesis were established using a twofold serial dilution series of CoA (0.024-50 μM) in ninety six well plates. Reactions were initiated by addition of 0.83 μM BpsA each well of a dilution series, and absorbance at 590 nm was measured continuously. Under these conditions, the pigment synthesis reaction undergoes an initial acceleration phase as apo-BpsA is converted to holo-BpsA by the PPTase. We made the assumption that maximal acceleration occurs at the point where conversion of BpsA into the holo form is occurring most rapidly; thus, the change in slope of the curve at this point represents the maximal velocity of the PPTase-catalysed reaction (FIG. 7). FIG. 8 shows a selection of raw, intermediate and fully processed data used in the derivation of kinetic parameters for PcpS. Kinetic analysis of each PPTase with BpsA as a variable substrate was also conducted. This was achieved as previously described, except using a two-fold serial dilution series of apo-BpsA with CoA concentration set at 25 μM. The kinetic parameters for PcpS, Sfp and PP 1183 derived using this method fit well with the Michaelis Menten model (FIG. 9) and are summarized in Table 1.2, below. A more detailed description of data analysis techniques and unit conversion calculations can be found in sections 4.11 and 4.16.

TABLE 1.2
Kinetic parameters for PPTases derived using a BpsA coupled assay.
For data where CoA is the variable substrate BpsA concentration was fixed at 0.83 μM and for
all enzymes except PP1183, six replicates were analysed for twelve different CoA concentrations
(n = 72). For PP1183 nine replicates were analysed for each substrate concentration (n = 108). For
data where BpsA is the variable substrate CoA concentration was fixed at 25 μM and three
replicates were analysed at twelve different BpsA concentrations (n = 36). Errors are presented as
+/− one standard error.

| | Kₘ (μM) | | k_{cat} (min⁻¹) | | k_{cat}/Kₘ (min⁻¹ · μM⁻¹) | |
|---|---|---|---|---|---|---|
| | CoA | BpsA | CoA | BpsA | CoA | BpsA |
| PcpS | 3.79 +/− 0.31 | 6.5 +/− 1.0 | 4.47 +/− 0.1 | 41.2 +/− 3.1 | 1.18 +/− 0.12 | 6.4 +/− 1.5 |
| Sfp | 0.62 +/− 0.07 | 3.3 +/− 0.2 | 0.128 +/− 0.004 | 2.14 +/− 0.04 | 0.21 +/− 0.05 | 0.65 +/− 0.04 |
| PP1183 | 11.17 +/− 1.32 | 8.6 +/− 0.9 | 2.208 +/− 0.1 | 11.1 +/− 0.7 | 0.197 +/− 0.07 | 1.29 +/− 0.2 |

### 1.7 Estimation of Kinetic Parameters for Other Carrier Protein Substrates Using a BpsA Coupled Assay

One limitation of the approach outlined above for determination of PPTase kinetic parameters is that it only provides information about a PPTase of interest with a single carrier protein substrate (that of BpsA). For different carrier proteins, a second assay capable of providing a relative measure of k_{cat}/Kₘ for a PPTase was developed. In this assay, BpsA and an activating PPTase were incubated in the presence of different concentrations of a purified recombinant (His6 tagged) carrier protein of interest. Four carrier proteins were employed in assessed using this assay, these were: The peptide carrier protein of BpsA (bPCP), the peptide carrier protein of the first module of the Pseudomonas aeruginosa NRPS PvdD (pPCP), the peptide carrier protein of the Microcysitis aeruginosa NRPS/PKS MycG (mPCP) and the aryl carrier protein from Nostoc punctiforme protein HetM (npArCP). The concentration of CoA (250 nM) used in these experiments was one quarter of the total concentration of BpsA (1 resulting in a situation in which BpsA and the subject carrier protein were competing for a limited pool of CoA. Following incubation indigoidine formation was initiated by the addition of ATP and L-gln and A₅₉₀ measured continuously in a microplate reader. As previously, the velocity of the indigoidine synthesis reaction was indicative of the amount of apo-BpsA converted to holo-BpsA during the incubation step. By setting up these competition reactions in replicates containing a serial dilution series of carrier protein it was possible to generate IC₅₀ curves for individual carrier protein/PPTase combinations. We derived IC₅₀ values for four different carrier proteins with three different PPTases. FIG. 10 shows a representative selection of IC₅₀ curves from these experiments. All IC₅₀ values derived are presented in Table 1.3, below. For a given PPTase/carrier protein the derived IC₅₀ values indicates the concentration of carrier protein that is required to compete for 50% of the available CoA. In order for the carrier protein to compete for 50% of the available CoA its average velocity of modification must be equal to the velocity of BpsA modification. Our method therefore provides a means of assessing velocity of carrier protein modification, a property which is dependent on k_{cat}/Kₘ for the PPTase-carrier protein interaction. By themselves, the derived IC₅₀ values provide information about velocity of carrier protein modification relative to BpsA, in order to convert these to absolute values we used the previously determined k_{cat} and Kₘ values for each PPTase with BpsA as a variable substrate as outlined in section 4.16.2. All IC₅₀ values were converted to estimates of k_{cat}/Kₘ for each PPTase-carrier protein combination; these are presented in Table 1.3, below.

TABLE 1.3
Relative affinities of PcpS, Sfp and PP1183 for four different carrier proteins
IC₅₀ values for isolated carrier protein mediated inhibition of BpsA activation are given. For all
PPTase/Carrier protein combinations six replicates were analysed for twelve different substrate
concentrations (n = 72). Ninety five percent confidence intervals are given in parentheses. k_{cat}/Kₘ
values given are estimates derived as outlined in section 4.16.2.

| | PcpS | | Sfp | | PP1183 | |
|---|---|---|---|---|---|---|
| | IC₅₀ | k_{cat}/Kₘ | IC₅₀ | k_{cat}/Kₘ | IC₅₀ | k_{cat}/Kₘ |
| | (μM) | (Min⁻¹ · μM⁻¹) | (μM) | (Min⁻¹ · μM⁻¹) | (μM) | (Min⁻¹ · μM⁻¹) |
| bPCP | 0.71 | 7.77 | 0.72 | 0.69 | 0.30 | 3.88 |
| | (0.66-0.77) | (7.22-8.35) | (0.68-0.76) | (0.64-0.73) | (0.25-0.36) | (3.66-4.58) |
| pPCP | 0.56 | 9.84 | 0.45 | 1.11 | 0.28 | 4.10 |
| | (0.50-0.63) | (8.80-11.0) | (0.42-0.48) | (1.03-1.19) | (0.25-0.32) | (3.24-4.64) |
| mPCP | 0.99 | 5.55 | 0.15 | 3.33 | 0.18 | 6.30 |
| | (0.86-1.15) | (4.80-6.42) | (0.14-0.16) | (3.14-3.54) | (0.16-0.21) | (5.55-7.16) |
| npArCP | 8.46 | 0.65 | 0.14 | 3.51 | 6.0 | 0.19 |
| | (7.11-10.06) | (0.54-0.78) | (0.13-0.15) | (3.32-3.70) | (4.74-7.59) | (0.24-0.15) |

### 1.8 Evaluation of PPTase Inhibition by 6-Nitroso-1,2-Benzopyrone

A BpsA coupled assay for evaluation of PPTase inhibitors provided was tested using the previously identified Sfp inhibitor 6-nitroso-1,2-benzopyrone (6-NOBP; FIG. 11). Each of the three PPTases was evaluated for inhibition across a two-fold serial dilution series of 6-NOBP (250-0.12 μM) with CoA concentration fixed at either 10 or 2.5 μM. As summarized in FIG. 11 and Table 1.4 (below), all three PPTases were inhibited by this compound, with IC₅₀ values ranging from 9.1-10.8 μM for the CoA concentration set at 5 μM; and 2.1-4.0 μM for the CoA concentration set at 1.25 μM. Also found was that pre-activated holo-BpsA was also inhibited by 6-NOBP, although inhibition of this NRPS in isolation was less than for the co-incubated PPTase/apo-BpsA reactions (IC₅₀ value for holo-BpsA of 29.2 μM). As outlined in Table 1.4 (below), conversion of IC₅₀ values using the previously established Kₘ values for each of the three PPTases results in K, values for 6-NOBP ranging from 0.5-7.5 μM for each enzyme. Derived Kᵢ values were most consistent between CoA concentrations when the Cheng-Prusoff equation for competitive inhibition was employed. It is worth noting that the Kᵢ values derived from the 1.25 μM CoA data sets are consistently lower than those derived from the 5 μM CoA data sets (P<0.05). This may indicate that 6-NOBP is not a competitive inhibitor, or alternatively may be an artefact arising from the co-inhibition of BpsA.

TABLE 1.4
Inhibition of PPTases by 6-NOBP
IC₅₀ values were derived using four parameter dose response function of
Graphpad Prism and converted to Ki values by substituting the previously
established Km values for each PPTase into the Cheng-Prusoff
equation for competitive inhibition. Ninety five percent confidence
intervals are given in parentheses.

| | IC₅₀ (μM) | | Kᵢ (μM) | |
|---|---|---|---|---|
| | 1.25 μM CoA | 5 μM CoA | 1.25 μM CoA | 5 μM CoA |
| PcpS | 4.049 (3.5-4.6) | 10.79 (8.6-13) | 3.1 (2.7-3.6) | 4.7 |
| | | | | (3.7-5.6) |
| Sfp | 2.113 (1.7-2.7) | 9.064 (8.4-9.8) | 0.7 (0.55-0.89) | 1.0 |
| | | | | (0.92-1.1) |
| PP1183 | 3.703 (3.3-4.2) | 9.805 (8.8-10.9) | 3.3 (3.0-3.7) | 6.8 |
| | | | | (6.1-7.5) |

1.9 Recovery of 6-NOBP and Novel PcpS Inhibitors from the LOPAC¹²⁸⁰ Compound Library

To demonstrate utility of the BpsA coupled assay in high throughput screening to recover PPTase inhibitors from chemical libraries, BpsA and PcpS were combined in a reaction master mix as described in section 4.17.2 and high-throughput liquid handling robots from the Victoria University of Wellington Chemical Genetics Suite were used to screen a subset of the LOPAC¹²⁸⁰ compound library to identify compounds that could inhibit PcpS. A graphical representation of the assay for a representative 96-well plate that included three negative control wells (no PPTase added), three positive control wells (DMSO added in place of a test inhibitor), 6-NOBP, and 79 other test compounds is pictured in FIG. 12. In this figure it can clearly be seen that 6-NOBP and one other unidentified weak PcpS inhibitor have been identified, together with an unidentified strong inhibitor of PcpS. Thus, the BpsA coupled assay is clearly effective in high throughput screening to recover PPTase inhibitors from chemical libraries. To further exemplify this, the novel PcpS inhibitor Bay11-7085 was recovered on a different plate (not shown) and tested across a range of inhibitory concentrations. The IC₅₀ curve for this compound is pictured in FIG. 13A. Although the K, for this compound for inhibition of PcpS has not yet been calculated, qualitatively it is clearly a stronger inhibitor of PcpS than 6-NOBP (FIG. 13B).

### Example 2

### Recovery of PPTase and Secondary Metabolite Genes from a Soil Derived Small Insert Environmental DNA Library Using the Unmodified bpsA Gene as a Reporter

### 2.1—Overview:

As previously described in example 1, the BpsA protein of S. lavendulae is reliant on activation by an exogenous PPTase for function in E. coli. As a result, the gene for BpsA, when expressed in E. coli, has the potential to serve as colorimetric reporter for the discovery of PPTases in eDNA libraries of undefined composition and diversity. Existing evidence suggests that PPTase enzymes are often located within or near secondary metabolite biosynthetic gene clusters where they serve to activate NRPS and PKS enzymes (Marahiel et al, 1997). As such BpsA also has the potential to serve as a reporter for discovery of novel secondary metabolite clusters (SMCs) by association with a PPTase gene. To demonstrate the utility of BpsA as a reporter for recovery of PPTase genes and associated secondary metabolite biosynthesis clusters a protocol for high throughput screening of eDNA was developed and used to recover novel PPTase genes from a soil derived small insert eDNA library. This library was exhaustively screened using the developed protocol and three complete PPTase genes were recovered. The eDNA derived fragment on which one of these genes was located was also found to contain partial sequences that were clearly indicative of an unknown secondary metabolite biosynthesis cluster (SMC markers).

2.2—Recovery of PPTase Genes and Associated SMC Markers from a Previously Constructed Soil Derived eDNA Library

For screening purposes, an E. coli strain harbouring a plasmid-encoded copy of the bpsA gene (Plasmid slBpsA) was used as a reporter. The eDNA library used for these experiments was provided by our collaborator Dr Nadia Skorupa Parachin of Lund University and is henceforth referred to as eDNA library 1. The library contained small insert genomic DNA fragments in the plasmid pRSET1 (Parachin and Gorwa-Grauslund, 2011). The genomic DNA was isolated from a soil sample and prepared so that the size inserts would be between 500 and 3000 bp. Library DNA was delivered to reporter cells by electroporation as described in Section 4.7.3.2 and approximately 3×10⁶ of the resulting clones were assessed for colour development on pigment production or autoinduction agar as described in Section 4.5 and 4.13. A total of 30 hits were identified from six independent transformations of eDNA library 1 and the plasmid-encoded eDNA fragment from these was isolated for sequence analysis as described in Section 4.14. FIG. 14 illustrates schematically how BpsA can be used to screen eDNA libraries for PPTase genes. FIG. 15 shows a representative screening plate on which a hit was recovered.

### 2.3—Sequence Analysis and Gene Identification

Isolated plasmid DNA from each of the hits recovered was sequenced using both the T7 promoter and T7 terminator primers. These primers anneal to priming sites that flank the region into which eDNA fragments are inserted into pRSET1 during library construction. This resulted in two sequence files for each hit, which were in most cases non-overlapping. Standard primer walking techniques were then used to obtain the remainder of the sequence for each insert. Preliminary analysis of the sequence files by alignment revealed that three unique eDNA fragments had been recovered from eDNA library 1 multiple times. A contiguous sequence file was assembled for each of these three hits (Table 2.1). Each of the unique nucleotide sequences was translated in all six reading frames and the resulting amino acid sequences analysed by BLAST search against the non-redundant protein sequence collection of NCBI. The translation and BLAST search were conducted using the online tool BLASTX. Sequence analysis enabled the identification of PPTase genes for all three fragments, as well as additional partial and complete genes isolated by virtue of proximity to the PPTase genes. The organisation and identity of partial and complete genes identified in each eDNA fragment is illustrated in FIG. 16 and Table 2.1. Of particular interest were two partial genes recovered on fragment 1, which indicated that of part of a PKS secondary metabolite cluster had been recovered.

TABLE 2.1^{†}
partial and complete genes found in eDNA fragments

| | Fragment | Top match from BLAST | Species from which top | Percentage |
|---|---|---|---|---|
| Gene | number | search | match was found | identity |
| TE | 1 | PKS KS-domain - partial | [symbol] sp. CCY0110 | 52% |
| PPTase 1 | 1 | 4′-phosphopantetheinyl | [symbol] | 43% |
| | | transferase | | |
| KS | 1 | PKS KS-domain - partial | [symbol] sp. CCY0110 | 52% |
| DUS | 2 | Dihydrouridine synthase | Nocardia farcinica | 55% |
| PPTase 2 | 2 | 4′-phosphopantetheinyl | Streptomyces sviceus | 35% |
| | | transferase | | |
| NTR | 2 | Nitroreductase | Nitrococcus mobilis | 50% |
| Mg | 3 | Magnesium Transporter | Xanthomonas campestris | 55% |
| PPTase 3 | 3 | 4′-phosphopantetheinyl | Burkholderia oklahomensis | 40% |
| | | transferase | | |

^{†}Abbreviated gene names are given in the left hand column, with FIG. 16 illustrating the layout of these genes on the three eDNA fragments. Fragments containing likely SMC marker genes are annotated in bold.

### Example 3

### Generation of Modified BpsA Derivatives and Their Application in Screening of Environmental DNA Libraries

### 3.1—Overview:

Existing biochemical evidence shows that not all PPTases are capable of recognising and activating all NRPS T-domains (Mootz et al, 2001). As such, the inventors hypothesized that alternative BpsA reporters in which the native T-domain of the enzyme is substituted for a foreign T-domain would be useful for enabling identification and recovery of a greater diversity of PPTase genes and associated SMC genes from environmental DNA samples. Described herein are experiments in which five modified derivatives of BpsA (mBpsAs) were generated by substituting the region of the bpsA gene encoding the T-domain, for T-domain nucleic acid sequences derived from other NRPS genes. In all cases these substitutions resulted in severe reduction or complete ablation of pigment synthesis capacity of the resulting recombinant enzyme in vivo. In order to restore function to the mBpsAs generated in domain swapping experiments, a directed evolution protocol was developed and implemented. This protocol, which relied on random mutagenesis of substituted T-domains coupled with selection for improved pigment synthesis in vivo, allowed improvement of the in vivo pigment synthesis capacity of the two modified BpsA derivatives to which it was applied. Two resulting evolved modified BpsA derivatives (emBpsAs) were subsequently used to re-screen an environmental DNA library which had already been exhaustively screened using a wild type BpsA reporter. Re-screening with this alternative reporter resulted in recovery of a set of seven hits, four of which were not found by previous screening with wild type BpsA. This experiment demonstrates the utility of emBpsA reporters for accessing greater diversity in eDNA libraries. The directed evolution protocols developed provide a means for generating many emBpsA derivatives that could be employed as alternative reporters in library screens. We also constructed and screened a second soil eDNA library, recovering 13 clones bearing genes with shared identity with known PPTases. Five of these clones strongly appeared to have been derived from secondary metabolite clusters, while others carried open reading frames encoding hypothetical proteins with no homology to any previously annotated proteins and may also have been derived from SMCs. We also recovered one clone bearing a single open reading frame that has no homology to known PPTases, but appears to be able to activate BpsA. This demonstrates that our eDNA screening method may also have utility in discovery of previously unknown PPTase families. In total we have now used wtBpsA and emBpsA derivatives to recover 21 hits from two soil eDNA libraries, at least 7 of which have clearly been derived from SMCs. This proves the utility of our genetic reporter system for enrichment of SMC-derived clones from metagenomic libraries.

### 3.2—Generation of Modified BpsA Derivatives by T-Domain Substitution

A detailed description of the method used for generation of mBpsA derivatives is given in Section 4.15. Briefly, a plasmid containing bpsA lacking its native T-domain (pBPSA3) was created. This plasmid contained restriction enzyme sites which were subsequently used to introduce foreign T-domain sequence. Foreign T-domains were then amplified from the chromosomal DNA of their host organisms and introduced into pBPSA3 to generate mBpsA derivatives which contained T-domains from foreign NRPS proteins in place of the native T-domain. Only the final embodiment of the T-domain swapping platform is described in this application. This embodiment is the result of rational in-silico design as well as trial and error, which are not described. Using this final platform, five different mBpsA derivatives were generated and subsequently assessed for in vivo pigment synthesis capacity in E. coli harbouring an activating PPTase. Table 3.1 gives a summary of the mBpsA derivatives generated as well as their in vivo pigment synthesis activities relative to wild type BpsA. The derivation of in vivo pigment synthesis activity was as described in 4.10.

TABLE 3.1
Summary of mBpsA derivatives generated in this study

| Name | Source of foreign T-domain sequence | Relative activity |
|---|---|---|
| slEntF | entF gene from E. coli W3110 | 5.14 +/− 0.28% |
| slPvdD1 | First module of pvdD gene from | 0% |
| | P. aeruginosa PAO1 | |
| slPvdD2 | Second module of pvdD gene from | ~5% |
| | P. aeruginosa PAO1 | |
| slPsT1 | Second module of pspph1926 gene of | ~5% |
| | P. syringae 1448a | |
| slDhbF | First module of dhbF gene from B. subtilis | 0% |
| | sub sp. subtilis | |

3.2—Development of a High Throughput Screening Process for Directed Evolution of Recombinant bpsA Genes

As outlined in Table 3.1, substitution of the native T-domain of BpsA for a foreign domain consistently reduced the efficiency of pigment synthesis in vivo. This result agrees with published structural and biochemical data which suggests that T-domains undergo specific interactions with other NRPS domains during the biosynthetic process (Koglin et al, 2006; Lai, Fischbach et al, 2006; Lai, Koglin et al. 2006; Zhou et al, 2006; Samel et al, 2007; Doekel et al, 2008; Frueh et al, 2008; Tanovic et al, 2008). Having established and tested a T-domain swapping platform, directed evolution experiments were conducted with the aim of improving function of mBpsA enzymes and identifying key residues involved in T-domain interaction with other BpsA domains. To achieve this, the substituted T-domains of these variants were randomly mutated using error prone (ep) PCR. The resulting products were then cloned into the BpsA T-domain swapping construct pBpsA3 to give a mutant library varying only in the sequence of the T-domain. Development of a robust, high throughput first tier screening procedure allowed qualitative identification of variants with improved T-domain function in BpsA. Improved variants were then subjected to a second tier screen to quantitatively assess pigment synthesis capacity in vivo. Based on the results of the second tier screen, selected improved clones were sequenced and the mutations responsible for improved function were determined. The process used for directed evolution of mBpsA derivatives is illustrated schematically in FIG. 17.

### 3.2.1—Development of a First Tier Agar Plate Based Screening System.

To develop a first tier screen for improved pigment synthesis capacity, priority was given to screening as many clones as possible, as quickly as possible and with a minimum of manual input. Avoidance of false positives and quantitative assessment of activity were given low priority as these issues could be addressed in a second, lower throughput screen. At this stage it had already been established that pigment production was readily detectable in BpsA expressing E. coli patched on to agar plates, and that even mutants with low in vivo pigment synthesis activity (<0.7% of WT) could be detected following 24-48 h incubation on pigment development agar. To facilitate screening however, pigmentation would have to be visible in single colonies immediately following transformation. This was now assessed.

Initial trials were run using E. coli harbouring a P. putida PPTase expression plasmid. A plasmid for the expression of oBpsA (a BpsA mutant with 21.7+/−2.7% of the wild type enzyme) was transformed into this background and plated on agar containing 100 mM L-Gln and 0.5 mM IPTG. Under these conditions, it was consistently found that no transformants could be recovered. If, however, IPTG was omitted from the plates, transformation with high efficiency was achieved. This result suggested that toxicity arising from indigoidine production rendering transformants non-viable in the presence of IPTG.

To circumvent this problem, a novel system for on-plate induction was developed. Transformed cells were plated on media containing antibiotics and L-Gln only, and incubated overnight to allow colonies to develop. Following colony development the entire agar slab was scooped from the plate and rested on the sterile lid. IPTG solution was then spread on the bottom of the empty plate and the agar slab replaced on top of the solution. Diffusion of IPTG into the medium then resulted in induction of protein synthesis in colonies on the top surface of the agar and development of strong pigmentation in individual colonies as illustrated in FIG. 18. We note that autoinduction medium as we describe in section 4.5 could equally have been used for this purpose.

### 3.2.2—Optimisation of Library Generation for Directed Evolution

An important consideration for directed evolution is the development of a reliable system for library generation. In this case libraries were generated by first conducting epPCR using a particular T-domain as template. The resulting PCR product was then introduced, via compatible restriction sites, into a plasmid borne copy of the bpsA gene, resulting in a library of bpsA genes with variant T-domain sequence. The most critical factor in this process is generation of high quality, restriction digested linear vector into which a digested PCR product can be introduced by ligation. In order to ensure that vector preparations for directed evolution were of consistently of high quality, a standardized protocol for preparation and quality assessment of digested linear vector was developed. This allowed consistent generation of libraries in which greater than 70% of plasmids contained the desired insert. The protocols developed for generation and quality assessment of vector for directed evolution are described in Sections 4.9.1.1 and 4.9.1.2 respectively.

3.3—Improvement of slPvdD1 Function by Directed Evolution

The mBpsA derivative slPvdD1 contains the T-domain from the first module of the P. aeruginosa PAO1 gene pvdD substituted in place of the native T-domain of BpsA. This substitution was found to be result in completely inactive enzyme. E. coli harbouring a plasmid for expression of slPvdD and an activating PPTase were unable to produce any discernable blue pigment on pigment development agar or in pigment development broth regardless of incubation time, activating PPTase or temperature as assessed by comparison to the same strain harbouring an empty plasmid in place of slPvdD1 (not shown). A library of slPvdD variants was generated by error prone PCR as described in Section 4.9.1 and an estimated 5-6×10⁵ clones were screened using an the agar plate based screening process described in Section 4.9.2.

For each hit recovered from the agar based screen, the time for pigmentation to develop was noted and only the fastest developing hits from each library were chosen for further assessment. A total of 44 putative improved variants of slPvdD recovered from the first tier screen were subjected to second, quantitative tier of screening. The second tier screening process, which is described in detail in Section 4.9.3, allowed accurate assessment of in vivo pigment synthesis levels for each clone by measurement of pigment levels in the supernatant of cultures grown in 96 well plates. Pigment levels were assessed by measuring supernatant absorbance at 590 nm. FIG. 19 illustrates the results from this second tier of screening graphically and indicates the improved clones for which sequence data was obtained. The most active clone recovered from improvement of slPvdD, designated 3kF0, was chosen for further improvement by a second round of epPCR and selection.

### 3.3.1—Second Round Evolution of SlPvdD1

For second round evolution the T-domain of clone 3kF0 was used as template for the epPCR reaction instead of wt PvdD T-domain. Transformation and screening were conducted as for the first round of evolution, and 22 hits were recovered from an estimated total of 600,000 clones screened. Screening proved more difficult than for the first round of evolution due to the higher levels of pigmentation of unimproved clones. Selection of improved clones required qualitative appraisal of relative pigmentation by naked eye, including subjective assessment of local variations in induction, presumably arising due to uneven diffusion of IPTG. Nonetheless, the number of false positives recovered proved to be low with 14 of the 22 hits recovered showing a clear improvement in function in the second tier of the screen (FIG. 20).

3.4—Improvement of slEntF Function by Directed Evolution

The mBpsA derivative slEntF contains the T-domain from the E. coli w3110 gene entF substituted in place of the native T-domain. This substitution was found to reduce in in vivo pigment synthesis activity to 5.14% of wild type BpsA. A library of slPvdD variants was generated by error prone PCR as described in Section 4.9.1 and an estimated 4-5×10⁵ clones were screened using an the agar plate based screening process described in Section 4.9.2. As for the second round evolution of slPvdD, the fact that unimproved slEntF exhibited some pigmentation on pigment development agar made selection of improved clones more difficult than for the other first round evolution experiments described in this chapter. Nonetheless, the screening procedure again proved to be robust, with 39 of the 46 hits recovered from the first tier showing elevated pigment synthesis in the second tier (FIG. 21). Of the 39 improved clones recovered, the sequence of 8 was determined. The most active of these was designated ENTFR1 and used as an emBpsA reporter for further library screening.

3.5—Re-Screening of eDNA Library 1 Using Evolved Modified BpsA Reporters

In order to determine whether screening and eDNA library with alternative BpsA reporters would lead to the recovery of previously undetected hits, eDNA library 1 which had already been exhaustively screened with wt BpsA was subjected to reassessment using two evolved modified BpsA reporters. The reporters used were the modified BpsA derivative isolated from PR2H6 (an emBpsA resulting from substitution of the first T-domain of pvdD in place of the native T-domain of bpsA, followed directed evolution; see FIG. 20); and ENTFR1 as described above. Screening was carried out as previously described, except library DNA was transformed into electrocompetent E. coli BL21 (DE3) cells harbouring a plasmid borne copy of PR2H6 or ENTFR1 instead of wtBpsA. Similar numbers of clones were assessed for each emBpsA reporter, as previously for the wt BpsA reporter (section 2.2). In total, 28 blue hits were recovered using the PR2H6 reporter, and 60 using the ENTFR1 reporter.

3.6—Sequence Analysis of Hits Recovered from Screening eDNA Library 1 Using Evolved Modified BpsA Reporters

Isolated plasmid DNA containing eDNA fragments from each of the hits was subjected to sequencing analysis using the T7 promoter and terminator primers. Sequences were completed by primer walking and sequencing files were then analysed using BLASTX as previously described in Section 2.3. The results from this analysis are summarized in Table 3.2 and FIG. 22. This analysis revealed that a total of five different eDNA fragments had been recovered using the PR2H6 reporter; and seven different eDNA fragments had been recovered using the ENTFR1 reporter. Each emBpsA reporter recovered the same fragments 1-3 that had previously been identified using the wt BpsA reporter (FIGS. 16 and 22); the PR2H6 reporter recovered two additional fragments (numbered 4 and 7; FIG. 22); and the ENTFR1 reporter recovered all of the same fragments as the PR2H6 reporter as well as two additional fragments (numbered 5 and 6; FIG. 22).

The organisation and identity of partial and complete genes identified in each eDNA fragment is illustrated in FIG. 22 and Table 3.2. In addition to fragment 1, previously noted as containing a partial PKS gene, fragment 7 showed strong evidence of being derived from an NRPS secondary metabolite cluster.

TABLE 3.2^{†}
annotation of partial and complete genes found in eDNA fragments from
eDNA library 1

| | | | | Reporter strain found in | | |
|---|---|---|---|---|---|---|
| | Top match from BLAST | Species from which top match | % | BpsA | | |
| Gene | search | was found | identity | wt | PR2H6 | ENTFR1 |
| TE | PKS KS-domain - | [symbol] sp. CCY0110 | 52% | Y | Y | Y |
| | partial | | | | | |
| PPTase 1 | 4′-phosphopantetheinyl | [symbol] | 43% | | | |
| | transferase | | | | | |
| KS | PKS KS-domain - | [symbol] sp. CCY0110 | 52% | | | |
| | partial | | | | | |
| DUS | Dihydrouridine synthase | Nocardia farcinica | 55% | Y | Y | Y |
| PPTase 2 | 4′-phosphopantetheinyl | Streptomyces sviceus | 35% | | | |
| | transferase | | | | | |
| NTR | Nitroreductase | Nitrococcus mobilis | 50% | | | |
| Mg | Magnesium Transporter | Xanthomonas campestris | 55% | Y | Y | Y |
| PPTase 3 | 4′-phosphopantetheinyl | Burkholderia oklahomensis | 40% | | | |
| | transferase | | | | | |
| PPTase 4 | 4′-phosphopantetheinyl | Agrobacterium vitus | 42% | N | Y | Y |
| | transferase | | | | | |
| SuHy | Sugar hydrolase | Stigmatella aurantiaca | 26% | | | |
| PHOS | phosphodiesterase | Dyadobacter fermentans | 38% | | | |
| PPTase 5 | 4′-phosphopantetheinyl | Microcoleus vaginatus FGP-2 | 39% | N | N | Y |
| | transferase | | | | | |
| Ac | adenylyl cyclase class- | Ralstonia eutropha JMP134 | 46% | | | |
| | 3/4/guanylyl cyclase | | | | | |
| ICD | putative integrase core | uncultured marine | 45% | | | |
| | domain protein | microorganism | | | | |
| 3-B-HSD | 3-beta-hydroxysteroid | Geobacter bemidjiensis Bem | 59% | N | N | Y |
| | dehydrogenase/ | | | | | |
| | isomerase family protein | | | | | |
| PPTase 6 | acyl carrier protein 4′- | Geobacter bemidjiensis Bem | 42% | | | |
| | phosphopantetheinyl | | | | | |
| | transferase | | | | | |
| HYP 1 | hypothetical protein | [symbol] SD1 | 34% | N | Y | Y |
| | PDI_0903 | | | | | |
| PPTase 7 | 4′-phosphopantetheinyl | [symbol] | 51% | | | |
| | transferase | ATCC 51888 | | | | |
| OciA | OciA (an NRPS) | [symbol] NIVA- | 53% | | | |
| | | CYA 116 | | | | |

^{†}Fragments are numbered according to the PPTase numbering indicated in each group of rows (e.g. PPTase 1 is on fragment 1;
PPTase 4 is on fragment 4). Abbreviated gene names are given in the left hand column, with FIG. 22 illustrating the layout of these genes on the seven eDNA fragments. Fragments containing likely SMC marker genes are annotated in bold. The three right-most columns indicate which reporter strain each fragment was recovered from.

3.7—Testing cross-reactivity of different eDNA clones in each reporter strain

Of the seven PPTase clones recovered from eDNA library 1, only three were identified using the wt BpsA reporter strain, and five with the PR2H6 reporter strain. Based on the predicted number of unique clones in eDNA library 1 (1.3×10⁵; Parachin and Gorwa-Grauslund, 2011) this library should have been screened with ˜20× coverage in each reporter strain. However, if this estimate of diversity was not accurate, or if certain clones were heavily over-represented relative to others, the differential recovery of clones using different reporters may have been due to random statistical variation rather than differential specificities of each reporter. To test this, each of the seven PPTase-containing eDNA clones recovered from eDNA library 1 was transformed into each of the three bpsA reporter strains. Cultures of transformed cells were diluted to a concentration of 500-3000 cfu/ml and 100 μl of each was plated in duplicate onto identical autoinduction agar plates. The time taken for blue colour to develop in colonies of each reporter strain was recorded qualitatively. The results of this “relative activity” analysis are portrayed in Table 3.3 below, with three ticks representing rapid development of blue coloration; two ticks moderate development of blue coloration; one tick slow/faint development of blue coloration; and a cross indicating that the PPTase carried by that clone was unable to induce formation of indigoidine in a given reporter strain.

TABLE 3.3
Cross-reactivity of different eDNA clones in each reporter strain

| | Reporter strain | | | | | |
|---|---|---|---|---|---|---|
| eDNA | found in | | | Relative activity in | | |
| clone | wt | | | each reporter strain | | |
| number | BpsA | PR2H6 | ENTFR1 | wt BpsA | PR2H6 | ENTFR1 |
| 1 | Y | Y | Y | ✓✓✓ | ✓✓ | ✓ |
| 2 | Y | Y | Y | ✓✓✓ | ✓✓✓ | ✓✓ |
| 3 | Y | Y | Y | ✓✓✓ | ✓✓ | ✓ |
| 4 | N | Y | Y | ✓✓ | ✓✓✓ | ✓✓ |
| 5 | N | N | Y | ✓ | ✓ | ✓ |
| 6 | N | N | Y | x | x | ✓ |
| 7 | N | Y | Y | x | ✓✓ | ✓ |

Our cross-reactivity analysis confirmed that our emBpsA reporters did indeed exhibit different specificities for different PPTases, enabling recovery of eDNA clones that would not have been recoverable using the wt BpsA reporter alone. However, this analysis also indicated that we had failed to recover two clones (eDNA clone numbers 4 and 5) that had moderate or slow/faint levels of indigoidine production with the wt BpsA reporter. This may have been because coloration of colonies by these two clones was substantially slower than for clones 1-3 and plates were monitored for an insufficient duration, or may have simply been due to chance variation.
3.8—Construction and preliminary screening of a second soil derived eDNA library

Following the successful screening of the library provided by Lund University, a second eDNA library was constructed in our laboratory. The eDNA used to construct this library was extracted from soil samples collected from a residential property in Wellington, New Zealand. Total community DNA was extracted and purified from the soil samples as described in Section 4.12.1. Following purification the DNA was partially digested with DNase A and fragments between 1-2 kb recovered by agarose gel electrophoresis and electro elution as described in Section 4.12.2. The fractionated DNA was then used to construct a plasmid based library (henceforth referred to as eDNA library 2) as described in Section 4.12.2-4.12.3. Library DNA was delivered to E. coli BL21(DE3) cells harbouring one of the three BpsA reporter plasmids as described in Section 4.7.3.2 and approximately 10⁶ of the resulting transformants were assessed for colour development on pigment production or autoinduction agar as described in Sections 4.5 and 4.13.

Although not yet quantified, eDNA library 2 appears to contain a much higher level of diversity than eDNA library 1. To date we have recovered a total of 13 unique clones that contain a gene with shared identity with a known PPTase (hits 1-13, FIG. 23). Five of these clones also bear genes that share identity with known secondary metabolite genes, strongly suggesting that these fragments were derived from a SMC (hits 1, 3, 4, 6, 7, FIG. 23; Table 3.4). This proves the utility of our genetic reporter system for enrichment of SMC-derived clones from metagenomic libraries. We also recovered an eDNA clone with a single open reading frame that bears no homology to any known PPTase (hit 14, FIG. 23; Table 3.4). This indicates that our eDNA screening method may also have utility in discovery of previously unknown PPTase families.

TABLE 3.4^{†}
annotation of partial and complete genes found in eDNA fragments from
eDNA library 2

| | | | | Reporter strain found in | | |
|---|---|---|---|---|---|---|
| | Top match from BLAST | Species from which top | Percentage | BpSA | | |
| Gene | search | match was found | identity | wt | PR2H6 | ENTFR1 |
| SimX4 | SimX4-like protein | [symbol] | 76% | N | Y | N |
| PPTase 1 | Phosphopantetheinyl | [symbol] | 67% | | | |
| | transferase | | | | | |
| HYP 1 | Hypothetical protein 1 | [symbol] | 77% | | | |
| HYP 2 | Hypothetical protein 2 | Leishmania infantum | 27% | Y | Y | Y |
| PPTase 2 | 4′-phosphopantetheinyl | Nostoc punctiforme | 39% | | | |
| | transferase | | | | | |
| CoA | long-chain-fatty-acid-- | Bacillus pseudofirmus | 42% | | | |
| | CoA ligase | | | | | |
| CLIP | CLIP-associating protein 1 | [symbol] | 33% | N | Y | N |
| PPTase 3 | 4′-phosphopantetheinyl | [symbol] | 45% | | | |
| | transferase | [symbol] | | | | |
| AufC | Polyketide Synthase | [symbol] | 32% | | | |
| CPT | cyclic peptide | [symbol] sp. | 63% | Y | Y | Y |
| | transporter subfamily | | | | | |
| PPTase 4 | 4′-phosphopantetheinyl | [symbol] | 50% | | | |
| | transferase | | | | | |
| HYP 3 | Hypothetical protein 3 | [symbol] | 40% | | | |
| PPTase 5 | 4′-phosphopantetheinyl | Roseovarius nubinhibens | 38% | Y | Y | N |
| | transferase | | | | | |
| UPGD | undecaprenyl-phosphate | Lautropia mirabilis | 61% | | | |
| | glucose | | | | | |
| | phosphotransferase | | | | | |
| NAG | NacetylglucosaminyldiphosphoundecaprenolN- | [symbol] | 51% | N | Y | N |
| | acety 1-beta-D- | [symbol] | | | | |
| | mannosaminyl | | | | | |
| | transferase | | | | | |
| PPTase 6 | 4′-phosphopantetheinyl | [symbol] | 41% | | | |
| | transferase | K12 | | | | |
| NRPS A | amino acid adenylation | [symbol] | 50% | | | |
| | domain protein | | | | | |
| GLYC | glycosyltransferase | [symbol] | 58% | N | Y | N |
| PPTase 7 | 4′-phosphopantetheinyl | [symbol] | 41% | | | |
| | transferase | | | | | |
| PPTase 8 | 4′-phosphopantetheinyl | Microcoleus | 45% | Y | Y | N |
| | transferase | chthonoplastes | | | | |
| HYP 5 | hypothetical protein | Ornithorhynchus anatinus | 36% | | | |
| PilT | twitching motility protein | Nitratiruptor sp. | 37% | Y | Y | N |
| | PilT | | | | | |
| Secr | type II secretion system | Desulfotomaculum | 40% | | | |
| | protein E | nigrificans | | | | |
| PPTase 9 | 4′-phosphopantetheinyl | Stigmatella aurantiaca | 38% | | | |
| | transferase | | | | | |
| PPTase | 4′-phosphopantetheinyl | Acidobacterium | 42% | Y | N | N |
| 10 | transferase MtaA | capsulatum | | | | |
| Mem | candidate membrane | Ramlibacter tataouinensis | 27% | | | |
| | protein | | | | | |
| HYP 6 | hypothetical protein | Cyanothece sp. ATCC | 42% | | | |
| | cce_2351 | 51142 | | | | |
| Hyp 7 | Hypothetical protein 6 | Coprinopsis cinerea | 28% | Y | N | N |
| Amp | AMP-dependent | Bacillus tusciae | 47% | | | |
| | synthetase and ligase | | | | | |
| MDB | molybdopterin binding | Desulfotomaculum | 59% | Y | N | N |
| | aldehyde oxidase and | reducens | | | | |
| | xanthine dehydrogenase | | | | | |
| PPTase | phosphopantetheine- | Microcoleus vaginatus | 39% | | | |
| 12 | protein transferase | | | | | |
| PBP | polysaccharide | Rhodoferax ferrireducens | 44% | N | N | Y |
| | biosynthesis protein | T118 | | | | |
| PPTase | 4′-phosphopantetheinyl | Candidatus Nitrospira | 36% | | | |
| 13 | transferase | defluvii | | | | |
| HYP 8 | hypothetical protein | Chlorella variabilis | 34% | | | |
| | CHLNCDRAFT_143110 | | | | | |
| Hit 14 | hypothetical protein | Haliangium ochraceum | 60% | N | Y | N |
| | Hoch_3616 | | | | | |

^{†}Fragments are numbered according to the PPTase numbering indicated in each group of rows (e.g. PPTase 1 is on fragment 1;
PPTase 4 is on fragment 4). Abbreviated gene names are given in the left hand column, with FIG. 22 illustrating the layout of these genes on the seven eDNA fragments. Fragments containing likely SMC marker genes are annotated in bold. The three right-most columns indicate which reporter strain each fragment was recovered from. Fragment 14 (hit 14) contains only a single open reading frame with no homology to any previously identified PPTases.

### 4—Material and Methods

### 4.1—General Reagents and Materials

Unless otherwise noted, all chemicals were obtained from Sigma Aldrich (St Louis, Mo.) and were of analytical quality. Hisbind™ protein purification resin and buffers were obtained from Novagen (Merck Biosciences, Darmstadt, Germany). IPTG (isopropyl β-beta-D-thiogalactoside) was obtained from Bioline (Taunton, Mass.).

### 4.2—Enzymes

Restriction endonucleases, NEBnext™ end repair kit and Phusion™ high fidelity polymerase were obtained from New England Biolabs (Ipswich, Mass.). Bioline Red™ Taq polymerase mastermix was obtained from Bioline (Taunton, Mass.). T4 DNA Ligase was obtained from Fermentas (Glen Burnie, Md.).

### 4.3—Bacterial Strains and Plasmids

### 4.3.1—Bacterial Strains

TABLE 4.1
E. coli strains used in this study

| Strain | Relevant characteristics* | Source |
|---|---|---|
| DH5α | supE44 Dlac U169 (Ø80 lacZ DM5) hsdR17 | Invitrogen |
| BL21 | F⁻ ompT gal dcm lon hsdS_{B}(r_{B}⁻ m_{B}⁻) λ(DE3) | Novagen |

*Standard genotype abbreviations are used, a list of these can be found at http://openwetware.org/wiki/E._coli_genotypes#Nomenclature_.26_Abbreviations

### 4.3.2—Plasmids

TABLE 4.2
Plasmids used in this study

| Plasmid | Relevant characteristics | Reference |
|---|---|---|
| pET28a(+) | LacI^{q}, T7prom, Kan^{R}, ColE1ori | Novagen |
| pCDFDuet1 | LacI^{q}, T7prom, spec^{R}, CDFori | Novagen |
| pET::BpsA | pET28a + bpsA (NdeI-HindIII) | This study |
| pSX::BpsA | pSX + bpsA (NdeI-HindIII) | This study |
| pBPSA3 | pCDFduet based staging vector allowing introduction of foreign T- | This study |
| | domains into bpsA | |
| slBPSA | pBPSA3 + T-domain from bpsA, expresses WT BpsA protein | This study |
| slPvdD | pBPSA3 + T-domain from first module of P. aeruginosa pvdD | This study |
| slEntF | pBPSA3 + T-domain from E. coli entF | This study |
| slDhbF | pBPSA3 + T-domain from first module of B. subtilis dhbF | This study |
| slPvdD2 | pBPSA3 + T-domain from second module of P. aeruginosa pvdD | |
| slPST | pBPSA3 + T-domain from second module of P. syringae | |
| | pspph1926 | |
| pET::KT | pET28a + P. putida KT2440 PPTase gene | This study |
| pET::sfp | pET28a + sfp PPTase gene from Bacillus subtilis | This study |
| pET::pcpS | pET28a + pcpS PPTase gene from P. aeruginosa PAO1 | This study |
| pCDF::KT | pCDF + P. putida KT2440 PPTase gene | This study |
| pCDF::sfp | pCDF + sfp from B. subtilis | This study |
| pCDF::pcpS | pCDF + pcpS PPTase gene from P. aeruginosa PAO1 | This study |

### 4.4—Oligonucleotide Primers

Primers were designed using Vector NTI® and ordered from IDT custom oligonucleotide service (Integrated DNA technologies, Coralville, Iowa). Primers were reconstituted to a final concentration of 100 μM in 1×TE buffer for storage at −20° C. Working stocks were prepared by dilution to 10 μM with sterile ddH₂O. The names and sequences of all primers used in this study are given in Table 4.3.

TABLE 4.3
Primers used in this study

| | |
|---|---|
| Primer name | |
| and function | Sequence (5′→3′) |

Primers for creation of pBPSA3

| | |
|---|---|
| pBPSA3_Lup | CCCCGGATCCGATGACTCTTCAGGAGACCAGCG (SEQ ID NO: 22) |
| pBPSA3_Ldwn | AGCTAAGCTTAGCTATGCATTGACCTGGTCGGAGGCGG (SEQ ID NO: 23) |
| pBPSA3_Rup | AGCTAAGCTTAGCTACTAGTCGCTTCGTCCGCCTGCACG (SEQ ID NO: 24) |
| pBPSA3_Rdwn | CCCCCTCGAGTCACTCGCCGAGCAGGTAGC (SEQ ID NO: 25) |

Primers for substitution of T-domains into pBPSA3

| | |
|---|---|
| slBPSAT_Fwd | AGCTCTGCAGAGCTCGTCGAGCGCCCCTTCGTCGCCCCGCGCACG (SEQ ID NO: 26) |
| slBPSAT_Rev | AGCTTCTAGACTCCTGGGCGACCTCGCGCTCCAGGCGGCGGGCCAG (SEQ ID NO: 27) |
| slPvdDT_Fwd | AGCTCTGCAGAGCTCGTCGAGCGCCCCTATCGAGCGCCCGGTAGC (SEQ ID NO: 28) |
| slPvdDT_Rev | AGCTTCTAGACTCCTGGGCGACCTCGCGTTCCAATCCCTGGGCGAA (SEQ ID NO: 29) |
| slDhbFT_Fwd | AGCTCTGCAGAGCTCGTCGAGCGCCCCGATCGGGCCCCGCGGACT (SEQ ID NO: 30) |
| slDhbFT_Fwd_Rev | GCTTCTAGACTCCTGGGCGACCTCGCGATCAAGATGGGCAGCGA (SEQ ID NO: 31) |
| slEntFT_Fwd | AGCTCTGCAGAGCTCGTCGAGCGCCCCGGGCGTGCGCCGAAAGCG (SEQ ID NO: 32) |
| slEntFT_Rev | AGCTTCTAGACTCCTGGGCGACCTCGCGATCAATAATCGTTGCCAG (SEQ ID NO: 33) |
| slEntBT_Fwd | AGCTCTGCAGAGCTCGTCGAGCGCCCCCTGCCAGCACCTATCCC (SEQ ID NO: 34) |
| slEntBT_Rev | AGCTTCTAGACTCCTGGGCGACCTCGCGGGAGAGTAGCTTCCAC (SEQ ID NO: 35) |

Primers for PPTase amplification

| | |
|---|---|
| PP_PPTase_fwd_NdeI | GGGGCATATGAACACACTTCCCGCCTGC (SEQ ID NO: 36) |
| PP_PPTase_rev_SalI | GGGGGTCGACTCAGACGCTGACCAGGCTCA (SEQ ID NO: 37) |
| pcpS_fwd_NdeI | GGGGCATATGCGCGCCATGAACGACCG (SEQ ID NO: 38) |
| pcpS_rev_SalI | GGGGGTCGACTCAGGCGCCGACCGCCACCA (SEQ ID NO: 39) |
| sfp_fwd_NdeI | GGGGCATATGAAGATTTACGGAATTTA (SEQ ID NO: 40) |
| sfp_rev_SalI | GGGGGTCGACTTATAAAAGCTCTTCGTACG (SEQ ID NO: 41) |

Restriction sites are indicated in bold, homology regions for overlap PCR are underlined.

### 4.5—Media

Unless otherwise noted all media components were dissolved in ddH₂O and sterilized by autoclaving.

### LB

LB was obtained as a premixed powder from Sigma Aldrich (St Louis, Mo.) and when reconstituted contained: 5 g/L yeast extract, 10 g/L bacto tryptone, 10 g/L NaCl.

### TYM Medium

2% bacto-tryptone, 0.5% yeast extract, 100 mM NaCl, 10 mM MgCl₂. MgCl₂ added after autoclaving from a sterile 1 M stock.

### ZYP5052 Autoinduction Medium

ZY Base: 10 g/L N—Z Amines AS, 5 g/L yeast extract
50×5052: 250 g/L glycerol (=200 ml/L), 25 g/L D-glucose, 100 g/L α-lactose

### 20×NPS: 66 g/L NH₄SO₂, 136 g/L KH₂PO₄, 142 g/L Na₂HPO₄

ZYP5052 was prepared immediately prior to use and contained (per litre): 929 ml ZY base, 20 ml 50×5052, 50 ml 20×NPS, 1 ml 1M MgSO₄. To prepare solid autoinduction media, agar powder was also added to a final concentration of 15 g/L.

### 4.5.1—Media Supplements

All antibiotic stocks were made to 1000× the media concentration indicated in Table 4.4. IPTG stocks were prepared to a final concentration of 100 mg/ml. Media supplements were dissolved in ddH₂O and filter sterilized using a 0.22 μM filter. Final media concentration of antibiotics were as listed in Table 4.4

TABLE 4.4
Antibiotics used for plasmid selection and maintenance in E. coli

| Antibiotic | Concentration (μg/ml) |
|---|---|
| Ampicillin | 100 |
| Kanamycin | 50 |
| Spectinomycin | 50 |

### 4.6—Growth and Maintenance of Bacteria

LB was used for routine growth and maintenance of E. coli strains. Unless otherwise noted, growth was conducted at 37° C. with aeration by shaking at 200 rpm. For strains harbouring one or more plasmid appropriate antibiotic(s) were included in the medium at the concentrations indicated in Table 4.4

### 4.7—Routine Molecular Biology

### 4.7.1—PCR Protocols

### Standard PCR Reactions

Where PCR products were to be used in downstream cloning applications, amplification was conducted using the New England Biolabs (Ipswich, Mass.)) Phusion™ high fidelity polymerase kit. For screening purposes and splicing by overlap PCR, Bioline Biomix Red™ Taq polymerase mastermix (Bioline, Taunton, Mass.). was employed. Standard reactions set up according to manufacturer's directions were usually sufficient for good amplification. In rare instances, systematic variation of the concentration of one or all of the following components was required to achieve amplification: MgCl₂, DNA template, primers, DMSO.

### Amplification Protocols

The following thermocycler protocols were used for amplification using Phusion or Biomix red. In rare cases it was necessary to systematically vary one or all of the cycle parameters to achieve amplification.

Phusion PCR protocol

| | | |
|---|---|---|
| 98° C. 60 s | | |
| 98° C. 15 s | | |
| 72° C. 30 s (−1° C./cycle) | {close oversize brace} | 10 cycles |
| 72° C. 30 s per kilobase | | |
| 98° C. 15 s | | |
| 62° C. 30 s | {close oversize brace} | 25 cycles |
| 72° C. 30 s per kilobase | | |
| 72° C. 5 min | | |
| 12° C. on hold | | |

Biomix red PCR protocol

| | | |
|---|---|---|
| 95° C. 5 min | | |
| 95° C. 30 s | | |
| 56° C. 30 s | {close oversize brace} | 10 cycles |
| 72° C. 1 min per kilobase | | |
| 95° C. 30 s | | |
| 52° C. 30 s | {close oversize brace} | 20 cycles |
| 72° C. 1 min per kilobase | | |
| 72° C. 10 min | | |
| 12° C. on hold | | |

### 4.7.2—Isolation Purification and Manipulation of DNA

### Isolation and Purification

Small-scale preparation of plasmid DNA was carried out using either the Zymo® (Zymo Research, Orange, Calif.) Zyppy™ or Qiagen (Valencia, Calif.) Minispin™ kit. Preparation of genomic DNA was achieved using a Qiagen DNeasy™ Kit (Qiagen, Valencia, Calif.). Deproteinisation and buffer exchange of PCR products, restriction digests and ligation mixtures was carried out using Zymogen Zymospin™ columns (Zymo Research, Orange, Calif.). In all cases the manufacturer's protocol was followed. Where necessary, DNA samples were quantified and assessed for purity using a Nanodrop™ spectrometer (Thermo Scientific, Wilmington, Del.) or by agarose gel electrophoresis.

### Restriction Digests

Unless otherwise noted, restriction digests were carried out according to the manufacturer's directions. Before use in ligations, digests were heat inactivated and cleaned using a Zymospin™ column (Zymo Research, Orange, Calif.).

### Ligations

Ligations were typically set up using a 1:6 molar ratio of vector:insert with enzyme and buffer concentration, as specified in the manufacturer's directions. Total DNA concentration was kept below 10 ng/μL. For transformation by electroporation, ligations were cleaned using a Zymospin™ column (Zymo Research, Orange, Calif.). Negative controls containing no insert DNA were set up and transformed in parallel to assess vector self-ligation and residual uncut vector DNA.

### 4.7.3—Preparation and Transformation of Competent Cells

4.7.3.1—Preparation and Transformation of Chemically Competent E. coli

This protocol is based on the method described in (Hanahan 1983).

### Reagents

TFBI: 30 mM KOAc, 50 mM MnCl₂, 100 mM KCl, 10 mM CaCl₂ 15% w/v, glycerol. pH adjusted to 5.8 with 0.2 M acetic acid, solution sterilized using a 0.22 μM filter.
TFBII: 10 mM Na-MOPS pH7.0, 10 mM KCl, 75 mM CaCl2 15% w/v glycerol. Solution sterilized using a 0.22 μM filter.

### Preparation

The desired strain was streaked onto TYM agar and incubated overnight at 37° C. A single colony was then inoculated into 3 ml TYM broth and incubated at 37° C./200 rpm for 12-16 h. 1 ml of this overnight was then used to inoculate 40 mL of TYM in a baffled, sterile 250 ml conical flask. The resulting culture was grown at 37° C./200 rpm until an OD 600 of 0.35-0.4 was reached at which point the culture was transferred into a sterile 50 ml tube and placed on ice for 5 min. Cells were then collected by centrifugation (2500 rcf, 4° C., 10 min) and supernatant decanted off. The resulting cell pellet was resuspended in 2 ml ice-cold TFBI by gentle pipetting before addition of a further 38 ml ice-cold TFBI. The cells were then kept on ice for 2 h and again harvested by centrifugation (2500 rcf, 2° C., 10 min). The resulting cell pellet was resuspended in 4 ml ice cold TFBII and 100 μL aliquots distributed into pre-chilled 1.5 ml microfuge tubes on ice. Cell aliquots were then snap-frozen using liquid nitrogen or a metal tube block cooled to −80° C. Frozen aliquots of cells were stored at −80° C. until needed. When appropriate for plasmid maintenance, antibiotics were added to TYM broth and agar.

### Transformation

Cell aliquots were removed from storage and placed on ice until thawed. ˜10 ng Plasmid DNA or up to 200 ng ligation mixture was then added in a volume not exceeding 1/10^{th} that of the cell aliquot. Following addition of DNA, the tube was flicked gently to mix and kept on ice for 30 min. Cells were then heat shocked by placing in a water bath at 42° C. for 2 min. Following heat shock cells were placed immediately on ice for 5 min, before the addition of 9 volumes sterile LB broth. Transformations were then incubated at 37° C./200 rpm for 45-60 min to recover. After recovery cells were plated on LB agar containing appropriate antibiotics. For plasmids aliquots of 100 μL were plated. For ligations an aliquot of 100 μl was plated followed by collection of remaining cells by centrifugation (13,000 rcf, 20 s). The supernatant was then decanted leaving ˜100 ul residual medium, in which the cell pellet was resuspended and then plated.

4.7.3.2—Preparation and Transformation of Electrocompetent E. coli

This protocol is based on the method described in (Sambrook and Russell 2006).

### Reagents

GYT: 0.25° A) bacto tryptone, 0.125% yeast extract, 10% v/v glycerol. Sterilise by autoclaving.

### Preparation

The desired strain was streaked onto LB agar and incubated overnight at 37° C. A single colony was then inoculated into 25 ml LB broth and incubated at 37° C./200 rpm for 12-16 h. 10 ml of the overnight culture was then used to inoculate 500 mL LB medium in a baffled, sterile 2000 ml conical flask. The resulting culture was grown at 37° C./200 rpm until an OD 600 of 0.35-0.4 was reached, at which point the culture was transferred into 10 sterile 50 ml tubes and placed on ice for 15-30 min with occasional swirling. Cells were then collected by centrifugation (1000 rcf, 30 min, 2° C.) and the pellets resuspended in a total volume of 500 mL ice cold sterile ddH₂O by gentle pipetting. Following this first washing step cells were again collected by centrifugation (1000 rcf, 30 min, 2° C.) and the resulting pellet resuspended in 250 ml ice-cold sterile 10% v/v glycerol. Cells were again pelleted by centrifugation (1000 rcf, 30 min, 2° C.) and the resulting pellet resuspended in 125 ml ice cold sterile 10% v/v glycerol. Cells were again collected by centrifugation and the pellet resuspended in 500 μL ice cold sterile GYT. The OD 600 of a 1/100 dilution of the cell mixture was then determined and GYT added to give a final concentration of 2-3×10¹⁰ cells/ml (OD 600 1.0=2.5×10⁸ cells/ml). 40 μL aliquots were then distributed into pre-chilled 1.5 ml microfuge tubes on ice. Cell aliquots were then snap-frozen using liquid nitrogen or a metal tube block cooled to −80° C. Frozen aliquots of cells were stored at −80° C. until needed. When appropriate for plasmid maintenance, antibiotics were added to LB broth and agar for growth steps.

### Transformation

Cell aliquots were removed from storage and placed on ice until thawed. 50 ng ligation/40 μL cell mixture was then added in a volume not exceeding 1/10^{th} that of the cell aliquot. Following addition of DNA, the tube was flicked gently to mix and the contents transferred to an ice cold, sterile 2 mM gap electroporation cuvette. Cells were then electroporated (2.5 kV, 25 μF, 100Ω), 1 ml SOC broth immediately added and the mixture transferred to a sterile 15 ml centrifuge. Cells were then incubated at 37° C./200 rpm for 1 h before plating on medium containing appropriate antibiotics. When likely transformation efficiency was unknown, 100 μl of neat transformation as well as 100 μL of a 1/10 and 1/100 dilution were plated to ensure single colonies were obtained.

### 4.7.3.3—Identification of Recombinant Clones

### Colony PCR

The first step for identification of clones harbouring desired inserts on a plate arising from transformation of a ligation was colony PCR. Standard Biomix red PCR reactions with a final volume of 15 μL were set up on ice for each clone to be screened. Where possible, a primer targeting plasmid sequence was used in combination with a primer targeting insert sequence to avoid non-specific amplification. Colonies to be screened were picked using a sterile pipette tip and streaked onto a numbered plate containing appropriate antibiotics. The residual bacteria on the tip were then transferred into a PCR reaction and released by gentle swirling. Reactions were then subjected to the Biomix red amplification protocol described in Section 4.7.1. Typically, two controls were included in each screen these were: a reaction in which a clone from a vector-only control plate was used as template and a reaction inoculated with a pipette tip that had been touched to a region of the vector+insert agar plate not containing any colonies. The former assessed non-specific amplification form plasmid and genomic DNA sequences, the latter amplification of residual PCR product spread onto the surface of a plate. Following amplification 7 μL aliquots of each reaction were analysed by agarose gel electrophoresis and 3 ml overnight cultures set up for clones showing amplification of the correct sized product.

### Conformation by Restriction Digest

Plasmid DNA from PCR positive clones was prepared from overnight cultures as described in Section 4.7.2. Digests of plasmid DNA from each clone were then set up as described in Section 4.7.2, using a combination of enzymes that would generate a diagnostic fragment in insert positive clones. Following incubation, digest were assessed for liberation of diagnostic fragment by agarose gel electrophoresis and plasmid DNA from positives sent for sequence analysis.

### Sequencing of DNA

Sequencing was performed by Macrogen Inc. (Seoul, Korea). DNA samples and primers for sequencing were prepared according to the company's specifications of concentration and purity. Sequence quality was assessed using Contig Express™ (Invitrogen, Carlsbad, Calif.) and insert identity confirmed by alignment of sequences against a template obtained from Genbank.

### 4.8—Protein Expression and Purification

### 4.8.1—Protein Expression

An overnight starter culture of the desired strain was grown in LB medium containing appropriate antibiotics and 0.4% w/v D-glucose. This starter culture was then used to inoculate ZYP5052 medium expression culture containing appropriate antibiotics. The volume of starter culture used to inoculate was 1/50 the amount of ZYP5052 used in the expression culture. The expression culture was then incubated at 16° C./250 rpm until an OD 600 of over 2.5 was reached, at this point expression levels of target protein were determined by SDS PAGE analysis. If expression was deemed sufficient for subsequent purification, the culture was harvested by centrifugation (4000 rcf, 4° C., 15 min).

### 4.8.2—Protein Purification by Ni-NTA Affinity Chromatography

### 4.8.2.1—Cell Lysis and Fraction Separation

For protein purification, cell pellets were gently resuspended in 1×Hisbind™ Binding buffer on ice and lysed by either French press or sonication. The amount of buffer used to resuspended pellets was 1/20 of the expression culture volume. For lysis by French press three passages at 40,000 psi were conducted, the French press chamber was chilled to 2° C. prior to lysis. For sonication, resuspended cells were placed in an ice bath and sonicated at 70% maximum output, with 50% duty cycle and 10 s bursts until consistency indicated sufficient lysis (usually 2-3 min). Following lysis, soluble and insoluble fractions were separated by centrifugation (17,000 rcf 2° C., 20 min) and the soluble fraction stored on ice until purification.

### 4.8.2.2—Standard Ni-NTA Affinity Chromatography

Unless otherwise noted, purification of 6His-tagged proteins was achieved using a Novagen Hisbind™ Ni-NTA chromatography kit, according to the manufactures directions. A final volume of 1.5 ml settled resin was used for purification unless otherwise noted. Eluted protein was captured in 1.5 ml fractions for analysis by SDS PAGE. The first time any protein was purified, flow-through from lysate application and all washing steps was also analysed by SDS PAGE to evaluate binding efficiency and wash stringency.

### 4.8.2.3—Special Amendments to Purification Procedures

For purification of BpsA it was found that protein binding to the column was weak resulting in protein elution during the second more stringent wash step. In order to achieve protein of good purity, it was necessary to fully saturate the column's binding capacity by applying an excess of cell lysate. To achieve this, a 2000 ml expression culture was set up using the auto induction protocol described in Section 4.8.1. The entire soluble fraction (100 ml) resulting from this culture was then applied to 7 ml pre-equilibrated Hisbind resin. Flow-through, from the soluble fraction was collected and again applied to the column. After this, purification was conducted according to the manufacturer's protocol except the volume of the first wash step was tripled and the second wash step eliminated. For purification of PPTase enzymes it was found that proteins precipitated immediately once eluted from the column. To prevent this 10% v/v glycerol was added to all His bind buffers and all buffers kept ice cold during the purification. Following elution all fractions were immediately desalted and stored at −20° C. in storage buffer containing 50% w/v glycerol.

### 4.8.2.4—Buffer Exchange, Storage and Quantification of Recombinant Proteins

Buffer exchange was achieved using GE Healthcare (UK) Hitrap™ desalting columns, according to the manufacturer's protocol. Storage buffer for proteins was 50 mM Tris-Cl 50% w/v glycerol, the pH of which was the same as that of the assay solution in which proteins were to be employed. Proteins were stored at −20° C. and kept on ice at all times when in use. Quantification of recombinant proteins was achieved using Bio-Rad (Bio-Rad, Hercules, Calif.) DC Kit according to the manufacturer's directions.

### 4.8.3—SDS-Polyacrylamide Gel Electrophoresis

SDS-PAGE was performed using 12% SDS-polyacrylamide gels prepared according to the method of Laemmli (Laemmli 1970). Gels were cast and run using the Bio-Rad (Hercules, Calif.) Protean II™ system, according to the manufacturer's directions. Preparation of samples, as well as gel staining and destaining, was carried out as described by Sambrook and Russell (Sambrook and Russell 2003).

### 4.9—Directed Evolution Protocols

The protocols used for directed evolution were continually refined throughout the course of this study and the final optimized version of each aspect is given below. These protocols allow the generation of libraries of 1-5×10⁶ insert containing clones from a single epPCR reaction. Libraries of this size were more than sufficient for recovery of multiple improved clones.

### 4.9.1—Library Generation

### 4.9.1.1—Vector Preparation

For preparation of vector for directed evolution, 16 μg of pBPSA3 plasmid DNA was heated to 70° C. for 20 min to relieve super-coiling and then digested with 50 U of each restriction enzyme in a final volume of 400 μL. Buffer composition and incubation temperature was as recommended by the enzyme manufacturer. After 5 h incubation, an additional 20 U of each enzyme was added, and the digest incubated for a further 12-14 h. Following this, digests were heat inactivated (80° C., 20 min) and purified using a 20 μg, capacity Zymospin® clean up column (Zymo Research, Orange, Calif.). Digested vector was eluted from the clean up column using 30 μL sterile ddH₂O and the concentration of the resulting solution determined. This was generally between 250-350 ng/μL. A 200 ng sample of vector was then analysed by agarose gel electrophoresis to ensure no degradation had occurred. Aliquots of 5 μL were then prepared and stored at −20° C.

### 4.9.1.2—Determination of Vector Quality

Before being employed in directed evolution experiments the quality of prepared vector was assessed. To achieve this, the native T-domain of bpsA was amplified using Phusion® high fidelity polymerase and prepared for ligation as described in Section 4.9.1.4. A control ligation was then set up as described in 4.9.1.5, except with digested bpsA T-domain in place of epPCR. The ligation was then transformed into chemically competent cells harbouring an activating PPTase and aliquots of the transformation mixture plated on pigment development agar (LB, 100 mM L-Gln, 0.5 mM IPTG). Plates were then incubated for 12-14 h at 37° C. before induction of protein expression and as described in 4.9.2. Plates were then left at room temperature for 12-16 h before determination of transformation efficiency and percentage of colonies containing insert. Determination of insert percentage was achieved by counting blue and white colonies on an entire plate (containing at least 200 colonies). Blue colonies indicated correct ligation of the T-domain insert into the prepared vector, white colonies indicated vector self ligation or out-of-frame ligation. If the percentage of blue colonies was 70 or higher, the vector was deemed of sufficient quality for use in directed evolution experiments.

### 4.9.1.3—Error Prone PCR

Error prone PCR was carried out using a Stratagene (Agilent, Santa Clara, Calif.) Mutazyme II® kit, according to the manufacturer's directions. Optimal error rate (as assessed by number of improved clones recovered) was achieved using 100 ng of purified PCR product as template per 50 μL reaction, with 30 amplification cycles. Template was prepared by amplification of the appropriate T-domain sequence using Phusion polymerase. Prior to thermocyling, reactions were divided into four to reduce the number of clonal mutations in the final library. Amplicon size and quality was assessed by agarose gel electrophoresis of a 3 μL aliquot.

### 4.9.1.4—Insert Preparation

50 μL epPCR reactions were purified using a 5 μg capacity Zymospin® column (Zymo Research, Orange, Calif.), with elution achieved using 20 μL sterile ddH₂O. The entire eluent was then digested for 5 h with 30 U of each enzyme, in a final volume of 50 μL. Buffer composition and incubation temperature was as recommended by the enzyme manufacturer. Following digest, reactions were heat inactivated (80° C., 20 min) and purified using 5 μg capacity Zymospin® column (Zymo Research, Orange, Calif.) (elution volume 20 μL, sdH₂O).

### 4.9.1.5—Ligation

Ligations were set up as described in Section 4.7.2 with the following amendments: Total amount of DNA per ligation was 1.0-1.5 μg. Ligations were incubated overnight at 16° C., after which an additional 2 U of ligase were added, and the reaction incubated for a further 8 h at 16° C. The optimal molar ratio of vector to insert for achieving maximum ligation efficiency was found to be 1:6.

### 4.9.1.6—Control Reactions

One positive and two negative control reactions were run for each directed evolution experiment. For the positive control, the native T-domain of BpsA was amplified using high-fidelity polymerase, digested and ligated and transformed using the same conditions as for ep-library generation. The purpose of the positive control was to give a reliable estimate of percent insert containing clones using the process described in Section 4.9.1.2. The first negative control reaction was set up in the same way, except using the T-domain to be evolved, amplified with high fidelity polymerase. Inclusion of this negative control was important to check for contamination of PCR reagents with WT bpsA DNA, which would result in a large number of false positives. The second negative control was a plasmid containing the original recombinant bpsA gene which the experiment aimed to improve. Colonies arising from transformation of this control were used as a reference point for determination of improved clones.

### 4.9.1.7—Transformation and Library Storage

Prior to transformation, ligations were purified using a 5 μg capacity Zymospin column (Zymo Research, Orange, Calif.) (elution volume 20 μL, sdH₂O). Purified ligations were then transformed into electrocompetent E. coli BL21 cells, harbouring a plasmid for expression of an activating PPTase. Electrocompetent cells were prepared and transformed as described in Section 4.7.2.2, with the following amendments: The volume of cells used was 60 μL per 50 ng ligation to be transformed. Cells and ligations were mixed on ice and then aliquoted into chilled cuvettes (80 μL/cuvette). Following electroporation, 1 ml SOC medium was immediately added to each cuvette. All cells for a single ligation were then pooled in a single 15 ml tube and recovered for 1 hr (37° C., 200 rpm). Following recovery, cells were mixed 1:1 with sterile 80% glycerol and 0.5 ml aliquots dispensed. These aliquots were then placed at −80° C. A single aliquot was then thawed on ice and serial dilutions plated on pigment development agar (LB, 100 mM L-Gln) containing appropriate antibiotics in order to determine the optimal plating volume for screening. Libraries stored as frozen transformation aliquots remain viable for at least 3 months.

### 4.9.2—First Tier Screening

For screening, cells were thawed on ice and plated on LB agar containing 100 mM L-Gln and strain appropriate antibiotics. The volume of cells used per plate was adjusted so that ˜5,000-10,000 insert containing clones would be present. After 12 h of incubation at 37° C., expression was induced in plates. Induction was achieved by removing the agar slab from the plate, evenly distributing 100 μL 2.5% w/v IPTG on the bottom of the plate and then replacing the agar slab on top of the IPTG solution. Plates were then incubated at ˜20° C. and monitored for colour development. Colonies which developed colouration before negative controls were taken to be hits. These putative improved clones were picked from plates and small scale overnight growth cultures set up for preparation of glycerol stocks. Due to the high density of colonies on a single plate, it was often impossible to recover improved clones without risk of contamination. In such cases, clones were picked as accurately as possible using a toothpick and resuspended in 100 μL GYT. Single colonies were obtained by streaking this suspension on a portion of an agar plate. Subsequent induction of this plate allowed recovery of improved clones without contaminants. Improved clones were then grown overnight in LB medium supplemented with 0.4% glucose and appropriate antibiotics. Duplicate glycerol stocks of improved clones were then prepared in a 96wp.

### 4.9.3—Second Tier Screening

For second tier screening, up to 45 improved clones, along with a positive and negative control, were stored as duplicate glycerol stocks in a 96wp. From this plate duplicate overnight cultures were established in LB supplemented with 0.4% glucose and appropriate antibiotics. Set up of overnight cultures was achieved in a 96wp using a 96-pin inoculating tool. Overnight cultures were grown for 16-20 h (37° C. 200 rpm) before transfer of 20 μl of each culture into a fresh well containing 130 μL LB supplemented with 115 mM L-Gln, 0.6 mM IPTG and appropriate antibiotics. Assay plates set up in this fashion contained duplicate cultures for each clone from two separate overnights. Assay plates were then wrapped in foil and incubated for 6-24 h (18° C., 200 rpm), depending on activity of improved clones. After incubation cells were collected at the corner of each well by centrifugation and OD 590 values determined using a microplate reader. Assays were repeated at least twice before determination of clones for sequence analysis.

4.10—Determination of In Vivo Pigment Synthesis Activity of mBpsA Derivatives Relative to Wild Type BpsA

In vivo pigment synthesis efficiencies for recombinant BpsA proteins described in Section 3.2 were derived from three separate assays. This was necessary due to the fact that pigment synthesis of wild type BpsA saturate before activity of mBpsA derivatives was detectable. Replicate cultures for determination of in vivo pigment synthesis activity were established from overnight starter cultures in a 96 wp as described in Section 4.9.3 The values presented are derived as follows: quadruplicate values for WT BpsA and oBpsA (mutant with reduced activity) were determined after 3 h, at which point pigment production was not saturated for WT. A separate quadruplicate assay was then run to obtain a value for slEntF by comparison to oBpsA after 12 h. Quadruplicate values for slPsT and slPvdD2 were then obtained in a separate assay by comparison to slEntF after 12 h.

### 4.11—Enzyme Kinetics

### 4.11.1—Activation of BpsA by 4′-PP Attachment

Pre-activation mix to bring about conversion of apo to holo BpsA contained 3.4 μM apo-BpsA, 0.25 μM PcpS, 10 mM MgCl₂, 100 μM CoA and 50 mM buffer (sodium phosphate buffer pH 7.8 or Tris-Cl pH 8.0) and was incubated for 20 min at 30° C.

### 4.11.2—Determination of Kinetic Parameters for BpsA

For derivation of kinetic parameters, triplicate two fold serial dilution series of L-gln were established in a final volume of 50 μL in a 96 wp. 100 μL reaction mix (5 mM ATP, 15 mM MgCl₂, 8 mM L-gln and 75 mM sodium phosphate buffer, pH 7.8) was then added to each well. Reactions were initiated by addition of 50 μL pre-activation mix to each well, followed by mixing at 1000 rpm for 10 s. A590 measurements were then taken every 6-10 s for ten minutes using an envision EnSpire® microplate reader. The resulting data was visualized and velocities derived using the Slope function of Microsoft Excel®. Kinetic parameters were derived from velocity values using Graphpad Prism®. For examination of the relationship between BpsA concentration and reaction velocity, a triplicate two fold serial dilution series of pre-activated BpsA was established in a 96 wp in a final volume of 50 μL, 100 μl reaction mix without L-gln or ATP was then added to each well. Reactions were initiated by addition of 50 μL 8 mM L-gln, 5 mM ATP to each well followed by mixing at 1000 rpm for 10 s. A₅₉₀ values were then recorded and data analysed as previously described.

### 4.11.3—Determination of Kinetic Parameters for PPTases

For examination of CoA as the variable substrate: triplicate two fold serial dilution series' of CoA were established in a final volume of 50 μL in a 96 wp. 100 μL reaction mix (5 mM ATP, 20 mM MgCl₂, 8 mM L-Gln and 100 mM sodium phosphate buffer, 0.08-0.25 μM PPTase, pH 7.8) was then added to each well. Reactions were then initiated by addition of 50 μL 1.66 μM apo-BpsA to each well followed by mixing at 1000 rpm for 10 s. A₅₉₀ values were then recorded as previously described. The entire experiment was repeated twice for examination of BpsA as the variable substrate: triplicate two fold serial dilution series of BpsA were established in a final volume of 50 μL in a 96 wp. 100 μL reaction mix (5 mM ATP, 20 mM MgCl₂, 8 mM L-Gln and 100 mM Tris-CL, 0.08-0.25 μM PPTase, pH 8.0) was then added to each well. Reactions were then initiated by addition of 50 μL 0.32-1.0 μM PPTase to each well followed by mixing at 1000 rpm for 10 s. A₅₉₀ values were then recorded as previously described. For derivation of PPTase velocity values, the slope between every 2-8 data points was measured across the entire data range with the resulting values forming a new data set (BpsA velocity). The slope between every 2-8 points in the BpsA velocity data set was then determined using the slope function of Microsoft Excel and the maximum value from the resulting data set for each reaction taken as a measure of PPTase velocity. It was necessary to vary the number of points between which slope values were taken due to variation in reaction velocity. For fast reactions smaller increments could be used, however for slow reactions noise from plate reader noise became a significant factor in point to point variation and created artificial fluctuations in measured slopes if too few points were used in slope determination. PPTase reaction velocities were initially obtained with the units ΔA590/s², these were converted to the standard units of amount of BpsA modified per second, which is the same as amount of CoA consumed per second, using the previously established linear relationship between [holo-BpsA] and reaction velocity. The calculations used for this conversion are outlined in section 4.16.1.

### 4.11.4—Carrier Protein Competition Assay

Two fold serial dilution series of carrier protein were established in a final volume of 50 μL in a 96 well microplate. 50 μL of a solution containing 304 apo-BpsA, 750 nM CoA, 40 mM MgCl₂, 400 mM Tris-Cl (pH 8.0) was then added to each well. PPTase reactions were then initiated by addition of 50 μL diluted PPTase solution per well (0.08-0.25 μM PPTase in ddH₂O). Plates were then mixed (1000 rpm, 30 s) and incubated for 15-30 min at 30° C. to allow PPTase reaction to proceed to completion. Varying incubation time over this range did not compromise reproducibility for any of the PPTase/carrier protein combinations we investigated. Following incubation, indigoidine synthesis was initiated by addition of 50 μL 4 mM L-Gln, 4 mM ATP to each well followed by mixing at 1000 rpm for 10 s. A₅₉₀ values were then recorded as previously described. Maximum velocity values for indigoidine synthesis were derived using the slope function of Microsoft excel and converted to % maximum velocity values using the fastest reaction recorded for a single triplicate experiment. For generation of IC₅₀ curves, data from two independent triplicate experiments was pooled and four parameter dose response curves fitted using the non-linear regression function of Graphpad Prism®. IC₅₀ values were converted to estimates of k_{cat}/Kₙ, as outlined in section 4.16.2.

4.12—Generation of a Soil Derived eDNA Library

### Reagents

### Lysis Buffer: 100 mM Tris-HCl, 100 mM Na EDTA, 1.5 M NaCl,

1% (w/v) cetyl trimethyl ammonium bromide, 2% (w/v) SDS, pH 8.0

### TE: 10 mM Tris, 1 mM EDTA, pH 8

4.12.1—Extraction and Purification Environmental DNA from a Soil Sample

Extraction and purification of DNA from a soil sample for the purpose of eDNA library generation was achieved following the protocol described in (Brady 2007) with slight modifications. Approximately 1 kg of soil was collected from four locations at a residential property in Wellington New Zealand. This sample was pooled and then passed through 1.0 cm and 0.25 cm mesh to remove rocks and other debris. Following this 250 g of soil was mixed with 300 ml lysis buffer and incubated at 70° C. for 2 h with inversion every 30 min to mix. The sample was then centrifuged (4000 g, 10 min, 4° C.) and the supernatant removed to fresh vessels. The sample was again centrifuged (4000 g, 20 min, 4° C.) and the volume of the resulting supernatant measured. 0.7 volumes 100% isopropanol were then added and the sample incubated at room temperature for 30 min to allow precipitation of DNA. Following precipitation the DNA was collected by centrifugation (6000 g, 30 min, 4° C.). 100 ml 70% ethanol was then added to the pellet and the sample was again centrifuged (6000 g, 10 min, 4° C.). The supernatant was then decanted and discarded and the pelleted DNA allowed to air dry at room temperature before being resuspended in 10 ml TE. 2.5 ml of the resulting crude eDNA preparation along with two flanking DNA size markers were then run on a 1% agarose gel that did not contain ethidium bromide at 100 V until the brown colouration of the soil sample had run of the end of the gel. High molecular weight eDNA was then located and excised from the gel and recovered by electroelution into 10 kDa cut off dialysis tubing according to the method described in (Brady 2007).

4.12.2—Partial DNase Digest Size Fractionation and End Repair of Purified eDNA

Following purification, pilot experiments were run in which aliquots of high molecular weight eDNA were digested with varying concentrations of DNaseA (New England Biolabs, Ipswich, Mass.) until conditions optimal for generation of fragments between 1-2 kb were determined. Reactions were then scaled up and the remaining sample digested under the conditions previously determined to be optimal. Reactions were terminated by addition of EDTA to a final concentration of 10 mM followed by heating to 70° C. for 20 min. Recovery of fragments between 1-2 kb was achieved by agarose gel electrophoresis and electroelution of the desired size range as described in (Brady 2007). Following electroelution 5 μg of size fractionated DNA was end repaired using a NEBnext™ end repair kit (New England. Biolabs, Ipswich, Mass.) according to the manufacturer's protocol. Following end repair reactions were purified using a Zymospin™ column (Zymo Research, Orange, Calif.) according to the manufacturer's directions. This preparation protocol was designed to generate randomly fragmented eDNA between 1-2 kb in size which was suitable for blunt end ligation into an appropriately prepared vector.

### 4.12.2—Vector Preparation

10 μg of the plasmid pETduet1 (Novagen, Merck Biosciences, Darmstadt, Germany) was prepared using a Zyppy™ miniprep kit and digested for 14 h with 50 U EcoRV-HF (New England Biolabs, Ipswich, Mass.) in a final volume of 200 μL according to the manufacturer's directions. Digests were then heat inactivated and treated with Antarctic phosphatase (New England Biolabs, Ipswich, Mass.) according the manufacturer's directions. Linear vector DNA was then purified using a Zymospin Column™ (Zymo Research, Orange, Calif.).

### 4.12.3—Library Generation

1 μg linear vector and 0.5 μg end repaired eDNA insert were incubated for 4 h at 22° C. with 20 U T4 DNA ligase (Fermentas, Glen Burnie, Md.) in a final volume of 150 μL 1×T4 ligase reaction buffer (Fermentas, Glen Burnie, Md.) supplemented with 5% v/v PEG 4000. Following incubation, ligated DNA was purified using a Zymospin™ column (Zymo Research, Orange, Calif.) and delivered to electrocompetent DH5αz cells as described in Section 4.7.2.2.

4.13—Screening of eDNA Library

The protocol used for eDNA library screening is essentially the same as that outlined in Sections 4.9.1.7 and 4.9.2 except electrocompetent BL21 cells did not contain an activating PPTase and instead contained the plasmid for expression of one of the three BpsA reporter genes. Following recovery, plasmid DNA was prepared from hits and isolation of the pRSET1 plasmid containing the eDNA insert for sequencing was achieved by transformation into chemically competent E. coli cells followed by selection on medium containing ampicillin only. Inserts were sequenced using both T7 promoter and T7 terminator primers which anneal to plasmid sequences immediately upstream and downstream of the insert respectively.

4.14—Isolation of Plasmid DNA Containing eDNA Fragments from Hits

Where possible, single colonies were picked directly from screening plates using a sterile toothpick and inoculated in to 0.5 ml LB medium supplemented with 100 μg/ml ampicillin and 0.4% w/v glucose. These cultures were incubated overnight and plasmid DNA prepared using a Zyppy™ miniprep kit. The plasmid DNA prepared in this fashion was a mixture of library and reporter plasmid constructs. It was therefore necessary to re-transform mixed plasmid samples into chemically competent DH5a cells and select for transformants on medium containing ampicillin only. Following re-transformation, a culture of bacteria harbouring isolated library DNA could be grown and plasmid DNA subsequently isolated for sequencing purposes. In some instances colonies presumed to be hits were contacting other colonies in their immediate vicinity, thereby preventing immediate establishment of a pure culture for plasmid DNA preparation. In such cases colonies were picked and resuspended in 100 μL GYT medium. 1-5 μL of the resulting suspension was then streaked on a pigment production agar plate and incubated for 12-16 h at 37° C. to obtain isolated single colonies. The resulting plate was then induced as described in 4.9.2 and isolated pigmented colonies were selected and grown overnight for plasmid DNA extraction as previously described.

### 4.15—Design and Construction of Modified BpsA Derivatives and Staging Vector for Directed Evolution

4.15.1—Design and Construction of T-Domain Swapping Vector pBPSA3

The strategy used to substitute foreign T-domains in place of the native T-domain of BpsA achieves a seamless transition between native BpsA sequence and substitute T-domain sequence, without introducing any additional amino acid changes due to restriction site introduction. This was achieved by creating a plasmid based copy of BpsA in which the native T-domain was absent and restriction sites were present that allowed substitution of foreign domains in its place. These restriction sites were chosen so as not to cause any change in the amino acid sequence of BpsA upon their introduction.

The program Vector NTI® (VNTI, Invitrogen, Carlsbad, Calif.) was used to identify silent restriction site candidates and design primers for generation of swapping plasmid as follows:

1) A list of restriction enzymes that do not cut the vector (pCDFduet1), bpsA gene or T-domain inserts was generated using the restriction report function of VNTI.
2) The resulting enzymes were then saved as a subset in the restriction enzyme data base of VNTI. Additional hybrid sequences which arise from the ligation of two separate restriction enzyme cuts producing compatible ends were entered into the database manually and were also saved in this subset.
3) Positions at which these restriction sites could be introduced were then determined using the mutagenesis function of VNTI. This resulted in the identification of a hybrid NsiI-PstI site upstream and a hybrid XbaI-SpeI site downstream of the region of T-domain substitution. Introduction of these hybrid sites did not alter amino acid sequence encoded at the splice point.
4) Primers were then designed to amplify the regions up and downstream of the T-domain of BpsA using the amplify selection function of VNTI. The products of amplification by these primers were cloned into pCDFduet to generate a staging vector. 5) For amplification of T-domains for substitution into the staging vector, the amplify selection function was again used. The BpsA sequence between the silent splice points and the desired point of T-domain introduction was added manually to the 5′ end of the up and downstream primers as illustrated in FIG. 24. The exact location of the transition points between BpsA sequence and that of an introduced T-domain is illustrated in the sequence files that accompany this application. Construction of pBPSA3 was achieved following standard molecular biology protocol (outlined in 4.7) as follows:
1) The amplification product of pBpsA3_Rup and pBpsA3_Rdwn was introduced via HindIII and XhoI restriction sites into the plasmid pCDFduet
2) The amplification product of pBpsA3_Lup and pBpsA3_Ldwn was introduced into the intermediate plasmid resulting from (1) via BamHI and HindII restriction sites to give the plasmid pBPSA3

### 4.15.2—Delineation of T-Domains by Structural Modelling and Sequence Alignment.

The nucleotide sequences of BpsA and the genes from which T-domain substitutions were derived were downloaded from public databases, typically NCBI www.ncbi.nlm.nih.gov/) or the pseudomonas genome data base (www.pseudomonas.com). These nucleotide sequences were then translated into the corresponding amino acid sequence using the tools available in VNTI or gentle. Standard bacterial genetic code was used for all translations. The T-domain of BpsA was delineated by generating a structural model of the T and TE domains of the enzyme using the pdb file 2roqA as a template. The core structural motifs of the BpsA T-domain were then annotated onto the amino acid and nucleotide sequence. The splice sites at which foreign domains were to be introduced were chosen so as to fall in putative disordered linker regions outside of the structural core of the T-domain. In order to define the region of amino acid sequence for a T-domain swap sequence alignment of the swap source against the T-domain of BpsA was performed using the alignment tools available in VNTI and/or Gentle. Default parameters were used for all sequence alignments.

### 4.16—Detailed Explanation of PPTase Kinetic Calculations

### 4.16.1—Unit Conversion for PPTase Kinetic Parameters

The raw data input into SigmaPlot for derivation of kinetic parameters for the PPTases PcpS and PP1183 was maximum change in indigoidine (ind) synthesis reaction rate (change in gradient per second) vs. concentration of CoA present in the reaction. The Vmax values given by SigmaPlot therefore had the units “Change in gradient per second” The assumptions made and calculations used to convert this standard enzyme kinetics units are given below. Analysis of ind synthesis reaction rates for various concentrations of ATP had previously revealed a good fit to the Michaelis-Menten equation. As such the assumption can be made that reaction rate for this enzyme is directly proportional to the concentration of active enzyme according to the equation:

V₀=K_{cat}[E][S]/Kₘ+[S]

For the reactions used in derivation of kinetic parameters of PPTases, [BpsA]=[E]=1.64 uM. [S]=[ATP]=1 mM. K_{cat} (BpsA)=241.34 min⁻¹, Kₘ (ATP/BpsA)=11.12 mM.

Substitution of these values into the above equation gives a theoretical maximum velocity of 32.487 μM/min if all BpsA present in the reaction is activated by the PPTase.

The gradient units for the data input into SigmaPlot were gradient of 1.0=change in A590 of 0.001/min.

The relationship between [indigoidine] and A590 for the path length used in the assay is A590 of 0.001=0.0854 uM indigoidine. Therefore the maximum gradient attainable if all BpsA is active is 32.487/0.0854=380.409.

A concentration of 1.64 μM BpsA in a 200 uL reaction means the amount of BpsA present is 328 pmoles.

Going back to the original assumption that velocity is proportional to concentration of active BpsA in a reaction. If 328 pmoles active BpsA give a gradient of 380.4 then theoretically the amount of active BpsA that would give a gradient of 1.0 is 380.4/328=1.1587 pmoles. This establishes a relationship between gradient and concentration of active BpsA which allows the conversion of “change in gradient per second values to” to amount of BpsA activated per second. For example if the instantaneous gradient were measured at a given point, and one second later and found to have increased by 1.0, the above relationship tells us that an extra 1.1587 pmoles of active BpsA are present.

This relationship was used to convert the units of the Vmax values given by SigmaPlot form “Change in gradient per second” to “pmoles BpsA activated per second”. Since BpsA has only one T-domain, one molecule of CoA is used to activate 1 molecule of BpsA and the amount of BpsA activated per second=amount of CoA consumed by PPTase per second.

4.16.2—Conversion of IC₅₀ to Values to Estimates of k_{cat} and Kₘ for Carrier Protein/PPTase Combinations

Derivations of estimates for k_{cat}/Kₘ values for each PPTase/carrier protein combination were achieved following the logic and equations outlined below. Assumptions and simplifications made are also noted.

The competition assays described can be thought of as consisting of two phases. The first of these is the competition phase, in which BpsA and the carrier protein (CP) to be characterized compete for a limited pool of CoA. The second phase is the production phase, in which a relative measure of holo BpsA produced during the competition phase is determined. Since we have shown experimentally that apparent indigoidine synthesis rate in aqueous solution, as determined by change in A₅₉₀, is directly proportional [holo-BpsA], a relative measure of [holo-BpsA] can achieved by measuring the rate of indigoidine synthesis upon addition of the L-gln and ATP.

The amount of holo BpsA produced is dependent on the average velocity of BpsA modification (V_{B}) compared to the average velocity of CP modification (V_{C}) during the competition phase. The IC₅₀ value for a given CP is the concentration which results in a 50% reduction of indigoidine synthesis velocity, and therefore a 50% reduction in the amount of holo BpsA formed during the competition phase of the assay. Therefore when [CP]=IC₅₀, V_{B}=V_{C} and 50% of the available CoA will be incorporated into BpsA, with the remaining 50% used in modification of the CP to be characterized.

Since PPTases are known to obey the Michaelis Menten model the situation when [CP]=IC₅₀ can be expressed as:

$$[ PPTase ] \frac{{K}_{catC} [ CP ]}{{K}_{mC} + [ CP ]} = [ PPTase ] \frac{{K}_{catB} [ BpsA ]}{{K}_{mB} + [ BpsA ]}$$

Substituting in CP and BpsA concs. and cancelling [PPTase term]

$$\frac{{K}_{catC} [ {IC}_{50} ]}{{K}_{mC} + [ {IC}_{50} ]} = \frac{{K}_{catB} [ 1 μM ]}{{K}_{mB} + [ 1 μM ]}$$

Substituting known k_{cat} and Kₘ with respect to BpsA for each PPTase results in a known constant (K_{BpsA}) for each PPTase.

$$\frac{{K}_{catC} [ {IC}_{50} ]}{{K}_{mC} + [ {IC}_{50} ]} = {K}_{BpsA}$$

For the sake of simplification and to allow derivation of an estimate, assume 10₅₀<<Kₘ so can simplify to

$$\frac{{K}_{catC}}{{K}_{mC}} = \frac{{K}_{BpsA}}{{IC}_{50}}$$

Although the assumption 10₅₀<<Kₘ may not be valid, this method nonetheless allows derivation of a sound estimate for k_{cat}/Kₘ for a particular PPTase/Carrier protein interaction.

### 4.17—Discovery and Characterization of PPTase Inhibitors

### 4.17.1—Assessment of PPTase Inhibition by 6-NOBP

Triplicate two fold serial dilution series (250-0.12 μM) of the previously identified PPTase inhibitor 6-NOBP were established in a final volume of 50 ml 10% DMSO in a 96 wp. 100 μL of reaction mix (20 or 5 μM CoA, 5 mM ATP, 20 mM MgCl₂, 8 mM L-Gln and 100 mM Tris-CL, 1.66 μM apo-BpsA, pH 7.8) was then added to each well. Reactions were then initiated by addition of 50 μL 0.32-1.0 μM PPTase in water to each well followed by mixing at 1000 rpm for 10 s. A₅₉₀ values were then recorded and PPTase velocities determined as previously described.

4.17.2—Screening of the Lopac¹²⁸⁰ Chemical Library to Identify Novel Inhibitors of the P. aeruginosa PPTase PcpS.

1.8 μL of each compound from a Lopac¹²⁸⁰ library plate was added to 304 of 10% DMSO in MQ using a CyBio CyBi-well, giving a final compound concentration of approximately 18 μM. Next 50 μL of a master mix containing 5 μM Co-enzyme A, 1.66 μM BpsA, 20 mM MgCl₂, 5 mM ATP, 8 mM L-Gln, 100 mM Tris-HCl pH 7.8 and MQ was added. To initiate the reaction 20 μL of PcpS at a final reaction concentration of 0.18 μM and MQ was added rapidly, using an automatic dispensing Pipette. The plate was then shaken for 10 seconds at 1000 rpm to mix the compounds. The plate was then read 25 times to measure the Absorbance at 590 nm (with 20 second intervals between each read) in an EnSpire plate reader at 25° C. Each plate had 80 compounds added to rows 2-11 leaving the first and last column empty. Negative and positive controls were established in wells in these columns, and used to monitor the reaction. The negative controls had 20 μL of MQ added instead of PcpS. The positive controls had 1.8 μL of DMSO added instead of a compound. Both control reactions were run in triplicate.

### 4.17.3—Assessment of PcpS Inhibition by Bay11-7085.

For IC₅₀ analysis a master mix containing: 5 μM CoA, 5 mM ATP, 20 mM MgCl₂, 8 mM L-Gln, 100 mM Tris-Cl pH 7.8, 1.66 μM BpsA was used to establish a reaction in 24 wells in a 96 well plate. Bay 11-7085 was then serially diluted from 20 μM to 0.625 μM across the wells. The reaction was initiated by the addition of PcpS to a final concentration of 0.18 μM and indigoidine production was then monitored at 590 nm. Each concentration was tested in triplicate and the average of the three wells was taken to calculate the maximum velocity. The maximum velocity in each well was expressed as a percentage of the maximum velocity in the wells where no Bay11-7085 was present. For qualitative assessment of Bay11-7085 mediated inhibition of PcpS relative to 6-NOBP, duplicate wells were established with either 20 μM of each inhibitor or with DMSO added instead of an inhibitor (positive control). For a negative control, ddH₂O was added instead of PcpS. Reactions were initiated by addition of 20 μL of PcpS at a final reaction concentration of 0.18 μM (or H₂O for the negative control) using an automatic dispensing Pipette. The plate was then shaken for 10 seconds at 1000 rpm to mix the compounds, and incubated on the benchtop until the positive control wells had gone dark blue.

### Table 5.1: Nucleotide and Amino Acid Sequences (SEQ ID NO: 1-SEQ ID NO: 63)

Table 5.1: SEQ ID NO: 1-SEQ ID NO: 21 are also presented in FIGS. 25 to 45. SEQ ID NO: 22-SEQ ID NO: 41: Primers used in this study as shown in Table 4.3. SEQ ID NO: 42-63—The nucleotide sequences for the inserted DNA fragments identified in eDNA clones 1-7 from eDNA library 1 as described in section 3.6, FIG. 22 and Table 3.2; and eDNA clones 1-14 from eDNA library 2 as described in section 3.8, FIG. 23 and Table 3.4

TABLE 5.1

| | |
|---|---|
| wtBpsA: polynucleotide (FIG. 25) | (SEQ ID NO: 1) |
| s1BpsA polynucleotide (FIG. 26) | (SEQ ID NO: 2) |
| s1BpsA polypeptide (FIG. 27) | (SEQ ID NO: 3) |
| s1PvdD polynucleotide (FIG. 28) | (SEQ ID NO: 4) |
| s1PvdD polypeptide (FIG. 29) | (SEQ ID NO: 5) |
| s1PvdD2 polynucleotide (FIG. 30) | (SEQ ID NO: 6) |
| s1PvdD2 polypeptide (FIG. 31) | (SEQ ID NO: 7) |
| s1Pst polynucleotide (FIG. 32) | (SEQ ID NO: 8) |
| s1Pst polypeptide (FIG. 33) | (SEQ ID NO: 9) |
| s1EntF polynucleotide (FIG. 34) | (SEQ ID NO: 10) |
| s1EntF polypeptide (FIG. 35) | (SEQ ID NO: 11) |
| s1DhbF polynucleotide (FIG. 36) | (SEQ ID NO: 12) |
| s1DhbF polypeptide (FIG. 37) | (SEQ ID NO: 13) |
| 5k5 polynucleotide (FIG. 38) | (SEQ ID NO: 14) |
| 5k5 polypeptide (FIG. 39) | (SEQ ID NO: 35) |
| Pr2H6 polynucleotide (FIG. 40) | (SEQ ID NO: 16) |
| Pr2H6 polypeptide (FIG. 41) | (SEQ ID NO: 17) |
| oBpsA polynucleotide (FIG. 42) | (SEQ ID NO: 18) |
| oBpsA polypeptide (FIG. 43) | (SEQ ID NO: 19) |
| wtBpsA T-domain polynucleotide (FIG. 44) | (SEQ ID NO: 20) |
| wtBpsA T-domain polynucleotide (FIG. 45) | (SEQ ID NO: 21) |
| pBPSA3_Lup polynucleotide | (SEQ ID NO: 22) |
| pBPSA3_Ldwn polynucleotide | (SEQ ID NO: 23) |
| pBPSA3_Rup) polynucleotide | (SEQ ID NO: 24) |
| pBPSA3_Rdwn polynucleotide | (SEQ ID NO: 25) |
| slBPSAT_Fwd polynucleotide | (SEQ ID NO: 26) |
| slBPSAT_Rev polynucleotide | (SEQ ID NO: 27) |
| slPvdDT_Fwd polynucleotide | (SEQ ID NO: 28) |
| slPvdDT_Rev polynucleotide | (SEQ ID NO: 29) |
| slDhbFT_Fwd polynucleotide | (SEQ ID NO: 30) |
| slDhbFT_Fwd_Rev polynucleotide | (SEQ ID NO: 31) |
| slEntFT_Fwd polynucleotide | (SEQ ID NO: 32) |
| slEntFT_Rev polynucleotide | (SEQ ID NO: 33) |
| slEntBT_Fwd polynucleotide | (SEQ ID NO: 34) |
| slEntBT_Rev polynucleotide | (SEQ ID NO: 35) |
| PP_PPTase_fwd_NdeI polynucleotide | (SEQ ID NO: 36) |
| PP_PPTase_rev_SalI polynucleotide | (SEQ ID NO: 37) |
| pcpS_fwd_NdeI polynucleotide | (SEQ ID NO: 38) |
| pcpS_rev_SalI polynucleotide | (SEQ ID NO: 39) |
| sfp_fwd_NdeI polynucleotide | (SEQ ID NO: 40) |
| sfp_rev_SalI polynucleotide | (SEQ ID NO: 41) |
| eDNA library 1 hit 1 insert | (SEQ ID NO: 42) |
| eDNA library 1 hit 2 insert | (SEQ ID NO: 43) |
| eDNA library 1 hit 3 insert | (SEQ ID NO: 44) |
| eDNA library 1 hit 4 insert | (SEQ ID NO: 45) |
| eDNA library 1 hit 5 insert | (SEQ ID NO: 46) |
| eDNA library 1 hit 6 forward contig | (SEQ ID NO: 47) |
| eDNA library 1 hit 6 reverse contig | (SEQ ID NO: 48) |
| eDNA library 1 hit 7 insert | (SEQ ID NO: 49) |
| eDNA library 2 hit 1 insert | (SEQ ID NO: 50) |
| eDNA library 2 hit 2 insert | (SEQ ID NO: 51) |
| eDNA library 2 hit 3 insert | (SEQ ID NO: 52) |
| eDNA library 2 hit 4 insert | (SEQ ID NO: 53) |
| eDNA library 2 hit 5 insert | (SEQ ID NO: 54) |
| eDNA library 2 hit 6 insert | (SEQ ID NO: 55) |
| eDNA library 2 hit 7 insert | (SEQ ID NO: 56) |
| eDNA library 2 hit 8 insert | (SEQ ID NO: 57) |
| eDNA library 2 hit 9 insert | (SEQ ID NO: 58) |
| eDNA library 2 hit 10 insert | (SEQ ID NO: 59) |
| eDNA library 2 hit 11 insert | (SEQ ID NO: 60) |
| eDNA library 2 hit 12 insert | (SEQ ID NO: 61) |
| eDNA library 2 hit 13 insert | (SEQ ID NO: 62) |
| eDNA library 2 hit 14 insert | (SEQ ID NO: 63) |

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

The invention consists in the foregoing and also envisages constructions of which the aforementioned gives examples only.

### REFERENCES

1. Altschul et al. (1997). Nuc. Acid Res 25:3389-3402,
2. Baltz, R. H., et al., “Combinatorial biosynthesis of lipopeptide antibiotics in Streptomyces roseosporus”. J Ind. Microbiol Biotechnol, 2006. 33(2): p. 66-74.
3. Barekzi N, Joshi S, Irwin S, et al. (2004). Genetic characterization of pcpS, encoding the multifunctional phosphopantetheinyl transferase of Pseudomonas aeruginosa. Microbiology 150:795-803.
4. Brady, S. F. (2007). “Construction of soil environmental DNA cosmid libraries and screening for clones that produce biologically active small molecules.” Nat. Protocols 2(5): 1297-1305.
5. Bowie et al., 1990, Science 247, 1306.
6. Caboche, S., et al., NORINE: a database of nonribosomal peptides. Nucl. Acids Res., 2007: p. gkm792.
7. Caboche, S., et al., Structural pattern matching of nonribosomal peptides. BMC Structural Biology, 2009. 9(1): p. 15.
8. Challis, G. L. and J. H. Naismith, Structural aspects of non-ribosomal peptide biosynthesis. Curr Opin Struct Biol, 2004. 14(6): p. 748-56.
9. Chalut C, Botella L, de Sousa-D'Auria C, et al. (2006). The nonredundant roles of two 4′-phosphopantetheinyl transferases in vital processes of Mycobacteria. Proc Natl Acad Sci USA 103:8511-6.
10. Doekel, S., M.-F. Coeffet-Le Gal, et al. (2008). “Non-ribosomal peptide synthetase module fusions to produce derivatives of daptomycin in Streptomyces roseosporus.” Microbiology 154(9): 2872-2880.
11. Donadio, S., P. Monciardini, and M. Sosio, Polyketide synthases and nonribosomal peptide synthetases: the emerging view from bacterial genomics. Natural Product Reports, 2007. 24: p. 1073-1109.
12. Du et al., FEMS Microbiology Letters, 2000, 189:171-175.
13. Duckworth B P, Aldrich C C. 2010. Development of a high-throughput fluorescence polarization assay for the discovery of phosphopantetheinyl transferase inhibitors. Anal Biochem 403:13-9.
14. Finking, R. and M. A. Marahiel, Biosynthesis of nonribosomal peptides 1. Annu Rev Microbiol, 2004. 58: p. 453-88.
15. Finking, R., J. Solsbacher, et al. (2002). “Characterization of a New Type of Phosphopantetheinyl Transferase for Fatty Acid and Siderophore Synthesis in Pseudomonas aeruginosa.” J. Biol. Chem. 277(52): 50293-50302
16. Foley T L, Young B S, Burkart M D. 2009. Phosphopantetheinyl transferase inhibition and secondary metabolism. FEBS J 276:7134-45.
17. Frueh, D. P., H. Arthanari, et al. (2008). “Dynamic thiolation-thioesterase structure of a non-ribosomal peptide synthetase.” Nature 454(7206): 903-906.
18. Gal, M.-F. C.-L., et al., Complementation of daptomycin dptA and dptD deletion mutations in trans and production of hybrid lipopeptide antibiotics. Microbiology, 2006. 152(10): p. 2993-3001.
19. Ginolhac, A., C. Jarrin, et al. (2004). “Phylogenetic Analysis of Polyketide Synthase I Domains from Soil Metagenomic Libraries Allows Selection of Promising Clones.” Appl. Environ. Microbiol. 70(9): 5522-5527.
20. Gu, J.-Q., et al., Structural Characterization of Daptomycin Analogues A21978C1-3(d-Asn11) Produced by a Recombinant Streptomyces roseosporus Strain. Journal of Natural Products, 2007. 70(2): p. 233-240.
21. Guzman et al., (1995) J. Bacteriol., 177(14), 4121-4130
22. Hanahan, D. (1983). “Studies on transformation of Escherichia coli with plasmids.”Journal of Molecular Biology 166(4): 557-580.
23. Huang, X. (1994) On Global Sequence Alignment. Computer Applications in the Biosciences 10, 227-235.
24. Kim J H, Feng Z, Bauer J D, Kallifidas D, Calle P Y, Brady S F. 2010. Cloning large natural product gene clusters from the environment: piecing environmental DNA gene clusters back together with TAR. Biopolymers 93(9):833-44.
25. Koglin, A., M. R. Mofid, et al. (2006). “Conformational Switches Modulate Protein Interactions in Peptide Antibiotic Synthetases.” Science 312(5771): 273-276.
26. Koglin, A. and C. T. Walsh, Structural insights into nonribosomal peptide enzymatic assembly lines. Natural Product Reports, 2009. 26(8): p. 987-1000.
27. Kuhn, R., M. P. Starr, et al. (1965). “Indigoidine and other bacterial pigments related to 3,3′-bipyridyl.” Archives of Microbiology 51(1): 71-84.
28. Laemmli, U. K. (1970). “Cleavage of structural proteins during the assembly of the head of bacteriophage T4.” Nature 227(259): 680-685.
29. Lai, J. R., M. A. Fischbach, et al. (2006). “A protein interaction surface in nonribosomal peptide synthesis mapped by combinatorial mutagenesis and selection.” 103(14): 5314-5319.
30. Lai, J. R., A. Koglin, et al. (2006). “Carrier Protein Structure and Recognition in Polyketide and Nonribosomal Peptide Biosynthesis.” Biochemistry 45(50): 14869-14879.
31. Marahiel, M. A., T. Stachelhaus, et al. (1997). “Modular Peptide Synthetases Involved in Nonribosomal Peptide Synthesis.” Chem. Rev. 97(7): 2651-2674. Marahiel, M. A. and L. O. Essen, Nonribosomal peptide synthetases: Mechanistic and structural aspects of essential domains. Methods in Enzymology, 2009. 458: p. 337-351.
32. Marahiel, M. A. and L. O Essen (2009). “Nonribosomal peptide synthetases: Mechanistic and structural aspects of essential domains.” Methods in Enzymology. 458: 337-351.
33. Meier, J. L. and M. D. Burkart, The chemical biology of modular biosynthetic enzymes. Chemical Society Reviews, 2009. 38(7): p. 2012-2045.
34. Miao, V., M.-F. Coeffet-LeGal, et al. (2005). “Daptomycin biosynthesis in Streptomyces roseosporus: cloning and analysis of the gene cluster and revision of peptide stereochemistry.” Microbiology 151(5): 1507-1523.
35. Miao, V., et al., Genetic Engineering in Streptomyces roseosporus to Produce Hybrid Lipopeptide Antibiotics. Chemistry & Biology, 2006. 13(3): p. 269-276.
36. Mootz, H. D., R. Finking, et al. (2001). “4′-Phosphopantetheine Transfer in Primary and Secondary Metabolism of Bacillus subtilis.” J. Biol. Chem. 276(40): 37289-37298.
37. Needleman, S. B. and Wunsch, C. D. (1970) J. Mol. Biol. 48, 443-453
38. Nguyen, K. T., et al., Genetically Engineered Lipopeptide Antibiotics Related to A54145 and Daptomycin with Improved Properties. Antimicrob. Agents Chemother., 2010: p. AAC.01307-09.
39. Nguyen, K. T., et al., Combinatorial biosynthesis of novel antibiotics related to daptomycin. Proceedings of the National Academy of Sciences, 2006. 103(46): p. 17462-17467.
40. Owen J G, J N Copp and D F Ackerley. 2011. Rapid and flexible biochemical assays for evaluating 4′-phosphopantetheinyl transferase activity. Biochemical Journal 436: 709-717.
41. Parachin N S, Gorwa-Grauslund M F. 2011. Isolation of xylose isomerases by sequence- and function-based screening from a soil metagenomic library. Biotechnol Biofuels 4:9.
42. Rice, P et al., EMBOSS: The European Molecular Biology Open Software Suite, Trends in Genetics June 2000, vol 16, No 6. pp. 276-277
43. Sambrook, J. and D. W. Russell (2003). Molecular Cloning: A Laboratory Manual, Cold Spring Harbour Laboratory Press.
44. Sambrook, J. and D. W. Russell (2006). “Transformation of E. coli by Electroporation.”Cold Spring Harbor Protocols 2006(2): pdb.prot3933-.
45. Samel, S. A., G. Schoenafinger, et al. (2007). “Structural and Functional Insights into a Peptide Bond-Forming Bidomain from a Nonribosomal Peptide Synthetase.” Structure 15(7): 781-792. Schweizer, H. P. and T. T. Hoang, An improved system for gene replacement and xylE fusion analysis in Pseudomonas aeruginosa. Gene, 1995. 158(1): p. 15-22.
46. Schwecke, T., J. F. Aparicio, et al. (1995). “The biosynthetic gene cluster for the polyketide immunosuppressant rapamycin.” Proceedings of the National Academy of Sciences of the United States of America 92(17): 7839-7843.
47. Seidle, H. F., R. D. Couch, et al. (2006). “Characterization of a nonspecific phosphopantetheinyl transferase from Pseudomonas syringae pv. syringae FF5.” Archives of Biochemistry and Biophysics 446(2): 167-174.
48. Stachelhaus, T. and M. A. Marahiel, Modular structure of genes encoding multifunctional peptide synthetases required for non-ribosomal peptide synthesis. FEMS Microbiology Letters, 1995. 125(1): p. 3-14.
49. Stein, T., et al., The Multiple Carrier Model of Nonribosomal Peptide Biosynthesis at Modular Multienzymatic Templates. J. Biol. Chem., 1996. 271(26): p. 15428-15435.
50. Studier, F. W. (2005). “Protein production by auto-induction in high-density shaking cultures.” Protein Expression and Purification 41(1): 207-234.
51. Sunbul, M., et al., Chapter 10 Using Phosphopantetheinyl Transferases for Enzyme Posttranslational Activation, Site Specific Protein Labeling and Identification of Natural Product Biosynthetic Gene Clusters from Bacterial Genomes, in Methods in Enzymology. 2009, Academic Press. p. 255-275.
52. Takahashi, H., T. Kumagai, et al. (2007). “Cloning and Characterization of a Streptomyces Single Module Type Non-ribosomal Peptide Synthetase Catalyzing a Blue Pigment Synthesis.” J. Biol. Chem. 282(12): 9073-9081.
53. Tanovic, A., S. A. Samel, et al. (2008). “Crystal Structure of the Termination Module of a Nonribosomal Peptide Synthetase.” Science 321(5889): 659-663.
54. Tatiana A. et al, FEMS Microbiol Lett. 174:247-250 (1999).
55. Vizcaíno, J. A., L. S., R. E. Cardoza, E. Monte, S. Gutiérrez, Detection of putative peptide synthetase genes in Trichoderma species: Application of this method to the cloning of a gene from T. harzianum CECT 2413. FEMS Microbiology Letters, 2005. 244(1): p. 139-148.
56. Walsh, C. T., The Chemical Versatility of Natural-Product Assembly Lines. Accounts of Chemical Research, 2007. 41(1): p. 4-10.
57. Walsh, et al. 2004, Science 303: pp. 1805-1810.
58. Yasgar A, Foley T L, Jadhav A, Inglese J, Burkart M D, Simeonov A. 2010. A strategy to discover inhibitors of Bacillus subtilis surfactin-type PPTase. Mol Biosyst 6:365-75.
59. Yin, J., P. D. Straight, et al. (2007). “Genome-Wide High-Throughput Mining of Natural-Product Biosynthetic Gene Clusters by Phage Display.” Chemistry & Biology 14(3): 303-312.
60. Zhou, Z., J. R. Lai, et al. (2006). “Interdomain Communication between the Thiolation and Thioesterase Domains of EntF Explored by Combinatorial Mutagenesis and Selection.” Chemistry & Biology 13(8): 869-879.
61. Zou Y, Yin J. 2009. Phosphopantetheinyl transferase catalyzed site-specific protein labeling with ADP conjugated chemical probes. J Am Chem. Soc. 131(22):7548-9

What we claim is:

1. A method of identifying a candidate nucleic acid (CNA) comprising one or more of the polynucleotide sequences selected from the group consisting of,
a) at least a part of a natural product gene cluster (NPGC),
b) at least a part of a secondary metabolite biosynthesis cluster (SMBC)
c) at least a part of a non ribosomal peptide (NRP), and/or polyketide (PK) biosynthesis cluster,
d) a polynucleotide sequence encoding at least one protein involved in NRP and/or PK biosynthesis,
e) a polynucleotide sequence encoding at least one protein involved in other secondary metabolite biosynthesis, and
f) a polynucleotide sequence encoding at least one phosphopantetheinyl transferase (PPTase),
the method comprising,
expressing said CNA or polynucleotide sequence to form at least one PPTase,
incubating said at least one PPTase with a non ribosomal peptide synthetase (NRPS), and detecting activation of said NRPS,
wherein said activation indicates that said CNA comprises at least one of a)-f).

2. A method of claim 1 wherein the method comprises the additional step of further characterizing the CNA to identify at least one of a)-f).

3. A method according to claim 1 or 2 wherein said expressing is in vivo.

4. A method according to any one of claims 1 to 3 wherein the NRPS is encoded by at least one of:
a. a polynucleotide encoding a BpsA synthetase,
b. a polynucleotide comprising a nucleotide sequence having at least 70% sequence identity with SEQ ID NO: 1,
c. a polynucleotide comprising SEQ ID NO: 1,
d. a polynucleotide consisting of a nucleotide sequence having at least 70% sequence identity with SEQ ID NO: 1,
e. a polynucleotide consisting of SEQ ID NO: 1,
f. a polynucleotide of any one of a-e above comprising a T-domain comprising at least 70% sequence identity with SEQ ID NO: 20
g. a polynucleotide of any one of a-e above comprising a T-domain consisting of at least 70% sequence identity with SEQ ID NO: 20

5. A method according to any one of claims 1 to 4 wherein the NRPS is a modified NRPS (mNRPS).

6. A method according to any one of claims 1 to 5 wherein the method comprises the additional step of characterizing at least one secondary metabolite produced due to the expression of said CNA.

7. A method of identifying a CNA comprising a polynucleotide sequence encoding a functional PPTase, the method comprising,
expressing said CNA or polynucleotide sequence to form a PPTase,
incubating said PPTase with a NRPS, and detecting activation of said NRPS, wherein said activation indicates that said CNA comprises a polynucleotide sequence encoding a functional PPTase.

8. A method of claim 7 wherein the NRPS is an NRPS as defined in claim 4 or 5 that is used to identify a CNA comprising a polynucleotide sequence that encodes a previously unknown PPTase.

9. A method according to claim 7 or 8 wherein the method comprises the additional steps,
expressing said CNA or polynucleotide sequence to form a PPTase,
incubating said PPTase with a NRPS, and
characterizing said PPTase either, by detecting activation of said NRPS in the presence or absence of purified T-domains, whereby the ability of a purified T-domain to compete with said NRPS for available coenzyme A (CoA) substrate indicates the relative affinity of said PPTase for said NRPS and said T-domain,
or, by incubating said PPTase with a range of NRP synthetases and comparing the activation of a range of NRP synthetases to activation of said NRPS.

10. A method according to claim 9 wherein the range of NRP synthetases comprises modified and/or chemically evolved NRP synthetases.

11. An mNRPS that is encoded by:
i. a polynucleotide sequence encoding a modified BpsA synthetase,
ii. a polynucleotide sequence variant of SEQ ID NO: 1 wherein said variant comprises at least 70% nucleotide sequence identity with SEQ ID NO: 1, or
iii. a polynucleotide sequence encoding a modified NRPS comprising a modified T-domain, wherein said T-domain is selected from the group consisting of:
a. a heterologous T-domain,
b. a homologous T-domain,
c. an exogenous T-domain,
d. an endogenous T-domain,
e. a T-domain encoded by a nucleotide sequence comprising at least 70% sequence identity with the T-domain of any one of SEQ ID NO: 1, 2, 4, 6, 8, 10, 12, 14, 16 and 18, and
f. a T-domain encoded by a nucleotide sequence of the T-domain of any one of the nucleotide sequences selected from the group consisting of SEQ ID NO: 1, 2, 4, 6, 8, 10, 12, 14, 16, and 18.

12. A modified NRPS (mNRPS) having:
i. an amino acid sequence that specifies a modified BpsA synthetase,
ii. an amino acid sequence variant of SEQ ID NO: 3 wherein said variant comprises at least 70% amino acid sequence identity with SEQ ID NO: 3, or
iii. an amino acid sequence that specifies a modified NRPS comprising a modified T-domain, wherein said T-domain is selected from the group consisting of:
a. a heterologous T-domain,
b. a homologous T-domain,
c. an exogenous T-domain,
d. an endogenous T-domain,
e. a T-domain specified by an amino acid sequence comprising at least 70% sequence identity with the T-domain of any one of SEQ ID NO: 3, 5, 7, 9, 11, 13, 15, 17 and 19, and
f. a T-domain encoded by a nucleotide sequence of the T-domain of any one of the nucleotide sequences selected from the group consisting of SEQ ID NO: 3, 5, 7, 9, 11, 13, 15, 17 and 19.

13. A mNRPS encoded by a polynucleotide sequence according to claim 11.

14. A mNRPS comprising a polypeptide sequence selected from the group consisting of a polypeptide sequence comprising at least 70% identity to SEQ ID NO: 3, 5, 7, 9, 11, 13, 15, 17, or 19, and a polypeptide sequence comprising SEQ ID NO: 3, 5, 7, 9, 11, 13, 15, 17 or 19.

15. A method of making a mNRPS comprising
a. modifying the nucleotide sequence encoding the T-domain of a polynucleotide encoding a BpsA synthetase to make a modified polynucleotide, and
b. expressing said modified polynucleotide under suitable conditions to form a mNRPS.

16. A method of claim 15 comprising the additional step of
c. isolating and purifying said mNRPS.

17. A method of claim 16 comprising the additional steps of
d. incubating said mNRPS with a PPTase, and
e. detecting the activation of said mNRPS by the PPTase,
wherein activation of said mNRPS confirms that said mNRPS is a modified NRPS that is activated by the PPTase.

18. A method of claim 17 comprising the additional steps of
f. characterizing the catalytic activity and specificity of a given PPTase for 4′-PP attachment to said mNRPS, and
g. comparing said catalytic activity and specificity to the catalytic activity and specificity of the same PPTase for 4′-PP attachment to another NRPS,
thereby identifying that the PPTase has different catalytic activity and/or specificity for the mNRPS.

19. A method of claim 17 or 18 where the given PPTase is a known PPTase, a wild type NRPS, an mNRPS according to any one of claims 11 to 14 or an mNRPS made according to any one of claims 15 to 18.

20. A method of characterizing the ability of a PPTase or suspected PPTase, to activate an NRPS, the method comprising
a. incubating said PPTase or suspected PPTase with an NRPS, and
b. detecting the activation of said NRPS,
thereby characterizing the PPTase or suspected PPTase as capable of activating said NRPS.

21. A method of claim 20 wherein the NRPS is an mNRPS and the method further comprises an additional step of
c. comparing the binding activity and specificity of the PPTase or suspected PPTase for the mNRPS with the binding activity and specificity of a corresponding wild type NRPS.

22. A method of characterizing the activity of a PPTase, the method comprising the steps of
combining said PPTase, a BpsA synthetase and the necessary substrates for phosphopantetheinylation of said BpsA synthetase in the presence of a carrier protein or peptide that acts as a competitor for one or more of the phosphopantetheinylation substrates,
incubating the resulting reaction,
adding the necessary substrates for an indigoidine synthesis reaction,
using a measurement tool to measure the indoigidine level produced by the synthesis reaction, and
computing the rate of indigoidine produced over a time period,
wherein the rate of indigoidine production is indicative of the amount of BpsA synthetase converted from apo to holo form and allows determination of the relative rate of carrier protein or peptide modification by the PPTase.

23. A method of claim 22 wherein said PPTase, BpsA synthetase and necessary substrates are combined in the presence of a range of known concentrations of said carrier protein or peptide competitor.

24. A method of claim 22 or 23 wherein said one or more of the phosphopantetheinylation substrates are present in a limiting amount.

25. A method of making a modified PPTase, the method comprising
a. expressing a modified PPTase from a polynucleotide sequence to form an expressed PPTase,
b. incubating the expressed PPTase with an NRPS, and
c. detecting the activation of the NRPS,
wherein activation of the NRPS confirms that said expressed PPTase is a functional modified PPTase.

26. A method of claim 25 wherein the polynucleotide sequence encoding the modified PPTase has been modified by error-prone PCR, targeted mutagenesis, or DNA shuffling.

27. A method of claim 25 or 26 further comprising a step of characterizing the activity of the modified PPTase.

28. An assay platform wherein a pigment synthesising enzyme acts as a reporter for PPTase activity in vivo.

29. An assay platform wherein a pigment synthesising enzyme acts as a reporter for PPTase activity in vitro.

30. A method of characterizing the rate of reaction of PPTases, the method comprising the steps of:
combining a pigment producing enzyme with a PPTase and substrates and co-factors required for both phosphopantetheinylation and pigment production,
using a measurement tool to measure the pigment level, and
computing the rate of pigment produced over a time period,
wherein the change in rate of pigment produced is proportional to the rate of reaction of the PPTase to be characterized.

31. A method of detecting a chemical modifier of PPTase activity, the method comprising the steps of
combining a pigment producing enzyme with a PPTase and substrates and co-factors required for both phosphopantetheinylation and pigment production in the presence of a chemical compound to be tested
using a measurement tool to measure the pigment produced,
computing the rate of pigment produced over a time period,
wherein if the rate of the reaction for the PPTase slows in the presence of the chemical, it is a candidate inhibitor, or if the rate of reaction increases, the chemical is a candidate accelerator.

32. A method of counter-screening a candidate inhibitor or candidate accelerator identified using the method of claim 31, wherein said candidate inhibitor or candidate accelerator is re-screened in reactions using pre-activated holo-BpsA, thereby confirming that the candidate inhibitor or accelerator modifies the function of the target PPTase.

33. A method of determining the rate of modification of any carrier protein or peptide substrate by any PPTase, the method comprising the steps of
combining a PPTase, a pigment producing enzyme and the necessary substrates for phosphopantetheinylation in the presence of a range of known concentrations of a carrier protein or peptide that acts as a competitor for one or more of the phosphopantetheinylation substrates which is in limited supply,
incubating the resulting reaction,
adding the necessary substrates for the pigment production reaction,
using a measurement tool to measure the pigment level, and
computing the rate of pigment produced over a time period,
wherein the rate of pigment production is indicative of the amount of pigment producing enzyme converted from apo to holo form and allows determination of the relative rate of carrier protein or peptide modification by the PPTase.

34. A method of claim 33 that allows different PPTase and carrier protein/peptide combinations that are highly active but do not show cross-reactivity with other highly active PPTase and carrier protein/peptide combinations to be adapted for efficient site-specific orthogonal labeling of proteins

35. A method of claim 34 that allows different PPTases to be recovered from the same eDNA library using different BpsA or modified BpsA synthetases according to the method of claim 7.

36. A method of claim 35 further comprising characterizing said different PPTases using the specific BpsA or modified BpsA synthetases that they were recovered with, thereby providing a basis for development of specific PPTase and carrier protein/peptide combinations to enable efficient site-specific orthogonal labeling of proteins.

37. A method of any one of claims 30 to 36 wherein the pigment producing enzyme may be modified, by swapping T-domains and evolving the resulting modified pigment producing enzyme that allows it to be converted into a substrate for any PPTase.

38. A method of claim 37 wherein the PPTase is selected from the group consisting of Sfp of B. subtilis subsp. spizizenii ATCC6633, PcpS of P. aeruginosa PAO1 and the putative PPTase PP1183 of P. putida KT2440.

39. A method of any one of claims 30 to 38 wherein the pigment producing enzyme is an NRPS or a PKS enzyme.

40. A method of claim 39 wherein the pigment producing enzyme is BpsA or a modified BpsA synthetase.

41. A method of any one of claims 30 to 40 wherein the substrate and cofactors may include CoA, Mg²⁺ and L-glutamine and Adenosine-5′-triphosphate (ATP).

42. A method of any one of claims 30 to 41 wherein the pigment is indigoidine.

43. A method to evaluate PPTase activity by monitoring acceleration of the rate of indigoidine synthesis.

44. A method of claim 43 where the acceleration of the rate of indigoidine synthesis may be used as a measure of the rate of 4′-PP attachment to apo-BpsA.


SEQUENCE LISTING (63 sequences)

SEQ ID NO: 1 - DNA, 3849 bp, Streptomyces lavendulae
atgactcttcaggagaccagcgtgctcgagcccaccctgcaggggaccaccacgttgcccggcctgctcgcccagcgggtggccgaacaccccgaggcgatcgcggtcgcctaccgggacgacaagctcaccttccgcgagctcgcgtccagaagcgcggccctcgccgactacctggagcacctcggtgtctccgccgacgactgcgtcggcctgttcgtcgagccgtcgatcgacctgatggtcggcgcctggggcatcctcaacgccggcgccgcgtacctgccgctgtccccggagtaccccgaggaccggctgcgctacatgatcgagaacagcgagacgaagatcatcctggcgcagcagcgcctggtgtcccgtctgcgcgagctcgcgccgaaggacgtcaccatcgtgaccctgcgcgagtccgaggccttcgtccgccccgagggcaccgaggccccggccgcccgcagcgcccgcccggacaccctcgcgtacgtcatctacacctccggcagcacgggcaagccgaagggtgtgatgatcgagcaccgcagcatcgtcaaccagctcggctggctgcgcgagacctacgcgatcgaccgcagcaaggtcatcctccagaagaccccgatgagcttcgacgccgcccagtgggagatcctctccccggccaacggcgccaccgtcgtcatgggcgccccgggcgtctacgccgaccccgagggcctcatcgagaccatcgtcaagcacaacgtgaccaccctccagtgcgtcccgacgctgctccagggtctgatcgacaccgagaagttccccgagtgcgtctccctccagcagatcttcagcggtggcgaggccctctcccgcctgctggcgatccagaccacgcaggagatgcccggccgggcgctcatcaacgtctacgggccgaccgagacgacgatcaactcgtcctcgttccccgtcgaccccgccgacctggacgagggaccgcagtccatctccatcggctccccggtgcacggcaccacgtaccacatccttgacaaggagaccctcaagccggtcggcgtcggtgagatcggcgagctgtacatcggcggcatccagctggcccgcggctacctgcaccgcgacgacctgaccgccgagcgcttcctggagatcgagctcgaggagggcgccgagcccgtccgcctgtacaagacgggcgacctcggccagtggaacaacgacggcaccgtgcagttcgccggccgcgccgacaaccaggtcaagctgcgcggctaccgcgtcgagctcgacgagatatccctggcgatcgagaaccacgactgggtccgcaacgccgccgtcatcgtcaagaacgacggccgcaccggcttccagaacctgatcgcctgcatcgagctgagcgagaaggaagccgccctgatggaccagggcaaccacggctcccaccacgcgtcgaagaagagcaagctccaggtcaaggcgcagctgtccaacccgggcctgcgcgacgacgccgagctggccgcccgcccggccttcgacctggagggcgccgagcccacccccgagcagcgcgcccgggtcttcgcccgcaagacgtaccgcttctacgagggcggcgccgtcacccaggccgacctgctgggcctgctgggcgccacggtcaccgccggctactcgcgcaaggcggccgacctggcccccgccgaactcggccagatcctgcgctggttcggccagtacatcagcgaggagcggctcctgccgaagtacggctacgcctccccgggcgcgctgtacgcgacgcagatgtacttcgagctggagggcgtcggcggtctgaagccgggctactactactaccagccggtccgccaccagctcgtcctcatcagcgagcgcgaggccaccggcaaggccacggcgcagatccacttcatcggcaagaagagcggcatcgagccggtctacaagaacaacatcctcgaggtcctggagatcgagaccggccacatggtcggcctcttcgagcagatcctgccggcctacggcctcgacatccacgaccgcgcctacgagccggccgtcaaggacctgctcgacgtcgccgacgaggactactacctgggcaccttcgagctggtcccgcacgcgggcgcgcgcgacgaccaggccgaggtctacgtccagacgcacggcggaaaggtcgccggcctgcccgagggccagtaccgctacgagaacggcgagctgacccgcttctcggacgacatcgtcctcaagaagcacgtcatcgcgatcaaccagtcggtgtaccaggccgccagcttcggcatcagcgtctacagccgcgccgaggaggagtggctgaagtacatcaccctcggcaagaagctccagcacctgatgatgaacgggctgaacctgggcttcatgtcctcgggctacagctccaagacgggcaacccgctgccggcctcgcgccgcatggacgccgtcctcggcgccaacggcgtcgacagcgccccgatgtacttcttcgtcggcggccgcatcagcgacgagcagatcggccacgagggcatgcgcgaggacagcgtccacatgcgcggtccggccgagctcatccgcgacgacctcgtcagcttcctcccggactacatgatccccaaccgggtcgtggtcttcgaccggctgccgctgtccgccaacggcaagatcgacgtcaaggcgctcgccgcctccgaccaggtcaacgccgagctcgtcgagcgccccttcgtcgccccgcgcacggagacggagaaggagatcgcggcggtctgggagaaggccctgcgccgcgagaacgcctccgtccaggacgacttcttcgagtcgggcggcaactcgctgatcgccgtcggcctcgtccgcgagctcaacgcgcgcctgggcgtctccctgccgctgcagagcgtcctggagtccccgaccatcgagaagctggcccgccgcctggagcgcgaggtcgcccaggagtcctcgcgcttcgtccgcctgcacgcggagaccggcaaggcccggcccgtgatctgctggccgggtctgggcggctacccgatgaacctgcgcagcctggccggcgagatcggcctcggccgctcgttctacggcgtccagtcctacggcatcaacgagggcgagaccccgtacgagaccatcaccgagatggccaagaaggacatcgaggccctcaaggagatccagccggccggcccctacaccctgtggggctactccttcggcgcccgcgtggccttcgagaccgcctaccagctggagcaggcgggcgagaaggtggacaacctcttcctgatcgccccgggctccccgaaggtgcgcgccgagaacggcaaggtgtggggccgcgaggcgtccttcgccaaccgcggctacaccacgatcctgttctcggtcttcaccggcaccatttccggtccggacctggaccggtgcctggagaccgtgacggacgaggcctccttcgccgagttcatcagcgagctcaagggaatcgacgtcgacctcgcccgccggatcatctcggtcgtgggccagacgtacgaattcgagtactccttccacgagctggccgagcgcaccctccaggcgccgatcagcatcttcaaggccgtgggcgacgactactcgttcctggagaacagcagcggctactcggccgagccgccgacggtcatcgacctcgacgccgaccactacagcctgctgcgcgaggacatcggcgagctggtgaagcacatccgctacctgctcggcgagtga

SEQ ID NO: 2 - DNA, 3849 bp, Artificial
  Nucleic acid construct
atgactcttcaggagaccagcgtgctcgagcccaccctgcaggggaccaccacgttgcccggcctgctcgcccagcgggtggccgaacaccccgaggcgatcgcggtcgcctaccgggacgacaagctcaccttccgcgagctcgcgtccagaagcgcggccctcgccgactacctggagcacctcggtgtctccgccgacgactgcgtcggcctgttcgtcgagccgtcgatcgacctgatggtcggcgcctggggcatcctcaacgccggcgccgcgtacctgccgctgtccccggagtaccccgaggaccggctgcgctacatgatcgagaacagcgagacgaagatcatcctggcgcagcagcgcctggtgtcccgtctgcgcgagctcgcgccgaaggacgtcaccatcgtgaccctgcgcgagtccgaggccttcgtccgccccgagggcaccgaggccccggccgcccgcagcgcccgcccggacaccctcgcgtacgtcatctacacctccggcagcacgggcaagccgaagggtgtgatgatcgagcaccgcagcatcgtcaaccagctcggctggctgcgcgagacctacgcgatcgaccgcagcaaggtcatcctccagaagaccccgatgagcttcgacgccgcccagtgggagatcctctccccggccaacggcgccaccgtcgtcatgggcgccccgggcgtctacgccgaccccgagggcctcatcgagaccatcgtcaagcacaacgtgaccaccctccagtgcgtcccgacgctgctccagggtctgatcgacaccgagaagttccccgagtgcgtctccctccagcagatcttcagcggtggcgaggccctctcccgcctgctggcgatccagaccacgcaggagatgcccggccgggcgctcatcaacgtctacgggccgaccgagacgacgatcaactcgtcctcgttccccgtcgaccccgccgacctggacgagggaccgcagtccatctccatcggctccccggtgcacggcaccacgtaccacatccttgacaaggagaccctcaagccggtcggcgtcggtgagatcggcgagctgtacatcggcggcatccagctggcccgcggctacctgcaccgcgacgacctgaccgccgagcgcttcctggagatcgagctcgaggagggcgccgagcccgtccgcctgtacaagacgggcgacctcggccagtggaacaacgacggcaccgtgcagttcgccggccgcgccgacaaccaggtcaagctgcgcggctaccgcgtcgagctcgacgagatatccctggcgatcgagaaccacgactgggtccgcaacgccgccgtcatcgtcaagaacgacggccgcaccggcttccagaacctgatcgcctgcatcgagctgagcgagaaggaagccgccctgatggaccagggcaaccacggctcccaccacgcgtcgaagaagagcaagctccaggtcaaggcgcagctgtccaacccgggcctgcgcgacgacgccgagctggccgcccgcccggccttcgacctggagggcgccgagcccacccccgagcagcgcgcccgggtcttcgcccgcaagacgtaccgcttctacgagggcggcgccgtcacccaggccgacctgctgggcctgctgggcgccacggtcaccgccggctactcgcgcaaggcggccgacctggcccccgccgaactcggccagatcctgcgctggttcggccagtacatcagcgaggagcggctcctgccgaagtacggctacgcctccccgggcgcgctgtacgcgacgcagatgtacttcgagctggagggcgtcggcggtctgaagccgggctactactactaccagccggtccgccaccagctcgtcctcatcagcgagcgcgaggccaccggcaaggccacggcgcagatccacttcatcggcaagaagagcggcatcgagccggtctacaagaacaacatcctcgaggtcctggagatcgagaccggccacatggtcggcctcttcgagcagatcctgccggcctacggcctcgacatccacgaccgcgcctacgagccggccgtcaaggacctgctcgacgtcgccgacgaggactactacctgggcaccttcgagctggtcccgcacgcgggcgcgcgcgacgaccaggccgaggtctacgtccagacgcacggcggaaaggtcgccggcctgcccgagggccagtaccgctacgagaacggcgagctgacccgcttctcggacgacatcgtcctcaagaagcacgtcatcgcgatcaaccagtcggtgtaccaggccgccagcttcggcatcagcgtctacagccgcgccgaggaggagtggctgaagtacatcaccctcggcaagaagctccagcacctgatgatgaacgggctgaacctgggcttcatgtcctcgggctacagctccaagacgggcaacccgctgccggcctcgcgccgcatggacgccgtcctcggcgccaacggcgtcgacagcgccccgatgtacttcttcgtcggcggccgcatcagcgacgagcagatcggccacgagggcatgcgcgaggacagcgtccacatgcgcggtccggccgagctcatccgcgacgacctcgtcagcttcctcccggactacatgatccccaaccgggtcgtggtcttcgaccggctgccgctgtccgccaacggcaagatcgacgtcaaggcgctcgccgcctccgaccaggtcaatgcagagctcgtcgagcgccccttcgtcgccccgcgcacggagacggagaaggagatcgcggcggtctgggagaaggccctgcgccgcgagaacgcctccgtccaggacgacttcttcgagtcgggcggcaactcgctgatcgccgtcggcctcgtccgcgagctcaacgcgcgcctgggcgtctccctgccgctgcagagcgtcctggagtccccgaccatcgagaagctggcccgccgcctggagcgcgaggtcgcccaggagtctagtcgcttcgtccgcctgcacgcggagaccggcaaggcccggcccgtgatctgctggccgggtctgggcggctacccgatgaacctgcgcagcctggccggcgagatcggcctcggccgctcgttctacggcgtccagtcctacggcatcaacgagggcgagaccccgtacgagaccatcaccgagatggccaagaaggacatcgaggccctcaaggagatccagccggccggcccctacaccctgtggggctactccttcggcgcccgcgtggccttcgagaccgcctaccagctggagcaggcgggcgagaaggtggacaacctcttcctgatcgccccgggctccccgaaggtgcgcgccgagaacggcaaggtgtggggccgcgaggcgtccttcgccaaccgcggctacaccacgatcctgttctcggtcttcaccggcaccatttccggtccggacctggaccggtgcctggagaccgtgacggacgaggcctccttcgccgagttcatcagcgagctcaagggaatcgacgtcgacctcgcccgccggatcatctcggtcgtgggccagacgtacgaattcgagtactccttccacgagctggccgagcgcaccctccaggcgccgatcagcatcttcaaggccgtgggcgacgactactcgttcctggagaacagcagcggctactcggccgagccgccgacggtcatcgacctcgacgccgaccactacagcctgctgcgcgaggacatcggcgagctggtgaagcacatccgctacctgctcggcgagtga

SEQ ID NO: 3 - protein, 1282 aa, Streptomyces lavendulae
MTLQETSVLEPTLQGTTTLPGLLAQRVAEHPEAIAVAYRDDKLTFRELASRSAALADYLEHLGVSADDCVGLFVEPSIDLMVGAWGILNAGAAYLPLSPEYPEDRLRYMIENSETKIILAQQRLVSRLRELAPKDVTIVTLRESEAFVRPEGTEAPAARSARPDTLAYVIYTSGSTGKPKGVMIEHRSIVNQLGWLRETYAIDRSKVILQKTPMSFDAAQWEILSPANGATVVMGAPGVYADPEGLIETIVKHNVTTLQCVPTLLQGLIDTEKFPECVSLQQIFSGGEALSRLLAIQTTQEMPGRALINVYGPTETTINSSSFPVDPADLDEGPQSISIGSPVHGTTYHILDKETLKPVGVGEIGELYIGGIQLARGYLHRDDLTAERFLEIELEEGAEPVRLYKTGDLGQWNNDGTVQFAGRADNQVKLRGYRVELDEISLAIENHDWVRNAAVIVKNDGRTGFQNLIACIELSEKEAALMDQGNHGSHHASKKSKLQVKAQLSNPGLRDDAELAARPAFDLEGAEPTPEQRARVFARKTYRFYEGGAVTQADLLGLLGATVTAGYSRKAADLAPAELGQILRWFGQYISEERLLPKYGYASPGALYATQMYFELEGVGGLKPGYYYYQPVRHQLVLISEREATGKATAQIHFIGKKSGIEPVYKNNILEVLEIETGHMVGLFEQILPAYGLDIHDRAYEPAVKDLLDVADEDYYLGTFELVPHAGARDDQAEVYVQTHGGKVAGLPEGQYRYENGELTRFSDDIVLKKHVIAINQSVYQAASFGISVYSRAEEEWLKYITLGKKLQHLMMNGLNLGFMSSGYSSKTGNPLPASRRMDAVLGANGVDSAPMYFFVGGRISDEQIGHEGMREDSVHMRGPAELIRDDLVSFLPDYMIPNRVVVFDRLPLSANGKIDVKALAASDQVNAELVERPFVAPRTETEKEIAAVWEKALRRENASVQDDFFESGGNSLIAVGLVRELNARLGVSLPLQSVLESPTIEKLARRLEREVAQESSRFVRLHAETGKARPVICWPGLGGYPMNLRSLAGEIGLGRSFYGVQSYGINEGETPYETITEMAKKDIEALKEIQPAGPYTLWGYSFGARVAFETAYQLEQAGEKVDNLFLIAPGSPKVRAENGKVWGREASFANRGYTTILFSVFTGTISGPDLDRCLETVTDEASFAEFISELKGIDVDLARRIISVVGQTYEFEYSFHELAERTLQAPISIFKAVGDDYSFLENSSGYSAEPPTVIDLDADHYSLLREDIGELVKHIRYLLGE

SEQ ID NO: 4 - DNA, 3849 bp, Artificial
  Nucleic acid construct
atgactcttcaggagaccagcgtgctcgagcccaccctgcaggggaccaccacgttgcccggcctgctcgcccagcgggtggccgaacaccccgaggcgatcgcggtcgcctaccgggacgacaagctcaccttccgcgagctcgcgtccagaagcgcggccctcgccgactacctggagcacctcggtgtctccgccgacgactgcgtcggcctgttcgtcgagccgtcgatcgacctgatggtcggcgcctggggcatcctcaacgccggcgccgcgtacctgccgctgtccccggagtaccccgaggaccggctgcgctacatgatcgagaacagcgagacgaagatcatcctggcgcagcagcgcctggtgtcccgtctgcgcgagctcgcgccgaaggacgtcaccatcgtgaccctgcgcgagtccgaggccttcgtccgccccgagggcaccgaggccccggccgcccgcagcgcccgcccggacaccctcgcgtacgtcatctacacctccggcagcacgggcaagccgaagggtgtgatgatcgagcaccgcagcatcgtcaaccagctcggctggctgcgcgagacctacgcgatcgaccgcagcaaggtcatcctccagaagaccccgatgagcttcgacgccgcccagtgggagatcctctccccggccaacggcgccaccgtcgtcatgggcgccccgggcgtctacgccgaccccgagggcctcatcgagaccatcgtcaagcacaacgtgaccaccctccagtgcgtcccgacgctgctccagggtctgatcgacaccgagaagttccccgagtgcgtctccctccagcagatcttcagcggtggcgaggccctctcccgcctgctggcgatccagaccacgcaggagatgcccggccgggcgctcatcaacgtctacgggccgaccgagacgacgatcaactcgtcctcgttccccgtcgaccccgccgacctggacgagggaccgcagtccatctccatcggctccccggtgcacggcaccacgtaccacatccttgacaaggagaccctcaagccggtcggcgtcggtgagatcggcgagctgtacatcggcggcatccagctggcccgcggctacctgcaccgcgacgacctgaccgccgagcgcttcctggagatcgagctcgaggagggcgccgagcccgtccgcctgtacaagacgggcgacctcggccagtggaacaacgacggcaccgtgcagttcgccggccgcgccgacaaccaggtcaagctgcgcggctaccgcgtcgagctcgacgagatatccctggcgatcgagaaccacgactgggtccgcaacgccgccgtcatcgtcaagaacgacggccgcaccggcttccagaacctgatcgcctgcatcgagctgagcgagaaggaagccgccctgatggaccagggcaaccacggctcccaccacgcgtcgaagaagagcaagctccaggtcaaggcgcagctgtccaacccgggcctgcgcgacgacgccgagctggccgcccgcccggccttcgacctggagggcgccgagcccacccccgagcagcgcgcccgggtcttcgcccgcaagacgtaccgcttctacgagggcggcgccgtcacccaggccgacctgctgggcctgctgggcgccacggtcaccgccggctactcgcgcaaggcggccgacctggcccccgccgaactcggccagatcctgcgctggttcggccagtacatcagcgaggagcggctcctgccgaagtacggctacgcctccccgggcgcgctgtacgcgacgcagatgtacttcgagctggagggcgtcggcggtctgaagccgggctactactactaccagccggtccgccaccagctcgtcctcatcagcgagcgcgaggccaccggcaaggccacggcgcagatccacttcatcggcaagaagagcggcatcgagccggtctacaagaacaacatcctcgaggtcctggagatcgagaccggccacatggtcggcctcttcgagcagatcctgccggcctacggcctcgacatccacgaccgcgcctacgagccggccgtcaaggacctgctcgacgtcgccgacgaggactactacctgggcaccttcgagctggtcccgcacgcgggcgcgcgcgacgaccaggccgaggtctacgtccagacgcacggcggaaaggtcgccggcctgcccgagggccagtaccgctacgagaacggcgagctgacccgcttctcggacgacatcgtcctcaagaagcacgtcatcgcgatcaaccagtcggtgtaccaggccgccagcttcggcatcagcgtctacagccgcgccgaggaggagtggctgaagtacatcaccctcggcaagaagctccagcacctgatgatgaacgggctgaacctgggcttcatgtcctcgggctacagctccaagacgggcaacccgctgccggcctcgcgccgcatggacgccgtcctcggcgccaacggcgtcgacagcgccccgatgtacttcttcgtcggcggccgcatcagcgacgagcagatcggccacgagggcatgcgcgaggacagcgtccacatgcgcggtccggccgagctcatccgcgacgacctcgtcagcttcctcccggactacatgatccccaaccgggtcgtggtcttcgaccggctgccgctgtccgccaacggcaagatcgacgtcaaggcgctcgccgcctccgaccaggtcaatgcagagctcgtcgagcgcccctatcgagcgcccggtagcgagctggagcagcgcatcgcagcgatctggtcggagatcctgggagtggaacgggtcggcctggacgacaacttcttcgaactgggcggtcattcgttgctggctacccgggtgatttctcgggttcgccaggagcagcagttggacgcaagcctgaaggcgttgttcgagcggccggttctggaagcgttcgcccagggattggaacgcgaggtcgcccaggagtctagtcgcttcgtccgcctgcacgcggagaccggcaaggcccggcccgtgatctgctggccgggtctgggcggctacccgatgaacctgcgcagcctggccggcgagatcggcctcggccgctcgttctacggcgtccagtcctacggcatcaacgagggcgagaccccgtacgagaccatcaccgagatggccaagaaggacatcgaggccctcaaggagatccagccggccggcccctacaccctgtggggctactccttcggcgcccgcgtggccttcgagaccgcctaccagctggagcaggcgggcgagaaggtggacaacctcttcctgatcgccccgggctccccgaaggtgcgcgccgagaacggcaaggtgtggggccgcgaggcgtccttcgccaaccgcggctacaccacgatcctgttctcggtcttcaccggcaccatttccggtccggacctggaccggtgcctggagaccgtgacggacgaggcctccttcgccgagttcatcagcgagctcaagggaatcgacgtcgacctcgcccgccggatcatctcggtcgtgggccagacgtacgaattcgagtactccttccacgagctggccgagcgcaccctccaggcgccgatcagcatcttcaaggccgtgggcgacgactactcgttcctggagaacagcagcggctactcggccgagccgccgacggtcatcgacctcgacgccgaccactacagcctgctgcgcgaggacatcggcgagctggtgaagcacatccgctacctgctcggcgagtga

SEQ ID NO: 5 - protein, 1282 aa, Artificial
  Synthetic construct
MTLQETSVLEPTLQGTTTLPGLLAQRVAEHPEAIAVAYRDDKLTFRELASRSAALADYLEHLGVSADDCVGLFVEPSIDLMVGAWGILNAGAAYLPLSPEYPEDRLRYMIENSETKIILAQQRLVSRLRELAPKDVTIVTLRESEAFVRPEGTEAPAARSARPDTLAYVIYTSGSTGKPKGVMIEHRSIVNQLGWLRETYAIDRSKVILQKTPMSFDAAQWEILSPANGATVVMGAPGVYADPEGLIETIVKHNVTTLQCVPTLLQGLIDTEKFPECVSLQQIFSGGEALSRLLAIQTTQEMPGRALINVYGPTETTINSSSFPVDPADLDEGPQSISIGSPVHGTTYHILDKETLKPVGVGEIGELYIGGIQLARGYLHRDDLTAERFLEIELEEGAEPVRLYKTGDLGQWNNDGTVQFAGRADNQVKLRGYRVELDEISLAIENHDWVRNAAVIVKNDGRTGFQNLIACIELSEKEAALMDQGNHGSHHASKKSKLQVKAQLSNPGLRDDAELAARPAFDLEGAEPTPEQRARVFARKTYRFYEGGAVTQADLLGLLGATVTAGYSRKAADLAPAELGQILRWFGQYISEERLLPKYGYASPGALYATQMYFELEGVGGLKPGYYYYQPVRHQLVLISEREATGKATAQIHFIGKKSGIEPVYKNNILEVLEIETGHMVGLFEQILPAYGLDIHDRAYEPAVKDLLDVADEDYYLGTFELVPHAGARDDQAEVYVQTHGGKVAGLPEGQYRYENGELTRFSDDIVLKKHVIAINQSVYQAASFGISVYSRAEEEWLKYITLGKKLQHLMMNGLNLGFMSSGYSSKTGNPLPASRRMDAVLGANGVDSAPMYFFVGGRISDEQIGHEGMREDSVHMRGPAELIRDDLVSFLPDYMIPNRVVVFDRLPLSANGKIDVKALAASDQVNAELVERPYRAPGSELEQRIAAIWSEILGVERVGLDDNFFELGGHSLLATRVISRVRQEQQLDASLKALFERPVLEAFAQGLEREVAQESSRFVRLHAETGKARPVICWPGLGGYPMNLRSLAGEIGLGRSFYGVQSYGINEGETPYETITEMAKKDIEALKEIQPAGPYTLWGYSFGARVAFETAYQLEQAGEKVDNLFLIAPGSPKVRAENGKVWGREASFANRGYTTILFSVFTGTISGPDLDRCLETVTDEASFAEFISELKGIDVDLARRIISVVGQTYEFEYSFHELAERTLQAPISIFKAVGDDYSFLENSSGYSAEPPTVIDLDADHYSLLREDIGELVKHIRYLLGE

SEQ ID NO: 6 - DNA, 3849 bp, Artificial
  Nucleic acid construct
atgactcttcaggagaccagcgtgctcgagcccaccctgcaggggaccaccacgttgcccggcctgctcgcccagcgggtggccgaacaccccgaggcgatcgcggtcgcctaccgggacgacaagctcaccttccgcgagctcgcgtccagaagcgcggccctcgccgactacctggagcacctcggtgtctccgccgacgactgcgtcggcctgttcgtcgagccgtcgatcgacctgatggtcggcgcctggggcatcctcaacgccggcgccgcgtacctgccgctgtccccggagtaccccgaggaccggctgcgctacatgatcgagaacagcgagacgaagatcatcctggcgcagcagcgcctggtgtcccgtctgcgcgagctcgcgccgaaggacgtcaccatcgtgaccctgcgcgagtccgaggccttcgtccgccccgagggcaccgaggccccggccgcccgcagcgcccgcccggacaccctcgcgtacgtcatctacacctccggcagcacgggcaagccgaagggtgtgatgatcgagcaccgcagcatcgtcaaccagctcggctggctgcgcgagacctacgcgatcgaccgcagcaaggtcatcctccagaagaccccgatgagcttcgacgccgcccagtgggagatcctctccccggccaacggcgccaccgtcgtcatgggcgccccgggcgtctacgccgaccccgagggcctcatcgagaccatcgtcaagcacaacgtgaccaccctccagtgcgtcccgacgctgctccagggtctgatcgacaccgagaagttccccgagtgcgtctccctccagcagatcttcagcggtggcgaggccctctcccgcctgctggcgatccagaccacgcaggagatgcccggccgggcgctcatcaacgtctacgggccgaccgagacgacgatcaactcgtcctcgttccccgtcgaccccgccgacctggacgagggaccgcagtccatctccatcggctccccggtgcacggcaccacgtaccacatccttgacaaggagaccctcaagccggtcggcgtcggtgagatcggcgagctgtacatcggcggcatccagctggcccgcggctacctgcaccgcgacgacctgaccgccgagcgcttcctggagatcgagctcgaggagggcgccgagcccgtccgcctgtacaagacgggcgacctcggccagtggaacaacgacggcaccgtgcagttcgccggccgcgccgacaaccaggtcaagctgcgcggctaccgcgtcgagctcgacgagatatccctggcgatcgagaaccacgactgggtccgcaacgccgccgtcatcgtcaagaacgacggccgcaccggcttccagaacctgatcgcctgcatcgagctgagcgagaaggaagccgccctgatggaccagggcaaccacggctcccaccacgcgtcgaagaagagcaagctccaggtcaaggcgcagctgtccaacccgggcctgcgcgacgacgccgagctggccgcccgcccggccttcgacctggagggcgccgagcccacccccgagcagcgcgcccgggtcttcgcccgcaagacgtaccgcttctacgagggcggcgccgtcacccaggccgacctgctgggcctgctgggcgccacggtcaccgccggctactcgcgcaaggcggccgacctggcccccgccgaactcggccagatcctgcgctggttcggccagtacatcagcgaggagcggctcctgccgaagtacggctacgcctccccgggcgcgctgtacgcgacgcagatgtacttcgagctggagggcgtcggcggtctgaagccgggctactactactaccagccggtccgccaccagctcgtcctcatcagcgagcgcgaggccaccggcaaggccacggcgcagatccacttcatcggcaagaagagcggcatcgagccggtctacaagaacaacatcctcgaggtcctggagatcgagaccggccacatggtcggcctcttcgagcagatcctgccggcctacggcctcgacatccacgaccgcgcctacgagccggccgtcaaggacctgctcgacgtcgccgacgaggactactacctgggcaccttcgagctggtcccgcacgcgggcgcgcgcgacgaccaggccgaggtctacgtccagacgcacggcggaaaggtcgccggcctgcccgagggccagtaccgctacgagaacggcgagctgacccgcttctcggacgacatcgtcctcaagaagcacgtcatcgcgatcaaccagtcggtgtaccaggccgccagcttcggcatcagcgtctacagccgcgccgaggaggagtggctgaagtacatcaccctcggcaagaagctccagcacctgatgatgaacgggctgaacctgggcttcatgtcctcgggctacagctccaagacgggcaacccgctgccggcctcgcgccgcatggacgccgtcctcggcgccaacggcgtcgacagcgccccgatgtacttcttcgtcggcggccgcatcagcgacgagcagatcggccacgagggcatgcgcgaggacagcgtccacatgcgcggtccggccgagctcatccgcgacgacctcgtcagcttcctcccggactacatgatccccaaccgggtcgtggtcttcgaccggctgccgctgtccgccaacggcaagatcgacgtcaaggcgctcgccgcctccgaccaggtcaatgcagagctcgtcgagcgcccctatcgagcgcccggtagcgagctggagcagcgcatcgcagcgatctgggcggagatcctgggagtggaacgggtcggcctggacgacaacttcttcgaactgggcggtcactcattgttgctgctgatgctcaaggagcggatcggcgatacctgccaggctacgctgagcatcagccaactgatgacccatgccagcgtcgcggaacaggcggcatgcatcgagcgcgaggtcgcccaggagtctagtcgcttcgtccgcctgcacgcggagaccggcaaggcccggcccgtgatctgctggccgggtctgggcggctacccgatgaacctgcgcagcctggccggcgagatcggcctcggccgctcgttctacggcgtccagtcctacggcatcaacgagggcgagaccccgtacgagaccatcaccgagatggccaagaaggacatcgaggccctcaaggagatccagccggccggcccctacaccctgtggggctactccttcggcgcccgcgtggccttcgagaccgcctaccagctggagcaggcgggcgagaaggtggacaacctcttcctgatcgccccgggctccccgaaggtgcgcgccgagaacggcaaggtgtggggccgcgaggcgtccttcgccaaccgcggctacaccacgatcctgttctcggtcttcaccggcaccatttccggtccggacctggaccggtgcctggagaccgtgacggacgaggcctccttcgccgagttcatcagcgagctcaagggaatcgacgtcgacctcgcccgccggatcatctcggtcgtgggccagacgtacgaattcgagtactccttccacgagctggccgagcgcaccctccaggcgccgatcagcatcttcaaggccgtgggcgacgactactcgttcctggagaacagcagcggctactcggccgagccgccgacggtcatcgacctcgacgccgaccactacagcctgctgcgcgaggacatcggcgagctggtgaagcacatccgctacctgctcggcgagtga

SEQ ID NO: 7 - protein, 1282 aa, Artificial
  Synthetic construct
MTLQETSVLEPTLQGTTTLPGLLAQRVAEHPEAIAVAYRDDKLTFRELASRSAALADYLEHLGVSADDCVGLFVEPSIDLMVGAWGILNAGAAYLPLSPEYPEDRLRYMIENSETKIILAQQRLVSRLRELAPKDVTIVTLRESEAFVRPEGTEAPAARSARPDTLAYVIYTSGSTGKPKGVMIEHRSIVNQLGWLRETYAIDRSKVILQKTPMSFDAAQWEILSPANGATVVMGAPGVYADPEGLIETIVKHNVTTLQCVPTLLQGLIDTEKFPECVSLQQIFSGGEALSRLLAIQTTQEMPGRALINVYGPTETTINSSSFPVDPADLDEGPQSISIGSPVHGTTYHILDKETLKPVGVGEIGELYIGGIQLARGYLHRDDLTAERFLEIELEEGAEPVRLYKTGDLGQWNNDGTVQFAGRADNQVKLRGYRVELDEISLAIENHDWVRNAAVIVKNDGRTGFQNLIACIELSEKEAALMDQGNHGSHHASKKSKLQVKAQLSNPGLRDDAELAARPAFDLEGAEPTPEQRARVFARKTYRFYEGGAVTQADLLGLLGATVTAGYSRKAADLAPAELGQILRWFGQYISEERLLPKYGYASPGALYATQMYFELEGVGGLKPGYYYYQPVRHQLVLISEREATGKATAQIHFIGKKSGIEPVYKNNILEVLEIETGHMVGLFEQILPAYGLDIHDRAYEPAVKDLLDVADEDYYLGTFELVPHAGARDDQAEVYVQTHGGKVAGLPEGQYRYENGELTRFSDDIVLKKHVIAINQSVYQAASFGISVYSRAEEEWLKYITLGKKLQHLMMNGLNLGFMSSGYSSKTGNPLPASRRMDAVLGANGVDSAPMYFFVGGRISDEQIGHEGMREDSVHMRGPAELIRDDLVSFLPDYMIPNRVVVFDRLPLSANGKIDVKALAASDQVNAELVERPYRAPGSELEQRIAAIWAEILGVERVGLDDNFFELGGHSLLLLMLKERIGDTCQATLSISQLMTHASVAEQAACIEREVAQESSRFVRLHAETGKARPVICWPGLGGYPMNLRSLAGEIGLGRSFYGVQSYGINEGETPYETITEMAKKDIEALKEIQPAGPYTLWGYSFGARVAFETAYQLEQAGEKVDNLFLIAPGSPKVRAENGKVWGREASFANRGYTTILFSVFTGTISGPDLDRCLETVTDEASFAEFISELKGIDVDLARRIISVVGQTYEFEYSFHELAERTLQAPISIFKAVGDDYSFLENSSGYSAEPPTVIDLDADHYSLLREDIGELVKHIRYLLGE

SEQ ID NO: 8 - DNA, 3849 bp, Artificial
  Nucleic acid construct
atgactcttcaggagaccagcgtgctcgagcccaccctgcaggggaccaccacgttgcccggcctgctcgcccagcgggtggccgaacaccccgaggcgatcgcggtcgcctaccgggacgacaagctcaccttccgcgagctcgcgtccagaagcgcggccctcgccgactacctggagcacctcggtgtctccgccgacgactgcgtcggcctgttcgtcgagccgtcgatcgacctgatggtcggcgcctggggcatcctcaacgccggcgccgcgtacctgccgctgtccccggagtaccccgaggaccggctgcgctacatgatcgagaacagcgagacgaagatcatcctggcgcagcagcgcctggtgtcccgtctgcgcgagctcgcgccgaaggacgtcaccatcgtgaccctgcgcgagtccgaggccttcgtccgccccgagggcaccgaggccccggccgcccgcagcgcccgcccggacaccctcgcgtacgtcatctacacctccggcagcacgggcaagccgaagggtgtgatgatcgagcaccgcagcatcgtcaaccagctcggctggctgcgcgagacctacgcgatcgaccgcagcaaggtcatcctccagaagaccccgatgagcttcgacgccgcccagtgggagatcctctccccggccaacggcgccaccgtcgtcatgggcgccccgggcgtctacgccgaccccgagggcctcatcgagaccatcgtcaagcacaacgtgaccaccctccagtgcgtcccgacgctgctccagggtctgatcgacaccgagaagttccccgagtgcgtctccctccagcagatcttcagcggtggcgaggccctctcccgcctgctggcgatccagaccacgcaggagatgcccggccgggcgctcatcaacgtctacgggccgaccgagacgacgatcaactcgtcctcgttccccgtcgaccccgccgacctggacgagggaccgcagtccatctccatcggctccccggtgcacggcaccacgtaccacatccttgacaaggagaccctcaagccggtcggcgtcggtgagatcggcgagctgtacatcggcggcatccagctggcccgcggctacctgcaccgcgacgacctgaccgccgagcgcttcctggagatcgagctcgaggagggcgccgagcccgtccgcctgtacaagacgggcgacctcggccagtggaacaacgacggcaccgtgcagttcgccggccgcgccgacaaccaggtcaagctgcgcggctaccgcgtcgagctcgacgagatatccctggcgatcgagaaccacgactgggtccgcaacgccgccgtcatcgtcaagaacgacggccgcaccggcttccagaacctgatcgcctgcatcgagctgagcgagaaggaagccgccctgatggaccagggcaaccacggctcccaccacgcgtcgaagaagagcaagctccaggtcaaggcgcagctgtccaacccgggcctgcgcgacgacgccgagctggccgcccgcccggccttcgacctggagggcgccgagcccacccccgagcagcgcgcccgggtcttcgcccgcaagacgtaccgcttctacgagggcggcgccgtcacccaggccgacctgctgggcctgctgggcgccacggtcaccgccggctactcgcgcaaggcggccgacctggcccccgccgaactcggccagatcctgcgctggttcggccagtacatcagcgaggagcggctcctgccgaagtacggctacgcctccccgggcgcgctgtacgcgacgcagatgtacttcgagctggagggcgtcggcggtctgaagccgggctactactactaccagccggtccgccaccagctcgtcctcatcagcgagcgcgaggccaccggcaaggccacggcgcagatccacttcatcggcaagaagagcggcatcgagccggtctacaagaacaacatcctcgaggtcctggagatcgagaccggccacatggtcggcctcttcgagcagatcctgccggcctacggcctcgacatccacgaccgcgcctacgagccggccgtcaaggacctgctcgacgtcgccgacgaggactactacctgggcaccttcgagctggtcccgcacgcgggcgcgcgcgacgaccaggccgaggtctacgtccagacgcacggcggaaaggtcgccggcctgcccgagggccagtaccgctacgagaacggcgagctgacccgcttctcggacgacatcgtcctcaagaagcacgtcatcgcgatcaaccagtcggtgtaccaggccgccagcttcggcatcagcgtctacagccgcgccgaggaggagtggctgaagtacatcaccctcggcaagaagctccagcacctgatgatgaacgggctgaacctgggcttcatgtcctcgggctacagctccaagacgggcaacccgctgccggcctcgcgccgcatggacgccgtcctcggcgccaacggcgtcgacagcgccccgatgtacttcttcgtcggcggccgcatcagcgacgagcagatcggccacgagggcatgcgcgaggacagcgtccacatgcgcggtccggccgagctcatccgcgacgacctcgtcagcttcctcccggactacatgatccccaaccgggtcgtggtcttcgaccggctgccgctgtccgccaacggcaagatcgacgtcaaggcgctcgccgcctccgaccaggtcaatgcagagctcgtcgagcgcccccacgtcgcgccacgtacggaattcgagcaacgtcttgccgcgttgtgggagcaggtgctgcaggtcgaacgggtagggctcaacgacaacttcttcgccctgggcggccactcgctgctggcggtttcgctggcaggcaggattcgcgagacgtttgatgtctccatcaaactgcatgatttcctgttgatgcagaccctcggtgagctggccgatttcatgcgtcgcgaggtcgcccaggagtctagtcgcttcgtccgcctgcacgcggagaccggcaaggcccggcccgtgatctgctggccgggtctgggcggctacccgatgaacctgcgcagcctggccggcgagatcggcctcggccgctcgttctacggcgtccagtcctacggcatcaacgagggcgagaccccgtacgagaccatcaccgagatggccaagaaggacatcgaggccctcaaggagatccagccggccggcccctacaccctgtggggctactccttcggcgcccgcgtggccttcgagaccgcctaccagctggagcaggcgggcgagaaggtggacaacctcttcctgatcgccccgggctccccgaaggtgcgcgccgagaacggcaaggtgtggggccgcgaggcgtccttcgccaaccgcggctacaccacgatcctgttctcggtcttcaccggcaccatttccggtccggacctggaccggtgcctggagaccgtgacggacgaggcctccttcgccgagttcatcagcgagctcaagggaatcgacgtcgacctcgcccgccggatcatctcggtcgtgggccagacgtacgaattcgagtactccttccacgagctggccgagcgcaccctccaggcgccgatcagcatcttcaaggccgtgggcgacgactactcgttcctggagaacagcagcggctactcggccgagccgccgacggtcatcgacctcgacgccgaccactacagcctgctgcgcgaggacatcggcgagctggtgaagcacatccgctacctgctcggcgagtga

SEQ ID NO: 9 - protein, 1282 aa, Artificial
  Synthetic construct
MTLQETSVLEPTLQGTTTLPGLLAQRVAEHPEAIAVAYRDDKLTFRELASRSAALADYLEHLGVSADDCVGLFVEPSIDLMVGAWGILNAGAAYLPLSPEYPEDRLRYMIENSETKIILAQQRLVSRLRELAPKDVTIVTLRESEAFVRPEGTEAPAARSARPDTLAYVIYTSGSTGKPKGVMIEHRSIVNQLGWLRETYAIDRSKVILQKTPMSFDAAQWEILSPANGATVVMGAPGVYADPEGLIETIVKHNVTTLQCVPTLLQGLIDTEKFPECVSLQQIFSGGEALSRLLAIQTTQEMPGRALINVYGPTETTINSSSFPVDPADLDEGPQSISIGSPVHGTTYHILDKETLKPVGVGEIGELYIGGIQLARGYLHRDDLTAERFLEIELEEGAEPVRLYKTGDLGQWNNDGTVQFAGRADNQVKLRGYRVELDEISLAIENHDWVRNAAVIVKNDGRTGFQNLIACIELSEKEAALMDQGNHGSHHASKKSKLQVKAQLSNPGLRDDAELAARPAFDLEGAEPTPEQRARVFARKTYRFYEGGAVTQADLLGLLGATVTAGYSRKAADLAPAELGQILRWFGQYISEERLLPKYGYASPGALYATQMYFELEGVGGLKPGYYYYQPVRHQLVLISEREATGKATAQIHFIGKKSGIEPVYKNNILEVLEIETGHMVGLFEQILPAYGLDIHDRAYEPAVKDLLDVADEDYYLGTFELVPHAGARDDQAEVYVQTHGGKVAGLPEGQYRYENGELTRFSDDIVLKKHVIAINQSVYQAASFGISVYSRAEEEWLKYITLGKKLQHLMMNGLNLGFMSSGYSSKTGNPLPASRRMDAVLGANGVDSAPMYFFVGGRISDEQIGHEGMREDSVHMRGPAELIRDDLVSFLPDYMIPNRVVVFDRLPLSANGKIDVKALAASDQVNAELVERPHVAPRTEFEQRLAALWEQVLQVERVGLNDNFFALGGHSLLAVSLAGRIRETFDVSIKLHDFLLMQTLGELADFMRREVAQESSRFVRLHAETGKARPVICWPGLGGYPMNLRSLAGEIGLGRSFYGVQSYGINEGETPYETITEMAKKDIEALKEIQPAGPYTLWGYSFGARVAFETAYQLEQAGEKVDNLFLIAPGSPKVRAENGKVWGREASFANRGYTTILFSVFTGTISGPDLDRCLETVTDEASFAEFISELKGIDVDLARRIISVVGQTYEFEYSFHELAERTLQAPISIFKAVGDDYSFLENSSGYSAEPPTVIDLDADHYSLLREDIGELVKHIRYLLGE

SEQ ID NO: 10 - DNA, 3849 bp, Artificial
  Nucleic acid construct
atgactcttcaggagaccagcgtgctcgagcccaccctgcaggggaccaccacgttgcccggcctgctcgcccagcgggtggccgaacaccccgaggcgatcgcggtcgcctaccgggacgacaagctcaccttccgcgagctcgcgtccagaagcgcggccctcgccgactacctggagcacctcggtgtctccgccgacgactgcgtcggcctgttcgtcgagccgtcgatcgacctgatggtcggcgcctggggcatcctcaacgccggcgccgcgtacctgccgctgtccccggagtaccccgaggaccggctgcgctacatgatcgagaacagcgagacgaagatcatcctggcgcagcagcgcctggtgtcccgtctgcgcgagctcgcgccgaaggacgtcaccatcgtgaccctgcgcgagtccgaggccttcgtccgccccgagggcaccgaggccccggccgcccgcagcgcccgcccggacaccctcgcgtacgtcatctacacctccggcagcacgggcaagccgaagggtgtgatgatcgagcaccgcagcatcgtcaaccagctcggctggctgcgcgagacctacgcgatcgaccgcagcaaggtcatcctccagaagaccccgatgagcttcgacgccgcccagtgggagatcctctccccggccaacggcgccaccgtcgtcatgggcgccccgggcgtctacgccgaccccgagggcctcatcgagaccatcgtcaagcacaacgtgaccaccctccagtgcgtcccgacgctgctccagggtctgatcgacaccgagaagttccccgagtgcgtctccctccagcagatcttcagcggtggcgaggccctctcccgcctgctggcgatccagaccacgcaggagatgcccggccgggcgctcatcaacgtctacgggccgaccgagacgacgatcaactcgtcctcgttccccgtcgaccccgccgacctggacgagggaccgcagtccatctccatcggctccccggtgcacggcaccacgtaccacatccttgacaaggagaccctcaagccggtcggcgtcggtgagatcggcgagctgtacatcggcggcatccagctggcccgcggctacctgcaccgcgacgacctgaccgccgagcgcttcctggagatcgagctcgaggagggcgccgagcccgtccgcctgtacaagacgggcgacctcggccagtggaacaacgacggcaccgtgcagttcgccggccgcgccgacaaccaggtcaagctgcgcggctaccgcgtcgagctcgacgagatatccctggcgatcgagaaccacgactgggtccgcaacgccgccgtcatcgtcaagaacgacggccgcaccggcttccagaacctgatcgcctgcatcgagctgagcgagaaggaagccgccctgatggaccagggcaaccacggctcccaccacgcgtcgaagaagagcaagctccaggtcaaggcgcagctgtccaacccgggcctgcgcgacgacgccgagctggccgcccgcccggccttcgacctggagggcgccgagcccacccccgagcagcgcgcccgggtcttcgcccgcaagacgtaccgcttctacgagggcggcgccgtcacccaggccgacctgctgggcctgctgggcgccacggtcaccgccggctactcgcgcaaggcggccgacctggcccccgccgaactcggccagatcctgcgctggttcggccagtacatcagcgaggagcggctcctgccgaagtacggctacgcctccccgggcgcgctgtacgcgacgcagatgtacttcgagctggagggcgtcggcggtctgaagccgggctactactactaccagccggtccgccaccagctcgtcctcatcagcgagcgcgaggccaccggcaaggccacggcgcagatccacttcatcggcaagaagagcggcatcgagccggtctacaagaacaacatcctcgaggtcctggagatcgagaccggccacatggtcggcctcttcgagcagatcctgccggcctacggcctcgacatccacgaccgcgcctacgagccggccgtcaaggacctgctcgacgtcgccgacgaggactactacctgggcaccttcgagctggtcccgcacgcgggcgcgcgcgacgaccaggccgaggtctacgtccagacgcacggcggaaaggtcgccggcctgcccgagggccagtaccgctacgagaacggcgagctgacccgcttctcggacgacatcgtcctcaagaagcacgtcatcgcgatcaaccagtcggtgtaccaggccgccagcttcggcatcagcgtctacagccgcgccgaggaggagtggctgaagtacatcaccctcggcaagaagctccagcacctgatgatgaacgggctgaacctgggcttcatgtcctcgggctacagctccaagacgggcaacccgctgccggcctcgcgccgcatggacgccgtcctcggcgccaacggcgtcgacagcgccccgatgtacttcttcgtcggcggccgcatcagcgacgagcagatcggccacgagggcatgcgcgaggacagcgtccacatgcgcggtccggccgagctcatccgcgacgacctcgtcagcttcctcccggactacatgatccccaaccgggtcgtggtcttcgaccggctgccgctgtccgccaacggcaagatcgacgtcaaggcgctcgccgcctccgaccaggtcaatgcagagctcgtcgagcgccccgggcgtgcgccgaaagcgggcagtgaaacgattatcgccgcggcattctcgtcgttgctggggtgtgacgtgcaggatgccgatgctgatttcttcgcgcttggcggtcattcgctactggcaatgaaactggcagcgcagttaagtcggcaggttgcccgccaggtgacgccggggcaagtgatggtcgcgtcaactgtcgccaaactggcaacgattattgatcgcgaggtcgcccaggagtctagtcgcttcgtccgcctgcacgcggagaccggcaaggcccggcccgtgatctgctggccgggtctgggcggctacccgatgaacctgcgcagcctggccggcgagatcggcctcggccgctcgttctacggcgtccagtcctacggcatcaacgagggcgagaccccgtacgagaccatcaccgagatggccaagaaggacatcgaggccctcaaggagatccagccggccggcccctacaccctgtggggctactccttcggcgcccgcgtggccttcgagaccgcctaccagctggagcaggcgggcgagaaggtggacaacctcttcctgatcgccccgggctccccgaaggtgcgcgccgagaacggcaaggtgtggggccgcgaggcgtccttcgccaaccgcggctacaccacgatcctgttctcggtcttcaccggcaccatttccggtccggacctggaccggtgcctggagaccgtgacggacgaggcctccttcgccgagttcatcagcgagctcaagggaatcgacgtcgacctcgcccgccggatcatctcggtcgtgggccagacgtacgaattcgagtactccttccacgagctggccgagcgcaccctccaggcgccgatcagcatcttcaaggccgtgggcgacgactactcgttcctggagaacagcagcggctactcggccgagccgccgacggtcatcgacctcgacgccgaccactacagcctgctgcgcgaggacatcggcgagctggtgaagcacatccgctacctgctcggcgagtga

SEQ ID NO: 11 - protein, 1282 aa, Artificial
  Synthetic construct
MTLQETSVLEPTLQGTTTLPGLLAQRVAEHPEAIAVAYRDDKLTFRELASRSAALADYLEHLGVSADDCVGLFVEPSIDLMVGAWGILNAGAAYLPLSPEYPEDRLRYMIENSETKIILAQQRLVSRLRELAPKDVTIVTLRESEAFVRPEGTEAPAARSARPDTLAYVIYTSGSTGKPKGVMIEHRSIVNQLGWLRETYAIDRSKVILQKTPMSFDAAQWEILSPANGATVVMGAPGVYADPEGLIETIVKHNVTTLQCVPTLLQGLIDTEKFPECVSLQQIFSGGEALSRLLAIQTTQEMPGRALINVYGPTETTINSSSFPVDPADLDEGPQSISIGSPVHGTTYHILDKETLKPVGVGEIGELYIGGIQLARGYLHRDDLTAERFLEIELEEGAEPVRLYKTGDLGQWNNDGTVQFAGRADNQVKLRGYRVELDEISLAIENHDWVRNAAVIVKNDGRTGFQNLIACIELSEKEAALMDQGNHGSHHASKKSKLQVKAQLSNPGLRDDAELAARPAFDLEGAEPTPEQRARVFARKTYRFYEGGAVTQADLLGLLGATVTAGYSRKAADLAPAELGQILRWFGQYISEERLLPKYGYASPGALYATQMYFELEGVGGLKPGYYYYQPVRHQLVLISEREATGKATAQIHFIGKKSGIEPVYKNNILEVLEIETGHMVGLFEQILPAYGLDIHDRAYEPAVKDLLDVADEDYYLGTFELVPHAGARDDQAEVYVQTHGGKVAGLPEGQYRYENGELTRFSDDIVLKKHVIAINQSVYQAASFGISVYSRAEEEWLKYITLGKKLQHLMMNGLNLGFMSSGYSSKTGNPLPASRRMDAVLGANGVDSAPMYFFVGGRISDEQIGHEGMREDSVHMRGPAELIRDDLVSFLPDYMIPNRVVVFDRLPLSANGKIDVKALAASDQVNAELVERPGRAPKAGSETIIAAAFSSLLGCDVQDADADFFALGGHSLLAMKLAAQLSRQVARQVTPGQVMVASTVAKLATIIDREVAQESSRFVRLHAETGKARPVICWPGLGGYPMNLRSLAGEIGLGRSFYGVQSYGINEGETPYETITEMAKKDIEALKEIQPAGPYTLWGYSFGARVAFETAYQLEQAGEKVDNLFLIAPGSPKVRAENGKVWGREASFANRGYTTILFSVFTGTISGPDLDRCLETVTDEASFAEFISELKGIDVDLARRIISVVGQTYEFEYSFHELAERTLQAPISIFKAVGDDYSFLENSSGYSAEPPTVIDLDADHYSLLREDIGELVKHIRYLLGE

SEQ ID NO: 12 - DNA, 3849 bp, Artificial
  Nucleic acid construct
atgactcttcaggagaccagcgtgctcgagcccaccctgcaggggaccaccacgttgcccggcctgctcgcccagcgggtggccgaacaccccgaggcgatcgcggtcgcctaccgggacgacaagctcaccttccgcgagctcgcgtccagaagcgcggccctcgccgactacctggagcacctcggtgtctccgccgacgactgcgtcggcctgttcgtcgagccgtcgatcgacctgatggtcggcgcctggggcatcctcaacgccggcgccgcgtacctgccgctgtccccggagtaccccgaggaccggctgcgctacatgatcgagaacagcgagacgaagatcatcctggcgcagcagcgcctggtgtcccgtctgcgcgagctcgcgccgaaggacgtcaccatcgtgaccctgcgcgagtccgaggccttcgtccgccccgagggcaccgaggccccggccgcccgcagcgcccgcccggacaccctcgcgtacgtcatctacacctccggcagcacgggcaagccgaagggtgtgatgatcgagcaccgcagcatcgtcaaccagctcggctggctgcgcgagacctacgcgatcgaccgcagcaaggtcatcctccagaagaccccgatgagcttcgacgccgcccagtgggagatcctctccccggccaacggcgccaccgtcgtcatgggcgccccgggcgtctacgccgaccccgagggcctcatcgagaccatcgtcaagcacaacgtgaccaccctccagtgcgtcccgacgctgctccagggtctgatcgacaccgagaagttccccgagtgcgtctccctccagcagatcttcagcggtggcgaggccctctcccgcctgctggcgatccagaccacgcaggagatgcccggccgggcgctcatcaacgtctacgggccgaccgagacgacgatcaactcgtcctcgttccccgtcgaccccgccgacctggacgagggaccgcagtccatctccatcggctccccggtgcacggcaccacgtaccacatccttgacaaggagaccctcaagccggtcggcgtcggtgagatcggcgagctgtacatcggcggcatccagctggcccgcggctacctgcaccgcgacgacctgaccgccgagcgcttcctggagatcgagctcgaggagggcgccgagcccgtccgcctgtacaagacgggcgacctcggccagtggaacaacgacggcaccgtgcagttcgccggccgcgccgacaaccaggtcaagctgcgcggctaccgcgtcgagctcgacgagatatccctggcgatcgagaaccacgactgggtccgcaacgccgccgtcatcgtcaagaacgacggccgcaccggcttccagaacctgatcgcctgcatcgagctgagcgagaaggaagccgccctgatggaccagggcaaccacggctcccaccacgcgtcgaagaagagcaagctccaggtcaaggcgcagctgtccaacccgggcctgcgcgacgacgccgagctggccgcccgcccggccttcgacctggagggcgccgagcccacccccgagcagcgcgcccgggtcttcgcccgcaagacgtaccgcttctacgagggcggcgccgtcacccaggccgacctgctgggcctgctgggcgccacggtcaccgccggctactcgcgcaaggcggccgacctggcccccgccgaactcggccagatcctgcgctggttcggccagtacatcagcgaggagcggctcctgccgaagtacggctacgcctccccgggcgcgctgtacgcgacgcagatgtacttcgagctggagggcgtcggcggtctgaagccgggctactactactaccagccggtccgccaccagctcgtcctcatcagcgagcgcgaggccaccggcaaggccacggcgcagatccacttcatcggcaagaagagcggcatcgagccggtctacaagaacaacatcctcgaggtcctggagatcgagaccggccacatggtcggcctcttcgagcagatcctgccggcctacggcctcgacatccacgaccgcgcctacgagccggccgtcaaggacctgctcgacgtcgccgacgaggactactacctgggcaccttcgagctggtcccgcacgcgggcgcgcgcgacgaccaggccgaggtctacgtccagacgcacggcggaaaggtcgccggcctgcccgagggccagtaccgctacgagaacggcgagctgacccgcttctcggacgacatcgtcctcaagaagcacgtcatcgcgatcaaccagtcggtgtaccaggccgccagcttcggcatcagcgtctacagccgcgccgaggaggagtggctgaagtacatcaccctcggcaagaagctccagcacctgatgatgaacgggctgaacctgggcttcatgtcctcgggctacagctccaagacgggcaacccgctgccggcctcgcgccgcatggacgccgtcctcggcgccaacggcgtcgacagcgccccgatgtacttcttcgtcggcggccgcatcagcgacgagcagatcggccacgagggcatgcgcgaggacagcgtccacatgcgcggtccggccgagctcatccgcgacgacctcgtcagcttcctcccggactacatgatccccaaccgggtcgtggtcttcgaccggctgccgctgtccgccaacggcaagatcgacgtcaaggcgctcgccgcctccgaccaggtcaatgcagagctcgtcgagcgccccagtgatcgggccccgcggactcctcaggaagagatattgtgtgacttgtttgcagaggttctcggtttggcacgcgtcggtatcgatgacagtttcttcgagcttgggggccattctcttcttgcagcccgtctgatgagccgcattcgcgaggtaatgggagccgaacttggtatcgccaagctctttgacgaaccgacagtagccggactcgctgcccatcttcgcgaggtcgcccaggagtctagtcgcttcgtccgcctgcacgcggagaccggcaaggcccggcccgtgatctgctggccgggtctgggcggctacccgatgaacctgcgcagcctggccggcgagatcggcctcggccgctcgttctacggcgtccagtcctacggcatcaacgagggcgagaccccgtacgagaccatcaccgagatggccaagaaggacatcgaggccctcaaggagatccagccggccggcccctacaccctgtggggctactccttcggcgcccgcgtggccttcgagaccgcctaccagctggagcaggcgggcgagaaggtggacaacctcttcctgatcgccccgggctccccgaaggtgcgcgccgagaacggcaaggtgtggggccgcgaggcgtccttcgccaaccgcggctacaccacgatcctgttctcggtcttcaccggcaccatttccggtccggacctggaccggtgcctggagaccgtgacggacgaggcctccttcgccgagttcatcagcgagctcaagggaatcgacgtcgacctcgcccgccggatcatctcggtcgtgggccagacgtacgaattcgagtactccttccacgagctggccgagcgcaccctccaggcgccgatcagcatcttcaaggccgtgggcgacgactactcgttcctggagaacagcagcggctactcggccgagccgccgacggtcatcgacctcgacgccgaccactacagcctgctgcgcgaggacatcggcgagctggtgaagcacatccgctacctgctcggcgagtga

SEQ ID NO: 13 - protein, 1282 aa, Artificial
  Syntheic construct
MTLQETSVLEPTLQGTTTLPGLLAQRVAEHPEAIAVAYRDDKLTFRELASRSAALADYLEHLGVSADDCVGLFVEPSIDLMVGAWGILNAGAAYLPLSPEYPEDRLRYMIENSETKIILAQQRLVSRLRELAPKDVTIVTLRESEAFVRPEGTEAPAARSARPDTLAYVIYTSGSTGKPKGVMIEHRSIVNQLGWLRETYAIDRSKVILQKTPMSFDAAQWEILSPANGATVVMGAPGVYADPEGLIETIVKHNVTTLQCVPTLLQGLIDTEKFPECVSLQQIFSGGEALSRLLAIQTTQEMPGRALINVYGPTETTINSSSFPVDPADLDEGPQSISIGSPVHGTTYHILDKETLKPVGVGEIGELYIGGIQLARGYLHRDDLTAERFLEIELEEGAEPVRLYKTGDLGQWNNDGTVQFAGRADNQVKLRGYRVELDEISLAIENHDWVRNAAVIVKNDGRTGFQNLIACIELSEKEAALMDQGNHGSHHASKKSKLQVKAQLSNPGLRDDAELAARPAFDLEGAEPTPEQRARVFARKTYRFYEGGAVTQADLLGLLGATVTAGYSRKAADLAPAELGQILRWFGQYISEERLLPKYGYASPGALYATQMYFELEGVGGLKPGYYYYQPVRHQLVLISEREATGKATAQIHFIGKKSGIEPVYKNNILEVLEIETGHMVGLFEQILPAYGLDIHDRAYEPAVKDLLDVADEDYYLGTFELVPHAGARDDQAEVYVQTHGGKVAGLPEGQYRYENGELTRFSDDIVLKKHVIAINQSVYQAASFGISVYSRAEEEWLKYITLGKKLQHLMMNGLNLGFMSSGYSSKTGNPLPASRRMDAVLGANGVDSAPMYFFVGGRISDEQIGHEGMREDSVHMRGPAELIRDDLVSFLPDYMIPNRVVVFDRLPLSANGKIDVKALAASDQVNAELVERPSDRAPRTPQEEILCDLFAEVLGLARVGIDDSFFELGGHSLLAARLMSRIREVMGAELGIAKLFDEPTVAGLAAHLREVAQESSRFVRLHAETGKARPVICWPGLGGYPMNLRSLAGEIGLGRSFYGVQSYGINEGETPYETITEMAKKDIEALKEIQPAGPYTLWGYSFGARVAFETAYQLEQAGEKVDNLFLIAPGSPKVRAENGKVWGREASFANRGYTTILFSVFTGTISGPDLDRCLETVTDEASFAEFISELKGIDVDLARRIISVVGQTYEFEYSFHELAERTLQAPISIFKAVGDDYSFLENSSGYSAEPPTVIDLDADHYSLLREDIGELVKHIRYLLGE

SEQ ID NO: 14 - DNA, 3849 bp, Artificial
  Nucleic acid construct
atgactcttcaggagaccagcgtgctcgagcccaccctgcaggggaccaccacgttgcccggcctgctcgcccagcgggtggccgaacaccccgaggcgatcgcggtcgcctaccgggacgacaagctcaccttccgcgagctcgcgtccagaagcgcggccctcgccgactacctggagcacctcggtgtctccgccgacgactgcgtcggcctgttcgtcgagccgtcgatcgacctgatggtcggcgcctggggcatcctcaacgccggcgccgcgtacctgccgctgtccccggagtaccccgaggaccggctgcgctacatgatcgagaacagcgagacgaagatcatcctggcgcagcagcgcctggtgtcccgtctgcgcgagctcgcgccgaaggacgtcaccatcgtgaccctgcgcgagtccgaggccttcgtccgccccgagggcaccgaggccccggccgcccgcagcgcccgcccggacaccctcgcgtacgtcatctacacctccggcagcacgggcaagccgaagggtgtgatgatcgagcaccgcagcatcgtcaaccagctcggctggctgcgcgagacctacgcgatcgaccgcagcaaggtcatcctccagaagaccccgatgagcttcgacgccgcccagtgggagatcctctccccggccaacggcgccaccgtcgtcatgggcgccccgggcgtctacgccgaccccgagggcctcatcgagaccatcgtcaagcacaacgtgaccaccctccagtgcgtcccgacgctgctccagggtctgatcgacaccgagaagttccccgagtgcgtctccctccagcagatcttcagcggtggcgaggccctctcccgcctgctggcgatccagaccacgcaggagatgcccggccgggcgctcatcaacgtctacgggccgaccgagacgacgatcaactcgtcctcgttccccgtcgaccccgccgacctggacgagggaccgcagtccatctccatcggctccccggtgcacggcaccacgtaccacatccttgacaaggagaccctcaagccggtcggcgtcggtgagatcggcgagctgtacatcggcggcatccagctggcccgcggctacctgcaccgcgacgacctgaccgccgagcgcttcctggagatcgagctcgaggagggcgccgagcccgtccgcctgtacaagacgggcgacctcggccagtggaacaacgacggcaccgtgcagttcgccggccgcgccgacaaccaggtcaagctgcgcggctaccgcgtcgagctcgacgagatatccctggcgatcgagaaccacgactgggtccgcaacgccgccgtcatcgtcaagaacgacggccgcaccggcttccagaacctgatcgcctgcatcgagctgagcgagaaggaagccgccctgatggaccagggcaaccacggctcccaccacgcgtcgaagaagagcaagctccaggtcaaggcgcagctgtccaacccgggcctgcgcgacgacgccgagctggccgcccgcccggccttcgacctggagggcgccgagcccacccccgagcagcgcgcccgggtcttcgcccgcaagacgtaccgcttctacgagggcggcgccgtcacccaggccgacctgctgggcctgctgggcgccacggtcaccgccggctactcgcgcaaggcggccgacctggcccccgccgaactcggccagatcctgcgctggttcggccagtacatcagcgaggagcggctcctgccgaagtacggctacgcctccccgggcgcgctgtacgcgacgcagatgtacttcgagctggagggcgtcggcggtctgaagccgggctactactactaccagccggtccgccaccagctcgtcctcatcagcgagcgcgaggccaccggcaaggccacggcgcagatccacttcatcggcaagaagagcggcatcgagccggtctacaagaacaacatcctcgaggtcctggagatcgagaccggccacatggtcggcctcttcgagcagatcctgccggcctacggcctcgacatccacgaccgcgcctacgagccggccgtcaaggacctgctcgacgtcgccgacgaggactactacctgggcaccttcgagctggtcccgcacgcgggcgcgcgcgacgaccaggccgaggtctacgtccagacgcacggcggaaaggtcgccggcctgcccgagggccagtaccgctacgagaacggcgagctgacccgcttctcggacgacatcgtcctcaagaagcacgtcatcgcgatcaaccagtcggtgtaccaggccgccagcttcggcatcagcgtctacagccgcgccgaggaggagtggctgaagtacatcaccctcggcaagaagctccagcacctgatgatgaacgggctgaacctgggcttcatgtcctcgggctacagctccaagacgggcaacccgctgccggcctcgcgccgcatggacgccgtcctcggcgccaacggcgtcgacagcgccccgatgtacttcttcgtcggcggccgcatcagcgacgagcagatcggccacgagggcatgcgcgaggacagcgtccacatgcgcggtccggccgagctcatccgcgacgacctcgtcagcttcctcccggactacatgatccccaaccgggtcgtggtcttcgaccggctgccgctgtccgccaacggcaagatcgacgtcaaggcgctcgccgcctccgaccaggtcaatgcagagctcgtcgagcgccccgggcgtgcgccgaaagcgggcagtgaaacgattatcgccgcggcattctcgtcgttgctggggtgtgacgtgcaggatgccgatgctgatttcttcgcgcttggcggtcattcgatactggcaatgaaactggcagcgcagttaagtcggcaggttgcccgccaggtgacgccggggcaagtgatggtcgcgtcaactgtcgccaaactggcaacgattattgatcgcgaggtcgcccaggagtctagtcgcttcgtccgcctgcacgcggagaccggcaaggcccggcccgtgatctgctggccgggtctgggcggctacccgatgaacctgcgcagcctggccggcgagatcggcctcggccgctcgttctacggcgtccagtcctacggcatcaacgagggcgagaccccgtacgagaccatcaccgagatggccaagaaggacatcgaggccctcaaggagatccagccggccggcccctacaccctgtggggctactccttcggcgcccgcgtggccttcgagaccgcctaccagctggagcaggcgggcgagaaggtggacaacctcttcctgatcgccccgggctccccgaaggtgcgcgccgagaacggcaaggtgtggggccgcgaggcgtccttcgccaaccgcggctacaccacgatcctgttctcggtcttcaccggcaccatttccggtccggacctggaccggtgcctggagaccgtgacggacgaggcctccttcgccgagttcatcagcgagctcaagggaatcgacgtcgacctcgcccgccggatcatctcggtcgtgggccagacgtacgaattcgagtactccttccacgagctggccgagcgcaccctccaggcgccgatcagcatcttcaaggccgtgggcgacgactactcgttcctggagaacagcagcggctactcggccgagccgccgacggtcatcgacctcgacgccgaccactacagcctgctgcgcgaggacatcggcgagctggtgaagcacatccgctacctgctcggcgagtga

SEQ ID NO: 15 - protein, 1282 aa, Artificial
  Synthetic construct
MTLQETSVLEPTLQGTTTLPGLLAQRVAEHPEAIAVAYRDDKLTFRELASRSAALADYLEHLGVSADDCVGLFVEPSIDLMVGAWGILNAGAAYLPLSPEYPEDRLRYMIENSETKIILAQQRLVSRLRELAPKDVTIVTLRESEAFVRPEGTEAPAARSARPDTLAYVIYTSGSTGKPKGVMIEHRSIVNQLGWLRETYAIDRSKVILQKTPMSFDAAQWEILSPANGATVVMGAPGVYADPEGLIETIVKHNVTTLQCVPTLLQGLIDTEKFPECVSLQQIFSGGEALSRLLAIQTTQEMPGRALINVYGPTETTINSSSFPVDPADLDEGPQSISIGSPVHGTTYHILDKETLKPVGVGEIGELYIGGIQLARGYLHRDDLTAERFLEIELEEGAEPVRLYKTGDLGQWNNDGTVQFAGRADNQVKLRGYRVELDEISLAIENHDWVRNAAVIVKNDGRTGFQNLIACIELSEKEAALMDQGNHGSHHASKKSKLQVKAQLSNPGLRDDAELAARPAFDLEGAEPTPEQRARVFARKTYRFYEGGAVTQADLLGLLGATVTAGYSRKAADLAPAELGQILRWFGQYISEERLLPKYGYASPGALYATQMYFELEGVGGLKPGYYYYQPVRHQLVLISEREATGKATAQIHFIGKKSGIEPVYKNNILEVLEIETGHMVGLFEQILPAYGLDIHDRAYEPAVKDLLDVADEDYYLGTFELVPHAGARDDQAEVYVQTHGGKVAGLPEGQYRYENGELTRFSDDIVLKKHVIAINQSVYQAASFGISVYSRAEEEWLKYITLGKKLQHLMMNGLNLGFMSSGYSSKTGNPLPASRRMDAVLGANGVDSAPMYFFVGGRISDEQIGHEGMREDSVHMRGPAELIRDDLVSFLPDYMIPNRVVVFDRLPLSANGKIDVKALAASDQVNAELVERPGRAPKAGSETIIAAAFSSLLGCDVQDADADFFALGGHSILAMKLAAQLSRQVARQVTPGQVMVASTVAKLATIIDREVAQESSRFVRLHAETGKARPVICWPGLGGYPMNLRSLAGEIGLGRSFYGVQSYGINEGETPYETITEMAKKDIEALKEIQPAGPYTLWGYSFGARVAFETAYQLEQAGEKVDNLFLIAPGSPKVRAENGKVWGREASFANRGYTTILFSVFTGTISGPDLDRCLETVTDEASFAEFISELKGIDVDLARRIISVVGQTYEFEYSFHELAERTLQAPISIFKAVGDDYSFLENSSGYSAEPPTVIDLDADHYSLLREDIGELVKHIRYLLGE

SEQ ID NO: 16 - DNA, 3849 bp, Artificial
  Nucleic acid construct
atgactcttcaggagaccagcgtgctcgagcccaccctgcaggggaccaccacgttgcccggcctgctcgcccagcgggtggccgaacaccccgaggcgatcgcggtcgcctaccgggacgacaagctcaccttccgcgagctcgcgtccagaagcgcggccctcgccgactacctggagcacctcggtgtctccgccgacgactgcgtcggcctgttcgtcgagccgtcgatcgacctgatggtcggcgcctggggcatcctcaacgccggcgccgcgtacctgccgctgtccccggagtaccccgaggaccggctgcgctacatgatcgagaacagcgagacgaagatcatcctggcgcagcagcgcctggtgtcccgtctgcgcgagctcgcgccgaaggacgtcaccatcgtgaccctgcgcgagtccgaggccttcgtccgccccgagggcaccgaggccccggccgcccgcagcgcccgcccggacaccctcgcgtacgtcatctacacctccggcagcacgggcaagccgaagggtgtgatgatcgagcaccgcagcatcgtcaaccagctcggctggctgcgcgagacctacgcgatcgaccgcagcaaggtcatcctccagaagaccccgatgagcttcgacgccgcccagtgggagatcctctccccggccaacggcgccaccgtcgtcatgggcgccccgggcgtctacgccgaccccgagggcctcatcgagaccatcgtcaagcacaacgtgaccaccctccagtgcgtcccgacgctgctccagggtctgatcgacaccgagaagttccccgagtgcgtctccctccagcagatcttcagcggtggcgaggccctctcccgcctgctggcgatccagaccacgcaggagatgcccggccgggcgctcatcaacgtctacgggccgaccgagacgacgatcaactcgtcctcgttccccgtcgaccccgccgacctggacgagggaccgcagtccatctccatcggctccccggtgcacggcaccacgtaccacatccttgacaaggagaccctcaagccggtcggcgtcggtgagatcggcgagctgtacatcggcggcatccagctggcccgcggctacctgcaccgcgacgacctgaccgccgagcgcttcctggagatcgagctcgaggagggcgccgagcccgtccgcctgtacaagacgggcgacctcggccagtggaacaacgacggcaccgtgcagttcgccggccgcgccgacaaccaggtcaagctgcgcggctaccgcgtcgagctcgacgagatatccctggcgatcgagaaccacgactgggtccgcaacgccgccgtcatcgtcaagaacgacggccgcaccggcttccagaacctgatcgcctgcatcgagctgagcgagaaggaagccgccctgatggaccagggcaaccacggctcccaccacgcgtcgaagaagagcaagctccaggtcaaggcgcagctgtccaacccgggcctgcgcgacgacgccgagctggccgcccgcccggccttcgacctggagggcgccgagcccacccccgagcagcgcgcccgggtcttcgcccgcaagacgtaccgcttctacgagggcggcgccgtcacccaggccgacctgctgggcctgctgggcgccacggtcaccgccggctactcgcgcaaggcggccgacctggcccccgccgaactcggccagatcctgcgctggttcggccagtacatcagcgaggagcggctcctgccgaagtacggctacgcctccccgggcgcgctgtacgcgacgcagatgtacttcgagctggagggcgtcggcggtctgaagccgggctactactactaccagccggtccgccaccagctcgtcctcatcagcgagcgcgaggccaccggcaaggccacggcgcagatccacttcatcggcaagaagagcggcatcgagccggtctacaagaacaacatcctcgaggtcctggagatcgagaccggccacatggtcggcctcttcgagcagatcctgccggcctacggcctcgacatccacgaccgcgcctacgagccggccgtcaaggacctgctcgacgtcgccgacgaggactactacctgggcaccttcgagctggtcccgcacgcgggcgcgcgcgacgaccaggccgaggtctacgtccagacgcacggcggaaaggtcgccggcctgcccgagggccagtaccgctacgagaacggcgagctgacccgcttctcggacgacatcgtcctcaagaagcacgtcatcgcgatcaaccagtcggtgtaccaggccgccagcttcggcatcagcgtctacagccgcgccgaggaggagtggctgaagtacatcaccctcggcaagaagctccagcacctgatgatgaacgggctgaacctgggcttcatgtcctcgggctacagctccaagacgggcaacccgctgccggcctcgcgccgcatggacgccgtcctcggcgccaacggcgtcgacagcgccccgatgtacttcttcgtcggcggccgcatcagcgacgagcagatcggccacgagggcatgcgcgaggacagcgtccacatgcgcggtccggccgagctcatccgcgacgacctcgtcagcttcctcccggactacatgatccccaaccgggtcgtggtcttcgaccggctgccgctgtccgccaacggcaagatcgacgtcaaggcgctcgccgcctccgaccaggtcaatgcagagctcgtcgagcgcccctatcgagcgcccggtagcgagctggagcagcgcatcgcagcgatctggtcggagatcctgggagtggaacgggtcggcctggacgacaacttcttcgaactgggcggtcattcgttgctggctatccgggtgatttctcgggttcgccaggagcagcagttggacgcaagcctgaaggcgttgatcgagtggccggttctggaagcgttcgcccagggattggaacgcgaggtcgcccaggagtctagtcgcttcgtccgcctgcacgcggagaccggcaaggcccggcccgtgatctgctggccgggtctgggcggctacccgatgaacctgcgcagcctggccggcgagatcggcctcggccgctcgttctacggcgtccagtcctacggcatcaacgagggcgagaccccgtacgagaccatcaccgagatggccaagaaggacatcgaggccctcaaggagatccagccggccggcccctacaccctgtggggctactccttcggcgcccgcgtggccttcgagaccgcctaccagctggagcaggcgggcgagaaggtggacaacctcttcctgatcgccccgggctccccgaaggtgcgcgccgagaacggcaaggtgtggggccgcgaggcgtccttcgccaaccgcggctacaccacgatcctgttctcggtcttcaccggcaccatttccggtccggacctggaccggtgcctggagaccgtgacggacgaggcctccttcgccgagttcatcagcgagctcaagggaatcgacgtcgacctcgcccgccggatcatctcggtcgtgggccagacgtacgaattcgagtactccttccacgagctggccgagcgcaccctccaggcgccgatcagcatcttcaaggccgtgggcgacgactactcgttcctggagaacagcagcggctactcggccgagccgccgacggtcatcgacctcgacgccgaccactacagcctgctgcgcgaggacatcggcgagctggtgaagcacatccgctacctgctcggcgagtga

SEQ ID NO: 17 - protein, 1282 aa, Artificial
  Synthetic construct
MTLQETSVLEPTLQGTTTLPGLLAQRVAEHPEAIAVAYRDDKLTFRELASRSAALADYLEHLGVSADDCVGLFVEPSIDLMVGAWGILNAGAAYLPLSPEYPEDRLRYMIENSETKIILAQQRLVSRLRELAPKDVTIVTLRESEAFVRPEGTEAPAARSARPDTLAYVIYTSGSTGKPKGVMIEHRSIVNQLGWLRETYAIDRSKVILQKTPMSFDAAQWEILSPANGATVVMGAPGVYADPEGLIETIVKHNVTTLQCVPTLLQGLIDTEKFPECVSLQQIFSGGEALSRLLAIQTTQEMPGRALINVYGPTETTINSSSFPVDPADLDEGPQSISIGSPVHGTTYHILDKETLKPVGVGEIGELYIGGIQLARGYLHRDDLTAERFLEIELEEGAEPVRLYKTGDLGQWNNDGTVQFAGRADNQVKLRGYRVELDEISLAIENHDWVRNAAVIVKNDGRTGFQNLIACIELSEKEAALMDQGNHGSHHASKKSKLQVKAQLSNPGLRDDAELAARPAFDLEGAEPTPEQRARVFARKTYRFYEGGAVTQADLLGLLGATVTAGYSRKAADLAPAELGQILRWFGQYISEERLLPKYGYASPGALYATQMYFELEGVGGLKPGYYYYQPVRHQLVLISEREATGKATAQIHFIGKKSGIEPVYKNNILEVLEIETGHMVGLFEQILPAYGLDIHDRAYEPAVKDLLDVADEDYYLGTFELVPHAGARDDQAEVYVQTHGGKVAGLPEGQYRYENGELTRFSDDIVLKKHVIAINQSVYQAASFGISVYSRAEEEWLKYITLGKKLQHLMMNGLNLGFMSSGYSSKTGNPLPASRRMDAVLGANGVDSAPMYFFVGGRISDEQIGHEGMREDSVHMRGPAELIRDDLVSFLPDYMIPNRVVVFDRLPLSANGKIDVKALAASDQVNAELVERPYRAPGSELEQRIAAIWSEILGVERVGLDDNFFELGGHSLLAIRVISRVRQEQQLDASLKALIEWPVLEAFAQGLEREVAQESSRFVRLHAETGKARPVICWPGLGGYPMNLRSLAGEIGLGRSFYGVQSYGINEGETPYETITEMAKKDIEALKEIQPAGPYTLWGYSFGARVAFETAYQLEQAGEKVDNLFLIAPGSPKVRAENGKVWGREASFANRGYTTILFSVFTGTISGPDLDRCLETVTDEASFAEFISELKGIDVDLARRIISVVGQTYEFEYSFHELAERTLQAPISIFKAVGDDYSFLENSSGYSAEPPTVIDLDADHYSLLREDIGELVKHIRYLLGE

SEQ ID NO: 18 - DNA, 3861 bp, Artificial
  Mutant BpsA
atgactcttcaggagaccagcgtgctcgagcccaccctgcaggggaccaccacgttgcccggcctgctcgcccagcgggtggccgaacaccccgaggcgatcgcggtcgcctaccgggacgacaagctcaccttccgcgagctcgcgtccagaagcgcggccctcgccgactacctggagcacctcggtgtctccgccgacgactgcgtcggcctgttcgtcgagccgtcgatcgacctgatggtcggcgcctggggcatcctcaacgccggcgccgcgtacctgccgctgtccccggagtaccccgaggaccggctgcgctacatgatcgagaacagcgagacgaagatcatcctggcgcagcagcgcctggtgtcccgtctgcgcgagctcgcgccgaaggacgtcaccatcgtgaccctgcgcgagtccgaggccttcgtccgccccgagggcaccgaggccccggccgcccgcagcgcccgcccggacaccctcgcgtacgtcatctacacctccggcagcacgggcaagccgaagggtgtgatgatcgagcaccgcagcatcgtcaaccagctcggctggctgcgcgagacctacgcgatcgaccgcagcaaggtcatcctccagaagaccccgatgagcttcgacgccgcccagtgggagatcctctccccggccaacggcgccaccgtcgtcatgggcgccccgggcgtctacgccgaccccgagggcctcatcgagaccatcgtcaagcacaacgtgaccaccctccagtgcgtcccgacgctgctccagggtctgatcgacaccgagaagttccccgagtgcgtctccctccagcagatcttcagcggtggcgaggccctctcccgcctgctggcgatccagaccacgcaggagatgcccggccgggcgctcatcaacgtctacgggccgaccgagacgacgatcaactcgtcctcgttccccgtcgaccccgccgacctggacgagggaccgcagtccatctccatcggctccccggtgcacggcaccacgtaccacatccttgacaaggagaccctcaagccggtcggcgtcggtgagatcggcgagctgtacatcggcggcatccagctggcccgcggctacctgcaccgcgacgacctgaccgccgagcgcttcctggagatcgagctcgaggagggcgccgagcccgtccgcctgtacaagacgggcgacctcggccagtggaacaacgacggcaccgtgcagttcgccggccgcgccgacaaccaggtcaagctgcgcggctaccgcgtcgagctcgacgagatatccctggcgatcgagaaccacgactgggtccgcaacgccgccgtcatcgtcaagaacgacggccgcaccggcttccagaacctgatcgcctgcatcgagctgagcgagaaggaagccgccctgatggaccagggcaaccacggctcccaccacgcgtcgaagaagagcaagctccaggtcaaggcgcagctgtccaacccgggcctgcgcgacgacgccgagctggccgcccgcccggccttcgacctggagggcgccgagcccacccccgagcagcgcgcccgggtcttcgcccgcaagacgtaccgcttctacgagggcggcgccgtcacccaggccgacctgctgggcctgctgggcgccacggtcaccgccggctactcgcgcaaggcggccgacctggcccccgccgaactcggccagatcctgcgctggttcggccagtacatcagcgaggagcggctcctgccgaagtacggctacgcctccccgggcgcgctgtacgcgacgcagatgtacttcgagctggagggcgtcggcggtctgaagccgggctactactactaccagccggtccgccaccagctcgtcctcatcagcgagcgcgaggccaccggcaaggccacggcgcagatccacttcatcggcaagaagagcggcatcgagccggtctacaagaacaacatcctcgaggtcctggagatcgagaccggccacatggtcggcctcttcgagcagatcctgccggcctacggcctcgacatccacgaccgcgcctacgagccggccgtcaaggacctgctcgacgtcgccgacgaggactactacctgggcaccttcgagctggtcccgcacgcgggcgcgcgcgacgaccaggccgaggtctacgtccagacgcacggcggaaaggtcgccggcctgcccgagggccagtaccgctacgagaacggcgagctgacccgcttctcggacgacatcgtcctcaagaagcacgtcatcgcgatcaaccagtcggtgtaccaggccgccagcttcggcatcagcgtctacagccgcgccgaggaggagtggctgaagtacatcaccctcggcaagaagctccagcacctgatgatgaacgggctgaacctgggcttcatgtcctcgggctacagctccaagacgggcaacccgctgccggcctcgcgccgcatggacgccgtcctcggcgccaacggcgtcgacagcgccccgatgtacttcttcgtcggcggccgcatcagcgacgagcagatcggccacgagggcatgcgcgaggacagcgtccacatgcgcggtccggccgagctcatccgcgacgacctcgtcagcttcctcccggactacatgatccccaaccgggtcgtggtcttcgaccggctgccgctgtccgccaacggcaagatcgacgtcaaggcgctcgccgcctccgaccaggtcaacgccgagctcgtcgagcgccccttcgtcgccccgcgcacggagacggagggtaccaaggagatcgcggcggtctgggagaaggccctgcgccgcgagaacgcctccgtccaggacgacttcttcgagtcgggcggcaactcgctgatcgccgtcggcctcgtccgcgagctcaacgcgcgcctgggcgtctccctgccgctgcagagcgtcctggagtccccgaccatcgagaagctggcccgccgcctggctagcgagcgcgaggtcgcccaggagtcctcgcgcttcgtccgcctgcacgcggagaccggcaaggcccggcccgtgatctgctggccgggtctgggcggctacccgatgaacctgcgcagcctggccggcgagatcggcctcggccgctcgttctacggcgtccagtcctacggcatcaacgagggcgagaccccgtacgagaccatcaccgagatggccaagaaggacatcgaggccctcaaggagatccagccggccggcccctacaccctgtggggctactccttcggcgcccgcgtggccttcgagaccgcctaccagctggagcaggcgggcgagaaggtggacaacctcttcctgatcgccccgggctccccgaaggtgcgcgccgagaacggcaaggtgtggggccgcgaggcgtccttcgccaaccgcggctacaccacgatcctgttctcggtcttcaccggcaccatttccggtccggacctggaccggtgcctggagaccgtgacggacgaggcctccttcgccgagttcatcagcgagctcaagggaatcgacgtcgacctcgcccgccggatcatctcggtcgtgggccagacgtacgaattcgagtactccttccacgagctggccgagcgcaccctccaggcgccgatcagcatcttcaaggccgtgggcgacgactactcgttcctggagaacagcagcggctactcggccgagccgccgacggtcatcgacctcgacgccgaccactacagcctgctgcgcgaggacatcggcgagctggtgaagcacatccgctacctgctcggcgagtga

SEQ ID NO: 19 - protein, 1286 aa, Artificial
  Mutant BpsA
MTLQETSVLEPTLQGTTTLPGLLAQRVAEHPEAIAVAYRDDKLTFRELASRSAALADYLEHLGVSADDCVGLFVEPSIDLMVGAWGILNAGAAYLPLSPEYPEDRLRYMIENSETKIILAQQRLVSRLRELAPKDVTIVTLRESEAFVRPEGTEAPAARSARPDTLAYVIYTSGSTGKPKGVMIEHRSIVNQLGWLRETYAIDRSKVILQKTPMSFDAAQWEILSPANGATVVMGAPGVYADPEGLIETIVKHNVTTLQCVPTLLQGLIDTEKFPECVSLQQIFSGGEALSRLLAIQTTQEMPGRALINVYGPTETTINSSSFPVDPADLDEGPQSISIGSPVHGTTYHILDKETLKPVGVGEIGELYIGGIQLARGYLHRDDLTAERFLEIELEEGAEPVRLYKTGDLGQWNNDGTVQFAGRADNQVKLRGYRVELDEISLAIENHDWVRNAAVIVKNDGRTGFQNLIACIELSEKEAALMDQGNHGSHHASKKSKLQVKAQLSNPGLRDDAELAARPAFDLEGAEPTPEQRARVFARKTYRFYEGGAVTQADLLGLLGATVTAGYSRKAADLAPAELGQILRWFGQYISEERLLPKYGYASPGALYATQMYFELEGVGGLKPGYYYYQPVRHQLVLISEREATGKATAQIHFIGKKSGIEPVYKNNILEVLEIETGHMVGLFEQILPAYGLDIHDRAYEPAVKDLLDVADEDYYLGTFELVPHAGARDDQAEVYVQTHGGKVAGLPEGQYRYENGELTRFSDDIVLKKHVIAINQSVYQAASFGISVYSRAEEEWLKYITLGKKLQHLMMNGLNLGFMSSGYSSKTGNPLPASRRMDAVLGANGVDSAPMYFFVGGRISDEQIGHEGMREDSVHMRGPAELIRDDLVSFLPDYMIPNRVVVFDRLPLSANGKIDVKALAASDQVNAELVERPFVAPRTETEGTKEIAAVWEKALRRENASVQDDFFESGGNSLIAVGLVRELNARLGVSLPLQSVLESPTIEKLARRLASEREVAQESSRFVRLHAETGKARPVICWPGLGGYPMNLRSLAGEIGLGRSFYGVQSYGINEGETPYETITEMAKKDIEALKEIQPAGPYTLWGYSFGARVAFETAYQLEQAGEKVDNLFLIAPGSPKVRAENGKVWGREASFANRGYTTILFSVFTGTISGPDLDRCLETVTDEASFAEFISELKGIDVDLARRIISVVGQTYEFEYSFHELAERTLQAPISIFKAVGDDYSFLENSSGYSAEPPTVIDLDADHYSLLREDIGELVKHIRYLLGE

SEQ ID NO: 20 - DNA, 225 bp, Streptomyces lavendulae
ttcgtcgccccgcgcacggagacggagaaggagatcgcggcggtctgggagaaggccctgcgccgcgagaacgcctccgtccaggacgacttcttcgagtcgggcggcaactcgctgatcgccgtcggcctcgtccgcgagctcaacgcgcgcctgggcgtctccctgccgctgcagagcgtcctggagtccccgaccatcgagaagctggcccgccgcctggag

SEQ ID NO: 21 - protein, 75 aa, Streptomyces lavendulae
FVAPRTETEKEIAAVWEKALRRENASVQDDFFESGGNSLIAVGLVRELNARLGVSLPLQSVLESPTIEKLARRLE

SEQ ID NO: 22 - DNA, 33 bp, Artificial
  Primer
ccccggatccgatgactcttcaggagaccagcg

SEQ ID NO: 23 - DNA, 38 bp, Artificial
  Primer
agctaagcttagctatgcattgacctggtcggaggcgg

SEQ ID NO: 24 - DNA, 39 bp, Artificial
  Primer
agctaagcttagctactagtcgcttcgtccgcctgcacg

SEQ ID NO: 25 - DNA, 30 bp, Artificial
  Primer
ccccctcgagtcactcgccgagcaggtagc

SEQ ID NO: 26 - DNA, 45 bp, Artificial
  Primer
agctctgcagagctcgtcgagcgccccttcgtcgccccgcgcacg

SEQ ID NO: 27 - DNA, 46 bp, Artificial
  Primer
agcttctagactcctgggcgacctcgcgctccaggcggcgggccag

SEQ ID NO: 28 - DNA, 45 bp, Artificial
  Primer
agctctgcagagctcgtcgagcgcccctatcgagcgcccggtagc

SEQ ID NO: 29 - DNA, 46 bp, Artificial
  Primer
agcttctagactcctgggcgacctcgcgttccaatccctgggcgaa

SEQ ID NO: 30 - DNA, 45 bp, Artificial
  Primer
agctctgcagagctcgtcgagcgccccgatcgggccccgcggact

SEQ ID NO: 31 - DNA, 44 bp, Artificial
  Primer
gcttctagactcctgggcgacctcgcgatcaagatgggcagcga

SEQ ID NO: 32 - DNA, 45 bp, Artificial
  Primer
agctctgcagagctcgtcgagcgccccgggcgtgcgccgaaagcg

SEQ ID NO: 33 - DNA, 46 bp, Artificial
  Primer
agcttctagactcctgggcgacctcgcgatcaataatcgttgccag

SEQ ID NO: 34 - DNA, 44 bp, Artificial
  Primer
agctctgcagagctcgtcgagcgccccctgccagcacctatccc

SEQ ID NO: 35 - DNA, 44 bp, Artificial
  Primer
agcttctagactcctgggcgacctcgcgggagagtagcttccac

SEQ ID NO: 36 - DNA, 28 bp, Artificial
  Primer
ggggcatatgaacacacttcccgcctgc

SEQ ID NO: 37 - DNA, 30 bp, Artificial
  Primer
gggggtcgactcagacgctgaccaggctca

SEQ ID NO: 38 - DNA, 27 bp, Artificial
  Primer
ggggcatatgcgcgccatgaacgaccg

SEQ ID NO: 39 - DNA, 30 bp, Artificial
  Primer
gggggtcgactcaggcgccgaccgccacca

SEQ ID NO: 40 - DNA, 27 bp, Artificial
  Primer
ggggcatatgaagatttacggaattta

SEQ ID NO: 41 - DNA, 30 bp, Artificial
  Primer
gggggtcgacttataaaagctcttcgtacg

SEQ ID NO: 42 - DNA, 1785 bp, Artificial
  Environmental DNA clone
ctcgatattttgtttactttaagaaggagatatacatatgcggggttctcatcatcatcatcatcatggtatggctagcatgactggtggacagcaaatgggtcgggatctgtacgacgatgacgataaggatcccgcctttacccgaatggagccgcttgttgaggcggtcgcggatgcgatccggccctacgtggacgtgccgtttgccctgtttggccacagcctcggggctgccctggcctttgagctggctcgggtcctcgacgtagagcccatccacctgttcgtgtcggcgcggcgggcaccgcacctgcccgagcggacgcctccgattgcacatcttcccagcgccgagttcatccggtcggtgcagcagcggtatgccgggattccacaggcagtcctggactctcccgatctcctcgatttgctgttgttccggctgcgggccgatttcgaagtactcgagaactaccgccacctcaccggcgccccgctcggttgtccgatttccgcattcggtggacgcgacgatctcaccgtcacggccgccgaactggccggctggagcgagcacacctccgcagggcttcgcacccggacgcttgcgggtcctcacctgtttctccaggaccagcgccaggcgatcctggccgcggttggagaggacctgagaatcgcgacgagcgctggcactcgggcggctgtatgaccaccgacttcggatgggccgtgtcccggggtaacgcgatcggtgcggcgatcgacgtgtggattgtctcgctcgatcggcccgcggccgagtgcagcgccctcgaggctgtgctctcggcgacgaaattacgcgtgcggaccggtttgtcttcgagcgcgatcgccggcggttcgttgctgccgcagcgcactccgccaggtgcttggcggctacgtcgccaccagtccgaacgctctggcgtttgcctacggtgaaaacggcaagccggccctcgcgggcgaggacagtgagctgcgattcaacgtctcgcactccggggagtatgccgcgattgccgtttcccgggccggagacgttggcgtcgacgtggagtgcacgacgcggactgtcgaattcgaggcgctcgcgaatcgcttcttctcgtccgacgaagctgccgaactcggcgcgcttcccgagagcagtcgccggcaggcgttcttcaactgctggacgcgaaaggaagcctatatcaaagccatcggtgacgggctggcgtgtccgctggacagctttgccgtcacgttcggcgcctctgaacgcccggcctttcgctggattgccggcgacgacgcggcgagctggcagttgtacgcgttcgaaccggtgccgggttacaccggtgctctcgcgatgcgcgcgcccgcggcgtcgatgacactgtgcgcattcgaaggaaccgagctgtgagccgcgagcccatcgccattgtcgggatcgggtgccgcttgccaggcgcgtcgaacgtcgccgaattgtggcaggcgctcgtcgagggccggcacgccgttggcgaagtaccggcctcgcgcttcgatatcgaggcgctgttccaccctacaccggcaactcctggccgcgtcatgtccaagtggggcgggttcctcgagaacatcgatgggttcgacgcgcagtacttcggcatttcgccgcgtgaggcggatcggctggatccgagctcgagatctgcagctggtaccatggaattcgaagcttgatccggctgctaacaaagcccgaaagaggca

SEQ ID NO: 43 - DNA, 2112 bp, Artificial
  Environmental DNA clone
cttctcgatattttgtttactttaagaaggagatatacatatgcggggttctcatcatcatcatcatcatggtatggctagcatgactggtggacagcaaatgggtcgggatctgtacgacgatgacgataaggatcccgacgtcgcgcgcgaagactacgccgccgtcgcctgcgcaattcagaatttcagtttggctttgtggagcgagggtatcggcaccaaatggagcaccggcggcgtgaccacgtcagccgagacttacgcggtgttgggtgcagactcgagcgccgaggagatcgtgggcttcgtttgggtcgggatcccggcgcagccagcgagcaaggccaagcgtcgtctcgacgtgggcgcggtcgtgcgacgcgtgccgtgaacgcgtccttgccttccgtcctggcgtcactgttacccagcgacgtcaaatgcgccgaaggttgcccggcggatctgggcacgagtgcgggcctgttcgatgcggagctgcaagccatcgccagagccgtcccctcgcgccggcagcagtacgccgctacgcggcaccttgcgcgcggcctgttctccgagttcggactaccgccccagccgttactcaatcgccccgaccgtagccctatctggcctgccggctatcacggcacggtaacccacaccgatagctggtgcggagtcgcgatagcgagtgagcgtacgctgaacgggattggcatcgacgcggagcgcgccaaaacgttggagctgggagtcgtggagcgagtgctgacacgcggcgagtggcgagccctcgaactcgctggtcacgacccgctgcagcttggagcgctctgcttttcggccaaggagagcgtgtacaagtgcgtgtttcccagtgttggacgtttcatagggttcaccgaagtcgagattgacctcgacttggacggccgcgtgttcaccgttcgcgccgtcgactcaggcttgcacgcgagtcacggcgcgacgcttcgagcactcgaaggccgcttcgagcaagccgccggtctgtggatcacgacggcggtgttacctgctgcgctacgggctccagaggatcaaccgccgtctagtgcggcctcgccgtgatcctccggcggagtagcgtcagccaagtcgtctaaatagccttcgggcaagctcaccgtttgcacgcctgagctcttgccgcgcggtacgcgcatcgagtcgggggggaagggtaattcggggttcaacttcgaagcgacggcttcgagctccgccaaggtcttcacctgcatgaacgcttgacgaacgcttgcgctgccgcgaaagcccttcgtgtaccaagtcgtgtggcgacggaacgcacgcaccgcgtggtcttcaccaaaccactgcgcgagcagacgtgcatgttgcaacatgatgcccgcaacctcccccaagttgggcgggttcggtggttctttgccgtcgaagaccgcggccaggtcgcggaacaaccacgggcgacccaggcaaccgcggcctacgaccacgccatcacaaccggtgtgacgcatcatgcgcagtgcgtcgtgcgcttcccagatgtcgccattgccgagcacggggatggtcaggctttgcttcaagcttgcgatcgcgtcccagcgtgcttcgccgtcgtagagttgggcagccgtgcgcgcgtgcaacgcgacggcgctacagcccgcttgttctgcaatacggccggtttgcagatcagtacgcaggtcgtcgctgacgcccatgcgaaacttgatcgtgacggggatggcgccggctccgccgaccgcggcgcgcacgatgttctcgagcaacttcggcttgagggggatcgccgcaccgccgcccttgcgcgtcactttgcgcacggggcacccgaagttcatgtcgatgtgatcgactttgccttcagcgacgaggcgcagcaccgcctcgcgcacgaagtgcggatcggttccgtaaagctgcaggctgcgcggtgtttcgtcgggcccgaagtcagccagcttgaggatccgagctcgagatctgcagctggtaccatggaattcgaagcttgatccggctgctaacaagctgaaagaagcc

SEQ ID NO: 44 - DNA, 1218 bp, Artificial
  Environmental DNA clone
cgcgcgtgggagtagacgtcgagaaagtggggcgcgcaccgctcgcagtggcggagcgttacttctcccccagaggaagtggcgcagttgcacgcattgcccgcgaaggctcaaccgcggcgcttcctgcagttgtggactctgaaagaggcatacctcaaggccatcggcaccgggctcgccggcggactgggtcgcatgagctttgtgttcgggacggccgaagattttcgcttcgaacgcgccgatgatgcggacgccgcgcgctggcaatttcgccagtacgaaatcggcgagcatcacaccctcgggctcgcagtccttccgcgcgcgggcgatgcacggctggacgtgaccttgcgcgagtttcgcggggactgaaccggccggttcaggtaaggaacaacgcgcccagccccagcaacgtggcgaacccaatgcagtcggtgaaggtggtgaggatcacgcctgccgacaacgccggatcgatgcgcatacgccgcagcgccagcggtacgaccacacccacagcggcagcggcgaggaggttgatcaatgtggccgcagcgatgatggcggcgattttccaggtgccataccagacgatggtcacggctccgacgacggcggcccagatgaggccgttgagcccgccgacggcgatttctttccacagcagccagcgcgcgttgacccactcgacctgcccgagcgcaatgccgcggatgatgagcgtgacggtctgcgtgccggcgatgccgcccatgctggcgacgatgggcatgagggccgcgagcgccgcgaccttttcgatggtgccttcgaagcggctgaccacggcggccgccatgaaggcggtgaccagattgatccccagccacaccaggcggcgctgcgtcgaagggatgacacccgcgaacaggtcttcttcgtcacgtagtccggccgcggacatgacttcgtgcgcggcttcttcgcggatgacgtccaccacgtcgtcgacagtgattcggccgagcagcttgccctggtcatcgacgacggcggcggacacgaggtcgcggtcctggaagagctgcgcaacctcgtgcgcattggtttcgggcgagatgcgcgatgcttgcggatcgaggatgtcgcccaccgatttttcaggtcctcggtcagcaggcgggaaagagcgacggtgcccatgtaccgatcgtcgcgatcgaccac

SEQ ID NO: 45 - DNA, 4829 bp, Artificial
  Environmental DNA clone
cnnnggaannnntcngaatattttgtttactttaagaaggagatatacatatgcggggttctcatcatcatcatcatcatggtatggctagcatgactggtggacagcaaatgggtcgggatctgtacgacgatgacgataaggatccgcgtgctgcaactgacgcgcggcggcaccttgggtgaggcggaggcctgccgcaagttcatcctcggcggctcgccggctggtcatcaactgttggtggtcagctcgccgtaccacacccggcgtgcgttcgccgtgttccgtcatgtgctgacgggcacggaagtcgaggtcggcgtcgccccggccacgctgttctcacaggcgcgacctgagctgtggtgggctgctccgtacgacaggtggtatgtggcctacgagtgggccgccacggtgtactacgcggttcgtcacgggatcctgccggtgatcggttaaggtgccaggacaaagtcgtgcgttcgccgagtagccatcgagacggtgcatccctgctgctgttgcgctggacgcgacagttctgcctggagctcgaccagccgctgccgttagctgctcaacacatcccgatcccacctgcgctcgaacgcgcagtaccgcgacgtcagacacaatttcgagcaggtcgatattgcgccctgagagctatggaagcactcgacccccggttggcccggcacaaggttgaacgcagcaccaccggagcgccagcctggccgccgggtattaccgggtcgatcactcataccgatgacttcgcttcggcagcggttgcctccacggctgaagtggcttctctcggaatcgacactgagcgagtcctttcggccgagcaggcgcgcgatgtcggcgacgtcgtcgcctcgtcgagcgaactacagcactgtcacgcggccggaatcacaccgctcggggggctcacgctggtgttctcggcgaaagagtcgattttcaagtgtctccatccactcgttggtcgcatgttcgagtttcatgacgttcgcctggtcagcgtcgatgtggctacgcagacgttccgcgcggagatcgtgacgacactcggctcggacttttcggcagggaccttcctcgaagggcgcttcgcgctcgagggtcaccggatgcacaccggtatctcgatcgaaaccccagaggcctttcgaaatcagggtcgctcgagttcgtaggcgccaacgtcgaacgcctcgccttgggggcgccgtgtacccgcgcggtcgtcgttgacctccacgatcgaaacgccggcgtccactgccgccgagtcgctgctcaatcggtagtcgtgtgttcgatcatcgacgaacgcgctcggccctacggcgagattcgactggtgtgccgcctcggcgggcttctggccgaggatgagtgtgttcagcaccgtcagcccatacggtttcgacgccgccgcctggaacacccgcccgacgccgttgccgatcgtgctgttccagatccgcaagttctgaatgtcgtcctcgtacctgatcgcggtcgccacgtcatacaccacggcgtttttcagggcgacccatgcgcctccgctcggcgcgccgcggagtctgaacgcaatttcagagtcgtgcaccgtgacgcgatcaatcactgcgtcgacatgctccttgatgttgaaggccgccatgttcgtgatcaggccgttacggaagccccacgcctcggtgtcgcgaatgtggatggtggcccgagcgaagctcgggctggctttcgtgtcgaccgcgttttcgcctggcaccacgccggccgcatacccgttctcgtccgtcgcgagaggcgcgagccagattctgttgcgctccagcaggacgtccgtccagcccggcgcagcgcggccggagtcaacttggaagccatctcctgagaacgtgtgaatctcggtgtcgcgaattgtaagacggcgcaccgcgcccgccacgatcccgtgagcgtcgctgcggccgtcaaccgggttgagcgcatgatgaatcaggcacccctccacgagcacgtcatatggcgctcccatgtcgatgagatcgcggctcgaccgtcgcacctcggcgttgcggagcgtgaaggtatttccaccgctactcacacggacggtgtcgtcggccccgtactggccatcgagcatcaggccgtcgacgatgatgttctcgtgatcgactgtgaggacgcgccccgaggcggacacgaggacaaaacctcgaccgccgagggcgcggatcgtaatcggcgcaccatccacgccgttccgcaccgtgcggattgactccgcgtaggtaccgggtcgaactgaaacgacatctcctggcatggcgacgttgagggcctgctggatacgtccaaagggcgcggtagccgtaccattgccggccgcgccagccgcgacgaaccactccgtgccgagcgcctggaggggcgccgccgccaagcagagtgccacgagcgcgaagagtcgaaaacgggaaggtgtcattccaggcgacagactttttgcagcgtccttcgcgctgcgtgtcgtacaggactctggggcagcaagcagtatcgacctcaatccagctgccgtcaaccggcacggagcgatcgccgcaacgcggggttctatcgggcgatcgcctctttgagcgcgcggctctcggcgcaacggagggcgatgccctccaacgctgcaagcgtcggcgcaagatccgctggcgtggcggggaccgagtatcgaccggccttgatgcctgagccataaaaacactacgggtacgtgcgtgtcgtagaggtgtgccgtgccgtgatcggcgtccgtggcgtgaagcacccagccagccttgggaaccacaatcaggtcgggacctcgaaccgggtgataaccgcgtcgatgcgccgtcaagacagcgtcggcggaaggcaccgtggacagcagatcgcgcttccaatacgcgctcgacgccagggatcgcagtggcggcccgcgtgacggcatcgagggtcgccgtgctggcgtgaacccgatcggcgacctcggagttcaggtagatctggtggacgtacatcctggcgatgtggacaccctttcccatcacgccggttagcgcagcctcaagcgatccccgcaacacgcgcggattgatacgcccggaatcagacccgaacgacggcctctgctccggcatgttcgaaacgccgtgaccgctcgagagcgccagaacgctcgagagcgccagaacatagccgcccggaccgaccgacgcgtcaagcctgtcgaggagcccccccagcgtcgcatccagacgaacgagggcgtcctgtactcatggctgtgtgggccgtagtcatggccgatcacgtcgggcgccgagaagccaatcgcgagcagatccggtgttgagccccgccaagctgcagcgagctcacagggcagccgccatttcccccaggtacgcatcgctccaaggagaacggagccagttccgggcagacagctcgtacggaaacacccgcggcgcgggctcactttaggaaggtcgtcgccgaacgtgtacgccgcatcgggcaggagtcgcgtccagagcgtgccgtaatcggccgccatcggatgtgctcgtacatacgcagccacctgcggccacggtgctttcctgtaggccgacgagcttgccccacgccgtcgctttcaggccacagggcgatcgtgctggcacggccgtggccagccagcatcaccgctgagcgagcctttccagagagcccacgacgcggacgcccggacccatctgcgccgccatctcatcgcccagggtcggcgccatcaacaatgcaggtccgtacttcccgctgagcgttccgccaatcccgattggcacgaccgagttgtcgtcagtgcagttgaccgtcttgtccgtcgcccgatcgaaccagctgttgtcgattatcccctgcgtcggtggcagtgtacccgtgctgatcgtggcgtgaccgccgcacgtcgaggtccgaccgaacgggtactgggcgttcgtgaagaccgcgccgtcgccgaagatacgcgtgaggccgcgcgaccattggctcttgtacagatcctccatgtccgcgcggaactgatcgatcaccacgatgacgactagcctcggcgcgtccctctcctggcgcgccggctgttgagcggaaatgcgggccaggagcaacagggctgccagtcccgtcacgccaaagctagcgaggacggtctcagacatcaactcccatttgcttatcggccgacgtccggtctgtacgacgagcccggtctgtatcacgagccgtccgcgcacttcagcgtccgtcgtgaaggtgaaactcaagctacggccgatgaatcctgcacctcccggtgacgagtacacgagtcattgaggtccagtgtaacgccggcgcgcagccctgaagggcgctctaacccttgtgaaattcggagtgccagaaccgcttcgagcgagtatccttcggagaacggagcccccagagcgttggcccgaagcgatggaacccgcgctgagccctgccctcagccggtcccgatgtgcaacatgggacctcggcccgatatgccgggtattggacaacgtgatctttggttactgagcctcttcccacttctacacactcgggggcacgccagattgcctcgggatctcacgccacactcctcgatttcgtcgagcgcgcggatccgagctcgagatctgcagctggtaccatggaattcgaagcttgatccggctgctaacaaagcccgaaagagg

SEQ ID NO: 46 - DNA, 3395 bp, Artificial
  Environmental DNA clone
nnngccaatagctctttcgggctttgttagcagccggatcaagcttcgaattccatggtaccagctgcagatctcgagctcggatcaatacggtccgcaagaacagtttgtaccgtgcttggaatgcgaatgctgtcgatcttcaggccggggcgatactgtcctttggtaccgaccaaaaacccgctctccaccagggaccgcacgctctcctcgacaaaaaaaggattgccttcggtccggttgatcaacaatcgtttaagagatgccagatcttcattgtcacccaataaaaaccgcaccaattcttccgcgctttgatctgataacgggtcgacgcgtatttgggtgtaataactcttatctgaccaagtgtggctgtagccaggtcggtgattaacgaggagcaacaaacgggttctaggcagactctccaccagatggtcgagaaacgcttgggtctcgctgtcgatccagtgaagatcttccataagcaacaaaatcggccgcttcaagctttcacggatcaccacccgcttaagagcgtcgaaagtcaggcgccgccgttgttgaggctccaggtattcgaattttttgatgagctcagaaatttcaatatcttttttattgccgaacgtattcgttttttccggattttcagcatccggtaatgcgtctagcagagctaaaacaggcggaatcgtgtcttttagtctggggtccaattcaattagatgcgtttggaccttgagccgaatcgtttcatggctttccccttcgtggatttgaaaatatcgccgaagcaattccattaacggataatacggggtggctttgccgtaagatgccgaagttgcttcaagcactgtccattccggaggtagatggctatgcgtgaactcgtagacgagccgggatttacccattcctgcttcgccacagagcgccaaaatctgcccgtgacctgaactcgcttgttccagcacacgcttgaatatttcggtttcgacagtcctaccgacaaacccactaagcccacgagcggcggcagcgtgtagtcgtttacgagccgcggtcactccagtaagttcaaaagtttcgatcggccgaacgaatcctttcgcctggagcaacccgagagaattgacctgaacaaaaccttctacttcgcgcagagttctcccggtcatgaagataccacccggggcggcgagctcctgcattcgcgccgcaagatggatcgaatccccgacggcgagatagtcaaagttaagatcgttatcgatggtacggacaaccacctcgcctgaattcaaaccgatacgtatctgcggtaactcagccgaaggaccgtttttgtcgccgatgcgccggagctgctgctgcatagccaatgccgcgtaacaggcgcgaactgcatgatcctcgtgagccagcggtgccccaaacagcgccatgataccgtcccccaagatatgattaacggttccttcataacgatgcaccgcctccatcatggctttcaggattggatctagtagcttctgcgcctcctcgggatcgagcccttccacaaggcgtgtggaatctttaatgtcggcaaataagatcgtcacctgctttcgctcaccttctaaagtgtggcgctcggcgaggattctttcggcaaggtgtttcggaataggtggctggcgtgctgaagtgctttgagctgtagtgctctgaattgctagtgttggtcggctaagtgactggccgcactttttacaaaactttgccccctggcgattaacctcaccgcagtttgaacacgcggtttctagcggcgtaccgcactcgtcacaaaaggccgcggtgtcaggattctgattcttacaacgagggcaattcacttcagcttctccgtgcttcagctagaccgttgtgtcagaatggcaacgcgtgctggttcggtaggcctaaagtagaagcttgtacatcttgtactatctcctgacgctttggaggcgaaattcaagggcagtgactaacatgcagccggcactcggcgccagagaaaaccatcgcgccagaaaagccgggtacagcgtctatatccaccatgcaccaacgagaggtttcctcgggagcccagccgatgcgcagcaatcttgcaggctcgcccggtgccagggatacgtcgaaatccctcagcggacagctcaacccgtccccgagagcttttacgaaagcttcctttcgggtccaacatttgaagaacgcttccgttcgtgacgtgtttggaagcgcctgaagtgctgtcagttcggccacggaaaagaaatttttagcgacgtcttccagaggaaaatcagcttgaatggattcaatgtccactcctacagatcgatcttgtgtcatcgcgtaaattgccgttccatgggaatgggccaggttgaactgaatcgccggttgaccgtcttgccgttttaacccgggctttccgtggctgccataagtgaagataatttcacggggtggcactcccaaataagagctcaagatgaccctcaacaatcctcgtccggtgacaaaccgctcgcggtgcttgtgaaagtgaaagcggtttgctctatcgacttcatcctcagcaagaatactttcgaaataactctgctcggaatgcggtcgatccaaacaagcgatgcggacatgtacatcctcttggcctagcagtgccacttcaatgccggtcatcgtgtgcaatttacttggaggtgacttgcatttcctaacatagtcctaatcaatcgatcaaacgaaatatttatccgtgtagtagcaacgtgaatctgtcaggggctaacggcaacgtgattatgtcagggtgggaggatgacaaccctgacaatgaaagacgaaaagcgattagacgtaatacaacgagtgttgcggcgcgagctgacggtggtgcaagccgcactggtgatgggagtaagcgaacggcagtgttaccggatcaaagcacgggtcgataaggccggagccaagggagtggttcacggtaaccgcggacggccctgtaaacgcaagaccacggagaagatggtgcggcgggttgtgcaattagcccgagggaaataccaaggcttcaacgatcaccatctgaccgagaagctcaaggaacaagaaaagatcgagctcagtcgagagaaagtccggcgtgttcttcgtgcaaacgggattggctcccccaggaagcgccgcgggatccttatcgtcatcgtcgtacagatcccgacccatttgctgtccaccagtcatgctagccataccatgatgatgatgatgatgagaaccccgcatatgtatatctccttcttaaagtaaacaaaatatctagagaanncnncnng

SEQ ID NO: 47 - DNA, 1625 bp, Artificial
  Environmental DNA clone
ggaattgctactttagaggagatatacatatgcggggttctcatcatcatcatcatcatggtatggctagcatgactggtggacagcaaatgggtcgggatctgtacgacgatgacgataaggatcctcgcggcggcggggctgccgccggtggagaagcgggtctccccgggaaccgcggtggcgctcgggttcattctcgagaaggcccacgcgtggctcggccgctcgggggagccccgcatgacccgctggctcgcgcgggagctgtcgaccgcgcactggttcgacctgaaggcggcgagacgggacctcggctacgagccgcgcgtcggcatcgccgaggggatggagcggctcgcgaaggcgctccgggacaagcggctgtgagcggaggctgcgacccgaacccggcgccgacgggactccgcgccgacgaggtgctcgtcgtgcggggatcgctcgacccggaggacgcctcgtccctcgaaccgctgctctccccggaggaggcggcccgcgcggcccgcttcgtccacgcgcgggagcgggcgcgctacacggtgtcgcgcgggcggctgcggatgctgctcgcggggctcgccggggccgacccgatggacctcgcgatcgagacgggggccgacgggaagccccgcgtcggcggcgactcgcggatccggttcaacatcagccactcgggcgcgctctgggcgtgcgcggtcgcgctgcagcgcgaggtggggctcgacctcgaggagatccgcccgggccgggaggtcgaccgcatcgcggagcgtaccttcgcccccgcggagacggccgcgatccgggcgctgccggaggccgagcgggccgcggcgttccaccgctgctggacgcggaaggaggcgtacctcaaggcgcggggcttcggcatcgccgaggcgatcccgctcgcgtcgttcgaaggggaagtgcggcccggcgtgccagcgcggctcctcgccacgcagcccgacccggcggacgcgggacggtggagcctgcacgacctggacctcgggcccggcttcgcggcggccgtctgcgtcgagggcgaggtgtccgcggtgcggatcaagtagagacaacggatcaccgcagagacgcagagacgcagagatgcgcagagaacgacaagcgccctggaacggcgagtcaacggatcaccacggagacacggagagcacggagtacccacggagaacggtcagagtccttggaacggcaaggcaacggatcaccacggagtcacggagtcacggagacacggagatccacggagaacggcgaaagccttggaacggccgagtcaacggaccactgcagaaacgcggggatgcgcagagagagacggtgcctggagacgacgaggatgcgctcgacggccaggcgggcaggaaagcgctccatagacccccggagtgcccagggggagggcgtccccgcgcgcgacctgaggcagagccgcaagatatcggatagtgatctgcagtgttggcattttagaaattatgggtatatctgggctttggacgtgtggcggatagtccggtcaattcgcgtcgaggggcgcgccgacanatagctacaagtctccagaa

SEQ ID NO: 48 - DNA, 2879 bp, Artificial
  Environmental DNA clone
nnnnnanncncttcntcgggctttgttagcagccggatcaagcttcgaattccatggtaccagctgcagatctcgagctcggatcccgacgacgcgccagggcgcctcctcgtgctcgtcgaggtcggcgcgggcgtcgaggccgtgctccgcggtgaacgtgtcccccaccttcagccccgtcgccgcggcggccgtcggcccgagcaccgcctcgaacgccttcgtctccgggtcgaacgggccgaacggggcgccctcggccgtctccaccgggacgccgtggcgcgcgtccatcccgaagagcgcgtccgtggtgccgacgacgcggaagccggcgtaggtgtcaccgaaggcgaagggcacggcgcgggtcacgcgcgggtccgccgccagagagcgccagcgcgactcggggatggtcccctggtcctcgcccacgtggtagacggcgccgagcacgatgcggagcgggtcccccttcgggccgacgacgaggtccgcggcgagcgcggccccctcgaagtgggcgcgggactcgcgccggagcgcgtccacggcggtgaccagcgcgacgccgagcgcgatggcgagcgtcgacaggagcacggcgctcccgcggaagcggagcgagcgcaggacgaagccctcgtgggtcacgcggccgcctccgccaggtcgcggatggcgaccgcggccggcaggtcggcgacgaccgcctggtcgtgcgtgacgacgacgagggtggccttctcctcctccgcgacgcggcgcaggagtgcgatcacctcgtcgcggcgcgcggcgtcgaggctccccgtgggctcgtcgcagaggagcagcgacgggcggttcatcacggcgcgggcgacggcgacgcgctgctgctcgccgagggagagcatcccgggccggtggcgggcgcggccggcgagcccgacgcgctcgaggagatggtccgcgcgcgcgccgtcctcccgcgaggcgcgcccggcgaagagcgccgccgcgagcaggttctccctcgcggtgagcgacgggaacaggttcagggactggaagacgagcccgatgcgctggccgcggaagcggtcgcgcgccgcctcgtccatggcgccgaggtccgcgccgagcacctcgacgcgcccgcggtcgggcacggagagccccgcgagcaccgacaggagcgtggacttcccggagccgctgtcgccgaccagcgcgacgtgggcccccgccgggagctcccagcgcgccacgcgcagcgccgtcaccgcggcgccgtcggggccggggtaggtgcgctccacaccctcgagccggatcgcgttctccgccacggggccattctcggcgcgcccgctcgggacgcctaggatcgtccacatggccccggacgaggacgtcgtcgagcgccagatcgggcgcaggtcccgcgggctcctccgcgtggagagccgctgcgcctggggctaccccgtcgccgtctcggtcgcgccgctcgtcgacgagcgccgcggccggggtccacgcgagccgttcccgacgacgttctggctcacctgcccgatcctcgtcgagcaggcttcccggatggaggccgcggggctcgtccgccgcctcgaaggaggaggccgccgccgacccggccctcgccgcgcgcatcgccgcggaccacgcgcgctacgccgccgagcgcgcgacgctcctcgacgcggacgcgcgggccgaggcgtcccgccgcgggctcctgccggtgatcgaccgatccggcgtcggcggcgtccgcgaccaccgctccgtgaagtgcctgcacgcgcacctcgcgcaccaccgcgcgcgcgggggcaacgcggtcgcccctcgagatggaggcccgccacggcctccgcgagtgcaccgaggccgaggtgcgctgccggggtttcggggaggcgccgccgcccgcggcccctacgatgccggccggaggtgccccgtgagccgagcctggaacgacgtccgccgcgtgctggtccccgtggacttctcggactcggcgcgtccgatcgtcgagtacgcgcgcggcatcgcgaaggaccggggcgcgtcggtggtcctcctccacgtggcgacgacgccgtcgtccgcgatcgagagcgcgaagagcaccgccgagttcctcgccgcggcggagagggagctcgcgtccctcgccgcgctcgtgaaggaggccggcatcgtcgtcgaggcgaaggtggtggtcggcgcggccgcccgggagatcgtccgctacgcgagggagtcgaaggccgacctcgtggtgatcggcacgcacggccgcacggggatgcggcacgtgttcctcgggtcggtggcggagaacgtcgtgcgcctctgcccgtgccccgtgatcacgctgcgcatgccggggttcgagatcgagcgcctgtagagccgaagagtcaacggatcaccgcagagacgcagagtcgcagagatgcgcagagaacgtctgcggcagcggcaacggatcaccacggaggcacagagcacacggaggacccacggagaacgacggaaggccttggaacgcgtggcaatagccacgaccagattccaaggactccgagatcactagagaggcaaaccaccacgacggcacgacgaacacgacgatccgacganggcgagagtcttcgaacgggccgcgtccggncgaccgcctccngctcgnnctccgtcgangggacaacggatgaagtccgnnnngatnnngagaaccattcnaggctcgnanagaccattnnnnnanccnngcgngaaggnacnnnaacctgnnnnnctngttnnntgcanntcnnnnnntttgngatgnctgcccnnntcnatgctannnt

SEQ ID NO: 49 - DNA, 5493 bp, Artificial
  Environmental DNA clone
nnttntttacctttagangagatatacatatgcggggttctcatcatcatcatcatcatggtatggctagcatgactggtggacagcaaatgggtcgggatctgtacgacgatgacgataaggatcctgacagccggcttcgagctcatccatcggcaaactatcaacatgctttggtacgggcacgagcttctggtgttttgcaagcgttcggcacagcccgcagcgcgagcgaggaagccatgaaactcatttcctgggccgctctcattctcgccgtcgcggcgaacatcggcgccaatacggcgttgaagagtgcggtcgcatcgctttccccccaggcgggtgaaagctcgatggtgcagctgttgcggaacggatatctgtgggtcgggctggcgctggccgcactgctcctcgtctcctatctcgtagcgattcggcacattccgatcagcgtcgcctacttggccgttaccacgttggccatggtcggccttgtcgtagtggacagcacgttcttcggcctgaagctcggcgttcacaatcttctgggcatcggactggtgatggtgggcttggcgctggtcctgaaaggcgtgtcgttgcggtgacagtgatccgccgccgtgcggctcaggtagggaagaatttcctattcgtacatcgatgctcccgcagggccagcggtgtgaagagcggtcaaattctgtaaatttgccgatcggactggactgcgagggggcgagcgcgggaatttcgggtagcgtcagccgaggcgcgttctggcgttaggggatttcattggcaggggggcgatgaggatgttgcatgtcgggttccgcgtggttaattcggcggagcgccgcgcatcgcgcgcgacgcaggatcggcgaacggcagcggaaatggcgctcccggtcgccgatggcgcggtggaactcgtgttggtccgcctggaggccgagcttgcggcaacgcacgaactggcgcggtgcttgtcccaagaccaacgagtcagagccggccggctcgtcttcgaacgcgatcgccgccgtttcatgatcgggcgcgcgcggctgcgccatcttctcgcggtgcgcctggggacgtgcgcggagctggtcgagctggactatgggccgcagggaaagccaagcctcgccggcggcttcgccgcgtccgacctgcgcttcaacgtgtcgcactgcggggatgtggcgctgtacgctttctcgcgctgccgggagatcggagtcgacgtcgaggcagtccgtgaaatgcccgacgccgacgacatcgtggagcgctttttctcgccgcgagaacgcgccgcctaccggcgcctcccggcgcgggagcgaccgcgcggcttcttccgctgctggacgcgcaaagaggcctttatcaaggcgctcggcagcggcttggcgtacccgctcccggacttcgattcgacccctgcgccgaggggctggagcctgcgcagcttcgtcccgggcccgggactggtcggcgcggtcgccgtctcaaccaaatgaaacactttacgatcggccttcgctccaccccggacttggccgaccacggtttccaggacatggccgtgctgcctggcgcactctacgtcgagcacgcactgcaaatgggcgcacggcgcgtgcgcaacgtgcgcttccgcaatccggtcatcctttcagctgacgacgcgctcattaccatttccgccgagcagggacgcaccgtattccgcgaagcgggcacgggcatcgccgccgagctcgagctcgagccgggcgcgcccgaggcgcgcgttcacccgtttccggtggaggaattcctggcgcaggcgcagccggtgaacgacttctacggtcggctgcgcgccaacggcaaccagtacggtccggcttttcagcgcgtaacttcgctctggcggcgcggggacgagatactgggaaagcttcgcaccccgtccttggacgcggcggtccagctgctcgcgcccttcatcatggaggaaggcaggaccttcgtattgcgctcgattgagcgggtcgaggtctacgcccctgaatttccggagacgctgtggggccacgccatacgacgcggcaacgtgcgcctcttcgatgactcgggcaagacctggctcgagctgtccggcgtggaattgagcctgctcgagcgcgtcgccggtaccaagctcgcgatcgccgccaacttcacggcagagccattggaggacgcgctgcgcttctggagcgatcagctcggcgagcgcctggagcctgaattcacctcctacgaccaggtgctgcagcagctcctcgacggcgcgtcggcgtttcagcgcaacgcgggcggcgtcaacgccgtactgctttcgctcgaggaatggggcggcggcagccggcaggcgctcgtcgacctggacaaggtgcgcgcgcagcgctgcttcggcgagcgcccacgccgtgtcctgccgaacggcctggagatcgtgcacctcaatgcctacgagaccgattacctgtatcgcgagatcttcgaggacgagtgctatctgcggcacggcattacgctgagtgaccgcgccacggtggtcgacatcggggcgaacatcgggctgttctcgctctttgtgatgagccgttcgcccggcgcgaagatctacgcgttcgagccgggcgcccgcggtatacgagcttctgaacgcgaactgcgaggcctacgacgaccacgcgcacgccttcaacgccagcgtcggcggcacggctggcagcgcgagcttcaccttctacgagaagtcctcggtattctcgggcttccattccgacgaaggcgacgatcgccgcgcggttcaggccgtggtgcgcagcatgctcgcggagaacgagctcgccgacgaccagacggaaaacgacgtcgccgcgctgaccgccgatcgtctgcgcggcacgcggcacgaatgccgcatcacgaccgtgtcggatctgatccgcgagcaggcgctcgggcgcatcgacctgctcaagatcgacgccgagaagagcgagctcgccatcctcgaaggcatcgcggaagaggactgggcgaagatcgcgcagctcgtggtcgagattcacgacccgagtggcgaggccgtgcggcgcatcgagcagatgctcgcggcgcgcggctaccgttgcgtggtcgagcagcaggcgcttctcgagcgcgcgggactctacaatctctacgcaactcggccgggcgcgcacgccgcgcgttatcccggcctgcagcgcagcgtcgaggacttctgtgccgcgctgcattcatatgcaagcagatcttccgtgccggttgtgctttgcatctgtccaagaaagagcgatcccgagctcgaccaggccgaagccgcgctgttgtcgcaggccggcgcgatcgcgaacgtccacgtcattggctcggcgacccttgccgagcgctacccagtcaacgactactacgatccccagacccatcgcgcggcgcgcattccgtacacacccaagtgctactccgcgatcggcaccgcgatcgcgcgcggcgtgttcggactgaaggcgcggccgtacaaggtgatcgtgctcgactgcgacaacacgctgtggaagggggtgtgcggcgaggacgggggtggagggcgtcgaggtcaccgcgggacaccgtgcgctgcaggatttcatgctgcgccagatgagcgccgggatgctgctctgcctgtgcagcaagaacaacccggccgacgcgctcgaagtcttcgagcggcgcatcgacatggtgctcaggcgcgagcacctcgcggcctgacggcctgaactggggacgcgaagctcgcgatctgagggcgctcgtccggcgagctcgacctcaggcttcgacagctttcatattcatcgacgacagcccgctcgagtgcgcgcagatgaggctgcactgtcccgaggtactgacgctcgagctgccgggcgagcccgagtcgttcgccgcattcctcgatcatgtctgggccttcgaccgcgcgcgcgtcacgcaggaggaccgcagccgcacgcgcatgtaccaggaagcgagcgagcgccggcgcttccgcagccaggcgctgtcgctcaaggagtttattgacggcttggggttgcgcgtcgagattgccgccgcggccgagggcgagttcgcgcgcatctcccagctcacgctgcgtaccaaccagttcaacttcaccgcccgccgccgctctgagcccgaagtgcgcgaattcctgcagcgccctggcgcgcattgcatgacggtgcgggtcgctgaccgcttcggcgactacgggctcgtcggcgtcctcatgtacgaaggcgactccgaccgctaccgggtcgacacgctgctgctgagctgccgggtacttgggcgcggcgtggagcacgcgctcgtcgcctggctcggcacccgcgccctcgccgacgggaaggctgtggtggagctgagctacgagcctagccagaagaaccgcccggcgcttgagttcctccaggagatcgccggcgactcgctgtccgttccggcgaagcggctggcagccgtgaagtacgaccccgacgaaaagcagtccgagcccgcggtgaaaaccgctgcagcgcctgccgccaagccggggtatcgctcggagctgctgcagcgtatcgccgatgagctgcgcgacaccgcgcggctggcgcaagcgatcgaggcgcacagcctcggcaagcaagtcgctcagggccccgcgacgctcgcgacgctgtgggcgagggtgctgggccgccccgaggtggggccagacgaaaactttttcgaggcgggcggcacctcgctgaaggcagtgcaacttgtcgcgctgatccgcaaggagctgaatcaatgggtctcggtcgtaagcgtgttcgagtgtcccacccttgcgtttctcagtgccaaggttggaacgccccccgaaagaccatctgccgtaccggcggcgctgctgcgcggccagcgccggcgccagaacgccgcccgcatgaagggttcctgagcatggacatgcgcaagcgcgaccgagccatggaccaggcgcccgcgaccgcagcagcgaacctgacacgcgtcgccgtcatcggcgccggcaacatcggcaccggcgtggctaccgacctggtgctgcacggaatcgacgcagtggtggtcgacctgtccgaggaggtcctgaaaagggcgcaggccgagattctgaagaacgtgcgcttcgcccccatgttctcgaagtcgttgccgagggtgagcccgcaacaggctgcgcaacgcctcgcgttgaccacgcgcctggaagacgtggcctcgtgcgacttcgtggtcgagaacgtgaccgaggaatgggagatccgagctcgagatctgcagctggtaccatggaattcgaagcttgatccggctgctaacaaagcccgaaagaangnt

SEQ ID NO: 50 - DNA, 2295 bp, Artificial
  Environmental DNA clone
gnnnnactntntaggggattgtgagcggataacaattccccatcttagtatattagttaagtataagaaggagatatacatatggcagatctcaattggatcgctttctacgggggtgtcgcgtgcggccgacagacggcgatgccggaaacctgctggccatcagcgacctgcacatcggctacgccgagaaccgcgccctggtcgagaacatgcgtcccgagacggaccaggactggctcctggtggccggtgacatcggggagatcgtcgaagacatccggtggaccctccggacgctcgccgaccgcttccgcaaggtcgtctgggtgccgggcaaccatgagctgtggacccatcccaaggatccggtccagctccgcggagtggcccgctacgagcatctggtcgccctgtgccgggagttgggcgtcatcacccccgaggacccctaccccgtctggaacgggccgggcggccccgccgtcgtcgcgccgctcttcctgctctacgactactccttcctgccgcagggctgtgccaccaaggcggaggggctcgcgtacgcccaggggaccggcatcgtgtgcacggacgagtacctgctgcacccggacccgtatccgagccgcgaggagtggtgccgggcccgggtggccgagaccgggcgcagactcgccgcggttcccgaggggctgccggtcatcccggtcaaccactatcccctggaccgtcacccgatggacgtcctgtggcaccccgagttcgccatgtggtgcggcacggcggcgaccgccgactggcaccgcaggttccgtgtcgagaccatggtctacggacatctgcacatcccccggaccacctggcacgagggtgtccggttcgaagaggtgtcggtgggttatccgcgagaatggcgtacgcgaccgggtccgccgggcacgctgcgccgcattctgccgatggaggtcaaagccggtgatggaggagctgcttccggagtcggtggtggtcgtggagagccacggtgacgatccggccggggacgcgagtctgtaccccgaggaactggcggtcctgacccgggccgtgggcaagcggcgccgcgagttcgccaccgtgcgcgtctgtgcccggcgcgccatggagaagctgggtgtggccccggggcccgtgctgccgggcgagcgcggggccccgcggtggccggagggcgtcatcggcagcatgacccactgcgagggctacggcgcggccgcgctcgcccgcgccggcgatctcgcctccctcggcatcgacgccgaaccccatctggcactccccgacgacgtcctggacgttgtcgccctgcccggtgagtcggaccgtctgaaccggttcggcgcccgggtacccgccgtccactgggaccggctgctgttcagcgccaaggagtcggtctacaaggcgtggttcccgctcaccggcaagtggctcgacttctccgaggcggacatcgacatctccctcgaccggatctccctcgaccggatcttcctagacagggcgcccgtcacggacgggctctcgggagacctgagggcccaactcctcgtcccgggacctgtggtggacggccttgtcgtccagaccttcaccggccgctggacggtccggggcggcctggtcgcgacggcggtcacggtcccgcacaccgggccgggcccggtgccggggtccggcgcctgaccgtacgtcgccaccccggcgcggtcccggcgccgactgcgcgcggtcagcccggttccgggcaccaggcgtgcagcagttcgaagaacgcgtcctcgttgcccgcgaggcccgccgccttcagggcccgttccgtctcggcgagcacggcgggcgggaccatgggtggccggcttccgtccggcgggccgtcgaaagccgctcgtaccgtctggaggaggcggaggtaggcctgcacggcggcgcgttcgtggtcggtcagtacggcggtcggcatcggacggctctccctggtcgacttgtcgtgacactggtcctgaacccagcttgccgtccgccactgacaatccggtttcggtccctggaggttcgcccgcttagcggaggcgaaacctcacggaaatccctggtggaacagggcccctacgctggaggggagagcaggggccgcccatcggccggccacgcgatcgctgacgtcggtaccctctggttctggtaaagaaaccgctgctgcgaaatttgaacgccagcacatggactcgtctactagcgcagctaatcccc

SEQ ID NO: 51 - DNA, 2157 bp, Artificial
  Environmental DNA clone
nnnnnncncnctatngggggattgtgagcggataacaattccccatcttagtatattagttaagtataagaaggagatatacatatggcagatctcaattggattaccctgccgaggtggagaacatgatttacacctacgcgggcgtgcaagaaaccatcgtgttcgcgataccggacccaaaacgcgggcaggtcctcggtgccgccgtcgttcctcgtcccggttcgagtctaaacgaaagagaactcttgacgtttctgcgcgccaatctcgccaatttcaaagtgcccgacagaattgtcgtgcgcgacgcgttgccgcgcacgtcatcggggaagacgattcgggacgcagagacactgctggcgacctaggcggatcggcgcatggcaaaggaaacggccggcgaacaattggttgcggcgatcagaaacgccgccggcatcattctcgccgttaccgaaaatcgtaactggatcgaagcttttctcgaaggcgatcgggtaatcgtcacaggcgtcgcgcgcaatgcattgtgtaaccgcgctgcgtgcggaatttgcggtcgtaaaatgtcaggctgaggccctccgcgagtggctaattcatgaagccatggtcgactgctcctgatccaattccgccgcttggtgcggacgcgcacgtctgggcggtggagttggacgactctggttttgacgccgcactcgggcacacccgcctttccgccgaggaacaagcgcgcgccgctagattcaagttcgacagagatcaaaaacgttatgtcatcgcgcatgccgcgctgcgcgatatcttggctggctacacaaatgccgacgccgcaagcttgcagttccgcgacggtccgaacggcaaaccgaagttggctccacctttcgacgcaagcggcatagagtttaacctttcacattcccacgaactcgcgctggttgcggtcaatcggaccggtgaaatcggtgttgatatcgaatttgctaaggacgattttgattttcttgaggttgcgaaccacttcttcacaaatcgcgaaggcgcggcgttgcgcgcgctgccatcggcgctgcagcgccgaggattttacaagtgttggaccagcaaagaagcgtttctaaaagccaagggcaccggtttgtcgggagaattagacgaggtggaaattacccttggcggcgagcatgtgcagatcacagcgaatgtaaccggttggtcgctggcggaattggatctcgccggcggctatgaagccgcgttggtcaccgaatgtagaccgatggaaattttctcctttcgctggagatcagcgaggtagtcaagtttctttgaaccttaatcccggcgggatcgtcagcaaaattgacctccgactccgaagcctggtagtaagtgaaaatggcccaggatttgattcatcgaggtttgctggcttcgatatagctcgctgacttgctgtcgacgcgctccacaaattcggggcagctctcctttttccaagcttttagcccgcaagccgttcccgttacgggctaaaggtttgccttgaccgctcggtggtaagcccctaaaattcgagcctcatgttttccgatctggcaaaatcgaggcgacttctgctcgccgccgttgttcttcctctcatcgcggcggtttttttcggattgaaaacgctgcgttcgccgggccaaccctttggcggcagcggcaaacccggcgtttccgggactaccgatgtcatcggagagatgaaagaagcgcttgccaaggagcgcgatcagctcgccgctctggagcgggaacagtcgcgctggaggctcgaagtcatcaaccggcgcaagctctatcaagatgggcaggtttccaaggaccaagttcacgaggccgagcaagcgttcattgcggcgctcaagcgcatgcatgcgacgcgcgattccgtgctggagatcgatatcgcgatcaccgaagctgtattgggtgaaaaggtcaatcgcctgcccgccctgccggttaacggttacagcgagatcggccggccacgcgatcgctgacgtcggtaccctcgagtctggtaaagaaaccgctgctgcgaaatttgaacgccagcacatggactcgtctactagcgcagcttaattaccg

SEQ ID NO: 52 - DNA, 1494 bp, Artificial
  Environmental DNA clone
nncgannctccttatagggggattgtgagcggataacaattccccatcttagtatattagttaagtataagaaggagatatacatatggcagatctcaattggatgcgccgacaacagcggatggcgcttgctggcggcaatgccaatcgccttgagcttgcccgatcggacgttgcctctccctccccgctgcccgaccctcaaatcgctcaagccgatgaaggctctgcggacacgttcgtgactcgcggcctagtccggccagactaaaccggaatcatttttgccgccacggcaggaccagtggcgatgctagtttgcgatagctgcagaaagccttgcaagcactgacgccacgagaaggacgattccggatcaatggactaccaagcgagcgcctgctcctccccctcgttcgttccccgcgccgaaatcgtcgtggcccggttggacacctaccgcgacgcggcgtgcaccgaatcagccctgctttcaggagcggagcgccatcgagcttcccggttcgtgcgggagcgcgatcgcgagcgcttcatcctcgggcgggccacgttgcggcggctgctgggggcccggctgcggatacctgcaagctcggtcgaggtggtcgtcggagcgcatggcaagccagaccttggacccgaactgcgcaggtccggcctgcggttcaatgtctcgcattgcgacgatgtcgctgcgttcggtttcgcgtggggccgggagatcggcgtcgatgtggaagcggtgcgcgcgattcccgacgctgattcgatcgcacagcagttcttctcgccgcgcgaagtcgacgcgtactttgcgcttccggccgggcaccggccgctgggcttcttcaattgctggacgcgcaaggaagcgttcgtcaaagccgtcggcgacgggcttggcttcgggctgcaccgattcgacgtgtccctcgatcctggcgctcccgcgcgcctgcttcgcgtcgacggcgacctgggcgagcaatccggctgggtgctccgcgatttcgatcccgcccccggtttcgtcggtgcagtcgtaacgcagtctgcgatggtgcactaggaaagactcgcgtgaaccactttgcccccctctctcccgccgtcgtccgtcgctcggatcggccaggagaggccttcgccgaattccgtatcggcatcggcgcgatgccgtggctcgccgatcatcgcgtacagggcctcacgatcctgccgggctcgctctatgtagaactggcgcggttggtcgagcacgagctgtgtggccgcgcaccgcagattgcgcgcaacctcagctttgatcggccgatcatcctcccgcggcaggagatcacggtgcacgccacagtgcgatcggccggccacgcgatcgctgacgtcggtaccctcgagtctggtaaagaaaccgctgctgcgaaatttgaacgccagcacatggactcgtctactagcgcagcttattattct

SEQ ID NO: 53 - DNA, 2560 bp, Artificial
  Environmental DNA clone
nnngnannnnnntagggggattgtgagcggataacaattccccatcttagtatattagttaagtataagaaggagatatacatatggcagatctcaattggattccccggtgttctccgacttctacctcttcgacggcctgctcgggctgggcggcgacgatctcgacgcgcgcgcccagcaacagctcgcgcgcctcggcctcgatcggaaagtccgggtcgagcgcggccggttctcgacgacggcgctctcgcagggccagcgcaagcgcctggccctgctcacggccttcctcgaggaccgccccgtgtacgtgttcgacgagtgggcggccgaccaggatgtccactaccgcgagatcttctattcccagatccttcccgagctgaagaccctgggcaagacggtgctcgtcatcagtcacgacgaccggtactaccatctcagcgaccgcgtgttgaagctggagtacggcgagctcgtgagatgaacgcggggggcgtggtgcacgacgaggatgcgggcgcccgctggcagggccgccgggagatcgccgtcgcgccggccgccaccgtcaccggtctggaggcgctccgggccggcgacattcacgtctggtacgtcgatctcgccgtcaccgccagcacggtggggtggctcgccgcgtgcctgtccggggacgaacgcgagcgggcctcccgcttcaagttcgagcgcgacgctcggcgcttcatggtggcgcgcagcgcgctgcgcaggctgctgggcggatacctcggtctggcgcctcacggtgtgaccttcacatacggagcgtacggcaagcccgcgcttgacggcgatcacgcggccctgagcttcaacctgtcccactcgggcgacgtcgcggtggtcgccgcgggctgggagcgcgccgtcggcgtcgacgtggagcagtaccggccgctccccgatctcgccgctctcgccgcgcgatcgttcgcgccgcgcgagcgggccgtgctcgacgcgttgccggaggcggaacggccggcggccttcttccgctgctggacgcgaaaggaagccttcatcaaggccacggggcaggggctggcgcaaccgctcgacgccttcgtggtctcgctcggcccggacgagccggctcgcttcctcgacatcgacggcgacccgggcgccctcgcccgctggacgctgcacgacgtgcagccgccgcccggctatgccggtgccctggtcgtggaacgcgcggtgcgcgccgtgtgcgggcgcgcgtggtccaccgcggaggcgggcgcgtgacggccgtccagcgcctcgtccacctcaggccatcgccgtgaagctctggcactcatcggtcttcgtgaggagtgcgggtatgcgtaacggcgtgctgactctcctgcttgtcctggctctcgtgctcgccgccgcccttcctcccgcgatcgctggacccgcggggggattcggcgtcctgggcgacagcagctccgatgaattccgggccgaggacaaccggggtggcaactatgccgcgacgaccctgaactgggtcgagctgctggtccgatatcgaggcctcgatttcggcgcgtgggccacgcgaagcgagccccgccggactggctacgaatacaactgggctcgatcgggcgcgcgggctgccgacctcatcgtgcaggggcaggcggtcggcctcgcccagcaggtggcggaaggccgggtctcctcggccgtgctgatgatcggcaccaacgactttgcgctcacgaacaacacgtatgccgagatctactccggggccctcagcggcgcggcgttgaccgcgaagatcgacggtatcgtcgccagcatcgcgcgctggctctggcccgatggcctgcggactgcctggattggcgccgccaccggttgtctcacccaccacgagcccgcgcttgaatccttggatgtcatacgcgagctcttcaccgcccgagaacgtcatgtacgacgtcaggacatacaccggtcgccttgtaccatacgaacgatctccgagattcgtggtgtgagtctcgctggtaccgcgatggcgtgtttgcacgcgcaggaccatatacggctctccttcactcaggtagctcatgtagtacgccaccgtctccggcgccccgccgccattgccgcgcaaatcgaggatgagagcatccgtgttcttcagaaacgcaaacgccgcgtcgatcgccttgctcgcaaacggtggcacaccgttgatctccagataaccgatgttgccggggaggatctgtgcacggcgaatggccccgttgagcttgtcgatctcaggcgccgcattgggcggcggccccggcggtggcccacccatgggcgttggcatctgcggctcaaatatcacgcgaaggtgtttgtcatcgaacgctctgcgcacgtcacggatcggccggccacgcgatcgctgacgtcggtaccctcgagtctggtaaagaaaccgctgctgcgaaatttgaacgccagcacatggactcgtctactagcgcagcttaatatg

SEQ ID NO: 54 - DNA, 1957 bp, Artificial
  Environmental DNA clone
nnnantgnnctaanaaanggggggattgtgagcggatacaattccccatcttagtatattagttaagtataagaaggagatatacatatggcagatctcaattggatcgccgccctgtcagcggatctgaccagcctgttcccgccaggtgcgattgcggctgcattgcgcggggccggcgataccgaatcgctttatgccgcggaggcgcggcacctgggacgcgccgttcaaaagcgcgctcaggagttcgccgccgggaggttgtgcgcccgcagggtgctgcatgaatttggcatcgagcattttcccatcgaagtgggagcgcatcggcaacctgtctggccggacactctggtgggaagcatcacgcataccgcgggtttttgtgccgccgtgatcgccccgagggcccgcttgcgcgccatcggcatcgattgcgagcttgcggccagcgtgcggcacgagctttggcgcggcatctgcacacccgcggaaactgcgtggctggtttcattgccggatcaagaacagccgcgcgccgcgaccctgatcttctccgcgaaagaagcgttctacaaatgccaattcagcgcaaccgaggaacacttgggattcatggatgccatcgtcgaggtgcccgagtgggaaggagcgcgggggcttgggcggttccgcatccgggctacgcgccgcatcgagctggaccgcttcacggccatgccgctcgagggccgctattcattccacgaggacttcgtcacggccggactggccatgccctgaagttccatcaataagccttgtcgtcgcgaaacaccttgacgaccgtcaacagaatgatcttgatatcgagcatcggcgaccaatggcgcaggtagtcgagatcgtagttgacgcgcgcttccatctcctcgagcttggacgtttctccgcggcagccattgacctgcgcgaggccggtgattcccggcaggaccttgtggcgcaccatgtagcccttgatgagcttgcggtattcctcgttatgcgcaaccgcatgcgggcgcgggcccacgaggctcatccgcccctgcagcacgttgatcagctgcggcaactcatccattgatgagcggcgcaatacggagccgaccggcgtgatgcgcgtgtcgctcttcgacgcttggcggatctgcgcgccgtcttccgtgacacgcatggtgcggaatttgtagacggcgatttcgcgcccgtccaggccgtagcggcgctgcttgaaaatgaccggaccgggagaagacagcttgaccacgatcgcaatgagcacgagcaagggcaacagcaacagcagaacgagggtgctgaagccgatgtcggtgagccgtttggccacgccccgataaccgtaaaaaggcgtttcgcacatggcgacgaccggaatgccgtgaatttcgcccgagcgcgcctggatcaggtcgaacacgaaaatgtccggcacgtagtagatcgacgccgtcgtgtcgcgcaggtcgtccaggagattcatgacgcgcttgacgtgccggatcggcagcgcaatgaagatgacatcggtgcggttatccttgacgtactgcgacatgtcgctcaaggagccgatgagctgggcatcggtttccattccgagccgatcgctgctgcgatcatcgaagaatccggagatctccacgcgcatgccgggattgttcttcaggcgccgggcaagctccaggctgctctgattgtagccggcgatgatggcgctgcgattgtcgaaggcgttcatcaaaaaacgccgcatgacctcctgcatcgccaaggtcacgcagatcagcgatcggccggccacgcgatcgctgacgtcggtaccctcgagtatggtaaagaaaccgctgctgcgaaatttgaacgccagcacatgactcgtctaccagcgcagcccaaaaaaacacctcat

SEQ ID NO: 55 - DNA, 2478 bp, Artificial
  Environmental DNA clone
cnntcgnnnntcctttttgggggantgtgagcggataacaattccccatcttagtatattagttaagtataagaaggagatatacatatggcagatctcaattggatccgccgctcttgatcggcggtctcgcgatggcgtggggttggctcggcgcggcgctgaaacatgaaccgcgctacggcgacggcgagttccgcaggttcctgcgttcgtaccaagtcgagtgcttgttcctcgggaaacggcgtgcgaccgcgcgtctcgacgcgcgtcaagccgcggtgtggcgcgcacccgacatcgtggaagcggacacggagccggcgcgcgcgcgctgaaccggagtggacgaactcacgacacgcacagggaagtccgggtcgctcggggcaggcgctgctccgaaggcgtcgacgctcgcgagcatcgatctcctcggcgtgcgcatccatcgcgtgaccgaagccgagagcgtggcgcacatccgcgcggagctcgacgcgcaccgcggtggatgggtggtcacgccgaacctcgatcatctgcgccgcctcgtgcgcgaccgcgagttcgccgacttgtgcggcggcgcggacgtcgtcgtcgcggacgggatgccgctcgtgtgggccgcgcgcgtgcaggggacgccgttgccggagcgcgtcgccggctcgaacttgatctggagcgtgtcgcgagccgccgcgcacgacgcgcgcaccgtctacttcctcggcggcgatcccgggaccgcggaacgcgccgcggacgtccttcgcgcgcgatttccgggtttcgtctgcgcgggcacgcattgtccgccgttcgggttcgagaaggacccgcggcagcgcgaggagatcgaaaatcgtctgcgggccgcgcgaccggacctcgtgtacgtcgcgctcggatcgccgaagcaggagcgtttgatcggggagctgcgcacgaagctcgcggaggatctcgcggcgacgtggtggctcggcatcggcatcagcttcagtttcgtcacgggtgacgtgcgccgcgcgccgagatggatgcaacgtctcggtctcgaatgggtccaccgcctcggacaggaaccacggcgtctcgcgcggcgctacctcgtcgacgatctgccgttcgccttcgcgttgttcacgcgtgcggcgcggcgccgagtggggggacgcgaagaatgagcgcggcactcgacatcgtctggcccgtcgcggacgcctcgtggaagctcacagccggctcgatcgcggtcttcgcggtcgatctcgacgccgtcgtcacgacgaaggacgtgtggagcgccgccgagcacgagcgcgccgggcgtctgcaccgcgaggtcgatcgcacgcgttttctggcggcgcgcgcgttgttgcgcggcgtattgtcgcgcgcgaccggccgcgatgcgcgcgagctcgacatcgtgccggggcggcgcaagcgtccgcgcctcgcgagcaacgacgaccccgacttcgatttcaacttggcgcacagcggtcccctcgcgatgtgcgccgtcagccgcgcggggcccgtcggcgtcgacgtcgaacaggaagtcgtgcccgacgacctcgacgcgctcgcggcacccgtgctgacgcccggcgagagcgagcgtttcgcgcgatgctccccggaagatcgtgcaggtttcttcttcgcggcgtggacgcggaaagaggccctgctcaaggcgctcggaaccggtttcgcgctcgcacccacgctggtggacgtcacgcacgacgacgtgcggctcgacgagcagctcgcgtcggcggagaagatcgctggggcgctgcgcgcggacctctcgcgagcttccacggaggccgatgtgcgctcgatcgagagctcgacgccgcgcgagtggaggatcgataccctacagcccgcgcgcggcttctcggcggccctggcgcgagctcgaatcgaagggtcgatcgagtgctggcgctgggcctgaacggccgataaagagcgtcgctcgtcgttcctgtcgtcgttgtcggtggcggagtgcacgagtaggatcccgccccacgttttccccgtcgcgcggcccgcgcggcccgaaccatgagccgaccgcaaacgcccgacgcgctgcccctgaccccgctgcaattggggatgctgttcgagcacctgagcgacgccggtcccggggtcgacatcgagcagatcgtgggcacgctgcgcgaggtcgtcgacgtcgccgcactcgaatcggcgctcatgcgcgtcctcgcgcggcacgaagcgttgcgcacgacgttccgttgggaaggtctggggacgccgcaacaggacgtccacgccgacgcgacatcggccggccacgcgatcgctgacgtcggtaccctcgagtctggtaaagaaaccgctgctgcgaaatttgaacgccagcacatggactcgtctactagcgcagcctaataccagccc

SEQ ID NO: 56 - DNA, 1581 bp, Artificial
  Environmental DNA clone
nntnnactnnntaggggattgtgagcggataacaattccccatcttagtatattagttaagtataagaaggagatatacatatggcagatctcaattggatggcccgcaggtgatgaggccgccacgtgtcgtcgctgtcgatgaaggcgatgaacgcggcgcggcccgcgtcacggccttgattccgcgcgccggcgcaaccctgcttgtgcgtgttctcgacgtatcgaatccgcggatcgcgatgcacgtactcgcgaaccacgtctcgcgtgccatcagtgctccggtcgtcgacgacgatcagctcccattcggagtaggtttgagccaacaccgagtcgatcgatcggggcaggagatgtgcgcgattgtaggtaggaagcacgatgctgacgaggggcgcggttgacacggtggtgttctggcgctactgattctacctttgcgtgcctgcatggctgctctgcttgacgtccacatcgtctaccgggtgacccgctatctggcggaccagcagctgtcggctgaacgggcgtgcctgtcggccgacgaagaggcgcgcgcggcgcagttcgcctttgccgacgaccggcgtgacttcattgccgcgcacgcgctgctccggcagacgctgtccgcacatgcgccgcgcgatccgtccgcatggacgttcagtcagggcccatacggcaaaccagtgctggcggacgggtccgatgaacgcactgcgctgacattcaacctgtctcacacccgcgggctggtggcatgcgtcgtcagtcggaacgtcgatgtcggcatcgacgtcgagcgtaccagccagagcgcggatgcgctggatgtcgcgcgcgaccggttctcgtcacgggaggtgaggtctctcgaggaatgcgaccacgccgcgcgctccgcgcgtttcactgagctgtggacgctcaaggaggcatacgccaaagccaaggggaccggtgtccttggcggcatggacgtatgcacgttcacgcttggaccgacagactcgctctgctgcgacgtggtggacaggggcggacccgatgcctggttgttcgccgtcttcggggttcttgactatcgtctcgccgtggctgtccatgaccccgagaaacgatgtggcgccctgtcggttcgtcacgctcgtgagcgcaatggcggtgatgaacgccagatgcggctacttcgaaggtcgaaaagtgtggtcgtgactgattgagcaggtcagtctacttcctcaacgctgcgtcccgcagcaacgcgcgaatctcggcggcagtcgcagggtcgacggggccgcctgcgccgccgcgacctgcggcaccgcgccctgcagtactagccccggcggctcggcccgcggcagccccgctatcgagcagatcgatcggcttcctgccgttagcatcaatgagttctggcgtggcgcctctctccagaagataccgaaccaatcggccggccacgcgatcgctgacgtcggtaccctcgagtctggtaaagaaaccgctgctgcgaaatttgaacgccagcacatggactcgtctactagcgcagctaatact

SEQ ID NO: 57 - DNA, 1667 bp, Artificial
  Environmental DNA clone
tgggtgagttcggaaaccgaaaccccaaccgccagtttacttctaggcgctcagccagagcggcaagtgacgagaaaaaatgtcccagaaaatggaagcgcaagaagccgatcgctcagacgaagggatgtgatgtgcttttctcatgtaaccccccgagtggagtgcgaagaaccgagatgcctgtcgtatggcgtcatcgaacagccagtctggcgcttagcatggacgaaattcacgtctggcgcgctgttctgaacgtgaaggcagcagacctggacaggttgcaagctaccctctcccacgatgaacgcctgcgcgttgcacgctttcgctttctgagggatcgacgatgtttcatcgcggcgcggggagttcttcggcatattctggcggtatacgtagggtgtgagccagctgctttgcggttttgctactccgggactggaaaaccagctttggcaaccgatgtcggagcgcccgatctgcgcttcaatctatcccactcacatgggctggctatctatgtcgttgcgcgtggccgggaagttggcgtcgacttggagagcattcagccccgtttcgtcgaggatcagattgcggaacgattcttctcagctcgagaagtctccgctctgcgtgctcttccgataagcactcaacccgaagcgtttttcaattgctggacacgaaaggaagcgtttgttaaggctaagggtctcggtctcacaatccctctaaatagctttgaggtctcgctgaccccaggccaaccggccgagctgctcagcttccacgctgatccgtcggaggcttctagatggaagcttgcagcacttgtaccgagcccagggtatattgcggccattgcggcggagggaaaggagtgggaaacgagactgttggaatggacggcgccatggacctaagagaatttgtatttccgacgatctttccgcctcgggttgtttggatcgcttgtacatggagcctctcctgagacgaaatctgaggtcaatcagtccagtcacccgaccgaccggcggcacccaattcctcgtcttcgttgtcctgccgggctaattccgaaaaagattctcctcgtacctatcctggggcacatcgtgcgcgaggttcgccattttgatccgggcgattggccgtaccggccgcgcgcattatcaagtgatggtccgcagctgaagattccgctgaatgatcgtcgattccgaagggaaggcagtcagtgttccggacggactccgagctataggagcgaagtgacgctgacgttctaaacggcagccaagcgattcgatttgtcaagagaagcgatccagagcgaaacgaaggtacaggtttgcccggcggaatggtgggaagggcagaggggcggcaggcccccttgccctgtcgattacggcagaatcccggagtgcgtcagagctccggtttggccgcttgagcttacatatggatttgtcgcccatctcagcgaatctcccatccagtagccttgcggcaaactggccaataaagtcggcgtgagcaaggatcttggtgttctacgcggaacagggtcagtgtgtctatgcagtagtctgtggattccatggggaatccagagattacctcgcaaccgtcgcccggtctgaagtattccctgc

SEQ ID NO: 58 - DNA, 1901 bp, Artificial
  Environmental DNA clone
aagccgacctcctatagggggattgtgagcggataacaattccccatcttagtatattagttaagtataagaaggagatatacatatggcagatctcaattggatgatcgggtcctcgatcgacaacacgtgatcggcgcgctcttcgttgatcaagttgatcagcgcggccatggtcgaggtcttgccgctgcccgcagggccggtgatcagcacgatgccgttgtggtgattgacgacgcgcgcggcgacgctgggcagcccgagctcggacaacaagcgcggctccgcggggatgaagtggaacacgccgcacggtccgcccagctggcgatagatgttggtgcggaagcggccgacgttcgcgaccgcataggtgaagtcggtctgcccgcgcgcctggaactcgtcgcgcgccacttgcggcatcgcgctcatgaacacggcctcggcgtcgcgcgccgagatcacgtccggcgcaagatcggtcaaggtgccgaagatgcgcgccttgatcggcatggtcgagtgcacgtgcacatcgctggcgtgcatgcctacggcgacgttgagaacgcggtcgagccattgcagccgctgcggcgcttcacgcggctgcgccggtagcttggcggtggcgggtggcggggccggcacggcaggtatcggcgcagcaggcggggcagggcgcgcggcttgctgcgcttgcttgagcaccttcggtgcgtgctcggcttgctgcgcgaggtattggcgctgggcttcgaccagctgcccgagctgcgtctcactgaggtagccgagctcgaccaggatcgcgccgaggcgcttcgcgggcgcgcggccctgcacttggaccgcgttcgccagctgctgcatgttgatcagcttgtagtgaacggccaatcgaccgagcagatccggcggtggctgagccatgaagccgatcgattgtcacacgcgttcgcgccgcttgaccattacgttgttgggcacccgtgtagtgaagccgaggtgcccgtccgaccccgccgtcaatgctaggctgcgcgcgtgatcacctcgctcgagccggacagcgtgcgggttcactacgcgtggaccgcgccgctggccgatccggagcgaatggcccggggcgtggctttgctgtcggacgccgagcgcgcgcgggtcgagcggtttcacttcgagcgcgaccgttacagctacgtgctggcgcacgcgctgctgcgcgctcagctcgcacagctgacgggcgtcgagccacgcgagctccgcttcgagcaagggctgcacgggcgcccggagctgtgttggccgctgccagagcgcgagcagccgcgcattcgcttcaacatctcgcacacccacgggctggtggcctgcgcgctgtgcctcgagcatgacgtgggcgtggacgtggagcatgccgagcgcaggctcgagatcgatcggctggcgcccaacgtgttctccgcgcacgagcgccaagcgttgtcggctttggccgatggcgggcgacgtactcgcttctttcagctgtggacgctcaaggaggcgtacatcaaggcccgcggcatgggcctggcgttgccgttgcgagagataacctttcacatcgacgggcgcgatcgtcccgggctcgagcttgggccagcgattgtcgatgatgcgacgcgctggtggctcgatgttcggccgctcggccagggtcacatgctcgcgctcgcgctcgccaccacgccgcgcagcgtgcatatcgccgagctcgaccccactgcggatcggccggccacgcgatcgctgacgtcggtaccctcgagtctggtaaagaaaccgctgctgcgaaatttgaacgccagcacatggactcgtctactagcgcagctaatagagtc

SEQ ID NO: 59 - DNA, 3158 bp, Artificial
  Environmental DNA clone
nnnngncntnctataggggggattgtgagcggataacaattccccatcttagtatattagttaagtataagaaggagatatacatatggcagatctcaattggatgaaaaagcgtgtttaggtcaagagtttaatccataggattaatctggacatgagtttgtatttttacaaaacttatatcaatagcgcccggtgggctgccgatcgtaattggttaacagccggcattattatcgtcgcaatcgggaacgccgtggctgcgttttgggcagaagatcatcataaatttcaacgaggtttgcttgattaaatatgccgagaatgacctgtgagcaggcataaccaatctgacgtttacgtctggtttcgcggcactgcagctcttgatggtgaggctgtgaggtcggccgaccaacatctatcggctgaagaacgggcccggcgcgatcgacttcacttcgacgccgaccgtcgcgatttcacaatcgcccacgatcttttgcgacgggcactttccatgtacgaagacgtgccccctaccgattggcgatttgctaccgacaactacggcaagccatcgatcgacagtaacaacccgcaactgcgggcattatcattcagcctttcacacactcgggggtgtgtcgcttgtgcgattacctctagcgcgccgcttggcgtggatgttcaacgaaccgatcaactacagtgcgtagaagaaattgcagatcgatacttctccgaaaaagaggcggcgtggttgcgccagtgttcaggtgaactgcgtaatatccgcttcgctgaattgtggacattgaaggaggcgtttttgaaggccgttggcgtcggcctttctggatcgctcgccaacatttcatttcggttcgaggagcacgcgcgcattgaggtttcgggaccgtcggctttcgaccctctcgagtggtatttcgctttattcgaacctgtttacgatgtgcggcttggtattgcgatccgcaacatggtgcgacctcgcttttttttgcgccaggatgaaggtcacggaccgattctccgcccctttcgtgtttcggcgtcggcggatcacaacacctgatcgaatttcaaagaagccgccgtcattctgattcttggcgctggagctgcgggtaggcctgcttagacaacctgatttagaaattagcgaaataggaaagcagacggcgtagactaccagacgctaagtcgttcgtcgccgccattcgctcgccaagagcactacacagatgccgcccatttctaaaacccaattccagcgaaagccgagaattttctggccaatgaagcgatcaatgtgatggaacgatgctgcgcgaatcagcacaaagccgataactaaagccgttcccaatattgcaaaccatgccgcgggcccagccctacgcgcccccactacgaaagccacgaccgcgaccgcgcagactgttgccacaaagacgatgaaatcgatttgcactaattcacgttgctcgtaccacccttgctggaatgctaccgtgcgaccgagttcggtaagtgctgattgaagatcgagctgcttgttgattcccagccccaagaacaagattgaaatggcaacccaaattttccaaccttgtgctcctttatgtcgggccgtcatccagcagctaattgcggccagaaaatacaaaacaactgttatccagccgccaattgttggatcacctatgccgggtgtccaatcaaaatgatgcaaccgcctgccctcgattttgtcggtgatcggccaaaaccacgccgtcaattgtccgagttcatcatagattgtcacaacccagcaaagcacggcccctgccctcgccctagccgccagtcccgcttgcctagtgtgtaatcgatgagagcgcgaaagctatgtggaacttgaagcgtcgcgactaagttgtattggctcgccgtcaggattctctggatggtccggcaccaggccaattgtctctttgactctgattctgcgtttcgtttcggtatgttcatttgatggtggtatccgcgctcccgcgcctgcccactccggtctcgcatgagctacaagaactcgtaatttttctgaggcccggtcgcgagaacgtgtccatgtttcggagtaaacgataagtcccaccagcggcagtaaccacgtcaggcgtgcctgatagcggtcatgcggtcccgacagggccccgcatatcaccgcatttgaaatctcgcagaaaagcacaacgaagaagaatagctttaactccggtgggatagccgtccaacgtctcgctaaacttgctcccgtaaatatgagagcaaaaattgccgccgtaaccgtggcccacgaaaatatcgcgatcggaaactttttctcaccaaccactgtctgcgcccaccgctcggcatgagcggacggtaaattggagatcgtctcgtcgctcacggcaagaaaatccgaaagtcctaccatcgttagttgggcaaacgcatcctttgcccagccaataatctgccccatcggatcataggccaggaccgccgcggcaaaccgatattgctcgattcccaattgcatgcgcttgtctaatggcgcaggtgaataaattccggcgtcgggagacgtctcccaaagaaacgcgtccgaatttgtaaggagcctatcggtgaattggcacacggcaaagtcagcctgcgggcatttttcgcgcaggtatctcgcaccagtgccatctgcaattaccctggccataagaacgggtggtcgagtaacccgagcgcccagcgacttctcgacaaccattgcaaagccaatttcgcttgcaaatccgattcccaccgcaaggccaagagctattatcccgacgcttgaaattgtcgccgtgaaaagacgggcaataagtgcagcactcaagaggacagtgacgatagctagatgcgttggatgaaacaccacagctgataccaagagtgacacccagaacagccgctcccaatttttcattcggtgagctagcgaaatcaatcggccggccacgcgatcgctgacgtcggtaccctcgagtctggtaaagaaaccgctgctgcgaaatttgaacgccagcacatggactcgtctactagcgcagctaatattc

SEQ ID NO: 60 - DNA, 2482 bp, Artificial
  Environmental DNA clone
acntnnntaggggattgtgagcggataacaattccccatcttagtatattagttaagtataagaaggagatatacatatggcagatctcaattggattgctgcgccagggcagtaggaacagcggtcagcaagacagttgtcagtaaccagagaacgactttcgatgacactattgcctccagtcattttgccttttacatttttttttgccttatctgatcacccgatccgcccgcgccaacacattcggcgggatcgtcacgcttatctgttaggggtccaacctaccgaaataaattgaccactgtccagaggaaatttgaaccttcaagagcgacccactgcgctctctgccgcccgtgtactcccaccctcttgcgcaatcgttgtaccagaaccgacgggtgtcggccctttcatctaaagctttgaagttgtggaataaacctgccacgcggggcccggaaaccctagtcgcggtaagcatccaagtgtcgcatgtttgctacaccgtgtagtcgttttgatactcccgcttggatagaagtcggattggcgggacacttactggggccggggaattgctgctgtggagttgttatggcgggcaaatcttgggacacctgataagaaaaaacctgaagcccgccctatccatgaacctggattccgggtcaccgcccggaatgacggacaaacgtaaatccctcgctttcgctcgggatgacgggcttcagcccgtcactttgcgtccttggcgcgagaaatatccgaatccgaaactaccagcttatttcggctgcgaatgatagcagcggatcggcaccggcggaccggcaacgacaagcgcggattcgtaagtttcgatcgggtccagctcggtcaaggaccagccttgcacgctggcgtgaatttggacttgatcctgagcgccaagggttatctcaacttcatccagctttgcctgacagcccagtgcccttggctttgagaaaagcatctttgctggtccagcatttgaaaaacgcttggcgctgcaaatcaggtggcagtccgcgcagctccttaacttccgtcgccgtgaagaatcgctcggcgatttcttggaacttgaagtccggtttgacgtactcgatgtcgacgccaatttcgccgacacgattgactgcgagcaaagccatttcattggaatgggatagattgaactcgatgccacccgctgccagcgccgccggcagccggggcttgccattgctgccgagttcgaaatacaacgtcgccgggtcaatctgtaaatagccgccgagaattttatgcagcgcgatatgggcaatcagatagcgccggcgatcacgctcgaaataaaagcggacagcgcgctcgcgctcgtcaggcgagagcaattcccgccctgtaacgagatcgacgttcgcatcgtcgagccgcaccgcccagacatggacagtctgttcaagcgctggaagttcggcaggtgcggccggccagatggtcataacaagaattttaccgcggatgatgcagaaattcggatttagaaatttctcccgcaaagcatgtcctgagcaaagtcgaaggagcgcaaagacgctaagtgacgggcaaagccagtcatcccgagcccgggcgagggatctaacaaagatttctccctccagtcgaaatgacatttttctcttcccttggcgtctttgcgcctttgcgggagataatccgagttagttccctagcagcgtcgcggcgtcacggatcgttttccccgatgtagtgcgcggcagcgattcgcggatgacgatgcggtctggcaccttgaagttggccagattggcgcgcaagaaagtcagcagctctttttcgacgagcgtcgctcccggccgcggcacgatggcggcgccgatcacttggccgcgcttggcctcggggatggcgaacacgatgttctcctgaaccccgggataagtgtaaatcatgttttccacttcggcgggatagacgttcaagccgccagtaatgatcatgtcgtcacgccgcccgagatggtagtaatacccatcggcttcgaccttgccgacatcgccggtgtagagccaaccgttgcggatacgttgcgcggtggcatcggcccggcgaaaataacccttcatgacatggggaccgcgggtgatgatctcaccggcctcgcccaccggcacttcttggtcgttttcatcgacgatgcgcacttccacgcccggcacggcgcggccgatgctgcccatcttggcttcggctttgtccgggtcgaccagggtgatcatcggcgaagtctccgtgagcccgtaaccttgaatcactttgatatgcggctgagcgatcggccggccacgcgatcgctgacgtcggtaccctcgagtctggtaaagaaaccgctgctgcgaaatttgaacgccagcacatggactcgtctactagcgcagctatact

SEQ ID NO: 61 - DNA, 2832 bp, Artificial
  Environmental DNA clone
cnncntntagggggaattgtgagcggataacaattccccatcttagtatattagttaagtataagaaggagatatacatatggcagatctcaattggatcgagggccagcgcccatgcatgccgatgtagtcatcctcgcgggtaaactcgagcttgaccggctgattggttactctggccaagaccgccgccaatatatttcgagaccacgcgcactctgctcagcggcaaaccgaagtctttagcgatgtccgtgcgcgcgttggagacgccttgcgtggaggcatagacggtcagcttgccggcatcccacttggcgatgctcacgcgccgttcgagctgggcgttgttggtgtgcttggtgatataggtgtcttcgaaagttcggtcggcttctttgaagccgttttcgagatcgccccacgattcctcgatgggcgcgctgaaagcgcctttgcccactgagagattgccgttcggagcgatcttcggcgcatcgggcttcaacgcttcattgggatcgagtacgtggggaagtttttcatacttgatatcgatcagcttgagcgcgtcctcggcgatatgccggtcggtggccgccaccgcggcaaccgcgtcgccgacgaagcacaccggattattgaaaaatgaagcgctggtgcgtgtggccgccgctcgaccacggcaccttggcgttttcatgatgaatgaccgctttcacgcccggcagtttttctgccttggaagtatcgatgctgacaatttgggcgtgcggtacgttgctgcgcagcacccgcgcgtagagcatgccgggaagttggatatcgccggtgtaatgcgcttggccggtgaccctctcataggcgtcgatgcgcggcgtctttttgccgacaatggaaaattctttttgtgcttgtgccataagagcctcccttagcgcgcctttgccgccacggaggcgcgccgcggcttgtccggcaatctgtgccggccctgcacaacaatgtgtcgggagtcttttttactctggccgaagcgagcaaggaactcagaatcaaaacgctcgcgcgcttaagcggactggaaagtcgcgaaatccggcgcggcagcgcctacctgctccactggtacggcgtgccgaaggcgcactatcgcgactctgaacagttgccgccgagctgggaaaagttgaaacaatcggcccgtgaagcgaagagctggctcgatcgaacttacatcgtcgagggcgcagcgaataccgatttgagggaaattgaaaagtacgtcggcgagctgcgcgctgcaggaaataacgaaaaactcttcgtcgtcatcgacgactgccagcgcctcggactgaacgaccaatcgatgacggatcgtctgccgatcgtcgctgacaagctgcaagaatcggcggtgaatttgaaaatgcccctattcgcaatctggcctaatcttggtgaggacgctgaatcccaaccgcaggcatggactgaaaaagttgtcagcccggatgtgattctcgtcctggaaaaagatcctgaacgaaacaaaacactcaccgaacccaatcaggcgatcattttgcacgtcgtaaaaaaccgccgcggtgaaaaaggcaggcttgctttcgatttttttccggcgttttcaagattcactgaagcgccatagccgctaaggttcccagcgatagagttggattgcagcgggctcgccttcgtacactaaagctccttcatagtcagcgacgacgttcaattctatcaacgaccagcccggcgcgttggcctcgatccgcgactgctcgcggctggcggcgaggcctatgtttatctcatccaagtcgcctgataggccggtgcccttcgctttaaggaacgcttccttgctcgtccaacatttataaaacgctcgccggcgcaggtgcgccggcagggcacgcagcgcagccacctcgccggcagtaaaaaatcgctcggcgacttcatcaaaggcaaaatctgcattgacaaactcgacgtcgataccgacttcacgagaacgagccacggcgagtaaggccgcgccgtgagaatgagaaagattgaacgctactcggcttcccgcctggggcgcgcctagttttggtttgccgtggccgccgagatcgaactgcaagtttccggggagagtctctgtatatcgcgccaagatcgagcgcaaagcagcgtgtgcgaccgtgaatagcaaacgatctttggcaaacttgaacctatcggcgcgagcctgttctgcagccgatagcagagcggcgtgtgcggctaagttgtcggcgtcggtttccgccgcctcaatcagccaaacatggatttcgtttgcatccaaaaggagaccggcaggtggcgaacgccaaacggatttgaaagattggttcggcacggttcttcgcattactttgcgccgaaccagctaaagatcaagttcccgtcgcagtctctgcccgaccgcaagcgagccgggatccttggcttccaccaacgccccatcgatatcgagaaccagccgttcatcggcgagctcttcaatgacgacagcgtcgtggcgcgtggcgggtcaattctcacagttatgctatcggcagcagagcgccgaacagctacccggaatccaggatttgtggagctatgggcaatcaggtgaaatcttggtgtggcgcgatccctgtcacgatcacatcggccggccacgcgatcgctgacgtcggtaccctcgagtctggtaaagaaaccgctgctgcgaaatttgaacgccagcacatggactcgtctactagcgcagctaatact

SEQ ID NO: 62 - DNA, 1890 bp, Artificial
  Environmental DNA clone
nnnnnnannntccctatnggggggattgtgagcggatacaattccccatcttagtatattagttaagtataagaaggagatatacatatggcagatctcaattggatcccgcacgatgcgcgcgaccgacaggacgaacatcaccgccacaagccggtcgtcgccgtagaacgccgctacccaccaggcgatggcgatgccgaccgcgcccgtcagcgtgccgatcgccacgttgaacgaccacaccgcgtcgaagtgttcgcgtcgggcatccgcgcgctgaatcaatgccgtctcgaggccgaacgcgccgagggcgtccagcagggcgatggctgacatcgcgagcgcaatcaggccgaaatccgcgggcacgaggaggcgggccagcacgatcacgctgacgaatccgaaggcccgatcgacgaccttgtacagcagcatccaggcgatgccgtgagccatcctgccgccgacggtgcgcccacgggtcatgcgagtcgccagcgatcggcgcagcggcggcgcgatcgatcgaaaccgatggtacgctttgctccagccttaacaatcgcatgcggacacggccggcacggcctgcgttcacgccctcgatcaggaacatggaaacggcatccccacgacgcggtgcgctggcccggccaccgcttgtctttgcacacatcgatgcgtcggcctgcgaccgtccaccggaaatcgtcgccatcggcccgcgagttgcgcacgtctgggccttcctgctcgacgcatccacggaatccggcaatacgtggcggcacgttctgtccagcgaggaaaaaacgcgcgccgatcgcttcgcgcacaccgtcaaccgcgaccgatggaccgttgcacgcggcgtgctccgccacctcctctcgcggtactgcggcgtaccgccgcacgcgattgcgttcgagcagggtgccaccggaaaaccctccatcgccggaggcagcgcggatcgaagcgtcgccttcaacctcgcgcactcgcatggccgcgcgttgctcgcggtggcccgtgatgcgcccgtcggcatcgacctcgaacaggagcgacgcgacttcgacccgatgccgctggcgaggcagttcttcttcgagcgcgaactcgaggcgatcgcctcggtcctggctgatcgtcagcgagaggcgttttttcgccattgggtcgccaaggagtcggtgctgaaggcacagggcgtcggcctgtcgtttccgctcgaccacttccacgtgtcctttgacgccgaccacacgattgcccgcgtccaatcgcgggatccccaaacgctgagtccggaatggatcgtccgcgttttgtcgcccgggacgggctggcacgccgcggtcaccgcgcgcggcgaggactgggacgtgcgcgtcatggcctgaccgcaccgtcacggcgcgtcatgcccgccgcggcaaggacgatcagcggcgccacggcaaacatgtgctgccggtcgccgctctcgaacacgccgaataccgctgcaccgcaaagcaacggatagacgagcggtcgatagtcgtaccgatcgcgctcgtcctggcgccgccagagaaggagcgaccgcacgcacagcgcggcaagcacacccaatacgccaacgatacgccgctcgcgatccggtaggccncatcgcgattcgctgcactttgcagtgcgtcatcnnntgccgccnnntgacgaagcacgctcanagcaccgtnncgtcatccgcnnnacgcgatcgctgacgtcgtaccctcgagtctgnnaagaancgntgctgcnnnnngnnnccagcnnatgnntcnnnactnnnnnnctnnnnacnntaannntnnnnnacnnatggacnnactngnnnnnaccnnn

SEQ ID NO: 63 - DNA, 950 bp, Artificial
  Environmental DNA clone
gncnnntntataggggattgtgagcggataacaattccccatcttagtatattagttaagtataagaaggagatatacatatggcagatctcaattggataactggtagggctagggattgtggaccggctggccggcgcgggattcgggctgctgcgcggattgttcgcggcggccgcggtgaccacggtccgcggctcggtcccttccggggtctcttcgttcgacacgctagaccttctacgaggttactcgtccttgccttccccggaccagaccgaggagtagccctcattttgctcgtaatattcactagttttctgctgatcgacgatcgccgcgtccccaccgagcgcctcggtcaggaacttggtctcgctcaggcagctgcctcccttgacgcccttcacctcgaaggtgacctcgcccttgggattgatgacgatctggatgtcctgtttgcggctcatgtgtcgacctttctactcggctgaccacttcgaaacgacgagacgaatcgacccatcctcctcgttcacctcttcctcgaccgagtaccccttctcctcacaagcctgtttgacgttgtgatactggtacttctgcgagacggcatccttgaactcctcttcggacgttccctgcgtcgtctccacgccccaccagtcagcagcgacctcgtaggtcccgtcctggttctgaacgatgccgatgtcgtagcggcccatgttcactttgccggccgccttgatggtgtcaccgcggtagccgcgcacggttacctgctggttctcctcggcccactcgacggtgtatttgaggtcctccagcgcctttttgatggcctgcaatcggccggccacgcgatcgctgacgtcggtaccctcgagtctggtaaagaaaccgctgctgcgaaatttgaacgccagcacatggactcgtctactagc